(19) United States
(12) Patent Application Publication
(10) Pub. No.: US 2025/0360089 A1
(43) Pub. Date: Nov. 27, 2025

(54) NANOPARTICLE FORMULATIONS FOR DELIVERY OF NUCLEIC ACID COMPLEXES

(71) Applicant: Translate Bio MA, Inc., Lexington, MA (US)

(72) Inventors: Fatih Ozsolak, Boston, MA (US); Balkrishen Bhat, Carlsbad, CA (US)

(73) Assignee: Translate Bio MA, Inc., Lexington, MA (US)

(21) Appl. No.: 19/192,470

(22) Filed: Apr. 29, 2025

Related U.S. Application Data
(63) Continuation of application No. 15/771,321, filed on Apr. 26, 2018, now Pat. No. 12,318,489.
(60) Provisional application No. 62/246,290, filed on Oct. 26, 2015.
(60) Provisional application No. 62/246,305, filed on Oct. 26, 2015.
(60) Provisional application No. 62/288,128, filed on Jan. 28, 2016.
(60) Provisional application No. 62/288,154, filed on Jan. 28, 2016.

(51) Int. Cl. A61K 9/51; A61K 9/00; A61K 9/127; A61K 9/16; A61K 9/19; A61K 47/10; A61K 47/14; A61L 24/04; B82Y 5/00; C07K 14/705; C07K 14/775; C12N 15/11

(52) U.S. Cl. CPC A61K 9/5153; A61K 9/0014; A61K 9/0019; A61K 9/0053; A61K 9/0073; A61K 9/127; A61K 9/1647; A61K 9/19; A61K 9/5123; A61K 47/10; A61K 47/14; A61L 24/046; B82Y 5/00; C07K 14/705; C07K 14/775; C12N 15/111; C12N 2310/11; C12N 2310/315; C12N 2310/3231; C12N 2310/346; C12N 2320/00

(57) ABSTRACT

Aspects of the disclosure relate to particle formulations, e.g., nanoparticle formulations, methods of making particle formulations, and methods for delivery of oligonucleotides and/or synthetic RNA, e.g., for increasing gene expression in a targeted manner. In some embodiments, compositions and methods are provided that are useful for posttranscriptionally altering protein and/or RNA levels in a targeted manner. Aspects of the disclosure described herein provide compositions and methods that are useful for protecting RNAs from degradation (e.g., exonuclease mediated degradation).

### CROSS-REFERENCE TO RELATED APPLICATIONS

This application is a Continuation of U.S. application Ser. No. 15/771,321, filed Apr. 26, 2018, entitled “NANOPARTICLE FORMULATIONS FOR DELIVERY OF NUCLEIC ACID COMPLEXES”, which is a national stage filing under 35 U.S.C. 371 of International Patent Application Serial No. PCT/US2016/058842, filed Oct. 26, 2016, entitled “NANOPARTICLE FORMULATIONS FOR DELIVERY OF NUCLEIC ACID COMPLEXES”, which claims the benefit under 35 U.S.C. § 119(e) of U.S. Provisional Application No. 62/246,305, filed on Oct. 26, 2015, U.S. Provisional Application No. 62/246,290, filed on Oct. 26, 2015, U.S. Provisional Application No. 62/288,154, filed on Jan. 28, 2016, and U.S. Provisional Application No. 62/288,128, filed on Jan. 28, 2016, the contents of each of which are incorporated herein by reference in its entirety.

### REFERENCE TO AN ELECTRONIC SEQUENCE LISTING

The contents of the electronic sequence listing (R069370037US03-SEQ-EMB.xml; Size: 1,052,817 bytes; and Date of Creation: Apr. 21, 2025) are herein incorporated by reference in its entirety.

### FIELD OF THE DISCLOSURE

The disclosure relates to particle formulations, e.g., nanoparticle formulations, as well as methods of using such formulations for delivering oligonucleotides and/or synthetic RNA.

### BACKGROUND OF THE DISCLOSURE

A considerable portion of human diseases can be treated by selectively altering protein and/or RNA levels of disease-associated transcription units (noncoding RNAs, protein-coding RNAs or other regulatory coding or noncoding genomic regions). Methods for inhibiting the expression of genes are known in the art and include, for example, antisense, RNAi and miRNA mediated approaches. Such methods may involve blocking translation of mRNAs or causing degradation of target RNAs. However, limited approaches are available for increasing the expression of genes. Furthermore, there is a need for effective formulation and delivery of agents useful for increasing the expression of genes in a subject.

### SUMMARY OF THE DISCLOSURE

Aspects of the disclosure described herein relate to formulations and methods for delivery of a stabilizing oligonucleotide that is useful for modulating nucleic acids. In some embodiments, formulations and methods provided herein are useful for protecting RNAs (e.g., RNA transcripts) from degradation (e.g., exonuclease mediated degradation). In some embodiments, the protected RNAs are present outside of cells. In some embodiments, the protected RNAs are present in cells. In some embodiments, formulations and methods are provided that are useful for posttranscriptionally altering protein and/or RNA levels in a targeted manner. In some embodiments, methods disclosed herein involve reducing or preventing degradation or processing of targeted RNAs thereby elevating steady state levels of the targeted RNAs, e.g., in cells. In some embodiments, methods disclosed herein may also or alternatively involve increasing translation or increasing transcription of targeted RNAs, thereby elevating levels of RNA and/or protein levels in a targeted manner. In some embodiments, formulations and methods provided herein are useful for delivering a stabilizing oligonucleotide to a cell or tissue of interest.

Aspects of the disclosure relate to a recognition that certain RNA degradation is mediated by exonucleases. In some embodiments, exonucleases may destroy RNA from its 3′ end and/or 5′ end. As used herein, the term “stabilizing oligonucleotide” refers to an oligonucleotide (oligo) that hybridizes with RNA at or near one or both ends. Without wishing to be bound by theory, in some embodiments, it is believed that one or both ends of RNA can be protected from exonuclease enzyme activity by contacting the RNA with such stabilizing oligonucleotides, thereby increasing stability and/or levels of the RNA. The ability to increase stability and/or levels of a RNA by targeting the RNA at or near one or both ends, as disclosed herein, is surprising in part because of the presence of endonucleases (e.g., in cells) capable of destroying the RNA through internal cleavage. Moreover, in some embodiments, it is surprising that a 5′ targeting oligonucleotide is effective alone (e.g., not in combination with a 3′ targeting oligonucleotide or in the context of a pseudocircularization oligonucleotide) at stabilizing RNAs or increasing RNA levels because in cells, for example, 3′ end processing exonucleases may be dominant (e.g., compared with 5′ end processing exonucleases). However, in some embodiments, 3′ targeting oligonucleotides are used in combination with 5′ targeting oligonucleotides, or alone, to stabilize a target RNA.

In some embodiments, where a targeted RNA is protein-coding, increases in steady state levels of the RNA result in concomitant increases in levels of the encoded protein. Thus, in some embodiments, stabilizing oligonucleotides (including 5′-targeting, 3′-targeting and pseudocircularization oligonucleotides) are provided herein that when delivered to cells increase protein levels of target RNAs. In some embodiments, not only are target RNA levels increased but the resulting translation products are also increased. In some embodiments, this result is surprising in part because of an understanding that for translation to occur ribosomal machinery requires access to certain regions of the RNA (e.g., the 5′ cap region, start codon, etc.) to facilitate translation.

In some embodiments, where the targeted RNA is non-coding, increases in steady state levels of the non-coding RNA result in concomitant increases activity associated with the non-coding RNA. For example, in instances where the non-coding RNA is an miRNA, increases in steady state levels of the miRNA may result in increased degradation of mRNAs targeted by the miRNA.

In some embodiments, stabilizing oligonucleotides are provided with chemistries suitable for delivery, hybridization and stability within cells to target and stabilize RNA transcripts. Furthermore, in some embodiments, stabilizing oligonucleotide chemistries are provided that are useful for controlling the pharmacokinetics, biodistribution, bioavailability and/or efficacy of the oligonucleotides.

In some aspects of the disclosure, methods are provided for stabilizing a synthetic RNA (e.g., a synthetic RNA that is to be delivered to a cell). In some embodiments, the methods involve contacting a synthetic RNA with one or more stabilizing oligonucleotides that bind to a 5′ region of the synthetic RNA and a 3′ region of the synthetic RNA and that when bound to the synthetic RNA form a circularized product with the synthetic RNA. In some embodiments, the synthetic RNA is contacted with the one or more stabilizing oligonucleotides outside of a cell. Aspects of the disclosure relate to a formulation comprising: a single stranded synthetic nucleic acid, one or more stabilizing oligonucleotides complementary with the single stranded synthetic nucleic acid, and a particle. In some embodiments, the single stranded synthetic nucleic acid is a synthetic RNA.

In other aspects of the disclosure, methods are provided for stabilizing a nucleic acid in a cell (e.g., a messenger RNA present in a cell). In some embodiments, the methods involve contacting a messenger RNA in a cell with one or more stabilizing oligonucleotides that bind to a 5′ region of the RNA and a 3′ region of the messenger RNA and that when bound to the messenger RNA form a circularized product with the messenger RNA. Accordingly, aspects of the disclosure also relate to a formulation comprising: one or more stabilizing oligonucleotides, and a particle. In some embodiments, the particle is a nanoparticle. In some embodiments, the nanoparticle is a lipid nanoparticle.

In some embodiments, the particle is a nanoparticle. In some embodiments, the nanoparticle is a lipid nanoparticle.

In some embodiments, the lipid nanoparticle includes: (a) one or more cationic lipids, (b) one or more non-cationic lipids, (c) one or more conjugated lipids that inhibits aggregation of particles, or a combination thereof. In some embodiments, the lipid nanoparticle includes (a) one or more cationic lipids. In some embodiments, the lipid nanoparticle includes (a) one or more cationic lipid and (b) one or more non-cationic lipids. In some embodiments, the lipid nanoparticle includes (a) one or more cationic lipids, (b) one or more non-cationic lipids, and (c) one or more conjugated lipids that inhibit aggregation of particle.

In some embodiments, the lipid nanoparticle includes a cationic lipid selected from N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9, 12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino) butanoate (MC3), 1, l′-(2-(4-(2-((2-(bis(2-hydroxydodecyl)amino)ethyl) (2-hydroxydodecyl)amino)ethyl) piperazin-1-yl)ethylazanediyl)didodecan-2-ol (Tech Gl), 2,2-Dilinoleyl-4-dimethylaminoethyl-[1,3]-dioxolane, β-L-arginyl-2, 3-L-diaminopropionic acid-N-palmityl-N-oleylamide trihydrochloride, N′,N′-dioctadecyl-N-4, 8-diaza-10-aminodecanoylglycine amide[71], 1,2-dilinoleyloxy-3-dimethylaminopropane, DLin-KC2-DMA, amino lipid 2,2-dilinoleyl-4-dimethylaminoethyl-[1,3]-dioxolane (DLin-KC2-DMA, 1), 1,2-distearloxy-/V,N-dimethylaminopropane (DSDMA), dilinoleylmethyl-4-dimethylaminobutyrate (DLin-MC3-DMA), DLin-D-DMA, C12-200, 98N12-5, (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimethyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimethylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyleptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimeth-yl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propa-n-2-amine, S—N,N-dimethyl-1-[(9Z,12Z)-octadeca-9,12-dien-1-yloxy]-3-(octy-loxy)propan-2-amine, 1-{2-[(9Z,12Z)-octadeca-9,12-dien-1-yloxy]-1-Roctyloxy)methyl]ethyl}pyrro-lidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azet-idine, (2S)-1-(hexyloxy)-N,N-dimethyl-3-[(9Z,12Z)-octadeca-9,12-dien-1-ylo-xy]propan-2-amine, (2S)-1-(heptyloxy)-N,N-dimethyl-3-[(9Z,12Z)-octadeca-9,12-dien-1-yloxy]pr-opan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-am-ine; (2S)—N,N-dimethyl-1-[(6Z,9Z,12Z)-octadeca-6,9,12-trien-1-yloxy]-3-(o-ctyloxy)propan-2-amine, (2S)-1-[(11Z,14Z)-icosa-11,14-dien-1-yloxy]-N,N-dimethyl-3-(pentyloxy)propa-n-2-amine, (2S)-1-(hexyloxy)-3-[(11Z,14Z)-icosa-11,14-dien-1-yloxy]-N,N-di-methylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)pr-opan-2-amine, (2S)-1-[(13Z,16Z)-docosa-13,16-dien-1-yloxy]-3-(hexyloxy)-N,N-dimethylpro-pan-2-amine, (2S)-1-[(13Z)-docos-13-en-1-yloxy]-3-(hexyloxy)-N,N-dimethylpropan-2-amin-e, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H (1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-di-en-1-yloxy]propan-2-amine, N,N-dimethyl-1-(octyloxy)-3-({8-[(1S,2S)-2-{[(1R,2R)-2-pentylcyclopropyl]-methyl}cyclopropyl]octyl}oxy)propan-2-amine, N,N-dimethyl-1-{[8-(2-oclylcyclopropyl)octyl]oxy}-3-(octyloxy)propan-2-am-ine and (11E,20Z,23Z)—N,N-dimethylnonacosa-11,20,2-trien-10-amine, 5-carboxyspermylglycine dioctaoleoylamide (“DOGS”), dipalmitoylphosphatidylethanolamine 5-carboxyspermyl-amide (“DPPES”), 1,2-dimyristyloxypropyl-3-dimethyl-hydroxy ethyl ammonium bromide (DMRIE), DMRIE-HP, Lipofectamine (DOSPA), 3b-(N—(N′,N′-dimethylaminoethane)-carbamoyl) cholesterol (“DC-Choi”), N-(1,2-dimyhstyloxyprop-3-yl)-N,N-dimethyl-N-hydroxyethyl ammonium bromide (“DMRIE”), 1,2-Dioleoyl-3-dimethylammonium-propane (“DODAP”), DMDMA, cationic lipid-based transfection reagents TransIT-TKO, LIPOFECTIN, Lipofectamine, OLIGOFECTAMINE or DHARMAFECT, DSDMA, DODMA, DLinDMA, DLenDMA, gamma-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA) (also known as 1-B1 1), or a mixture thereof.

In some embodiments, the lipid nanoparticle includes a non-cationic lipid, wherein the non-cationic lipid is an anionic lipid. In some embodiments, the lipid nanoparticle includes a non-cationic lipid, wherein the non-cationic lipid is a neutral lipid.

In some embodiments, the lipid nanoparticle includes a non-cationic lipid selected from distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids, or a mixture thereof.

In some embodiments, the anionic lipid is 1,2-dioleoyl-sn-glycero-3-phospho-(1′-rac-glycerol).

In some embodiments, the neutral lipid is 1,2-dioleoyl-sn-glycero-3-phosphatidylcholine.

In some embodiments, the lipid nanoparticle includes a conjugated lipid that inhibits aggregation of particles. In some embodiments, the conjugated lipid is a PEG lipid. In some embodiments, the PEG lipid is a PEG-diacylglycerol (DAG), a PEG-dialkyloxypropyl (DAA), a PEG-phospholipid, a PEG-ceramide (Cer), or a mixture thereof. In some embodiments, PLGA is conjugated to a lipid-terminating PEG forming PLGA-DSPE-PEG. In some embodiments, a PEG lipid is selected from PEG-c-DOMG and 1,2-Dimyristoyl-sn-glycerol, methoxypolyethylene Glycol (PEG-DMG), 1,2-Distearoyl-sn-glycerol, methoxypolyethylene Glycol (PEG-DSG), PEG-c-DOMG, 1,2-Distearoyl-sn-glycerol, methoxypolyethylene glycol (PEG-DSG) 1,2-Dipalmitoyl-sn-glycerol, methoxypolyethylene glycol (PEG-DPG), PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers (e.g., ATTA-lipid conjugates), and mixtures thereof. In some embodiments, the PEG is a PEG-dilauryloxypropyl (C12), a PEG-dimyristyloxypropyl (C14), a PEG-dipalmityloxypropyl (C16), a PEG-distearyloxypropyl (C18), PEG-c-DOMG, PEG-DMG, or a mixture thereof.

In some embodiments, the conjugated PEG lipid is coupled to the surface of the lipid nanoparticle. In some embodiments, the PEG lipid is coupled to the surface of the lipid nanoparticle by an oxime linkage. In some embodiments, the conjugated PEG lipid is susceptible to decomposition in an acidic environment.

In some embodiments, formulations are provided in which the lipid nanoparticle comprises more than one cationic lipid, more than one non-cationic lipid, more than one conjugated lipid, or a combination thereof.

In some embodiments, formulations are provided in which the one or more stabilizing oligonucleotides are substantially encapsulated within an aqueous interior of the lipid nanoparticle.

In some embodiments, the lipid nanoparticle is about 50 to 150 nm in diameter. In some embodiments, the lipid nanoparticle is about 20-50 nm in diameter. In some embodiments, the lipid nanoparticle is about 30 nm in diameter.

In some embodiments, formulations are provided in which the lipid to stabilizing oligonucleotide ratio (mass/mass ratio; w/w ratio) is from about 1:1 to about 50:1, from about 1:1 to about 25:1, from about 10:1 to about 14:1, from about 3:1 to about 15:1, from about 4:1 to about 10:1, from about 5:1 to about 9:1, or about 6:1 to about 9:1.

In some embodiments, formulations are provided in which the particle is a microsphere. In some embodiments, the microsphere comprises poly(lactic-co-glycolic acid) (PLGA). In some embodiments, the microsphere particle is about 4 and 20 μm in diameter.

In some embodiments, formulations are provided in which the particle includes a polymer. In some embodiments, the polymer comprises a layer of a hydrogel or surgical sealant. In some embodiments, the polymer is PLGA, ethylene vinyl acetate, poloxamer, GELSITE®, or a combination thereof.

In some embodiments, formulations are provided which further include protamine or calcium phosphate. In some embodiments, formulations are provided which further include hyaluronic acid. In some embodiments, formulations are provided which further include polyglutamate.

In some embodiments, formulations are provided in which the particle comprises a lipoprotein or lipoprotein mimetic. In some embodiments, the lipoprotein is HDL, LDL, or a combination thereof.

In some embodiments, formulations are provided in which the particle comprises a lipidoid. In some embodiments, the lipidoid is penta[3-(1-laurylaminopropionyl)]-triethylenetetramine hydrochloride (TETA-5LAP; 98N12-5), C12-200, MD1, or a combination thereof.

In some embodiments, the stabilizing oligonucleotide is a modified oligonucleotide. In some embodiments, the modified oligonucleotide comprises a modified sugar moiety, a modified internucleoside linkage, or a modified nucleotide, or a combination thereof.

In some embodiments, the stabilizing oligonucleotide is a mixmer. In some embodiment, the stabilizing oligonucleotide is a morpholino.

In some embodiments, the synthetic RNA comprises a transcription start site.

In some embodiments, the one or more stabilizing oligonucleotides comprises an oligonucleotide of 8 to 50 nucleotides in length and comprising a region of complementarity that is complementary with at least 5 contiguous nucleotides of the synthetic RNA, wherein the nucleotide at the 3′-end of the region of complementarity is complementary with a nucleotide within 10 nucleotides of the transcription start site of the synthetic RNA, wherein the oligonucleotide comprises nucleotides linked by at least one modified internucleoside linkage or at least one bridged nucleotide.

In some embodiments, the one or more stabilizing oligonucleotides comprises an oligonucleotide comprising two regions of complementarity each of which is complementary with at least 5 contiguous nucleotides of the synthetic RNA, wherein the nucleotide at the 3′-end of the first region of complementary is complementary with a nucleotide within 100 nucleotides of the transcription start site of the synthetic RNA and wherein the second region of complementarity is complementary with a region of the synthetic RNA that ends within 300 nucleotides of the 3′-end of the RNA transcript.

In some embodiments, the one or more stabilizing oligonucleotides comprises an oligonucleotide comprising the general formula 5′-X₁—X₂-3′, wherein X₁ comprises 5 to 20 nucleotides that have a region of complementarity that is complementary with at least 5 contiguous nucleotides of a synthetic RNA, wherein the nucleotide at the 3′-end of the region of complementary of X₁ is complementary with the nucleotide at the transcription start site of the RNA transcript; and X₂ comprises 1 to 20 nucleotides.

In some embodiments, the synthetic RNA has a 7-methylguanosine cap at its 5′-end. In some embodiments, the synthetic RNA has a 7-methylguanosine cap, and the nucleotide at the 3′-end of the region of complementary of X₁ is complementary with the nucleotide of the synthetic RNA that is immediately internal to the 7-methylguanosine cap.

In some embodiments, at least the first nucleotide at the 5′-end of X₂ is a pyrimidine complementary with guanine. In some embodiments, the second nucleotide at the 5′-end of X₂ is a pyrimidine complementary with guanine. In some embodiments, X₂ comprises the formula 5′-Y₁—Y₂—Y₃-3′, wherein X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, Y₁, Y₂ and Y₃ independently comprise 1 to 10 nucleotides. In some embodiments, Y₃ comprises, at a position immediately following the 3′-end of the stem region, a pyrimidine complementary with guanine. In some embodiments, the pyrimidine complementary with guanine is cytosine.

In some embodiments, X₂ comprises a region of complementarity that is complementary with at least 5 contiguous nucleotides of the synthetic RNA that do not overlap the region of the synthetic RNA that is complementary with the region of complementarity of X₁. In some embodiments, the region of complementarity of X₂ is within 100 nucleotides of a polyadenylation junction of the synthetic RNA. In some embodiments, the region of complementarity of X₂ is complementary with the synthetic RNA immediately adjacent to or overlapping the polyadenylation junction of the synthetic RNA. In some embodiments, X₂ further comprises at least 2 consecutive pyrimidine nucleotides complementary with adenine nucleotides of the poly(A) tail of the synthetic RNA.

In some embodiments, the synthetic RNA is an mRNA, non-coding RNA, long non-coding RNA, miRNA, or snoRNA or any other suitable RNA.

In some embodiments, the synthetic RNA is an mRNA transcript, and wherein X₂ comprises a region of complementarity that is complementary with at least 5 contiguous nucleotides in the 3′-UTR of the transcript.

In some embodiments, formulations are provided in which the one or more stabilizing oligonucleotides comprises an oligonucleotide of 10 to 50 nucleotides in length having a first region complementary with at least 5 consecutive nucleotides of the 5′-UTR of a synthetic RNA, and a second region complementary with at least 5 consecutive nucleotides of the 3′-UTR, poly(A) tail, or overlapping the polyadenylation junction of the synthetic RNA. In some embodiments, the first of the at least 5 consecutive nucleotides of the 5′-UTR of the stabilizing oligonucleotide is within 10 nucleotides of the 5′-methylguanosine cap of the synthetic RNA. In some embodiments, the second region of the stabilizing oligonucleotide is complementary with at least 5 consecutive nucleotides overlapping the polyadenylation junction.

In some embodiments, the stabilizing oligonucleotide further comprises 2-20 nucleotides that link the 5′ end of the first region with the 3′ end of the second region. In some embodiments, the stabilizing oligonucleotide further comprises 2-20 nucleotides that link the 3′ end of the first region with the 5′ end of the second region. In some embodiments, the oligonucleotide is 10 to 50 nucleotide in length. In some embodiments, the oligonucleotide is 9 to 20 nucleotide in length.

In some embodiments, formulations are provided in which the one or more stabilizing oligonucleotides comprises an oligonucleotide comprising the general formula 5′-X1-X2-3′, wherein X1 comprises 2 to 20 pyrimidine nucleotides that form base pairs with adenine; and X2 comprises a region of complementarity that is complementary with at least 3 contiguous nucleotides of a poly-adenylated synthetic RNA, wherein the nucleotide at the 5′-end of the region of complementary of X2 is complementary with the nucleotide of the synthetic RNA that is immediately internal to the poly-adenylation junction of the synthetic RNA.

In some embodiments, formulations are provided in which the one or more stabilizing oligonucleotides comprises: a first oligonucleotide having 5 to 25 nucleotides linked through internucleoside linkages, and a second oligonucleotide having 5 to 25 nucleotides linked through internucleoside linkages, wherein the first oligonucleotide is complementary with at least 5 consecutive nucleotides within 100 nucleotides of the 5′-end of a synthetic RNA and wherein the second oligonucleotide is complementary with at least 5 consecutive nucleotides within 100 nucleotides of the 3′-end of a synthetic RNA.

In some embodiments, the first oligonucleotide and second oligonucleotide are joined by a linker that is not an oligonucleotide having a sequence complementary with the synthetic RNA. In some embodiments, the linker is an oligonucleotide. In some embodiments, the linker is a polypeptide.

In some embodiments, the synthetic RNA encodes a protein. In some embodiments, the synthetic RNA comprises one or more modified nucleotides.

In some embodiments, the formulation comprises a first and a second stabilizing oligonucleotide.

In some embodiments, the first stabilizing oligonucleotide comprises a region of complementarity to a 5′ region of the synthetic RNA and the second stabilizing oligonucleotide comprises a region of complementarity to a 3′ region of the synthetic RNA. In some embodiments, the first stabilizing oligonucleotide is covalently linked with the second stabilizing oligonucleotide. In some embodiments, the first stabilizing oligonucleotide and second stabilizing oligonucleotide are covalently linked through an internucleoside linkage. In some embodiments, the first stabilizing oligonucleotide and second stabilizing oligonucleotide are covalently linked through an oligonucleotide. In some embodiments, the first stabilizing oligonucleotide and second stabilizing oligonucleotide are covalently linked through a linker.

In some embodiments, the synthetic RNA is circularized. In some embodiments, the synthetic RNA has a 7-methylguanosine cap at its 5′-end.

In some embodiments, the first stabilizing oligonucleotide comprises a region of complementarity that is complementary with the synthetic RNA at a position within 10 nucleotides of the first nucleotide at the 5′ end of the synthetic RNA.

In some embodiments, the synthetic RNA comprises a 5′-methylguanosine cap, and wherein the first stabilizing oligonucleotide comprises a region of complementarity that is complementary with the synthetic RNA at a position within 10 nucleotides of the nucleotide immediately internal to the 5′-methylguanosine cap.

In some embodiments, the second stabilizing oligonucleotide comprises a region of complementarity that is complementary with the synthetic RNA at a position within 250 nucleotides of the 3′ end of the synthetic RNA.

In some embodiments, the synthetic RNA comprises a 3′-poly(A) tail, and wherein the second stabilizing oligonucleotide comprises a region of complementarity that is complementary with the synthetic RNA at a position within 100 nucleotides of the polyadenylation junction of the synthetic RNA.

In some embodiments, the region of complementarity of the second stabilizing oligonucleotide is immediately adjacent to or overlapping the polyadenylation junction of the synthetic RNA.

In some embodiments, the synthetic RNA is an RNA transcript. In some embodiments, the synthetic RNA is a functional RNA. In some embodiments, the synthetic RNA is up to 10 kb in length.

In some embodiments, the synthetic RNA comprises one or more modified nucleotides. In some embodiments, the one or more modified nucleotides are selected from the group consisting of: 2′-amino-2′-deoxynucleotide, 2′-azido-2′-deoxynucleotide, 2′-fluoro-2′-deoxynucleotide, 2′-O-methyl-nucleotide, 2′ sugar super modifier, 2′-modified thermostability enhancer, 2′-fluoro-2′-deoxyadenosine-5′-triphosphate, 2′-fluoro-2′-deoxycytidine-5′-triphosphate, 2′-fluoro-2′-deoxyguanosine-5′-triphosphate, 2′-fluoro-2′-deoxyuridine-5′-triphosphate, 2′-O-methyladenosine-5′-triphosphate, 2′-O-methylcytidine-5′-triphosphate, 2′-O-methylguanosine-5′-triphosphate, 2′-O-methyluridine-5′-triphosphate, pseudouridine-5′-triphosphate, 2′-O-methylinosine-5′-triphosphate, 2′-amino-2′-deoxycytidine-5′-triphosphate, 2′-amino-2′-deoxyuridine-5′-triphosphate, 2′-azido-2′-deoxycytidine-5′-triphosphate, 2′-azido-2′-deoxyuridine-5′-triphosphate, 2′-O-methylpseudouridine-5′-triphosphate, 2′-O-methyl-5-methyluridine-5′-triphosphate, 2′-azido-2′-deoxyadenosine-5′-triphosphate, 2′-amino-2′-deoxyadenosine-5′-triphosphate, 2′-fluoro-thymidine-5′-triphosphate, 2′-azido-2′-deoxyguanosine-5′-triphosphate, 2′-amino-2′-deoxyguanosine-5′-triphosphate, and N4-methylcytidine-5′-triphosphate.

In some aspects of the disclosure, methods are provided for delivering a synthetic RNA to a cell, the method comprising delivering to a cell a formulation of the present disclosure. In some embodiments, the increase in the level of gene expression results from an increase in the level of a protein encoded by the synthetic RNA. In some embodiments, the increase in the level of protein encoded by the synthetic RNA is at least a 50% increase compared with an appropriate cell to which the formulation was not delivered. In some embodiments, the cell is in vitro. In some embodiments, the cell is in vivo.

In some embodiments, the one or more stabilizing oligonucleotides comprises an oligonucleotide comprising two regions of complementarity each of which is complementary with at least 5 contiguous nucleotides of an RNA transcript, wherein the nucleotide at the 3′-end of the first region of complementary is complementary with a nucleotide within 100 nucleotides of the transcription start site of the RNA transcript and wherein the second region of complementarity is complementary with a region of the RNA transcript that ends within 300 nucleotides of the 3′-end of the RNA transcript.

In some embodiments, the one or more stabilizing oligonucleotides comprises an oligonucleotide comprising the general formula 5′-X₁—X₂-3′, wherein X₁ comprises 5 to 20 nucleotides that have a region of complementarity that is complementary with at least 5 contiguous nucleotides of an RNA transcript, wherein the nucleotide at the 3′-end of the region of complementary of X₁ is complementary with the nucleotide at the transcription start site of the RNA transcript; and X₂ comprises 1 to 20 nucleotides.

In some embodiments, the RNA transcript has a 7-methylguanosine cap at its 5′-end. In some embodiments, the RNA transcript has a 7-methylguanosine cap, and the nucleotide at the 3′-end of the region of complementary of X₁ is complementary with the nucleotide of the RNA transcript that is immediately internal to the 7-methylguanosine cap.

In some embodiments, at least the first nucleotide at the 5′-end of X₂ is a pyrimidine complementary with guanine. In some embodiments, the second nucleotide at the 5′-end of X₂ is a pyrimidine complementary with guanine. In some embodiments, X₂ comprises the formula 5′-Y₁—Y₂—Y₃-3′, wherein X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, Y₁, Y₂ and Y₃ independently comprise 1 to 10 nucleotides. In some embodiments, Y₃ comprises, at a position immediately following the 3′-end of the stem region, a pyrimidine complementary with guanine. In some embodiments, the pyrimidine complementary with guanine is cytosine.

In some embodiments, X₂ comprises a region of complementarity that is complementary with at least 5 contiguous nucleotides of the RNA transcript that do not overlap the region of the RNA transcript that is complementary with the region of complementarity of X₁. In some embodiments, the region of complementarity of X₂ is within 100 nucleotides of a polyadenylation junction of the RNA transcript. In some embodiments, the region of complementarity of X₂ is complementary with the RNA transcript immediately adjacent to or overlapping the polyadenylation junction of the RNA transcript. In some embodiments, X₂ further comprises at least 2 consecutive pyrimidine nucleotides complementary with adenine nucleotides of the poly(A) tail of the RNA transcript.

In some embodiments, the RNA transcript is an mRNA, non-coding RNA, long non-coding RNA, miRNA, or snoRNA or any other suitable RNA.

In some embodiments, the RNA transcript is an mRNA transcript, and wherein X₂ comprises a region of complementarity that is complementary with at least 5 contiguous nucleotides in the 3′-UTR of the transcript.

In some embodiments, formulations are provided in which the one or more stabilizing oligonucleotides comprises an oligonucleotide of 10 to 50 nucleotides in length having a first region complementary with at least 5 consecutive nucleotides of the 5′-UTR of an mRNA transcript, and a second region complementary with at least 5 consecutive nucleotides of the 3′-UTR, poly(A) tail, or overlapping the polyadenylation junction of the mRNA transcript. In some embodiments, the first of the at least 5 consecutive nucleotides of the 5′-UTR of the stabilizing oligonucleotide is within 10 nucleotides of the 5′-methylguanosine cap of the mRNA transcript. In some embodiments, the second region of the stabilizing oligonucleotide is complementary with at least 5 consecutive nucleotides overlapping the polyadenylation junction.

In some embodiments, the stabilizing oligonucleotide further comprises 2-20 nucleotides that link the 5′ end of the first region with the 3′ end of the second region. In some embodiments, the stabilizing oligonucleotide further comprises 2-20 nucleotides that link the 3′ end of the first region with the 5′ end of the second region. In some embodiments, the oligonucleotide is 10 to 50 nucleotide in length. In some embodiments, the oligonucleotide is 9 to 20 nucleotide in length.

In some embodiments, formulations are provided in which the one or more stabilizing oligonucleotides comprises an oligonucleotide comprising the general formula 5′-X1-X2-3′, wherein X1 comprises 2 to 20 pyrimidine nucleotides that form base pairs with adenine; and X2 comprises a region of complementarity that is complementary with at least 3 contiguous nucleotides of a poly-adenylated RNA transcript, wherein the nucleotide at the 5′-end of the region of complementary of X2 is complementary with the nucleotide of the RNA transcript that is immediately internal to the poly-adenylation junction of the RNA transcript.

In some embodiments, formulations are provided in which the one or more stabilizing oligonucleotides comprises: a first oligonucleotide having 5 to 25 nucleotides linked through internucleoside linkages, and a second oligonucleotide having 5 to 25 nucleotides linked through internucleoside linkages, wherein the first oligonucleotide is complementary with at least 5 consecutive nucleotides within 100 nucleotides of the 5′-end of an RNA transcript and wherein the second oligonucleotide is complementary with at least 5 consecutive nucleotides within 100 nucleotides of the 3′-end of an RNA transcript.

In some embodiments, the first oligonucleotide and second oligonucleotide are joined by a linker that is not an oligonucleotide having a sequence complementary with the RNA transcript. In some embodiments, the linker is an oligonucleotide. In some embodiments, the linker is a polypeptide.

In some aspects of the disclosure, methods are provided for increasing gene expression in a cell, the method comprising delivering to a cell a formulation of the present disclosure. In some embodiments, the increase in the level of gene expression results from an increase in the level of a protein encoded by an mRNA that is stabilized by the one or more stabilizing oligonucleotides. In some embodiments, the increase in the level of protein encoded by an mRNA is at least a 50% increase compared with an appropriate cell to which the formulation was not delivered. In some embodiments, the cell is in vitro. In some embodiments, the cell is in vivo.

In some aspects of the disclosure, methods are provided for treating a condition or disease associated with decreased levels of an RNA transcript in a subject, the method comprising administering a formulation of the present disclosure to the subject. In some embodiments, the formulation is administered via topical, oral, or parenteral administration. In some embodiments, the parental administration is intravenous, subcutaneous, intraperitoneal, intramuscular, intrathecal, or intraventricular administration.

In some aspects of the disclosure, kits are provided, which include a container housing a formulation of the present disclosure.

### BRIEF DESCRIPTION OF THE FIGURES

FIG. 1 is an illustration depicting exemplary oligo designs for targeting 3′ RNA ends. The first example shows oligos complementary to the 3′ end of RNA, before the polyA-tail. The second example shows oligos complementary to the 3′ end of RNA with a 5′ T-stretch to hybridize to a polyA tail.

FIG. 2 is an illustration depicting exemplary oligos for targeting 5′ RNA ends. The first example shows oligos complementary to the 5′ end of RNA. The second example shows oligos complementary to the 5′ end of RNA, the oligo having 3′ overhang residues to create a RNA-oligo duplex with a recessed end. Overhang can include a combination of nucleotides including, but not limited to, C to potentially interact with a 5′ methylguanosine cap and stabilize the cap further.

FIG. 3A is an illustration depicting exemplary oligos for targeting 5′ RNA ends and exemplary oligos for targeting 5′ and 3′ RNA ends. The example shows oligos with loops to stabilize a 5′ RNA cap or oligos that bind to a 5′ and 3′ RNA end to create a pseudo-circularized RNA.

FIG. 3B is an illustration depicting exemplary oligo-mediated RNA pseudo-circularization. The illustration shows an LNA mixmer oligo binding to the 5′ and 3′ regions of an exemplary RNA.

### DETAILED DESCRIPTION OF CERTAIN EMBODIMENTS OF THE DISCLOSURE

Formulations and methods disclosed herein are useful in a variety of different contexts in which it is desirable to protect RNAs from degradation, including protecting RNAs inside or outside of cells. In some embodiments, formulations and methods are provided that are useful for posttranscriptionally altering protein and/or RNA levels in cells in a targeted manner. For example, formulations and methods are provided that involve reducing or preventing degradation or processing of targeted RNAs thereby elevating steady state levels of the targeted RNAs. In some embodiments, the stability of an RNA is increased by protecting one or both ends (5′ or 3′ ends) of the RNA from exonuclease activity, thereby increasing stability of the RNA.

In some embodiments, methods of increasing gene expression are provided. As used herein the term, “gene expression” refers generally to the level or representation of a product of a gene in a cell, tissue or subject. It should be appreciated that a gene product may be an RNA transcript or a protein, for example. An RNA transcript may be protein coding. An RNA transcript may be non-protein coding, such as, for example, a long non-coding RNA, a long intergenic non-coding RNA, a non-coding RNA, an miRNA, a small nuclear RNA (snRNA), or other functional RNA. In some embodiments, formulations and methods for increasing gene expression may involve increasing stability of a RNA transcript, and thereby increasing levels of the RNA transcript in the cell. Formulations and methods for of increasing gene expression may alternatively or in addition involve increasing transcription or translation of RNAs. In some embodiments, other mechanisms of manipulating gene expression may be involved in methods disclosed herein.

In some embodiments, methods provided herein involve delivering to a cell one or more sequence specific oligonucleotides that hybridize with an RNA transcript at or near one or both ends, thereby protecting the RNA transcript from exonuclease mediated degradation. In embodiments where the targeted RNA transcript is protein-coding, increases in steady state levels of the RNA typically result in concomitant increases in levels of the encoded protein. In embodiments where the targeted RNA is non-coding, increases in steady state levels of the non-coding RNA typically result in concomitant increases in activity associated with the non-coding RNA.

In some embodiments, approaches disclosed herein based on regulating RNA levels and/or protein levels using oligonucleotides targeting RNA transcripts by mechanisms that increase RNA stability and/or translation efficiency may have several advantages over other types of oligos or compounds, such as oligonucleotides that alter transcription levels of target RNAs using cis or noncoding based mechanisms. For example, in some embodiments, lower concentrations of oligos may be used when targeting RNA transcripts in the cytoplasm as multiple copies of the target molecules exist. In contrast, in some embodiments, oligos that target transcriptional processes may need to saturate the cytoplasm and before entering nuclei and interacting with corresponding genomic regions, of which there are only one/two copies per cell, in many cases. In some embodiments, response times may be shorter for RNA transcript targeting because RNA copies need not to be synthesized transcriptionally. In some embodiments, a continuous dose response may be easier to achieve. In some embodiments, well defined RNA transcript sequences facilitate design of oligonucleotides that target such transcripts. In some embodiments, oligonucleotide design approaches provided herein, e.g., designs having sequence overhangs, loops, and other features facilitate high oligo specificity and sensitivity compared with other types of oligonucleotides, e.g., certain oligonucleotides that target transcriptional processes.

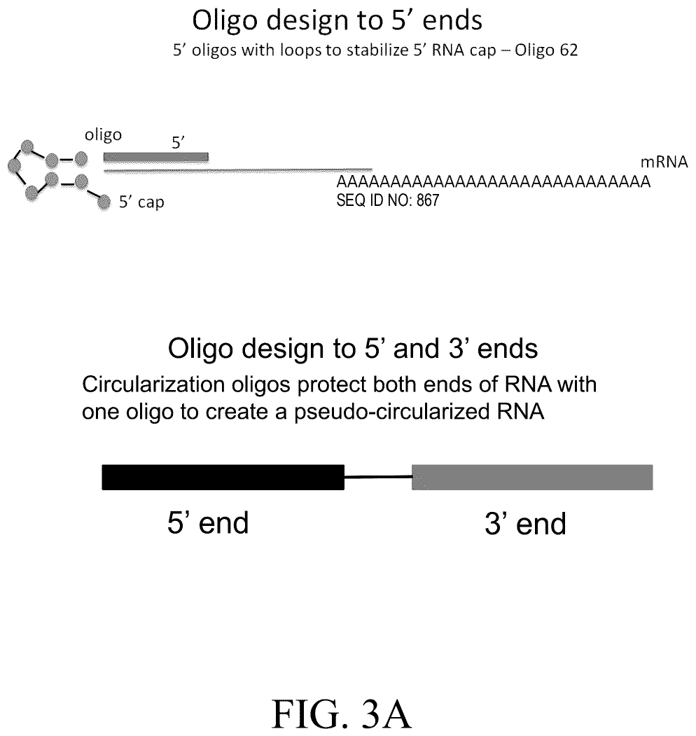

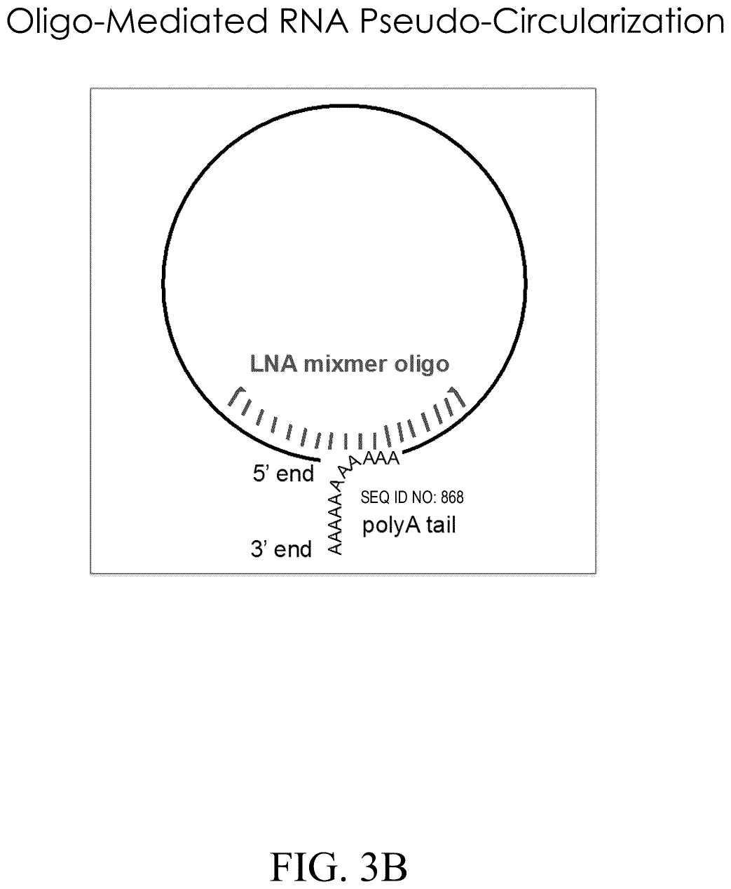

In some embodiments, methods provided herein involve use of oligonucleotides that stabilize a nucleic acid (e.g., an RNA) by hybridizing at a single stranded 5′ and/or 3′ region of the nucleic acid. In some embodiments, oligonucleotides that prevent or inhibit degradation of an nucleic acid by hybridizing with the nucleic acid may be referred to herein as “stabilizing oligonucleotides.” In some examples, such oligonucleotides hybridize with an RNA and prevent or inhibit exonuclease mediated degradation of the RNA. Inhibition of exonuclease mediated degradation includes, but is not limited to, reducing the extent of degradation of a particular RNA by exonucleases. For example, an exonuclease that processes only single stranded RNA may cleave a portion of the RNA up to a region where an oligonucleotide is hybridized with the RNA because the exonuclease cannot effectively process (e.g., pass through) the duplex region. Thus, in some embodiments, using an oligonucleotide that targets a particular region of an RNA makes it possible to control the extent of degradation of the RNA by exonucleases up to that region. For example, use of an oligonucleotide that hybridizes at an end of an RNA may reduce or eliminate degradation by an exonuclease that processes only single stranded RNAs from that end. For example, use of an oligonucleotide that hybridizes at the 5′ end of an RNA may reduce or eliminate degradation by an exonuclease that processes single stranded RNAs in a 5′ to 3′ direction. Similarly, use of an oligonucleotide that hybridizes at the 3′ end of an RNA may reduce or eliminate degradation by an exonuclease that processes single stranded RNAs in a 3′ to 5′ direction. In some embodiments, lower concentrations of an oligo may be used when the oligo hybridizes at both the 5′ and 3′ regions of the RNA. In some embodiments, an oligo that hybridizes at both the 5′ and 3′ regions of the RNA protects the 5′ and 3′ regions of the RNA from degradation (e.g., by an exonuclease). In some embodiments, an oligo that hybridizes at both the 5′ and 3′ regions of the RNA creates a pseudo-circular RNA (e.g., a circularized RNA with a region of the poly A tail that protrudes from the circle, see FIG. 3B). In some embodiments, a pseudo-circular RNA is translated at a higher efficiency than a non-pseudo-circular RNA.

In some embodiments, an oligonucleotide may be used that comprises multiple regions of complementarity with an RNA, such that at one region the oligonucleotide hybridizes at or near the 5′ end of the RNA and at another region it hybridizes at or near the 3′ end of the RNA, thereby preventing or inhibiting degradation of the RNA by exonucleases at both ends. In some embodiments, when an oligonucleotide hybridizes both at or near the 5′ end of an RNA and at or near the 3′ end of the RNA a circularized complex results that is protected from exonuclease mediated degradation. In some embodiments, when an oligonucleotide hybridizes both at or near the 5′ end of an mRNA and at or near the 3′ end of the mRNA, the circularized complex that results is protected from exonuclease mediated degradation and the mRNA in the complex retains its ability to be translated into a protein.

As used herein the term, “synthetic RNA” refers to a RNA produced through an in vitro transcription reaction or through artificial (non-natural) chemical synthesis. In some embodiments, a synthetic RNA is an RNA transcript. In some embodiments, a synthetic RNA encodes a protein. In some embodiments, the synthetic RNA is a functional RNA (e.g., a lncRNA, miRNA, etc.). In some embodiments, a synthetic RNA comprises one or more modified nucleotides. In some embodiments, a synthetic RNA is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a synthetic RNA is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

As used herein, the term “RNA transcript” refers to an RNA that has been transcribed from a nucleic acid by a polymerase enzyme. An RNA transcript may be produced inside or outside of cells. For example, an RNA transcript may be produced from a DNA template encoding the RNA transcript using an in vitro transcription reaction that utilizes recombination or purified polymerase enzymes. An RNA transcript may also be produced from a DNA template (e.g., chromosomal gene, an expression vector) in a cell by an RNA polymerase (e.g., RNA polymerase I, II, or III). In some embodiments, the RNA transcript is a protein coding mRNA. In some embodiments, the RNA transcript is a non-coding RNA (e.g., a tRNA, rRNA, snoRNA, miRNA, ncRNA, long-noncoding RNA, shRNA). In some embodiments, RNA transcript is up to 0.5 kilobases (kb), 1 kb, 1.5 kb, 2 kb, 2.5 kb, 3 kb, 4 kb, 5 kb, 6 kb, 7 kb, 8 kb, 9 kb, 10 kb, 15 kb, 20 kb, 25 kb, 30 kb or more in length. In some embodiments, a RNA transcript is in a range of 0.1 kb to 1 kb, 0.5 kb to 2 kb, 0.5 kb to 10 kb, 1 kb to 5 kb, 2 kb to 5 kb, 1 kb to 10 kb, 3 kb to 10 kb, 5 kb to 15 kb, or 1 kb to 30 kb in length.

In some embodiments, the RNA transcript is capped post-transcriptionally, e.g., with a 7′-methylguanosine cap. In some embodiments, the 7′-methylguanosine is added to the RNA transcript by a guanylyltransferase during transcription (e.g., before the RNA transcript is 20-50 nucleotides long.) In some embodiments, the 7 ‘-methylguanosine is linked to the first transcribed nucleotide through a 5’-5′ triphosphate bridge. In some embodiments, the nucleotide immediately internal to the cap is an adenosine that is N6 methylated. In some embodiments, the first and second nucleotides immediately internal to the cap of the RNA transcript are not 2′-O-methylated. In some embodiments, the first nucleotide immediately internal to the cap of the RNA transcript is 2′-O-methylated. In some embodiments, the second nucleotide immediately internal to the cap of the RNA transcript is 2′-O-methylated. In some embodiments, the first and second nucleotides immediately internal to the cap of the RNA transcript are 2′-O-methylated.

In some embodiments, the RNA transcript is a non-capped transcript (e.g., a transcript produced from a mitochondrial gene). In some embodiments, the RNA transcript is a nuclear RNA that was capped but that has been decapped. In some embodiments, decapping of an RNA is catalyzed by the decapping complex, which may be composes of Dcp1 and Dcp2, e.g., that may compete with eIF-4E to bind the cap. In some embodiments, the process of RNA decapping involves hydrolysis of the 5′ cap structure on the RNA exposing a 5′ monophosphate. In some embodiments, this 5′ monophosphate is a substrate for the exonuclease XRN1. Accordingly, in some embodiments, an oligonucleotide that targets the 5′ region of an RNA may be used to stabilize (or restore stability) to a decapped RNA, e.g., protecting it from degradation by an exonuclease such as XRN1.

In some embodiments, in vitro transcription (e.g., performed via a T7 RNA polymerase or other suitable polymerase) may be used to produce an RNA transcript. In some embodiments transcription may be carried out in the presence of anti-reverse cap analog (ARCA) (TriLink Cat. #N-7003). In some embodiments, transcription with ARCA results in insertion of a cap (e.g., a cap analog (mCAP)) on the RNA in a desirable orientation.

In some embodiments, transcription is performed in the presence of one or more modified nucleotides (e.g., pseudouridine, 5-methylcytosine, etc.), such that the modified nucleotides are incorporated into the RNA transcript. It should be appreciated that any suitable modified nucleotide may be used, including, but not limited to, modified nucleotides that reduced immune stimulation, enhance translation and increase nuclease stability. Non-limiting examples of modified nucleotides that may be used include: 2′-amino-2′-deoxynucleotide, 2′-azido-2′-deoxynucleotide, 2′-fluoro-2′-deoxynucleotide, 2′-O-methyl-nucleotide, 2′ sugar super modifier, 2′-modified thermostability enhancer, 2′-fluoro-2′-deoxyadenosine-5′-triphosphate, 2′-fluoro-2′-deoxycytidine-5′-triphosphate, 2′-fluoro-2′-deoxyguanosine-5′-triphosphate, 2′-fluoro-2′-deoxyuridine-5′-triphosphate, 2′-O-methyladenosine-5′-triphosphate, 2′-O-methylcytidine-5′-triphosphate, 2′-O-methylguanosine-5′-triphosphate, 2′-O-methyluridine-5′-triphosphate, pseudouridine-5′-triphosphate, 2′-O-methylinosine-5′-triphosphate, 2′-amino-2′-deoxycytidine-5′-triphosphate, 2′-amino-2′-deoxyuridine-5′-triphosphate, 2′-azido-2′-deoxycytidine-5′-triphosphate, 2′-azido-2′-deoxyuridine-5′-triphosphate, 2′-O-methylpseudouridine-5′-triphosphate, 2′-O-methyl-5-methyluridine-5′-triphosphate, 2′-azido-2′-deoxyadenosine-5′-triphosphate, 2′-amino-2′-deoxyadenosine-5′-triphosphate, 2′-fluoro-thymidine-5′-triphosphate, 2′-azido-2′-deoxyguanosine-5′-triphosphate, 2′-amino-2′-deoxyguanosine-5′-triphosphate, and N4-methylcytidine-5′-triphosphate. In one embodiment, RNA degradation or processing can be reduced/prevented to elevate steady state RNA and, at least for protein-coding transcripts, protein levels. In some embodiments, a majority of degradation of RNA transcripts is done by exonucleases. In such embodiments, these enzymes start destroying RNA from either their 3′ or 5′ ends. By protecting the ends of the RNA transcripts from exonuclease enzyme activity, for instance, by hybridization of sequence-specific blocking oligonucleotides with proper chemistries for proper delivery, hybridization and stability within cells, RNA stability may be increase, along with protein levels for protein-coding transcripts.

In some embodiments, for the 5′ end, oligonucleotides may be used that are fully/partly complementary to 10-20 nts of the RNA 5′ end. In some embodiments, such oligonucleotides may have overhangs to form a hairpin (e.g., the 3′ nucleotide of the oligonucleotide can be, but not limited to, a C to interact with the mRNA 5′ cap's G nucleoside) to protect the RNA 5′ cap. In some embodiments, all nucleotides of an oligonucleotide may be complementary to the 5′ end of an RNA transcript, with or without few nucleotide overhangs to create a blunt or recessed 5′RNA-oligo duplex. In some embodiments, for the 3′ end, oligonucleotides may be partly complementary to the last several nucleotides of the RNA 3′ end, and optionally may have a poly(T)-stretch to protect the poly(A) tail from complete degradation (for transcripts with a poly(A)-tail). In some embodiments, similar strategies can be employed for other RNA species with different 5′ and 3′ sequence composition and structure (such as transcripts containing 3′ poly(U) stretches or transcripts with alternate 5′ structures). In some embodiments, oligonucleotides as described herein, including, for example, oligonucleotides with overhangs, may have higher specificity and sensitivity to their target RNA end regions compared to oligonucleotides designed to be perfectly complementary to RNA sequences, because the overhangs provide a destabilizing effect on mismatch regions and prefer binding in regions that are at the 5′ or 3′ ends of the RNAs. In some embodiments, oligonucleotides that protect the very 3′ end of the poly(A) tail with a looping mechanism (e.g., TTTTTTTTTTGGTTTTCC, SEQ ID NO: 458). In some embodiments, this latter approach may nonspecifically target all protein-coding transcripts. However, in some embodiments, such oligonucleotides, may be useful in combination with other target-specific oligos.

In some embodiments, methods provided herein involve the use of an oligonucleotide that comprises a region of complementarity that is complementary with the RNA transcript at a position at or near the first transcribed nucleotide of the RNA transcript. In some embodiments, an oligonucleotide (e.g., an oligonucleotide that stabilizes an RNA transcript) comprises a region of complementarity that is complementary with the RNA transcript (e.g., with at least 5 contiguous nucleotides) at a position that begins within 100 nucleotides, within 50 nucleotides, within 30 nucleotides, within 20 nucleotides, within 10 nucleotides or within 5 nucleotides of the 5′-end of the transcript. In some embodiments, an oligonucleotide (e.g., an oligonucleotide that stabilizes an RNA transcript) comprises a region of complementarity that is complementary with the RNA transcript (e.g., with at least 5 contiguous nucleotides of the RNA transcript) at a position that begins at the 5′-end of the transcript. In some embodiments, an oligonucleotide (e.g., an oligonucleotide that stabilizes an RNA transcript) comprises a region of complementarity that is complementary with an RNA transcript at a position within a region of the 5′ untranslated region (5′ UTR) of the RNA transcript spanning from the transcript start site to 50, 100, 150, 200, 250, 500 or more nucleotides upstream from a translation start site (e.g., a start codon, AUG, arising in a Kozak sequence of the transcript).

In some embodiments, an RNA transcript is poly-adenylated. Polyadenylation refers to the post-transcriptional addition of a polyadenosine (poly(A)) tail to an RNA transcript. Both protein-coding and non-coding RNA transcripts may be polyadenylated. Poly(A) tails contain multiple adenosines linked together through internucleoside linkages. In some embodiments, a poly(A) tail may contain 10 to 50, 25 to 100, 50 to 200, 150 to 250 or more adenosines. In some embodiments, the process of polyadenlyation involves endonucleolytic cleavage of an RNA transcript at or near its 3′-end followed by one by one addition of multiple adenosines to the transcript by a polyadenylate polymerase, the first of which adenonsines is added to the transcript at the 3′ cleavage site. Thus, often a polyadenylated RNA transcript comprises transcribed nucleotides (and possibly edited nucleotides) linked together through internucleoside linkages that are linked at the 3′ end to a poly(A) tail. The location of the linkage between the transcribed nucleotides and poly(A) tail may be referred to herein as, a “polyadenylation junction.” In some embodiments, endonucleolytic cleavage may occur at any one of several possible sites in an RNA transcript. In such embodiments, the sites may be determined by sequence motifs in the RNA transcript that are recognized by endonuclease machinery, thereby guiding the position of cleavage by the machinery. Thus, in some embodiments, polyadenylation can produce different RNA transcripts from a single gene, e.g., RNA transcripts have different polyadenylation junctions. In some embodiments, length of a poly(A) tail may determine susceptibility of the RNA transcript to enzymatic degradation by exonucleases with 3′-5′ processing activity. In some embodiments, oligonucleotides that target an RNA transcript at or near its 3′ end target a region overlapping a polyadenylation junction. In some embodiments, such oligonucleotides may have at least 3, 4, 5, 6, 7, 8, 9, 10, 11, 12 or more nucleotides that are complementary with the transcribed portion of the transcript (5′ to the junction). In some embodiments, it is advantageous to have a limited number of nucleotides (e.g., T, U) complementary to the polyA side of the junction. In some embodiments, having a limited number of nucleotides complementary to the polyA side of the junction it is advantageous because it reduces toxicity associated with cross hybridization of the oligonucleotide to the polyadenylation region of non-target RNAs in cells. In some embodiments, the oligonucleotide has only 1, 2, 3, 4, 5, or 6 nucleotides complementary to the poly A region.

In some embodiments, methods provided herein involve the use of an oligonucleotide that hybridizes with a target RNA transcript at or near its 3′ end and prevents or inhibits degradation of the RNA transcript by 3′-5′ exonucleases. For example, in some embodiments, RNA stabilization methods provided herein involve the use of an oligonucleotide that comprises a region of complementarity that is complementary with the RNA transcript at a position within 100 nucleotides, within 50 nucleotides, within 30 nucleotides, within 20 nucleotides, within 10 nucleotides, within 5 nucleotides of the last transcribed nucleotide of the RNA transcript. In a case where the RNA transcript is a polyadenylated transcript, the last transcribed nucleotide of the RNA transcript is the first nucleotide upstream of the polyadenylation junction. In some embodiments, RNA stabilization methods provided herein involve the use of an oligonucleotide that comprises a region of complementarity that is complementary with the RNA transcript at a position immediately adjacent to or overlapping the polyadenylation junction of the RNA transcript. In some embodiments, RNA stabilization methods provided herein involve the use of an oligonucleotide that comprises a region of complementarity that is complementary with the RNA transcript within the poly(A) tail.

### Particle Formulation, Delivery, and Dosing

Nucleic acids, e.g., stabilizing oligonucleotides and/or synthetic RNAs, described herein can be formulated with a particle for administration to a subject for treating a condition associated with decreased levels of expression of gene or instability or low stability of an RNA transcript that results in decreased levels of expression of a gene (e.g., decreased protein levels or decreased levels of functional RNAs, such as miRNAs, snoRNAs, lncRNAs, etc.). It should be understood that the formulations, compositions and methods can be practiced with any of the oligonucleotides and/or synthetic RNAs disclosed herein.

Any of the formulations, excipients, vehicles, etc. disclosed herein may be adapted or used to facilitate delivery of nucleic acids, e.g., stabilizing oligonucleotides and/or synthetic RNAs (e.g., linear or circularized synthetic RNAs), to a cell. Formulations, excipients, vehicles, etc. disclosed herein may be adapted or used to facilitate delivery of a nucleic acid to a cell in vitro or in vivo. Nucleic acids, e.g., stabilizing oligonucleotides and/or synthetic RNAs, may be formulated with a particle as described herein. For example, nucleic acids may be formulated with a nanoparticle, poly(lactic-co-glycolic acid) (PLGA) microsphere, lipidoid, lipoplex, liposome, polymer, carbohydrate (including simple sugars), cationic lipid, a fibrin gel, a fibrin hydrogel, a fibrin glue, a fibrin sealant, fibrinogen, thrombin, rapidly eliminated lipid nanoparticles (reLNPs) and combinations thereof. In some embodiments, a nucleic acid, e.g., stabilizing oligonucleotides and/or synthetic RNAs (e.g., linear or circularized synthetic RNAs), may be delivered to a cell gymnotically. In some embodiments, nucleic acids, e.g., stabilizing oligonucleotides and/or synthetic RNAs, may be conjugated with factors that facilitate delivery to cells. In some embodiments, a nucleic acid, e.g., stabilizing oligonucleotide and/or synthetic RNA, is conjugated with a carbohydrate, such as GalNac, or other targeting moiety.

In some aspects, a particle of the present disclosure is a nanoparticle, e.g., lipid nanoparticle. In some embodiments, a lipid nanoparticle may include one or more cationic lipids, one or more non-cationic lipids, one or more conjugated lipids that inhibits aggregation of particles, or a combination thereof. Such lipids can be used alone or in combination. In some embodiments, a lipid nanoparticle comprises more than one cationic lipid, more than one non-cationic lipid, more than one conjugated lipid, or a combination thereof. Exemplary components that may be present in the formulations and methods of the present disclosure are described below.

In some embodiments, a particle of the present disclosure includes a cationic lipid. As used herein, the term “cationic lipid” refers to a positively charged lipid. In some embodiments, cationic lipids can have certain features including a head group, one or more hydrophobic tails, and a linker between the head group and the one or more tails. The head group may include an amine. Under certain conditions, the amine nitrogen can be a site of positive charge. The cationic lipid for use in accordance with the present disclosure may be any appropriate cationic lipid for particle formulation, e.g., nanoparticle formulation, e.g., lipid nanoparticle formulation. For example, without limitation, the cationic lipid may be N,N-dioleyl-N,N-dimethylammonium chloride (DODAC), N,N-distearyl-N,N-dimethylammonium bromide (DDAB), N—(I-(2,3-dioleoyloxy)propyl)-N,N,N-trimethylammonium chloride (DOTAP), N—(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino) acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino) ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)—N,N-dimethyl-2,2-di((9Z,12Z)-octadeca-9, 12-dienyl)tetrahydro-3aH-cyclopenta[d][1,3]dioxol-5-amine (ALN100), (6Z,9Z,28Z,31Z)-heptatriaconta-6,9,28,31-tetraen-19-yl 4-(dimethylamino) butanoate (MC3), 1, l′-(2-(4-(2-((2-(bis(2-hydroxydodecyl)amino)ethyl) (2-hydroxydodecyl)amino)ethyl) piperazin-1-yl)ethylazanediyl)didodecan-2-ol (Tech Gl), 2,2-Dilinoleyl-4-dimethylaminoethyl-[1,3]-dioxolane, β-L-arginyl-2, 3-L-diaminopropionic acid-N-palmityl-N-oleylamide trihydrochloride, N′,N′-dioctadecyl-N-4, 8-diaza-10-aminodecanoylglycine amide[71], 1,2-dilinoleyloxy-3-dimethylaminopropane, DLin-KC2-DMA, amino lipid 2,2-dilinoleyl-4-dimethylaminoethyl-[1,3]-dioxolane (DLin-KC2-DMA, 1), 1,2-distearloxy-/V,N-dimethylaminopropane (DSDMA), dilinoleylmethyl-4-dimethylaminobutyrate (DLin-MC3-DMA), DLin-D-DMA, C12-200, 98N12-5, (20Z,23Z)—N,N-dimethylnonacosa-20,23-dien-10-amine, (17Z,20Z)—N,N-dimemylhexacosa-17,20-dien-9-amine, (1Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimethyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimethylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyleptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, (13Z,16Z)—N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimeth-yl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propa-n-2-amine, S—N,N-dimethyl-1-[(9Z,12Z)-octadeca-9,12-dien-1-yloxy]-3-(octy-loxy)propan-2-amine, 1-{2-[(9Z,12Z)-octadeca-9,12-dien-1-yloxy]-1-Roctyloxy)methyl]ethyl}pyrro-lidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azet-idine, (2S)-1-(hexyloxy)-N,N-dimethyl-3-[(9Z,12Z)-octadeca-9,12-dien-1-ylo-xy]propan-2-amine, (2S)-1-(heptyloxy)-N,N-dimethyl-3-[(9Z,12Z)-octadeca-9,12-dien-1-yloxy]pr-opan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-am-ine; (2S)—N,N-dimethyl-1-[(6Z,9Z,12Z)-octadeca-6,9,12-trien-1-yloxy]-3-(o-ctyloxy)propan-2-amine, (2S)-1-[(11Z,14Z)-icosa-11,14-dien-1-yloxy]-N,N-dimethyl-3-(pentyloxy)propa-n-2-amine, (2S)-1-(hexyloxy)-3-[(11Z,14Z)-icosa-11,14-dien-1-yloxy]-N,N-di-methylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)pr-opan-2-amine, (2S)-1-[(13Z,16Z)-docosa-13,16-dien-1-yloxy]-3-(hexyloxy)-N,N-dimethylpro-pan-2-amine, (2S)-1-[(13Z)-docos-13-en-1-yloxy]-3-(hexyloxy)-N,N-dimethylpropan-2-amin-e, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H (1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-di-en-1-yloxy]propan-2-amine, N,N-dimethyl-1-(octyloxy)-3-({8-[(1S,2S)-2-{[(1R,2R)-2-pentylcyclopropyl]-methyl}cyclopropyl]octyl}oxy)propan-2-amine, N,N-dimethyl-1-{[8-(2-oclylcyclopropyl)octyl]oxy}-3-(octyloxy)propan-2-am-ine and (11E,20Z,23Z)—N,N-dimethylnonacosa-11,20,2-trien-10-amine, 5-carboxyspermylglycine dioctaoleoylamide (“DOGS”), dipalmitoylphosphatidylethanolamine 5-carboxyspermyl-amide (“DPPES”), 1,2-dimyristyloxypropyl-3-dimethyl-hydroxy ethyl ammonium bromide (DMRIE), DMRIE-HP, Lipofectamine (DOSPA), 3b-(N-(N′,N′-dimethylaminoethane)-carbamoyl) cholesterol (“DC-Choi”), N-(1,2-dimyhstyloxyprop-3-yl)-N,N-dimethyl-N-hydroxyethyl ammonium bromide (“DMRIE”), 1,2-Dioleoyl-3-dimethylammonium-propane (“DODAP”), DMDMA, cationic lipid-based transfection reagents TransIT-TKO, LIPOFECTIN, Lipofectamine, OLIGOFECTAMINE or DHARMAFECT, DSDMA, DODMA, DLinDMA, DLenDMA, gamma-DLenDMA, DLin-K-DMA, DLin-K-C2-DMA (also known as DLin-C2K-DMA, XTC2, and C2K), DLin-K-C3-DM A, DLin-K-C4-DMA, DLen-C2K-DMA, y-DLen-C2K-DMA, DLin-M-C2-DMA (also known as MC2), DLin-M-C3-DMA (also known as MC3) and (DLin-MP-DMA) (also known as 1-B1 1) or a mixture thereof. In some embodiments, the cationic lipid may comprise from about 20 mol percent to about 50 mol percent or about 40 mol percent of the total lipid present in the particle.

The non-cationic lipid may be any appropriate non-cationic lipid for particle, e.g., nanoparticle, e.g., lipid nanoparticle, formulation. For example, the non-cationic lipid may be an anionic lipid or a neutral lipid. Anionic lipids suitable for use in particles of the present disclosure include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids. In some embodiments, the anionic lipid is 1,2-dioleoyl-sn-glycero-3-phospho-(1′-rac-glycerol).

A neutral lipid generally refers to a lipid which exists either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. In some embodiments, the neutral lipid component is a lipid having two acyl groups, (e.g., diacylphosphatidylcholine and diacylphosphatidylethanolamine). Lipids having a variety of acyl chain groups of varying chain length and degree of saturation are available or may be isolated or synthesized by well-known techniques. In some embodiments, lipids contain saturated fatty acids with carbon chain lengths in the range of C₁₀ to C₂₀. In some embodiments, lipids with mono or diunsaturated fatty acids with carbon chain lengths in the range of C₁₀ to C₂₀ may be used. Additionally, lipids having mixtures of saturated and unsaturated fatty acid chains can be used. For example, a neutral lipid for use in accordance with the present disclosure is DOPE, DSPC, POPC, DPPC or any related phosphatidylcholine. In some embodiments, a neutral lipid may be composed of sphingomyelin, dihydrosphingomyeline, or phospholipids with other head groups, such as serine and inositol. In some embodiments, the neutral lipid is 1,2-dioleoyl-sn-glycero-3-phosphatidylcholine.

In some embodiments, exemplary non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids or a mixture thereof. In some embodiments, the anionic lipid is 1,2-dioleoyl-sn-glycero-3-phospho-(1′-rac-glycerol). In some embodiments, the neutral lipid is 1,2-dioleoyl-sn-glycero-3-phosphatidylcholine.

Particles of the present disclosure may include a lipid that inhibits and/or reduces aggregation of particles during formation. Examples of lipids that reduce aggregation of particles during formation include polyethylene glycol (PEG)-modified lipids, monosialoganglioside Gm1, and polyamide oligomers (“PAO”) such as (described in U.S. Pat. No. 6,320,017). Other compounds with uncharged, hydrophilic, steric-barrier moieties, which prevent aggregation during formulation, like PEG, Gm1 or ATTA, can also be coupled to lipids for use as in accordance with the disclosure. Exemplary ATTA-lipids are described, e.g., in U.S. Pat. No. 6,320,017, and exemplary PEG-lipid conjugates are described, e.g., in U.S. Pat. Nos. 5,820,873, 5,534,499 and 5,885,613.

In some embodiments, the conjugated lipid is a PEG lipid. In some embodiments, the PEG lipid is a PEG-diacylglycerol (DAG), a PEG-dialkyloxypropyl (DAA), a PEG-phospholipid, a PEG-ceramide (Cer), or a mixture thereof. As a non-limiting example, PLGA may be conjugated to a lipid-terminating PEG forming PLGA-DSPE-PEG, PEG lipid is selected from PEG-c-DOMG and 1,2-Dimyristoyl-sn-glycerol, methoxypolyethylene Glycol (PEG-DMG), 1,2-Distearoyl-sn-glycerol, methoxypolyethylene Glycol (PEG-DSG), PEG-c-DOMG, 1,2-Distearoyl-sn-glycerol, methoxypolyethylene glycol (PEG-DSG) 1,2-Dipalmitoyl-sn-glycerol, methoxypolyethylene glycol (PEG-DPG), PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides, cationic PEG lipids, polyoxazoline (POZ)-lipid conjugates, polyamide oligomers (e.g., ATTA-lipid conjugates), and mixtures thereof. In some embodiments, the PEG is a PEG-dilauryloxypropyl (C12), a PEG-dimyristyloxypropyl (C14), a PEG-dipalmityloxypropyl (C16), a PEG-distearyloxypropyl (C18), PEG-c-DOMG, PEG-DMG, or a mixture thereof.

In some embodiments, the conjugated lipid that prevents aggregation of particles may be from 0% to about 20%, e.g., about 1% to about 15%, e.g., about 2% of the total lipid present in the particle (by mole percent of lipids). In some embodiments, the nucleic acid-lipid particle further includes cholesterol at, e.g., about 10 mol percent to about 60 mol percent or about 48 mol percent of the total lipid present in the particle.

In some embodiments, the conjugated PEG lipid is coupled to the surface of the lipid nanoparticle. In some embodiments, the PEG lipid is coupled to the surface of the lipid nanoparticle by an oxime linkage. In some embodiments, the conjugated PEG lipid is susceptible to decomposition in an acidic environment.

In some embodiments, the lipid nanoparticles of the present disclosure have a mean diameter of about 50 nm to about 150 nm, e.g., about 60 nm to about 130 nm, about 70 nm to about 110 nm, about 60 nm to about 80 nm. In some embodiments, the lipid nanoparticles of the present disclosure have a mean diameter of about 20-50 nm. In some embodiments, the lipid nanoparticles of the present disclosure have a mean diameter of about 30 nm. In some embodiments, the particle size is at least 30 nm, at least 40 nm, at least 50 nm, at least 60 nm, at least 70 nm, at least 80 nm, at least 90 nm, at least 100 nm, at least 110 nm, or at least 120 nm. In some embodiments, the particle size range is about at least 50 nm to about at least 110 nm, about at least 60 nm to about at least 100 nm, or about at least 50 nm to about at least 80 nm.

In some embodiments, nucleic acids, e.g., stabilizing oligonucleotide and/or synthetic RNA, are encapsulated in the particle of the present disclosure. In some embodiments, encapsulated nucleic acids are resistant in aqueous solution to degradation with a nuclease. In some embodiments, the nucleic acid, e.g., stabilizing oligonucleotide and/or synthetic RNA, encapsulated in the particles is not significantly degraded after exposure to serum or a nuclease assay that would significantly degrade free nucleic acid. Nuclease degradation of nucleic acids may be determined by an Oligreen® assay (Invitrogen Corporation, Carlsbad, CA), which is an ultra-sensitive fluorescent nucleic acid stain for quantitating oligonucleotides and single-stranded DNA in solution. In some embodiments, nucleic acids, e.g., stabilizing oligonucleotide and/or synthetic RNA, are at least 50%, at least 60%, at least 70%, at least 80%, at least 90%, at least 95%, or at least 97% encapsulated in the particle.

In one embodiment, the lipid to drug ratio (mass/mass ratio; w/w ratio) (e.g., lipid to dsRNA ratio) will be in the range of from about 1:1 to about 50:1, from about 1:1 to about 25:1, from about 10:1 to about 14:1, from about 3:1 to about 15:1, from about 4:1 to about 10:1, from about 5:1 to about 9:1, or about 6:1 to about 9:1.

In some embodiments, a particle of the present disclosure comprises a polymer. In some embodiments, the polymer comprises a layer of a hydrogel or surgical sealant. In some embodiments, the polymer is PLGA, ethylene vinyl acetate, poloxamer, GELSITE®, or a combination thereof. In some embodiments, a particle of the present disclosure comprises a fibrin sealant.

In some embodiments, it is desirable to precondense the nucleic acid, e.g., stabilizing oligonucleotide and/or synthetic RNA, into the core of a particle described herein. In some embodiments, a heper cationic polymer, such as protamine is included in the formulation. In some embodiments, protamine interacts with nucleic acid to form a negatively charged compact core.

In some embodiments, calcium phosphate is included in a formulation described herein. In some embodiments, hyaluronic acid is included in a formulation described herein. In some embodiments, polyglutamate is included in a formulation described herein

In some embodiments, a particle of the disclosure comprises a lipoprotein or lipoprotein mimetic, or fragment thereof. In some embodiments, the lipoprotein is HDL, LDL, or a combination thereof.

In some aspects, a particle in accordance with the present disclosure comprises a lipidoid. The synthesis of lipidoids has been extensively described and formulations containing these compounds are particularly suited for delivery of modified nucleic acid molecules or mmRNA (see Mahon et al., Bioconjug Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties). Complexes, micelles, liposomes or particles can be prepared containing a lipidoid and therefore, can result in an effective delivery of the nucleic acids, e.g., stabilizing oligonucleotides and/or synthetic RNAs, of the present disclosure. In some embodiments, the lipidoid is penta[3-(1-laurylaminopropionyl)]-triethylenetetramine hydrochloride (TETA-5LAP; 98N12-5), C12-200, MD1, or a combination thereof. The lipidoid formulations can include particles comprising, e.g., 3 or 4 or more components in addition to stabilizing oligonucleotide and/or synthetic RNA. As an example, formulations with certain lipidoids, include, but are not limited to, 98N12-5 and may contain 42% lipidoid, 48% cholesterol and 10% PEG (C14 alkyl chain length). As another example, formulations with certain lipidoids, include, but are not limited to, C12-200 and may contain 50% lipidoid, 10% disteroylphosphatidyl choline, 38.5% cholesterol, and 1.5% PEG-DMG.

In some embodiments, a formulation comprises a rapidly eliminated lipid nanoparticle (reLNP). In some embodiments, a reLNP comprises a reLNP lipid, a fusogenic lipid, cholesterol and a PEG lipid at a molar ratio of 50:10:38.5:1.5 (reLNP lipid:fusogenic lipid:cholesterol:PEG lipid). The fusogenic lipid may be DSPC and the PEG lipid may be PEG-c-DOMG. The reLNP lipid may be DLin-DMA with an internal or terminal ester or DLin-MC3-DMA with an internal or terminal ester. In some embodiments, the total lipid to modified mRNA weight ratio is between 10:1 and 30:1.

The formulations may conveniently be presented in unit dosage form and may be prepared by any methods well known in the art of pharmacy. The amount of active ingredient (e.g., an oligonucleotide or compound of the disclosure) which can be combined with a carrier material to produce a single dosage form will vary depending upon the host being treated, the particular mode of administration, e.g., intradermal or inhalation. The amount of active ingredient which can be combined with a carrier material to produce a single dosage form will generally be that amount of the compound which produces a therapeutic effect, e.g. tumor regression.

Pharmaceutical formulations of this disclosure can be prepared according to any method known to the art for the manufacture of pharmaceuticals. Such formulations can contain sweetening agents, flavoring agents, coloring agents and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated nucleic acid, e.g., oligonucleotide and/or synthetic RNA, and particle composition can assume a variety of states. In some examples, the composition is at least partially crystalline, uniformly crystalline, and/or anhydrous (e.g., less than 80, 50, 30, 20, or 10% water). In another example, a nucleic acid, e.g., oligonucleotide and/or synthetic RNA, is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition) as described herein. Generally, a nucleic acid, e.g., oligonucleotide and/or synthetic RNA, composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

A nucleic acid, e.g., oligonucleotide and/or synthetic RNA, preparation can be formulated or administered (together or separately) in combination with another agent, e.g., another therapeutic agent or an agent that stabilizes an oligonucleotide, e.g., a protein that complexes with oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

In one embodiment, an formulation comprising an oligonucleotide includes another oligonucleotide, e.g., a second oligonucleotide that modulates expression of a second gene or a second oligonucleotide that modulates expression of the first gene. Still other preparation can include at least 3, 5, ten, twenty, fifty, or a hundred or more different oligonucleotide species. Such oligonucleotides can mediated gene expression with respect to a similar number of different genes. In one embodiment, an oligonucleotide preparation includes at least a second therapeutic agent (e.g., an agent other than an oligonucleotide).

### Ligand Modified Particles

In some aspects, a particle of the present disclosure may be conjugated to a targeting ligand that targets the particle to a particular ligand binding molecule, e.g., a ligand binding molecule present in a particular tissue or on the surface of a particular cell. Any suitable ligand may be used, including protein, lipid, polynucleotide, polysaccharide, or other molecules. In some embodiments, without limitation, the ligand is an antibody or antigen binding fragment thereof, a small molecule, a drug, a chemical, an ion, an aptamer, an oligonucleotide, a single stranded oligonucleotide, DNA, RNA, a peptide, a protein, a neuropeptide, or a neurotransmitter.

In some embodiments, a targeting ligand may be conjugated to a particle directly, e.g., through a direct covalent linkage. However, in some embodiments, a targeting ligand may be conjugated to a particle indirectly, e.g., via another molecule, such as a linker or adaptor molecule. As used herein, the term “particle linker” refers to any linker that is conjugated to a particle. In some embodiments, a targeting ligand is conjugated to a particle (e.g., a lipid nanoparticle) via a particle linker. Any suitable particle linker may be used. In some embodiments, a particle linker is conjugated to a targeting ligand. In some embodiments, the particle linker is a hetero-bifunctional molecule, such as polyethylene glycol (PEG) (e.g., as described herein and/or in U.S. Pat. Nos. 5,820,873, 5,534,499 and/or 5,885,613, incorporated by reference herein, in their entireties). In some embodiments, the particle linker is a monosialoganglioside (Gm1/GM1/GM-1), a polyamide oligomer (e.g., PAO e.g., ATTA, e.g., N-(ω-azido-octa-(14′-amino-3′,6′,9′,12′-tetraoxatetradecanoyl))-1,2-distearoyl-sn-glycero-3-phosphoethanolamine (ATTA-DPSE)) (e.g., as described in U.S. Pat. Nos. 6,320,017 and/or 6,586,559, incorporated by reference herein, in their entireties). GM1 is a member of the ganglio series of gangliosides which contain one sialic acid residue. GM1 impacts neuronal plasticity and repair mechanisms and the release of neurotrophins in the brain. Polyamides are cell permeable, synthetic oligomers. In some embodiments, the particle is conjugated to a mixture of particle linkers. In some embodiments, the particle is conjugated to a targeting ligand. In some embodiments, the particle is conjugated to a mixture of targeting ligands. In some embodiments, the particle is conjugated to a mixture of particle linkers and targeting ligands.

In some embodiments the particle is a nanoparticle. In some embodiments, the nanoparticle is a polymeric nanoparticle, micelle, dendrimer, or liposome as described herein.

In some aspects, a nucleic acid complex (e.g., containing a stabilizing oligonucleotide and synthetic mRNA) or an oligonucleotide (e.g., stabilizing oligonucleotide) formulation disclosed herein can be targeted to a specific cell or tissue type of interest via a ligand conjugated to a particle or via a ligand conjugated to a particle linker, which is conjugated to the particle, wherein the ligand targets a receptor of the cell or tissue of interest. For example, in some embodiments, a ligand can be mannose, which can be conjugated directly to a particle or conjugated to a particle linker that is conjugated to the particle, and the mannose can bind to a mannose receptor on the surface of a macrophage or dendritic cell. In other embodiments, a ligand can be glutamate, which can be conjugated directly to a particle or conjugated to a particle linker that is conjugated to the particle, and the glutamate can bind to a glutamate receptor on the surface of a central nervous system (CNS) cell, e.g. neuron or glial cell, or dendrite of a postsynaptic cell.

In other embodiments, a ligand can be PD-L1 or PD-L2, which can be conjugated directly to a particle or conjugated to a particle linker that is conjugated to the particle, and the PD-L1 or PD-L2 ligand can bind to a PD-1 receptor on the surface of an antigen presenting cell (APC), e.g., a dendritic cell, or a T cell. However, other appropriate ligands may be used for targeting to a particular receptor.

In some embodiments, without limitation, the ligand is a ligand of a PD-1 receptor (e.g., PD-L1 or PD-L2), a CTLA4 receptor, a glutamate receptor, mannose receptor, natural killer group 2D (NKG2D) receptor, chemokine receptor (e.g., CCL19 or CCL21 ligands of CCR7 receptor, or CXCL9 or CXCL10 ligands of CXCR3 receptor), pattern recognition receptor (PRR), Toll-like receptor (TLR), killer activated receptor (KAR), killer inhibitor receptor (KIR), complement receptor, Fc receptor, B cell receptor, or T cell receptor, acetylcholine (ACh) receptor (e.g., nicotine acetylcholine receptor (nAChR) or muscarinic or metabotropic acetylcholine receptor (mAChR)), metabotropic receptor (e.g., G-protein coupled receptor or G-protein coupled receptor linked to a second messenger or second messenger system), ionotropic receptor (e.g., N-methyl D-aspartate (NMDA), amino methyl propionic acid (AMPA), Kainite), transferrin receptor, adenosine receptor (AR), 5-HT receptor (5-HT₁, 5-HT₂, or 5-HT₃), insulin receptor, γ-aminobutyric acid, 4-aminobutanoic acid (GABA) receptor, dopamine receptor (including D1-type and D2-type), or adrenergic receptor (α or β). In some embodiments, the ligand is glutamate, aspartate, mannose, N-acetylglucosamine, fucose, an NKG2D ligand (e.g. MICA or MICB), B7-H1 (PD-L1), B7-DC (PD-L2), CD80, CD86, delta-like ligand 4 (DLL4), transferrin, insulin, Apo-protein, IGF-1, leptin, NMDA, AMPA, GABA, bicuculline, picrotoxinin, dopamine, nicotine, muscarine, acetylcholine, adenosine, serotonin, phenylephrine, adenylate cyclase, or melatonin. In some embodiments, a ligand facilitates blood brain barrier transport (e.g., a transferrin ligand may be used to target a transferrin binding receptor in cells of the brain vasculature to facilitate blood brain barrier transport). In some embodiments, the ligand facilitates blood brain barrier transport via receptor-mediated transcytosis.

In some embodiments, without limitation, the ligand receptor is a PD-1 receptor, CTLA4 receptor, glutamate receptor, mannose receptor, natural killer group 2D (NKG2D) receptor, chemokine receptor, pattern recognition receptor (PRR), Toll-like receptor (TLR), killer activated receptor (KAR), killer inhibitor receptor (KIR), complement receptor, Fc receptor, B cell receptor, or T cell receptor, acetylcholine (ACh) receptor (e.g., nicotine acetylcholine receptor (nAChR) or muscarinic or metabotropic acetylcholine receptor (mAChR)), metabotropic receptor (e.g., G-protein coupled receptor or G-protein coupled receptor linked to a second messenger or second messenger system), ionotropic receptor (e.g., N-methyl D-aspartate (NMDA), amino methyl propionic acid (AMPA), Kainite), transferrin receptor, adenosine receptor (AR), a 5-HT receptor (5-HT₁, 5-HT₂, or 5-HT₃), insulin receptor, γ-aminobutyric acid, 4-aminobutanoic acid (GABA) receptor, dopamine receptor (including D1-type and D2-type), or adrenergic receptor (a or B).

In some embodiments the cell or tissue to be targeted is in vitro. In some embodiments the cell or tissue to be targeted is in vivo. In some embodiments, without limitation, the cell or tissue that has the ligand receptor is a cell or tissue of the immune system or central nervous system (CNS). In some embodiments, the tissue is a tissue of the central nervous system, muscle, or immune system. In some embodiments, the cell is an antigen presenting cell (APC), e.g., dendritic cell, macrophage, or B cell, a T cell, cytotoxic T cell, natural killer (NK) cell, T helper (Tₕ) cell, neuron, projection neuron, interneuron, glial cell, microglial cell, astrocyte, oligodendrocyte, ependymal cell, radial glial cell, or dendrite. In some embodiments, the cell is from a cell line that was derived from a normal, transformed or immortalized cell, e.g., a normal, transformed or immortalized immune cell or central nervous system cell.

Other cells may be targeted by using appropriate cell targeting ligands. For example, in some embodiments, cells to be targeted are stem cells, e.g., embryonic stem cells, mesenchymal stem cells, hematopoietic stem cells, cancer stem cells, stromal cells etc. In some embodiments, cells to be targeted are epithelial cells (e.g., corneal epithelial cells, mammary epithelial cells, etc.), fibroblasts, myoblasts (e.g., human skeletal myoblasts), keratinocytes, endothelial cells (e.g., vascular endothelial cells), neural cells, smooth muscle cells, marrow cells, bone cells (e.g., osteocytes, osteoblasts, osteoclasts) hematopoietic cells (e.g., monocytes, macrophages, megakaryocytes, etc.) or placental cells.

### Oligonucleotides

Oligonucleotides provided herein are useful for stabilizing RNAs by inhibiting or preventing degradation of the RNAs (e.g., degradation mediated by exonucleases). Such oligonucleotides may be referred to as “stabilizing oligonucleotides.” In some embodiments, oligonucleotides hybridize at a 5′ and/or 3′ region of the RNA resulting in duplex regions that stabilize the RNA by preventing degradation by exonucleotides having single strand processing activity.

In some embodiments, oligonucleotides are provided having a region complementary with at least 5 consecutive nucleotides of a 5′ region of an RNA transcript. In some embodiments, oligonucleotides are provided having a region complementary with at least 5 consecutive nucleotides of a 3′-region of an RNA transcript. In some embodiments, oligonucleotides are provided having a first region complementary with at least 5 consecutive nucleotides of a 5′ region of an RNA transcript, and a second region complementary with at least 5 consecutive nucleotides of a 3′-region of an RNA transcript.

In some embodiments, oligonucleotides are provided having a region complementary with at least 5 consecutive nucleotides of the 5′-UTR of an mRNA transcript. In some embodiments, oligonucleotides are provided having a region complementary with at least 5 consecutive nucleotides of the 3′-UTR, poly(A) tail, or overlapping the polyadenylation junction of the mRNA transcript. In some embodiments, oligonucleotides are provided having a first region complementary with at least 5 consecutive nucleotides of the 5′-UTR of an mRNA transcript, and a second region complementary with at least 5 consecutive nucleotides of the 3′-UTR, poly(A) tail, or overlapping the polyadenylation junction of the mRNA transcript.

In some embodiments, oligonucleotides are provided that have a region of complementarity that is complementary to an RNA transcript in proximity to the 5′-end of the RNA transcript. In such embodiments, the nucleotide at the 3′-end of the region of complementarity of the oligonucleotides may be complementary with the RNA transcript at a position that is within 10 nucleotides, within 20 nucleotides, within 30 nucleotides, within 40 nucleotides, within 50 nucleotides, or within 100 nucleotides, within 200 nucleotides, within 300 nucleotides, within 400 nucleotides or more of the transcription start site of the RNA transcript.

In some embodiments, oligonucleotides are provided that have a region of complementarity that is complementary to an RNA transcript in proximity to the 3′-end of the RNA transcript. In such embodiments, the nucleotide at the 3′-end and/or 5′ end of the region of complementarity may be complementary with the RNA transcript at a position that is within 10 nucleotides, within 20 nucleotides, within 30 nucleotides, within 40 nucleotides, within 50 nucleotides, within 100 nucleotides, within 200 nucleotides, within 300 nucleotides, within 400 nucleotides or more of the 3′-end of the RNA transcript. In some embodiments, if the target RNA transcript is polyadenylated, the nucleotide at the 3′-end of the region of complementarity of the oligonucleotide may be complementary with the RNA transcript at a position that is within 10 nucleotides, within 20 nucleotides, within 30 nucleotides, within 40 nucleotides, within 50 nucleotides, within 100 nucleotides, within 200 nucleotides, within 300 nucleotides, within 400 nucleotides or more of polyadenylation junction. In some embodiments, an oligonucleotide that targets a 3′ region of an RNA comprises a region of complementarity that is a stretch of pyrimidines (e.g., 4 to 10 or 5 to 15 thymine nucleotides) complementary with adenines.

In some embodiments, combinations of 5′ targeting and 3′ targeting oligonucleotides are contacted with a target RNA. In some embodiments, the 5′ targeting and 3′ targeting oligonucleotides a linked together via a linker (e.g., a stretch of nucleotides non-complementary with the target RNA). In some embodiments, the region of complementarity of the 5′ targeting oligonucleotide is complementary to a region in the target RNA that is at least 2, 5, 10, 20, 50, 100, 500, 1000, 5000, 10000 nucleotides upstream from the region of the target RNA that is complementary to the region of complementarity of the 3′ end targeting oligonucleotide.

In some embodiments, oligonucleotides are provided that have the general formula 5′-X₁—X₂-3′, in which X₁ has a region of complementarity that is complementary with an RNA transcript (e.g., with at least 5 contiguous nucleotides of the RNA transcript). In some embodiments, the nucleotide at the 3′-end of the region of complementary of X₁ may be complementary with a nucleotide in proximity to the transcription start site of the RNA transcript. In some embodiments, the nucleotide at the 3′-end of the region of complementary of X₁ may be complementary with a nucleotide that is present within 1, 2, 3, 4, 5, 6, 7, 8, 9, or 10 nucleotides of the transcription start site of the RNA transcript. In some embodiments, the nucleotide at the 3′-end of the region of complementary of X₁ may be complementary with the nucleotide at the transcription start site of the RNA transcript.

In some embodiments, X₁ comprises 5 to 10 nucleotides, 5 to 15 nucleotides, 5 to 25 nucleotides, 10 to 25 nucleotides, 5 to 20 nucleotides, or 15 to 30 nucleotides. In some embodiments, X₁ comprises 5, 6, 7, 8, 9, 10, 11, 12, 13, 14, 15, 16, 17, 18, 19, 20, 25, 30 or more nucleotides. In some embodiments, the region of complementarity of X₁ may be complementary with at least 4, at least 5, at least 6, at least 7, at least 8, at least 9, or at least 10 contiguous nucleotides of the RNA transcript. In some embodiments, the region of complementarity of X₁ may be complementary with 4, 5, 6, 7, 8, 9, 10, 11,12, 13, 14, 15, 16, 17, 18, 19, 20 or more contiguous nucleotides of the RNA transcript.

In some embodiments, X₂ is absent. In some embodiments, X₂ comprises 1 to 10, 1 to 20 nucleotides, 1 to 25 nucleotides, 5 to 20 nucleotides, 5 to 30 nucleotides, 5 to 40 nucleotides, or 5 to 50 nucleotides. In some embodiments, X₂ comprises 5, 6, 7, 8, 9, 10, 11, 12, 13, 14, 15, 16, 17, 18, 19, 20, 25, 30, 35, 40, 45, 50 or more nucleotides. In some embodiments, X₂ comprises a region of complementarity complementary with at least 4, at least 5, at least 6, at least 7, at least 8, at least 9, or at least 10 contiguous nucleotides of the RNA transcript. In some embodiments, X₂ comprises a region of complementarity complementary with 4, 5, 6, 7, 8, 9, 10, 11,12, 13, 14, 15, 16, 17, 18, 19, 20 or more contiguous nucleotides of the RNA transcript.

In some embodiments, the RNA transcript has a 7-methylguanosine cap at its 5′-end. In some embodiments, the nucleotide at the 3′-end of the region of complementary of X₁ is complementary with the nucleotide of the RNA transcript that is immediately internal to the 7-methylguanosine cap or in proximity to the cap (e.g., with 10 nucleotides of the cap). In some embodiments, at least the first nucleotide at the 5′-end of X₂ is a pyrimidine complementary with guanine (e.g., a cytosine or analogue thereof). In some embodiments, the first and second nucleotides at the 5′-end of X₂ are pyrimidines complementary with guanine. Thus, in some embodiments, at least one nucleotide at the 5′-end of X₂ is a pyrimidine that may form stabilizing hydrogen bonds with the 7-methylguanosine of the cap.

In some embodiments, X₂ forms a stem-loop structure. In some embodiments, X₂ comprises the formula 5′-Y₁—Y₂—Y₃-3′, in which X₂ forms a stem-loop structure having a loop region comprising the nucleotides of Y₂ and a stem region comprising at least two contiguous nucleotides of Y₁ hybridized with at least two contiguous nucleotides of Y₃. In some embodiments, the stem region comprises 1-6, 1-5, 2-5, 1-4, 2-4 or 2-3 nucleotides. In some embodiments, the stem region comprises LNA nucleotides. In some embodiments, the stem region comprises 1-6, 1-5, 2-5, 1-4, 2-4 or 2-3 LNA nucleotides. In some embodiments, Y₁ and Y₃ independently comprise 2 to 10 nucleotides, 2 to 20 nucleotides, 2 to 25 nucleotides, or 5 to 20 nucleotides. In some embodiments, Y₁ and Y₃ independently comprise 2, 3, 4, 5, 6, 7, 8, 9, 10, 11, 12, 13, 14, 15, 16, 17, 18, 19, 20, 25 or more nucleotides. In some embodiments, Y₂ comprises 3 to 10 nucleotides, 3 to 15 nucleotides, 3 to 25 nucleotides, or 5 to 20 nucleotides. In some embodiments, Y₂ comprises 3, 4, 5, 6, 7, 8, 9, 10, 11, 12, 13, 14, 15, 16, 17, 18, 19, 20, 25 or more nucleotides. In some embodiments, Y₂ comprises 2-8, 2-7, 2-6, 2-5, 3-8, 3-7, 3-6, 3-5 or 3-4 nucleotides. In some embodiments, Y₂ comprises at least one DNA nucleotide. In some embodiments, the nucleotides of Y₂ comprise at least one (e.g., 1, 2, 3, 4, 5, 6, 7, 8, 9, 10, 11, 12, 13, 14, 15, 16, 17, 18, 19, 20, 25 or more adenines). In some embodiments, Y₃ comprises 1-5, 1-4, 1-3 or 1-2 nucleotides following the 3′ end of the stem region. In some embodiments, the nucleotides of Y₃ following the 3′ end of the stem region are DNA nucleotides. In some embodiments, Y₃ comprises a pyrimidine complementary with guanine (e.g., cytosine or an analogue thereof). In some embodiments, Y₃ comprises one or more (e.g., two) pyrimidines complementary with guanine at a position following the 3′-end of the stem region (e.g., 1, 2, 3 or more nucleotide after the 3′-end of the stem region). Thus, in embodiments where the RNA transcript is capped, Y₃ may have a pyrimidine that forms stabilizing hydrogen bonds with the 7-methylguanosine of the cap.

In some embodiments, X₁ and X₂ are complementary with non-overlapping regions of the RNA transcript. In some embodiments, X₁ comprises a region complementary with a 5′ region of the RNA transcript and X₂ comprises a region complementary with a 3′ region of the RNA transcript. For example, if the RNA transcript is polyadenylated, X₂ may comprise a region of complementarity that is complementary with the RNA transcript at a region within 100 nucleotides, within 50 nucleotides, within 25 nucleotides or within 10 nucleotides of the polyadenylation junction of the RNA transcript. In some embodiments, X₂ comprises a region of complementarity that is complementary with the RNA transcript immediately adjacent to or overlapping the polyadenylation junction of the RNA transcript. In some embodiments, X₂ comprises at least 2 consecutive pyrimidine nucleotides (e.g., 5 to 15 pyrimidine nucleotides) complementary with adenine nucleotides of the poly(A) tail of the RNA transcript.

In some embodiments, oligonucleotides are provided that comprise the general formula 5′-X₁—X₂-3′, in which X₁ comprises at least 2 nucleotides that form base pairs with adenine (e.g., thymidines or uridines or analogues thereof); and X₂ comprises a region of complementarity that is complementary with at least 3 contiguous nucleotides of a poly-adenylated RNA transcript, wherein the nucleotide at the 5′-end of the region of complementary of X₂ is complementary with the nucleotide of the RNA transcript that is immediately internal to the poly-adenylation junction of the RNA transcript. In such embodiments, X₁ may comprises 2 to 10, 2 to 20, 5 to 15 or 5 to 25 nucleotides and X₂ may independently comprises 2 to 10, 2 to 20, 5 to 15 or 5 to 25 nucleotides.

In some embodiments, compositions are provided that comprise a first oligonucleotide comprising at least 5 nucleotides (e.g., of 5 to 25 nucleotides) linked through internucleoside linkages, and a second oligonucleotide comprising at least 5 nucleotides (e.g., of 5 to 25 nucleotides) linked through internucleoside linkages, in which the first oligonucleotide is complementary with at least 5 consecutive nucleotides in proximity to the 5′-end of an RNA transcript and the second oligonucleotide is complementary with at least 5 consecutive nucleotides in proximity to the 3′-end of an RNA transcript. In some embodiments, the 5′ end of the first oligonucleotide is linked with the 3′ end of the second oligonucleotide. In some embodiments, the 3′ end of the first oligonucleotide is linked with the 5′ end of the second oligonucleotide. In some embodiments, the 5′ end of the first oligonucleotide is linked with the 5′ end of the second oligonucleotide. In some embodiments, the 3′ end of the first oligonucleotide is linked with the 3′ end of the second oligonucleotide.

In some embodiments, the first oligonucleotide and second oligonucleotide are joined by a linker. The term “linker” generally refers to a chemical moiety that is capable of covalently linking two or more oligonucleotides. In some embodiments, a linker is resistant to cleavage in certain biological contexts, such as in a mammalian cell extract, such as an endosomal extract. However, in some embodiments, at least one bond comprised or contained within the linker is capable of being cleaved (e.g., in a biological context, such as in a mammalian extract, such as an endosomal extract), such that at least two oligonucleotides are no longer covalently linked to one another after bond cleavage. In some embodiments, the linker is not an oligonucleotide having a sequence complementary with the RNA transcript. In some embodiments, the linker is an oligonucleotide (e.g., 2-8 thymines). In some embodiments, the linker is a polypeptide. Other appropriate linkers may also be used, including, for example, linkers disclosed in International Patent Application Publication WO 2013/040429 A1, published on Mar. 21, 2013, and entitled MULTIMERIC ANTISENSE OLIGONUCLEOTIDES, and in International Patent Application Publication WO 2014043544 A1, published on Mar. 20, 2014, and entitled MULTIMERIC ANTISENSE OLIGONUCLEOTIDES. The contents of these publications relating to linkers are incorporated herein by reference in their entireties.

An oligonucleotide may have a region of complementarity with a target RNA transcript (e.g., a mammalian mRNA transcript) that has less than a threshold level of complementarity with every sequence of nucleotides, of equivalent length, of an off-target RNA transcript. For example, an oligonucleotide may be designed to ensure that it does not have a sequence that targets RNA transcripts in a cell other than the target RNA transcript. The threshold level of sequence identity may be 50%, 60%, 70%, 80%, 85%, 90%, 95%, 99% or 100% sequence identity.

An oligonucleotide may be complementary to RNA transcripts encoded by homologues of a gene across different species (e.g., a mouse, rat, rabbit, goat, monkey, etc.) In some embodiments, oligonucleotides having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of an oligonucleotide is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a target RNA. In some embodiments, the region of complementarity is complementary with at least 8 consecutive nucleotides of a target RNA.

Complementary, as the term is used in the art, refers to the capacity for precise pairing between two nucleotides. For example, if a nucleotide at a certain position of an oligonucleotide is capable of hydrogen bonding with a nucleotide at a corresponding position of a target RNA, then the nucleotide of the oligonucleotide and the nucleotide of the target RNA are complementary to each other at that position. The oligonucleotide and target RNA are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the oligonucleotide and target RNA. For example, if a base at one position of an oligonucleotide is capable of hydrogen bonding with a base at the corresponding position of a target RNA, then the bases are considered to be complementary to each other at that position. 100% complementarity is not required.

An oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a target RNA. In some embodiments an oligonucleotide may contain 1, 2 or 3 base mismatches compared to the portion of the consecutive nucleotides of the target RNA. In some embodiments an oligonucleotide may have up to 3 mismatches over 15 bases, or up to 2 mismatches over 10 bases.

In some embodiments, a complementary nucleic acid sequence need not be 100% complementary to that of its target to be specifically hybridizable. In some embodiments, an oligonucleotide for purposes of the present disclosure is specifically hybridizable with a target RNA when hybridization of the oligonucleotide to the target RNA prevents or inhibits degradation of the target RNA, and when there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, an oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80 or more nucleotides in length. In some embodiments, the oligonucleotide is 8 to 50, 10 to 30, 9 to 20, 15 to 30 or 8 to 80 nucleotides in length.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa.

In some embodiments, an oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches. Contiguous runs of three or more Gs or Cs may not be preferable in some embodiments. Accordingly, in some embodiments, the oligonucleotide does not comprise a stretch of three or more guanosine nucleotides.

An oligonucleotide may have a sequence that is has greater than 30% G-C content, greater than 40% G-C content, greater than 50% G-C content, greater than 60% G-C content, greater than 70% G-C content, or greater than 80% G-C content. An oligonucleotide may have a sequence that has up to 100% G-C content, up to 95% G-C content, up to 90% G-C content, or up to 80% G-C content. In some embodiments, GC content of an oligonucleotide is preferably between about 30-60%.

It is to be understood that any oligonucleotide provided herein can be excluded.

In some embodiments, it has been found that oligonucleotides disclosed herein may increase stability of a target RNA by at least about 50% (e.g. 150% of normal or 1.5 fold), or by about 2 fold to about 5 fold. In some embodiments, stability (e.g., stability in a cell) may be increased by at least about 15 fold, 20 fold, 30 fold, 40 fold, 50 fold or 100 fold, or any range between any of the foregoing numbers. In some embodiments, increased mRNA stability has been shown to correlate to increased protein expression. Similarly, in some embodiments, increased stability of non-coding positively correlates with increased activity of the RNA.

It is understood that any reference to uses of oligonucleotides or other molecules throughout the description contemplates use of the oligonucleotides or other molecules in preparation of a pharmaceutical composition or medicament for use in the treatment of condition or a disease associated with decreased levels or activity of a RNA transcript. Thus, as one nonlimiting example, this aspect of the disclosure includes use of oligonucleotides or other molecules in the preparation of a medicament for use in the treatment of disease, wherein the treatment involves posttranscriptionally altering protein and/or RNA levels in a targeted manner.

Methods for identifying transcript start sites and polyadenylation junctions are known in the art and may be used in selecting oligonucleotides that specifically bind to these regions for stabilizing RNA transcripts. In some embodiments, 3′ end oligonucleotides may be designed by identifying RNA 3′ ends using quantitative end analysis of poly-A tails. In some embodiments, 5′ end oligonucleotides may be designed by identifying 5′ start sites using Cap analysis gene expression (CAGE). Appropriate methods are disclosed, for example, in Ozsolak et al. Comprehensive Polyadenylation Site Maps in Yeast and Human Reveal Pervasive Alternative Polyadenylation. Cell. Volume 143, Issue 6, 2010, Pages 1018-1029; Shiraki, T, et al., Cap analysis gene expression for high-throughput analysis of transcriptional starting point and identification of promoter usage. Proc Natl Acad Sci USA. 100 (26): 15776-81. 2003 Dec. 23; and Zhao, X, et al., (2011). Systematic Clustering of Transcription Start Site Landscapes. PLOS ONE (Public Library of Science) 6 (8): e23409, the contents of each of which are incorporated herein by reference. Other appropriate methods for identifying transcript start sites and polyadenylation junctions may also be used, including, for example, RNA-Paired-end tags (PET) (See, e.g., Ruan X, Ruan Y. Methods Mol Biol. 2012; 809:535-62); use of standard EST databases; RACE combined with microarray or sequencing, PAS-Seq (See, e.g., Peter J. Shepard, et al., RNA. 2011 April; 17(4): 761-772); and 3P-Seq (See, e.g., Calvin H. Jan, Nature. 2011 Jan. 6; 469(7328): 97-101; and others.

Exemplary oligonucleotides targeting the 5′ and/or 3′ ends of RNAs are provided in Example 2 and Tables 3-13.

### Oligonucleotide Modifications

In some embodiments, oligonucleotides are provided with chemistries suitable for delivery, hybridization and stability within cells to target and stabilize RNA transcripts. Furthermore, in some embodiments, oligonucleotide chemistries are provided that are useful for controlling the pharmacokinetics, biodistribution, bioavailability and/or efficacy of the oligonucleotides. Accordingly, oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not induce substantial cleavage or degradation of the target RNA; do not cause substantially complete cleavage or degradation of the target RNA; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; and may have improved endosomal exit.

Oligonucleotides that are designed to interact with RNA to modulate gene expression are a distinct subset of base sequences from those that are designed to bind a DNA target (e.g., are complementary to the underlying genomic DNA sequence from which the RNA is transcribed).

Any of the oligonucleotides disclosed herein may be linked to one or more other oligonucleotides disclosed herein by a linker, e.g., a cleavable linker.

Oligonucleotides of the disclosure can be stabilized against nucleolytic degradation such as by the incorporation of a modification, e.g., a nucleotide modification. For example, nucleic acid sequences of the disclosure include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). As another example, the nucleic acid sequence can include at least one 2′-O-methyl-modified nucleotide, and in some embodiments, all of the nucleotides include a 2′-O-methyl modification. In some embodiments, the nucleic acids are “locked,” e.g., comprise nucleic acid analogues in which the ribose ring is “locked” by a methylene bridge connecting the 2′-O atom and the 4′-C atom.

Any of the modified chemistries or formats of oligonucleotides described herein can be combined with each other, and that one, two, three, four, five, or more different types of modifications can be included within the same molecule.

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States patent or patent application Publications: U.S. Pat. Nos. 7,399,845, 7,741,457, 8,022,193, 7,569,686, 7,335,765, 7,314,923, 7,335,765, and 7,816,333, US20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often an oligonucleotide has one or more nucleotide analogues. For example, an oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. An oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues. The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group.

The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ligands of the asialoglycoprotein receptor (ASGPR), such as GalNac, or dynamic polyconjugates and variants thereof at its 5′ or 3′ end.

Preferably an oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the disclosure may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, an oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (e.g., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′ alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41 (14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

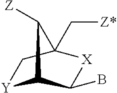

where X and Y are independently selected among the groups —O—,
—S—, —N(H)—, N(R)—, —CH₂— or —CH— (if part of a double bond),
—CH₂—O—, —CH₂—S—, —CH₂—N(H)—, —CH₂—N(R)—, —CH₂—CH₂— or —CH₂—CH— (if part of a double bond),
—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligonucleotides described herein comprises at least one LNA unit according any of the formulas

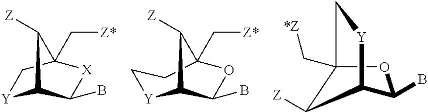

wherein Y is —O—, —S—, —NH—, or N(R^{H}); Z and Z* are independently selected among an internucleoside linkage, a terminal group or a protecting group; B constitutes a natural or non-natural nucleotide base moiety, and R^{H} is selected from hydrogen and C1-4-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the disclosure comprises internucleoside linkages selected from -0-P(O)₂—O—, —O—P(O,S)—O—, -0-P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, -0-PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Other examples of LNA units are shown below:

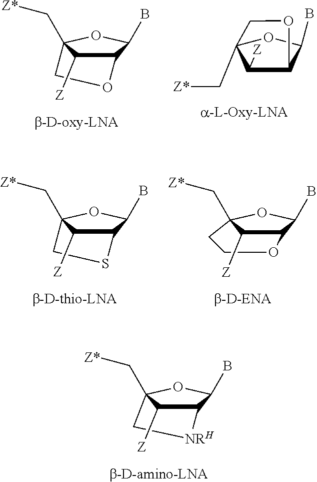

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃ OCH₃, OCH₃O(CH₂)ₙ CH₃, O(CH₂)ₙ NH₂ or O(CH₂)ₙ CH₃ where n is from 1 to about 10; C1 to C10 lower alkyl, alkoxyalkoxy, substituted lower alkyl, alkaryl or aralkyl; Cl; Br; CN; CF₃; OCF₃; O—, S—, or N-alkyl; O—, S—, or N-alkenyl; SOCH₃; SO₂ CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)](Martin et al, Helv. Chim. Acta, 1995, 78, 486). Other preferred modifications include 2′-methoxy (2′-O—CH3), 2′-propoxy (2′-OCH₂ CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino) adenine, 2-(imidazolylalkyl) adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl) adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the disclosure. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928 and 5,688,941, each of which is herein incorporated by reference.

These moieties or conjugates can include conjugate groups covalently bound to functional groups such as primary or secondary hydroxyl groups. Conjugate groups of the disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

In some embodiments, oligonucleotide modification include modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of an oligonucleotide. In some embodiments, an oligonucleotide comprises a biotin moiety conjugated to the 5′ nucleotide.

In some embodiments, an oligonucleotide comprises locked nucleic acids (LNA), ENA modified nucleotides, 2′-O-methyl nucleotides, or 2′-fluoro-deoxyribonucleotides. In some embodiments, an oligonucleotide comprises alternating deoxyribonucleotides and 2′-fluoro-deoxyribonucleotides. In some embodiments, an oligonucleotide comprises alternating deoxyribonucleotides and 2′-O-methyl nucleotides. In some embodiments, an oligonucleotide comprises alternating deoxyribonucleotides and ENA modified nucleotides. In some embodiments, an oligonucleotide comprises alternating deoxyribonucleotides and locked nucleic acid nucleotides. In some embodiments, an oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, an oligonucleotide comprises phosphorothioate internucleotide linkages. In some embodiments, an oligonucleotide comprises phosphorothioate internucleotide linkages between at least two nucleotides. In some embodiments, an oligonucleotide comprises phosphorothioate internucleotide linkages between all nucleotides.

It should be appreciated that an oligonucleotide can have any combination of modifications as described herein.

The oligonucleotide may comprise a nucleotide sequence having one or more of the following modification patterns.
(a) (X)Xxxxxx, (X)xXxxxx, (X)xxXxxx, (X)xxxXxx, (X)xxxxXx and (X)xxxxxX,
(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXX, (X)xxxXxX and (X)xxxxXX,
(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXXX, (X)XxXXxx, (X)XxxXXx (X)XxxxXX, (X)xXxXXx, (X)xXxxXX, (X)xxXxXX, (X)xXxXxX and (X)XxXxXx,
(d) (X)xxXXX, (X)xXxXXX, (X)xXXxXX, (X)xXXXXX, (X)xXXXXx, (X)XxxXXXX, (X)XxXxXX, (X)XxXXXX, (X)XxXXx, (X)XXxxXX, (X)XXxXxX, (X)XXXXXx, (X)XXXxxX, (X)XXXxXx, and (X)XXXXxx,
(e) (X)xXXXXX, (X)XxXXXX, (X)XXxXXX, (X)XXXxXX, (X)XXXXXX and (X)XXXXXx, and
(f) XXXXXX, XxXXXXX, XXXXXXX, XXXXXXX, XXXXXXX, XXXXXXX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

Exemplary oligonucleotide modifications are also provided in Example 2 and Table 14.

### RNA and RNA Transcripts

In some embodiments, an RNA transcript targeted by an oligonucleotide disclosed herein is an RNA transcript of a eukaryotic cell. In some embodiments, an RNA transcript targeted by an oligonucleotide disclosed herein is an RNA transcript of a cell of a vertebrate. In some embodiments, an RNA transcript targeted by an oligonucleotide disclosed herein is an RNA transcript of a cell of a mammal, e.g., a primate cell, mouse cell, rat cell, or human cell. In some embodiments, an RNA transcript targeted by an oligonucleotide disclosed herein is an RNA transcript of a cardiomyocyte. In some embodiments, an RNA transcript targeted by an oligonucleotide disclosed herein is an RNA transcribed in the nucleus of a cell. In some embodiments, an RNA transcript targeted by an oligonucleotide disclosed herein is an RNA transcribed in a mitochondrion of a cell. In some embodiments, an RNA transcript targeted by an oligonucleotide disclosed herein is an RNA transcript transcribed by a RNA polymerase II enzyme.

In some embodiments, an RNA transcript targeted by an oligonucleotide disclosed herein is an mRNA expressed from a gene, including, but not limited to, ABCA1, APOA1, ATP2A2, BDNF, FXN, HBA2, HBB, HBD, HBE1, HBG1, HBG2, SMN, UTRN, PTEN, MECP2, and FOXP3, ABCA4, ABCB11, ABCB4, ABCG5, ABCG8, ADIPOQ, ALB, APOE, BCL2L11, BRCA1, CD274, CEP290, CFTR, EPO, F7, F8, FLI1, FMR1, FNDC5, GCH1, GCK, GLPIR, GRN, HAMP, HPRT1, IDO1, IGF1, IL10, IL6, KCNMA1, KCNMB1, KCNMB2, KCNMB3, KCNMB4, KLF1, KLF4, LDLR, MSX2, MYBPC3, NANOG, NF1, NKX2-1, NKX2-1-AS1, PAH, PTGS2, RB1, RPS14, RPS19, SCARB1, SERPINF1, SIRT1, SIRT6, SMAD7, ST7, STAT3, TSIX, and XIST. RNA transcripts for these and other genes may be selected or identified experimentally, for example, using RNA sequencing (RNA-Seq) or other appropriate methods. RNA transcripts may also be selected based on information in public databases such as in UCSC, Ensembl and NCBI genome browsers and others. Non-limiting examples of RNA transcripts for certain genes are listed in Table 1.

TABLE 1
Non-limiting examples of RNA transcripts for certain genes

| GENE | | | |
|---|---|---|---|
| SYMBOL | MRNA | SPECIES | GENE NAME |
| ABCA1 | NM_013454 | Mus | ATP-binding cassette, sub-family A (ABC1), |
| | | musculus | member 1 |
| ABCA1 | NM_005502 | Homo | ATP-binding cassette, sub-family A (ABC1), |
| | | sapiens | member 1 |
| ABCA4 | NM_007378 | Mus | ATP-binding cassette, sub-family A (ABC1), |
| | | musculus | member 4 |
| ABCA4 | NM_000350 | Homo | ATP-binding cassette, sub-family A (ABC1), |
| | | sapiens | member 4 |
| ABCB11 | NM_003742 | Homo | ATP-binding cassette, sub-family B |
| | | sapiens | (MDR/TAP), member 11 |
| ABCB11 | NM_021022 | Mus | ATP-binding cassette, sub-family B |
| | | musculus | (MDR/TAP), member 11 |
| ABCB4 | NM_018850 | Homo | ATP-binding cassette, sub-family B |
| | | sapiens | (MDR/TAP), member 4 |
| ABCB4 | NM_000443 | Homo | ATP-binding cassette, sub-family B |
| | | sapiens | (MDR/TAP), member 4 |
| ABCB4 | NM_018849 | Homo | ATP-binding cassette, sub-family B |
| | | sapiens | (MDR/TAP), member 4 |
| ABCB4 | NM_008830 | Mus | ATP-binding cassette, sub-family B |
| | | musculus | (MDR/TAP), member 4 |
| ABCG5 | NM_022436 | Homo | ATP-binding cassette, sub-family G (WHITE), |
| | | sapiens | member 5 |
| ABCG5 | NM_031884 | Mus | ATP-binding cassette, sub-family G (WHITE), |
| | | musculus | member 5 |
| ABCG8 | NM_026180 | Mus | ATP-binding cassette, sub-family G (WHITE), |
| | | musculus | member 8 |
| ABCG8 | NM_022437 | Homo | ATP-binding cassette, sub-family G (WHITE), |
| | | sapiens | member 8 |
| ADIPOQ | NM_009605 | Mus | adiponectin, C1Q and collagen domain |
| | | musculus | containing |
| ADIPOQ | NM_004797 | Homo | adiponectin, C1Q and collagen domain |
| | | sapiens | containing |
| ALB | NM_000477 | Homo | albumin |
| | | sapiens | |
| ALB | NM_009654 | Mus | albumin |
| | | musculus | |
| APOA1 | NM_000039 | Homo | apolipoprotein A-I |
| | | sapiens | |
| APOA1 | NM_009692 | Mus | apolipoprotein A-I |
| | | musculus | |
| APOE | NM_009696 | Mus | apolipoprotein E |
| | | musculus | |
| APOE | XM_001724655 | Homo | hypothetical LOC100129500; apolipoprotein E |
| | | sapiens | |
| APOE | XM_001722911 | Homo | hypothetical LOC100129500; apolipoprotein E |
| | | sapiens | |
| APOE | XM_001724653 | Homo | hypothetical LOC100129500; apolipoprotein E |
| | | sapiens | |
| APOE | NM_000041 | Homo | hypothetical LOC100129500; apolipoprotein E |
| | | sapiens | |
| APOE | XM_001722946 | Homo | hypothetical LOC100129500; apolipoprotein E |
| | | sapiens | |
| ATP2A2 | NM_009722 | Mus | ATPase, Ca++ transporting, cardiac muscle, |
| | | musculus | slow twitch 2 |
| ATP2A2 | NM_001110140 | Mus | ATPase, Ca++ transporting, cardiac muscle, |
| | | musculus | slow twitch 2 |
| ATP2A2 | NM_001135765 | Homo | ATPase, Ca++ transporting, cardiac muscle, |
| | | sapiens | slow twitch 2 |
| ATP2A2 | NM_170665 | Homo | ATPase, Ca++ transporting, cardiac muscle, |
| | | sapiens | slow twitch 2 |
| ATP2A2 | NM_001681 | Homo | ATPase, Ca++ transporting, cardiac muscle, |
| | | sapiens | slow twitch 2 |
| BCL2L11 | NM_006538 | Homo | BCL2-like 11 (apoptosis facilitator) |
| | | sapiens | |
| BCL2L11 | NM_207002 | Homo | BCL2-like 11 (apoptosis facilitator) |
| | | sapiens | |
| BCL2L11 | NM_138621 | Homo | BCL2-like 11 (apoptosis facilitator) |
| | | sapiens | |
| BCL2L11 | NM_207680 | Mus | BCL2-like 11 (apoptosis facilitator) |
| | | musculus | |
| BCL2L11 | NM_207681 | Mus | BCL2-like 11 (apoptosis facilitator) |
| | | musculus | |
| BCL2L11 | NM_009754 | Mus | BCL2-like 11 (apoptosis facilitator) |
| | | musculus | |
| BDNF | NM_001143816 | Homo | brain-derived neurotrophic factor |
| | | sapiens | |
| BDNF | NM_001143815 | Homo | brain-derived neurotrophic factor |
| | | sapiens | |
| BDNF | NM_001143814 | Homo | brain-derived neurotrophic factor |
| | | sapiens | |
| BDNF | NM_001143813 | Homo | brain-derived neurotrophic factor |
| | | sapiens | |
| BDNF | NM_001143812 | Homo | brain-derived neurotrophic factor |
| | | sapiens | |
| BDNF | NM_001143806 | Homo | brain-derived neurotrophic factor |
| | | sapiens | |
| BDNF | NM_001143811 | Homo | brain-derived neurotrophic factor |
| | | sapiens | |
| BDNF | NM_001143805 | Homo | brain-derived neurotrophic factor |
| | | sapiens | |
| BDNF | NM_001143810 | Homo | brain-derived neurotrophic factor |
| | | sapiens | |
| BDNF | NM_001709 | Homo | brain-derived neurotrophic factor |
| | | sapiens | |
| BDNF | NM_170735 | Homo | brain-derived neurotrophic factor |
| | | sapiens | |
| BDNF | NM_170734 | Homo | brain-derived neurotrophic factor |
| | | sapiens | |
| BDNF | NM_170733 | Homo | brain-derived neurotrophic factor |
| | | sapiens | |
| BDNF | NM_170732 | Homo | brain-derived neurotrophic factor |
| | | sapiens | |
| BDNF | NM_170731 | Homo | brain-derived neurotrophic factor |
| | | sapiens | |
| BDNF | NM_001143809 | Homo | brain-derived neurotrophic factor |
| | | sapiens | |
| BDNF | NM_001143807 | Homo | brain-derived neurotrophic factor |
| | | sapiens | |
| BDNF | NM_001143808 | Homo | brain-derived neurotrophic factor |
| | | sapiens | |
| BDNF | NM_007540 | Mus | brain derived neurotrophic factor |
| | | musculus | |
| BDNF | NM_001048141 | Mus | brain derived neurotrophic factor |
| | | musculus | |
| BDNF | NM_001048142 | Mus | brain derived neurotrophic factor |
| | | musculus | |
| BDNF | NM_001048139 | Mus | brain derived neurotrophic factor |
| | | musculus | |
| BRCA1 | NM_009764 | Mus | breast cancer 1 |
| | | musculus | |
| BRCA1 | NM_007296 | Homo | breast cancer 1, early onset |
| | | sapiens | |
| BRCA1 | NM_007300 | Homo | breast cancer 1, early onset |
| | | sapiens | |
| BRCA1 | NM_007297 | Homo | breast cancer 1, early onset |
| | | sapiens | |
| BRCA1 | NM_007303 | Homo | breast cancer 1, early onset |
| | | sapiens | |
| BRCA1 | NM_007298 | Homo | breast cancer 1, early onset |
| | | sapiens | |
| BRCA1 | NM_007302 | Homo | breast cancer 1, early onset |
| | | sapiens | |
| BRCA1 | NM_007299 | Homo | breast cancer 1, early onset |
| | | sapiens | |
| BRCA1 | NM_007304 | Homo | breast cancer 1, early onset |
| | | sapiens | |
| BRCA1 | NM_007294 | Homo | breast cancer 1, early onset |
| | | sapiens | |
| BRCA1 | NM_007305 | Homo | breast cancer 1, early onset |
| | | sapiens | |
| BRCA1 | NM_007295 | Homo | breast cancer 1, early onset |
| | | sapiens | |
| CD274 | NM_014143 | Homo | CD274 molecule |
| | | sapiens | |
| CD274 | NM_021893 | Mus | CD274 antigen |
| | | musculus | |
| CEP290 | NM_025114 | Homo | centrosomal protein 290 kDa |
| | | sapiens | |
| CEP290 | NM_146009 | Mus | centrosomal protein 290 |
| | | musculus | |
| CFTR | NM_000492 | Homo | cystic fibrosis transmembrane conductance |
| | | sapiens | regulator (ATP-binding cassette sub-family C, |
| | | | member 7) |
| CFTR | NM_021050 | Mus | cystic fibrosis transmembrane conductance |
| | | musculus | regulator homolog |
| EPO | NM_000799 | Homo | erythropoietin |
| | | sapiens | |
| EPO | NM_007942 | Mus | erythropoietin |
| | | musculus | |
| F7 | NM_000131 | Homo | coagulation factor VII (serum prothrombin |
| | | sapiens | conversion accelerator) |
| F7 | NM_019616 | Homo | coagulation factor VII (serum prothrombin |
| | | sapiens | conversion accelerator) |
| F7 | NM_010172 | Mus | coagulation factor VII |
| | | musculus | |
| F8 | NM_019863 | Homo | coagulation factor VIII, procoagulant |
| | | sapiens | component |
| F8 | NM_000132 | Homo | coagulation factor VIII, procoagulant |
| | | sapiens | component |
| F8 | NM_001161373 | Mus | coagulation factor VIII |
| | | musculus | |
| F8 | NM_001161374 | Mus | coagulation factor VIII |
| | | musculus | |
| F8 | NM_007977 | Mus | coagulation factor VIII |
| | | musculus | |
| FLI1 | NM_002017 | Homo | Friend leukemia virus integration 1 |
| | | sapiens | |
| FLI1 | NM_001167681 | Homo | Friend leukemia virus integration 1 |
| | | sapiens | |
| FLI1 | NM_008026 | Mus | Friend leukemia integration 1 |
| | | musculus | |
| FMR1 | NM_008031 | Mus | fragile X mental retardation syndrome 1 |
| | | musculus | homolog |
| FMR1 | NM_002024 | Homo | fragile X mental retardation 1 |
| | | sapiens | |
| FNDC5 | NM_001171941 | Homo | fibronectin type III domain containing 5 |
| | | sapiens | |
| FNDC5 | NM_153756 | Homo | fibronectin type III domain containing 5 |
| | | sapiens | |
| FNDC5 | NM_001171940 | Homo | fibronectin type III domain containing 5 |
| | | sapiens | |
| FNDC5 | NM_027402 | Mus | fibronectin type III domain containing 5 |
| | | musculus | |
| FOXP3 | NM_054039 | Mus | forkhead box P3 |
| | | musculus | |
| FOXP3 | NM_001114377 | Homo | forkhead box P3 |
| | | sapiens | |
| FOXP3 | NM_014009 | Homo | forkhead box P3 |
| | | sapiens | |
| FXN | NM_001161706 | Homo | frataxin |
| | | sapiens | |
| FXN | NM_181425 | Homo | frataxin |
| | | sapiens | |
| FXN | NM_000144 | Homo | frataxin |
| | | sapiens | |
| FXN | NM_008044 | Mus | frataxin |
| | | musculus | |
| GCH1 | NM_008102 | Mus | GTP cyclohydrolase 1 |
| | | musculus | |
| GCH1 | NM_000161 | Homo | GTP cyclohydrolase 1 |
| | | sapiens | |
| GCH1 | NM_001024070 | Homo | GTP cyclohydrolase 1 |
| | | sapiens | |
| GCH1 | NM_001024071 | Homo | GTP cyclohydrolase 1 |
| | | sapiens | |
| GCH1 | NM_001024024 | Homo | GTP cyclohydrolase 1 |
| | | sapiens | |
| GCK | NM_010292 | Mus | glucokinase |
| | | musculus | |
| GCK | NM_000162 | Homo | glucokinase (hexokinase 4) |
| | | sapiens | |
| GCK | NM_033508 | Homo | glucokinase (hexokinase 4) |
| | | sapiens | |
| GCK | NM_033507 | Homo | glucokinase (hexokinase 4) |
| | | sapiens | |
| GLP1R | NM_021332 | Mus | glucagon-like peptide 1 receptor; similar to |
| | | musculus | glucagon-like peptide-1 receptor |
| GLP1R | XM_001471951 | Mus | glucagon-like peptide 1 receptor; similar to |
| | | musculus | glucagon-like peptide-1 receptor |
| GLP1R | NM_002062 | Homo | glucagon-like peptide 1 receptor |
| | | sapiens | |
| GRN | NM_002087 | Homo | granulin |
| | | sapiens | |
| GRN | NM_008175 | Mus | granulin |
| | | musculus | |
| HAMP | NM_021175 | Homo | hepcidin antimicrobial peptide |
| | | sapiens | |
| HAMP | NM_032541 | Mus | hepcidin antimicrobial peptide |
| | | musculus | |
| HBA2 | NM_000517 | Homo | hemoglobin, alpha 2; hemoglobin, alpha 1 |
| | | sapiens | |
| HBA2 | NM_000558 | Homo | hemoglobin, alpha 2; hemoglobin, alpha 1 |
| | | sapiens | |
| HBB | NM_000518 | Homo | hemoglobin, beta |
| | | sapiens | |
| HBB | XM_921413 | Mus | hemoglobin beta chain complex |
| | | musculus | |
| HBB | XM_903245 | Mus | hemoglobin beta chain complex |
| | | musculus | |
| HBB | XM_921395 | Mus | hemoglobin beta chain complex |
| | | musculus | |
| HBB | XM_903244 | Mus | hemoglobin beta chain complex |
| | | musculus | |
| HBB | XM_903246 | Mus | hemoglobin beta chain complex |
| | | musculus | |
| HBB | XM_909723 | Mus | hemoglobin beta chain complex |
| | | musculus | |
| HBB | XM_921422 | Mus | hemoglobin beta chain complex |
| | | musculus | |
| HBB | XM_489729 | Mus | hemoglobin beta chain complex |
| | | musculus | |
| HBB | XM_903242 | Mus | hemoglobin beta chain complex |
| | | musculus | |
| HBB | XM_903243 | Mus | hemoglobin beta chain complex |
| | | musculus | |
| HBB | XM_921400 | Mus | hemoglobin beta chain complex |
| | | musculus | |
| HBD | NM_000519 | Homo | hemoglobin, delta |
| | | sapiens | |
| HBE1 | NM_005330 | Homo | hemoglobin, epsilon 1 |
| | | sapiens | |
| HBG1 | NM_000559 | Homo | hemoglobin, gamma A |
| | | sapiens | |
| HBG2 | NM_000184 | Homo | hemoglobin, gamma G |
| | | sapiens | |
| HPRT1 | NM_000194 | Homo | hypoxanthine phosphoribosyltransferase 1 |
| | | sapiens | |
| IDO1 | NM_008324 | Mus | indoleamine 2,3-dioxygenase 1 |
| | | musculus | |
| IDO1 | NM_002164 | Homo | indoleamine 2,3-dioxygenase 1 |
| | | sapiens | |
| IGF1 | NM_001111284 | Homo | insulin-like growth factor 1 (somatomedin C) |
| | | sapiens | |
| IGF1 | NM_001111285 | Homo | insulin-like growth factor 1 (somatomedin C) |
| | | sapiens | |
| IGF1 | NM_001111283 | Homo | insulin-like growth factor 1 (somatomedin C) |
| | | sapiens | |
| IGF1 | NM_000618 | Homo | insulin-like growth factor 1 (somatomedin C) |
| | | sapiens | |
| IGF1 | NM_001111274 | Mus | insulin-like growth factor 1 |
| | | musculus | |
| IGF1 | NM_010512 | Mus | insulin-like growth factor 1 |
| | | musculus | |
| IGF1 | NM_184052 | Mus | insulin-like growth factor 1 |
| | | musculus | |
| IGF1 | NM_001111276 | Mus | insulin-like growth factor 1 |
| | | musculus | |
| IGF1 | NM_001111275 | Mus | insulin-like growth factor 1 |
| | | musculus | |
| IL10 | NM_000572 | Homo | interleukin 10 |
| | | sapiens | |
| IL10 | NM_010548 | Mus | interleukin 10 |
| | | musculus | |
| IL6 | NM_031168 | Mus | interleukin 6 |
| | | musculus | |
| IL6 | NM_000600 | Homo | interleukin 6 (interferon, beta 2) |
| | | sapiens | |
| KCNMA1 | NM_002247 | Homo | potassium large conductance calcium- |
| | | sapiens | activated channel, subfamily M, alpha |
| | | | member 1 |
| KCNMA1 | NM_001161352 | Homo | potassium large conductance calcium- |
| | | sapiens | activated channel, subfamily M, alpha |
| | | | member 1 |
| KCNMA1 | NM_001014797 | Homo | potassium large conductance calcium- |
| | | sapiens | activated channel, subfamily M, alpha |
| | | | member 1 |
| KCNMA1 | NM_001161353 | Homo | potassium large conductance calcium- |
| | | sapiens | activated channel, subfamily M, alpha |
| | | | member 1 |
| KCNMA1 | NM_010610 | Mus | potassium large conductance calcium- |
| | | musculus | activated channel, subfamily M, alpha |
| | | | member 1 |
| KCNMB1 | NM_031169 | Mus | potassium large conductance calcium- |
| | | musculus | activated channel, subfamily M, beta |
| | | | member 1 |
| KCNMB1 | NM_004137 | Homo | potassium large conductance calcium- |
| | | sapiens | activated channel, subfamily M, beta |
| | | | member 1 |
| KCNMB2 | NM_028231 | Mus | potassium large conductance calcium- |
| | | musculus | activated channel, subfamily M, beta |
| | | | member 2 |
| KCNMB2 | NM_005832 | Homo | potassium large conductance calcium- |
| | | sapiens | activated channel, subfamily M, beta |
| | | | member 2 |
| KCNMB2 | NM_181361 | Homo | potassium large conductance calcium- |
| | | sapiens | activated channel, subfamily M, beta |
| | | | member 2 |
| KCNMB3 | NM_171829 | Homo | potassium large conductance calcium- |
| | | sapiens | activated channel, subfamily M beta member 3 |
| KCNMB3 | NM_171828 | Homo | potassium large conductance calcium- |
| | | sapiens | activated channel, subfamily M beta member 3 |
| KCNMB3 | NM_001163677 | Homo | potassium large conductance calcium- |
| | | sapiens | activated channel, subfamily M beta member 3 |
| KCNMB3 | NM_014407 | Homo | potassium large conductance calcium- |
| | | sapiens | activated channel, subfamily M beta member 3 |
| KCNMB3 | NM_171830 | Homo | potassium large conductance calcium- |
| | | sapiens | activated channel, subfamily M beta member 3 |
| KCNMB3 | XM_001475546 | Mus | potassium large conductance calcium- |
| | | musculus | activated channel, subfamily M, beta |
| | | | member 3 |
| KCNMB3 | XM_912348 | Mus | potassium large conductance calcium- |
| | | musculus | activated channel, subfamily M, beta |
| | | | member 3 |
| KCNMB4 | NM_021452 | Mus | potassium large conductance calcium- |
| | | musculus | activated channel, subfamily M, beta |
| | | | member 4 |
| KCNMB4 | NM_014505 | Homo | potassium large conductance calcium- |
| | | sapiens | activated channel, subfamily M, beta |
| | | | member 4 |
| KLF1 | NM_010635 | Mus | Kruppel-like factor 1 (erythroid) |
| | | musculus | |
| KLF1 | NM_006563 | Homo | Kruppel-like factor 1 (erythroid) |
| | | sapiens | |
| KLF4 | NM_010637 | Mus | Kruppel-like factor 4 (gut) |
| | | musculus | |
| KLF4 | NM_004235 | Homo | Kruppel-like factor 4 (gut) |
| | | sapiens | |
| LAMA1 | NM_005559.3 | Homo | laminin, alpha 1 |
| | | sapiens | |
| LAMA1 | NM_008480.2 | Mus | laminin, alpha 1 |
| | | musculus | |
| LDLR | NM_000527 | Homo | low density lipoprotein receptor |
| | | sapiens | |
| LDLR | NM_010700 | Mus | low density lipoprotein receptor |
| | | musculus | |
| MBNL1 | NM_021038.3, | Homo | muscleblind-like splicing regulator 1 |
| | NM_020007.3, | sapiens | |
| | NM_207293.1, | | |
| | NM_207294.1, | | |
| | NM_207295.1, | | |
| | NM_207296.1, | | |
| | NM_207297.1 | | |
| MBNL1 | NM_001253708.1, | Mus | muscleblind-like 1 (Drosophila) |
| | NM_001253709.1, | musculus | |
| | NM_001253710.1, | | |
| | NM_001253711.1, | | |
| | NM_001253713.1, | | |
| | NM_020007.3 | | |
| MECP2 | NM_010788 | Mus | methyl CpG binding protein 2 |
| | | musculus | |
| MECP2 | NM_001081979 | Mus | methyl CpG binding protein 2 |
| | | musculus | |
| MECP2 | NM_001110792 | Homo | methyl CpG binding protein 2 (Rett |
| | | sapiens | syndrome) |
| MECP2 | NM_004992 | Homo | methyl CpG binding protein 2 (Rett |
| | | sapiens | syndrome) |
| MERTK | NM_006343.2 | Homo | MER proto-oncogene, tyrosine kinase |
| | | sapiens | |
| MERTK | NM_008587.1 | Mus | c-mer proto-oncogene tyrosine kinase |
| | | musculus | |
| MSX2 | NM_013601 | Mus | similar to homeobox protein; homeobox, |
| | | musculus | msh-like 2 |
| MSX2 | XM_001475886 | Mus | similar to homeobox protein; homeobox, |
| | | musculus | msh-like 2 |
| MSX2 | NM_002449 | Homo | msh homeobox 2 |
| | | sapiens | |
| MYBPC3 | NM_008653 | Mus | myosin binding protein C, cardiac |
| | | musculus | |
| MYBPC3 | NM_000256 | Homo | myosin binding protein C, cardiac |
| | | sapiens | |
| NANOG | NM_024865 | Homo | Nanog homeobox pseudogene 8; Nanog |
| | | sapiens | homeobox |
| NANOG | XM_001471588 | Mus | similar to Nanog homeobox; Nanog |
| | | musculus | homeobox |
| NANOG | NM_028016 | Mus | similar to Nanog homeobox; Nanog |
| | | musculus | homeobox |
| NANOG | NM_001080945 | Mus | similar to Nanog homeobox; Nanog |
| | | musculus | homeobox |
| NF1 | NM_000267 | Homo | neurofibromin 1 |
| | | sapiens | |
| NF1 | NM_001042492 | Homo | neurofibromin 1 |
| | | sapiens | |
| NF1 | NM_001128147 | Homo | neurofibromin 1 |
| | | sapiens | |
| NF1 | NM_010897 | Mus | neurofibromatosis 1 |
| | | musculus | |
| NKX2-1 | NM_001079668 | Homo | NK2 homeobox 1 |
| | | sapiens | |
| NKX2-1 | NM_003317 | Homo | NK2 homeobox 1 |
| | | sapiens | |
| NKX2-1 | XM_002344771 | Homo | NK2 homeobox 1 |
| | | sapiens | |
| NKX2-1 | NM_009385 | Mus | NK2 homeobox 1 |
| | | musculus | |
| NKX2-1 | NM_001146198 | Mus | NK2 homeobox 1 |
| | | musculus | |
| PAH | NM_008777 | Mus | phenylalanine hydroxylase |
| | | musculus | |
| PAH | NM_000277 | Homo | phenylalanine hydroxylase |
| | | sapiens | |
| PTEN | NM_000314 | Homo | phosphatase and tensin homolog; |
| | | sapiens | phosphatase and tensin homolog |
| | | | pseudogene 1 |
| PTEN | NM_177096 | Mus | phosphatase and tensin homolog |
| | | musculus | |
| PTEN | NM_008960 | Mus | phosphatase and tensin homolog |
| | | musculus | |
| PTGS2 | NM_011198 | Mus | prostaglandin-endoperoxide synthase 2 |
| | | musculus | |
| PTGS2 | NM_000963 | Homo | prostaglandin-endoperoxide synthase 2 |
| | | sapiens | (prostaglandin G/H synthase and |
| | | | cyclooxygenase) |
| RB1 | NM_009029 | Mus | retinoblastoma 1 |
| | | musculus | |
| RB1 | NM_000321 | Homo | retinoblastoma 1 |
| | | sapiens | |
| RPS14 | NM_020600 | Mus | predicted gene 6204; ribosomal protein S14 |
| | | musculus | |
| RPS14 | NM_001025071 | Homo | ribosomal protein S14 |
| | | sapiens | |
| RPS14 | NM_005617 | Homo | ribosomal protein S14 |
| | | sapiens | |
| RPS14 | NM_001025070 | Homo | ribosomal protein S14 |
| | | sapiens | |
| RPS19 | XM_204069 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_991053 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_905004 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001005575 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | NM_023133 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_994263 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001481027 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_913504 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_001479631 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_902221 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | XM_893968 | Mus | predicted gene 4327; predicted gene 8683; |
| | | musculus | similar to 40S ribosomal protein S19; |
| | | | predicted gene 4510; predicted gene 13143; |
| | | | predicted gene 9646; ribosomal protein S19; |
| | | | predicted gene 9091; predicted gene 6636; |
| | | | predicted gene 14072 |
| RPS19 | NM_001022 | Homo | ribosomal protein S19 pseudogene 3; |
| | | sapiens | ribosomal protein S19 |
| SCARB1 | NM_016741 | Mus | scavenger receptor class B, member 1 |
| | | musculus | |
| SCARB1 | NM_001082959 | Homo | scavenger receptor class B, member 1 |
| | | sapiens | |
| SCARB1 | NM_005505 | Homo | scavenger receptor class B, member 1 |
| | | sapiens | |
| SERPINF1 | NM_011340 | Mus | serine (or cysteine) peptidase inhibitor, clade |
| | | musculus | F, member 1 |
| SERPINF1 | NM_002615 | Homo | serpin peptidase inhibitor, clade F (alpha-2 |
| | | sapiens | antiplasmin, pigment epithelium derived |
| | | | factor), member 1 |
| SIRT1 | NM_001159590 | Mus | sirtuin 1 (silent mating type information |
| | | musculus | regulation 2, homolog) 1 (S. cerevisiae) |
| SIRT1 | NM_019812 | Mus | sirtuin 1 (silent mating type information |
| | | musculus | regulation 2, homolog) 1 (S. cerevisiae) |
| SIRT1 | NM_001159589 | Mus | sirtuin 1 (silent mating type information |
| | | musculus | regulation 2, homolog) 1 (S. cerevisiae) |
| SIRT1 | NM_012238 | Homo | sirtuin (silent mating type information |
| | | sapiens | regulation 2 homolog) 1 (S. cerevisiae) |
| SIRT1 | NM_001142498 | Homo | sirtuin (silent mating type information |
| | | sapiens | regulation 2 homolog) 1 (S. cerevisiae) |
| SIRT6 | NM_016539 | Homo | sirtuin (silent mating type information |
| | | sapiens | regulation 2 homolog) 6 (S. cerevisiae) |
| SIRT6 | NM_001163430 | Mus | sirtuin 6 (silent mating type information |
| | | musculus | regulation 2, homolog) 6 (S. cerevisiae) |
| SIRT6 | NM_181586 | Mus | sirtuin 6 (silent mating type information |
| | | musculus | regulation 2, homolog) 6 (S. cerevisiae) |
| SMAD7 | NM_005904 | Homo | SMAD family member 7 |
| | | sapiens | |
| SMAD7 | NM_001042660 | Mus | MAD homolog 7 (Drosophila) |
| | | musculus | |
| SMN1 | NM_000344.3 | Homo | Survival Motor Neuron 1 |
| | | sapiens | |
| SMN1 | NM_022874.2 | Homo | Survival Motor Neuron 1 |
| | | sapiens | |
| SMN2 | NM_017411.3 | Homo | Survival Motor Neuron 2 |
| | NM_022875.2 | sapiens | |
| | NM_022876.2 | | |
| | NM_022877.2 | | |
| SSPN | NM_001135823.1, | Homo | sarcospan |
| | NM_005086.4 | sapiens | |
| SSPN | NM_010656.2 | Homo | sarcospan |
| | | sapiens | |
| ST7 | NM_021908 | Homo | suppression of tumorigenicity 7 |
| | | sapiens | |
| ST7 | NM_018412 | Homo | suppression of tumorigenicity 7 |
| | | sapiens | |
| STAT3 | NM_213660 | Mus | similar to Stat3B; signal transducer and |
| | | musculus | activator of transcription 3 |
| STAT3 | XM_001474017 | Mus | similar to Stat3B; signal transducer and |
| | | musculus | activator of transcription 3 |
| STAT3 | NM_213659 | Mus | similar to Stat3B; signal transducer and |
| | | musculus | activator of transcription 3 |
| STAT3 | NM_011486 | Mus | similar to Stat3B; signal transducer and |
| | | musculus | activator of transcription 3 |
| STAT3 | NM_213662 | Homo | signal transducer and activator of |
| | | sapiens | transcription 3 (acute-phase response factor) |
| STAT3 | NM_003150 | Homo | signal transducer and activator of |
| | | sapiens | transcription 3 (acute-phase response factor) |
| STAT3 | NM_139276 | Homo | signal transducer and activator of |
| | | sapiens | transcription 3 (acute-phase response factor) |
| UTRN | NM_007124 | Homo | utrophin |
| | | sapiens | |
| UTRN | NM_011682 | Mus | utrophin |
| | | musculus | |
| NFE2L2 | NM_001145412.2, | Homo | nuclear factor, erythroid 2-like 2 |
| | NM_001145413.2, | sapiens | |
| | NM_006164.4 | | |
| NFE2L2 | NM_010902.3 | Mus | nuclear factor, erythroid 2-like 2 |
| | | musculus | |
| ACTB | NM_001101.3 | Homo | actin, beta |
| | | sapiens | |
| ACTB | NM_007393.3 | Mus | actin, beta |
| | | musculus | |
| ANRIL | NR_003529.3, | Homo | CDKN2B antisense RNA 1 (also called |
| | NR_047532.1, | sapiens | CDKN2B) |
| | NR_047533.1, | | |
| | NR_047534.1, | | |
| | NR_047535.1, | | |
| | NR_047536.1, | | |
| | NR_047538.1, | | |
| | NR_047539.1, | | |
| | NR_047540.1, | | |
| | NR_047541.1, | | |
| | NR_047542.1, | | |
| | NR_047543.1 | | |
| HOTAIR | NR_003716.3, | Homo | HOX transcript antisense RNA |
| | NR_047517.1, | sapiens | |
| | NR_047518.1 | | |
| HOTAIR | NR_047528.1 | Mus | HOX transcript antisense RNA |
| | | musculus | |
| DINO | JX993265 | Homo | Damage Induced NOncoding |
| | | sapiens | |
| DINO | JX993266 | Mus | Damage Induced NOncoding |
| | | musculus | |
| HOTTIP | NR_037843.3 | Homo | HOXA distal transcript antisense RNA |
| | | sapiens | |
| HOTTIP | NR_110441.1, | Mus | Hoxa distal transcript antisense RNA |
| | NR_110442.1 | musculus | |
| NEST | NR_104124.1 | Homo | Homo sapiens IFNG antisense RNA 1 (IFNG- |
| | | sapiens | AS1), transcript variant 1, long non-coding |
| | | | RNA. |
| NEST | NR_104123.1 | Mus | Theiler's murine encephalomyelitis virus |
| | | musculus | persistence candidate gene 1 |

### Synthetic RNAs

In some aspects of the disclosure, formulations and methods are provided for stabilizing a synthetic RNA (e.g., a synthetic RNA that is to be delivered to a cell). In some embodiments, the formulations and methods involve contacting a synthetic RNA with one or more stabilizing oligonucleotides that bind to a 5′ region of the synthetic RNA and a 3′ region of the synthetic RNA and that when bound to the synthetic RNA form a circularized product with the synthetic RNA. In some embodiments, the synthetic RNA is contacted with the one or more stabilizing oligonucleotides outside of a cell.

In some embodiments, synthetic RNAs are provided with chemistries suitable for delivery, hybridization and stability within cells which are targeted and stabilized by a stabilizing oligonucleotide, e.g., as described herein. Furthermore, in some embodiments, synthetic RNA chemistries are provided that are useful for controlling the pharmacokinetics, biodistribution, bioavailability and/or efficacy of the synthetic RNAs. Accordingly, synthetic RNAs described herein may be modified, e.g., comprise a modified sugar moiety, a modified internucleoside linkage, a modified nucleotide and/or combinations thereof. In addition, the synthetic RNAs may exhibit one or more of the following properties: do not induce substantial cleavage or degradation of the target RNA and/or stabilizing oligonucleotide; do not cause substantially complete cleavage or degradation of the target RNA and/or stabilizing oligonucleotide; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified synthetic RNAs; are not toxic to cells or mammals; and may have improved endosomal exit.

### Methods for Modulating Gene Expression

In one aspect, the disclosure relates to methods for modulating (e.g., increasing) stability of RNA transcripts in cells. The cells can be in vitro, ex vivo, or in vivo. The cells can be in a subject who has a disease resulting from reduced expression or activity of the RNA transcript or its corresponding protein product in the case of mRNAs. In some embodiments, methods for modulating stability of RNA transcripts in cells comprise delivering to the cell a formulation comprising a stabilizing oligonucleotide and a particle, e.g., nanoparticle. In some embodiments, delivery of a formulation comprising a stabilizing oligonucleotide and a particle as described herein to the cell results in an increase in stability of a target RNA that is at least 5%, 10%, 20%, 30%, 40%, 50%, 60%, 70%, 80%, 90%, 100%, 200% or more greater than a level of stability of the target RNA in a control cell. An appropriate control cell may be a cell to which a stabilizing oligonucleotide has not been delivered or to which a negative control has been delivered (e.g., a scrambled oligo, a carrier, etc.).

Another aspect of the disclosure provides methods of treating a disease or condition associated with low levels of a particular RNA in a subject. Accordingly, in some embodiments, methods are provided that comprise administering to a subject (e.g. a human) a formulation comprising a stabilizing oligonucleotide and a particle as described herein to increase mRNA stability in cells of the subject for purposes of increasing protein levels. In some embodiments, the increase in protein levels is at least 5%, 10%, 20%, 30%, 40%, 50%, 60%, 70%, 80%, 90%, 100%, 200%, or more, higher than the amount of a protein in the subject (e.g., in a cell or tissue of the subject) before administering or in a control subject which has not been administered the stabilizing oligonucleotide or that has been administered a negative control (e.g., a scrambled oligo, a carrier, etc.). In some embodiments, methods are provided that comprise administering to a subject (e.g. a human) a formulation comprising a stabilizing oligonucleotide and a particle as described herein to increase stability of non-coding RNAs in cells of the subject for purposes of increasing activity of those non-coding RNAs.

A subject can include a non-human mammal, e.g. mouse, rat, guinea pig, rabbit, cat, dog, goat, cow, or horse. In preferred embodiments, a subject is a human. Stabilizing oligonucleotides may be employed as therapeutic moieties in the treatment of disease states in animals, including humans. Stabilizing oligonucleotides can be useful therapeutic modalities that can be configured to be useful in treatment regimes for the treatment of cells, tissues and animals, especially humans.

For therapeutics, an animal, preferably a human, suspected of having a disease associated with low levels of an RNA or protein is treated by administering a formulation comprising a stabilizing oligonucleotide and a particle in accordance with this disclosure. For example, in one non-limiting embodiment, the methods comprise the step of administering to the animal in need of treatment, a therapeutically effective amount of an formulation as described herein. Table 2 listed examples of diseases or conditions that may be treated by targeting mRNA transcripts with stabilizing oligonucleotides. In some embodiments, cells used in the methods disclosed herein may, for example, be cells obtained from a subject having one or more of the conditions listed in Table 2, or from a subject that is a disease model of one or more of the conditions listed in Table 2.

TABLE 2
Examples of diseases or conditions treatable with oligonucleotides targeting
associated mRNA.

| Gene | Disease or conditions |
|---|---|
| FXN | Friedreich's Ataxia |
| SMN | Spinal muscular atrophy (SMA) types I-IV, Amyotrophic Lateral |
| | Sclerosis (ALS) |
| UTRN | Muscular dystrophy (MD) (e.g., Duchenne's muscular dystrophy, |
| | Becker's muscular dystrophy, myotonic dystrophy) |
| HEMOGLOBIN | Anemia, microcytic anemia, sickle cell anemia and/or thalassemia (e.g., |
| | alpha-thalassemia, beta-thalaseemia, delta-thalessemia), beta-thalaseemia |
| | (e.g., thalassemia minor/intermedia/major) |
| ATP2A2 | Cardiac conditions (e.g., congenital heart disease, aortic aneurysms, |
| | aortic dissections, arrhythmia, cardiomyopathy, and congestive heart |
| | failure), Darier-White disease and Acrokeratosis verruciformi |
| APOA1/ | Dyslipidemia (e.g. Hyperlipidemia) and atherosclerosis (e.g. coronary |
| ABCA1 | artery disease (CAD) and myocardial infarction (MI)) |
| PTEN | Cancer, such as, leukemias, lymphomas, myelomas, carcinomas, |
| | metastatic carcinomas, sarcomas, adenomas, nervous system cancers and |
| | genito-urinary cancers. In some embodiments, the cancer is adult and |
| | pediatric acute lymphoblastic leukemia, acute myeloid leukemia, |
| | adrenocortical carcinoma, AIDS-related cancers, anal cancer, cancer of |
| | the appendix, astrocytoma, basal cell carcinoma, bile duct cancer, |
| | bladder cancer, bone cancer, osteosarcoma, fibrous histiocytoma, brain |
| | cancer, brain stem glioma, cerebellar astrocytoma, malignant glioma, |
| | ependymoma, medulloblastoma, supratentorial primitive |
| | neuroectodermal tumors, hypothalamic glioma, breast cancer, male |
| | breast cancer, bronchial adenomas, Burkitt lymphoma, carcinoid tumor, |
| | carcinoma of unknown origin, central nervous system lymphoma, |
| | cerebellar astrocytoma, malignant glioma, cervical cancer, childhood |
| | cancers, chronic lymphocytic leukemia, chronic myelogenous leukemia, |
| | chronic myeloproliferative disorders, colorectal cancer, cutaneous T-cell |
| | lymphoma, endometrial cancer, ependymoma, esophageal cancer, Ewing |
| | family tumors, extracranial germ cell tumor, extragonadal germ cell |
| | tumor, extrahepatic bile duct cancer, intraocular melanoma, |
| | retinoblastoma, gallbladder cancer, gastric cancer, gastrointestinal |
| | stromal tumor, extracranial germ cell tumor, extragonadal germ cell |
| | tumor, ovarian germ cell tumor, gestational trophoblastic tumor, glioma, |
| | hairy cell leukemia, head and neck cancer, hepatocellular cancer, |
| | Hodgkin lymphoma, non-Hodgkin lymphoma, hypopharyngeal cancer, |
| | hypothalamic and visual pathway glioma, intraocular melanoma, islet |
| | cell tumors, Kaposi sarcoma, kidney cancer, renal cell cancer, laryngeal |
| | cancer, lip and oral cavity cancer, small cell lung cancer, non-small cell |
| | lung cancer, primary central nervous system lymphoma, Waldenstrom |
| | macroglobulinema, malignant fibrous histiocytoma, medulloblastoma, |
| | melanoma, Merkel cell carcinoma, malignant mesothelioma, squamous |
| | neck cancer, multiple endocrine neoplasia syndrome, multiple myeloma, |
| | mycosis fungoides, myelodysplastic syndromes, myeloproliferative |
| | disorders, chronic myeloproliferative disorders, nasal cavity and |
| | paranasal sinus cancer, nasopharyngeal cancer, neuroblastoma, |
| | oropharyngeal cancer, ovarian cancer, pancreatic cancer, parathyroid |
| | cancer, penile cancer, pharyngeal cancer, pheochromocytoma, |
| | pineoblastoma and supratentorial primitive neuroectodermal tumors, |
| | pituitary cancer, plasma cell neoplasms, pleuropulmonary blastoma, |
| | prostate cancer, rectal cancer, rhabdomyosarcoma, salivary gland cancer, |
| | soft tissue sarcoma, uterine sarcoma, Sezary syndrome, non-melanoma |
| | skin cancer, small intestine cancer, squamous cell carcinoma, squamous |
| | neck cancer, supratentorial primitive neuroectodermal tumors, testicular |
| | cancer, throat cancer, thymoma and thymic carcinoma, thyroid cancer, |
| | transitional cell cancer, trophoblastic tumors, urethral cancer, uterine |
| | cancer, uterine sarcoma, vaginal cancer, vulvar cancer, or Wilms tumor |
| BDNF | Amyotrophic lateral sclerosis (ALS, also known as Lou Gehrig's |
| | disease), Alzheimer's Disease (AD), and Parkinson's Disease (PD), |
| | Neurodegeneration |
| MECP2 | Rett Syndrome, MECP2-related severe neonatal encephalopathy, |
| | Angelman syndrome, or PPM-X syndrome |
| FOXP3 | Diseases or disorders associated with aberrant immune cell (e.g., T cell) |
| | activation, e.g., autoimmune or inflammatory diseases or disorders. |
| | Examples of autoimmune diseases and disorders that may be treated |
| | according to the methods disclosed herein include, but are not limited to, |
| | Acute Disseminated Encephalomyelitis (ADEM), Acute necrotizing |
| | hemorrhagic leukoencephalitis, Addison's disease, |
| | Agammaglobulinemia, Alopecia areata, Amyloidosis, Ankylosing |
| | spondylitis, Anti-GBM/Anti-TBM nephritis, Antiphospholipid syndrome |
| | (APS), Autoimmune angioedema, Autoimmune aplastic anemia, |
| | Autoimmune dysautonomia, Autoimmune hepatitis, Autoimmune |
| | hyperlipidemia, Autoimmune immunodeficiency, Autoimmune inner ear |
| | disease (AIED), Autoimmune myocarditis, Autoimmune oophoritis, |
| | Autoimmune pancreatitis, Autoimmune retinopathy, Autoimmune |
| | thrombocytopenic purpura (ATP), Autoimmune thyroid disease, |
| | Autoimmune urticaria, Axonal & neuronal neuropathies, Balo disease, |
| | Behcet's disease, Bullous pemphigoid, Cardiomyopathy, Castleman |
| | disease, Celiac disease, Chagas disease, Chronic inflammatory |
| | demyelinating polyneuropathy (CIDP), Chronic recurrent multifocal |
| | ostomyelitis (CRMO), Churg-Strauss syndrome, Cicatricial |
| | pemphigoid/benign mucosal pemphigoid, inflammatory bowel disease |
| | (e.g., Crohn's disease or Ulcerative colitis), Cogans syndrome, Cold |
| | agglutinin disease, Congenital heart block, Coxsackie myocarditis, |
| | CREST disease, Essential mixed cryoglobulinemia, Demyelinating |
| | neuropathies, Dermatitis herpetiformis, Dermatomyositis, Devic's |
| | disease (neuromyelitis optica), Discoid lupus, Dressler's syndrome, |
| | Endometriosis, Eosinophilic esophagitis, Eosinophilic fasciitis, Erythema |
| | nodosum, Experimental allergic encephalomyelitis, Evans syndrome, |
| | Fibrosing alveolitis, Giant cell arteritis (temporal arteritis), Giant cell |
| | myocarditis, Glomerulonephritis, Goodpasture's syndrome, |
| | Granulomatosis with Polyangiitis (GPA) (formerly called Wegener's |
| | Granulomatosis), Graves' disease, Guillain-Barre syndrome, Hashimoto's |
| | encephalitis, Hashimoto's thyroiditis, Hemolytic anemia, Henoch- |
| | Schonlein purpura, Herpes gestationis, Hypogammaglobulinemia, |
| | Idiopathic thrombocytopenic purpura (ITP), IgA nephropathy, IgG4- |
| | related sclerosing disease, Immunoregulatory lipoproteins, Inclusion |
| | body myositis, Interstitial cystitis, IPEX (Immunodysregulation, |
| | Polyendocrinopathy, and Enteropathy, X-linked) syndrome, Juvenile |
| | arthritis, Juvenile diabetes (Type 1 diabetes), Juvenile myositis, |
| | Kawasaki syndrome, Lambert-Eaton syndrome, Leukocytoclastic |
| | vasculitis, Lichen planus, Lichen sclerosus, Ligneous conjunctivitis, |
| | Linear IgA disease (LAD), systemic lupus erythematosus (SLE), chronic |
| | Lyme disease, Meniere's disease, Microscopic polyangiitis, Mixed |
| | connective tissue disease (MCTD), Mooren's ulcer, Mucha-Habermann |
| | disease, Multiple sclerosis, Myasthenia gravis, Myositis, Narcolepsy, |
| | Neuromyelitis optica (Devic's), Neutropenia, Ocular cicatricial |
| | pemphigoid, Optic neuritis, Palindromic rheumatism, PANDAS |
| | (Pediatric Autoimmune Neuropsychiatric Disorders Associated with |
| | Streptococcus), Paraneoplastic cerebellar degeneration, Paroxysmal |
| | nocturnal hemoglobinuria (PNH), Parry Romberg syndrome, |
| | Parsonnage-Turner syndrome, Pars planitis (peripheral uveitis), |
| | Pemphigus, Peripheral neuropathy, Perivenous encephalomyelitis, |
| | Pernicious anemia, POEMS syndrome, Polyarteritis nodosa, Type I, II, |
| | & III autoimmune polyglandular syndromes, Polymyalgia rheumatica, |
| | Polymyositis, Postmyocardial infarction syndrome, Postpericardiotomy |
| | syndrome, Progesterone dermatitis, Primary biliary cirrhosis, Primary |
| | sclerosing cholangitis, Psoriasis, Psoriatic arthritis, Idiopathic pulmonary |
| | fibrosis, Pyoderma gangrenosum, Pure red cell aplasia, Raynauds |
| | phenomenon, Reactive Arthritis, Reflex sympathetic dystrophy, Reiter's |
| | syndrome, Relapsing polychondritis, Restless legs syndrome, |
| | Retroperitoneal fibrosis, Rheumatic fever, Rheumatoid arthritis, |
| | Sarcoidosis, Schmidt syndrome, Scleritis, Scleroderma, Sjogren's |
| | syndrome, Sperm & testicular autoimmunity, Stiff person syndrome, |
| | Subacute bacterial endocarditis (SBE), Susac's syndrome, Sympathetic |
| | ophthalmia, Takayasu's arteritis, Temporal arteritis/Giant cell arteritis, |
| | Thrombocytopenic purpura (TTP), Tolosa-Hunt syndrome, Transverse |
| | myelitis, Type 1 diabetes, Undifferentiated connective tissue disease |
| | (UCTD), Uveitis, Vasculitis, Vesiculobullous dermatosis, Vitiligo, and |
| | Wegener's granulomatosis (also called Granulomatosis with Polyangiitis |
| | (GPA)). Further examples of autoimmune disease or disorder include |
| | inflammatory bowel disease (e.g., Crohn's disease or Ulcerative colitis), |
| | IPEX syndrome, Multiple sclerosis, Psoriasis, Rheumatoid arthritis, SLE |
| | or Type 1 diabetes. Examples of inflammatory diseases or disorders that |
| | may be treated according to the methods disclosed herein include, but are |
| | not limited to, Acne Vulgaris, Appendicitis, Arthritis, Asthma, |
| | Atherosclerosis, Allergies (Type 1 Hypersensitivity), Bursitis, Colitis, |
| | Chronic Prostatitis, Cystitis, Dermatitis, Glomerulonephritis, |
| | Inflammatory Bowel Disease, Inflammatory Myopathy (e.g., |
| | Polymyositis, Dermatomyositis, or Inclusion-body Myositis), |
| | Inflammatory Lung Disease, Interstitial Cystitis, Meningitis, Pelvic |
| | Inflammatory Disease, Phlebitis, Psoriasis, Reperfusion Injury, |
| | Rheumatoid Arthritis, Sarcoidosis, Tendonitis, Tonsilitis, Transplant |
| | Rejection, and Vasculitis. In some embodiments, the inflammatory |
| | disease or disorder is asthma. |

### Route of Delivery

A formulation comprising a stabilizing oligonucleotide and a particle may be delivered to a subject by a variety of routes. Exemplary routes include: intravenous, intradermal, topical, rectal, parenteral, anal, intravaginal, intranasal, pulmonary, ocular. The term “therapeutically effective amount” is the amount of stabilizing oligonucleotide present in the composition that is needed to provide the desired level of gene expression (e.g., by stabilizing RNA transcripts) in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

In some embodiments, formulations comprising stabilizing oligonucleotide molecules and particles of the disclosure can be incorporated into pharmaceutical compositions suitable for administration. In some embodiments, such formulations may include one or more species of stabilizing oligonucleotide and a particle, and a pharmaceutically acceptable carrier, e.g., to the extent such a carrier is compatible with desired oligonucleotide molecules and particles interactions. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The formulations of the present disclosure may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration.

The route and site of administration may be chosen to enhance targeting. For example, to target muscle cells, intramuscular injection into the muscles of interest would be a logical choice. Lung cells might be targeted by administering a formulation in aerosol form. The vascular endothelial cells could be targeted by coating a balloon catheter with a formulation of the present disclosure and mechanically introducing the oligonucleotide.

Topical administration refers to the delivery to a subject by contacting the formulation directly to a surface of the subject. The most common form of topical delivery is to the skin, but a formulation disclosed herein can also be directly applied to other surfaces of the body, e.g., to the eye, a mucous membrane, to surfaces of a body cavity or to an internal surface. As mentioned above, the most common topical delivery is to the skin. The term encompasses several routes of administration including, but not limited to, topical and transdermal. These modes of administration typically include penetration of the skin's permeability barrier and efficient delivery to the target tissue or stratum. Topical administration can be used as a means to penetrate the epidermis and dermis and ultimately achieve systemic delivery of the composition. Topical administration can also be used as a means to selectively deliver oligonucleotides to the epidermis or dermis of a subject, or to specific strata thereof, or to an underlying tissue.

Formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Transdermal delivery is a valuable route for the administration of lipid soluble therapeutics. The dermis is more permeable than the epidermis and therefore absorption is much more rapid through abraded, burned or denuded skin. Inflammation and other physiologic conditions that increase blood flow to the skin also enhance transdermal adsorption. Absorption via this route may be enhanced by the use of an oily vehicle (inunction) or through the use of one or more penetration enhancers. Other effective ways to deliver a formulation disclosed herein via the transdermal route include hydration of the skin and the use of controlled release topical patches. The transdermal route provides a potentially effective means to deliver a formulation disclosed herein for systemic and/or local therapy. In addition, iontophoresis (transfer of ionic solutes through biological membranes under the influence of an electric field), phonophoresis or sonophoresis (use of ultrasound to enhance the absorption of various therapeutic agents across biological membranes, notably the skin and the cornea), and optimization of vehicle characteristics relative to dose position and retention at the site of administration may be useful methods for enhancing the transport of topically applied compositions across skin and mucosal sites.

Both the oral and nasal membranes offer advantages over other routes of administration. For example, formulations administered through these membranes may have a rapid onset of action, provide therapeutic plasma levels, avoid first pass effect of hepatic metabolism, and avoid exposure of the oligonucleotides to the hostile gastrointestinal (GI) environment. Additional advantages include easy access to the membrane sites so that the formulation can be applied, localized and removed easily.

In oral delivery, formulations can be targeted to a surface of the oral cavity, e.g., to sublingual mucosa which includes the membrane of ventral surface of the tongue and the floor of the mouth or the buccal mucosa which constitutes the lining of the cheek. The sublingual mucosa is relatively permeable thus giving rapid absorption and acceptable bioavailability of many agents. Further, the sublingual mucosa is convenient, acceptable and easily accessible.

A formulation may also be administered to the buccal cavity of a human being by spraying into the cavity, without inhalation, from a metered dose spray dispenser, a mixed micellar pharmaceutical formulation as described above and a propellant. In one embodiment, the dispenser is first shaken prior to spraying the pharmaceutical formulation and propellant into the buccal cavity.

Formulations for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g. injection into a tumor).

Formulations for parenteral administration may include sterile aqueous solutions which may also contain buffers, diluents and other suitable additives. Intraventricular injection may be facilitated by an intraventricular catheter, for example, attached to a reservoir. For intravenous use, the total concentration of solutes should be controlled to render the preparation isotonic.

Any of the formulations described herein can be administered to ocular tissue. For example, the formulations can be applied to the surface of the eye or nearby tissue, e.g., the inside of the eyelid. For ocular administration, ointments or droppable liquids may be delivered by ocular delivery systems known to the art such as applicators or eye droppers. Such formulations can include mucomimetics such as hyaluronic acid, chondroitin sulfate, hydroxypropyl methylcellulose or poly(vinyl alcohol), preservatives such as sorbic acid, EDTA or benzylchronium chloride, and the usual quantities of diluents and/or carriers. A formulation can also be administered to the interior of the eye, and can be introduced by a needle or other delivery device which can introduce it to a selected area or structure.

Pulmonary delivery formulations can be delivered by inhalation by the patient of a dispersion so that the composition, preferably oligonucleotides, within the dispersion can reach the lung where it can be readily absorbed through the alveolar region directly into blood circulation. Pulmonary delivery can be effective both for systemic delivery and for localized delivery to treat diseases of the lungs.

Pulmonary delivery can be achieved by different approaches, including the use of nebulized, aerosolized, micellular and dry powder-based formulations. Delivery can be achieved with liquid nebulizers, aerosol-based inhalers, and dry powder dispersion devices. Metered-dose devices are preferred. One of the benefits of using an atomizer or inhaler is that the potential for contamination is minimized because the devices are self-contained. Dry powder dispersion devices, for example, deliver agents that may be readily formulated as dry powders. A formulation may be stably stored as lyophilized or spray-dried powders by itself or in combination with suitable powder carriers. The delivery of a composition for inhalation can be mediated by a dosing timing element which can include a timer, a dose counter, time measuring device, or a time indicator which when incorporated into the device enables dose tracking, compliance monitoring, and/or dose triggering to a patient during administration of the aerosol medicament.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is said to be “respirable.” Preferably the average particle size is less than about 10 μm in diameter preferably with a relatively uniform spheroidal shape distribution. More preferably the diameter is less than about 7.5 μm and most preferably less than about 5.0 μm. Usually the particle size distribution is between about 0.1 μm and about 5 μm in diameter, particularly about 0.3 μm to about 5 μm.

The term “dry” means that the formulation has a moisture content below about 10% by weight (% w) water, usually below about 5% w and preferably less it than about 3% w. A dry composition can be such that the particles are readily dispersible in an inhalation device to form an aerosol.

The types of pharmaceutical excipients that are useful as carrier include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar oligonucleotide formulation may be achieved through metered dose spray devices with propellants such as tetrafluoroethane, heptafluoroethane, dimethylfluoropropane, tetrafluoropropane, butane, isobutane, dimethyl ether and other non-CFC and CFC propellants.

Exemplary devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to an oligonucleotide formulation, e.g., a device can release insulin.

In one embodiment, unit doses or measured doses of a formulation are dispensed by an implanted device. The device can include a sensor that monitors a parameter within a subject. For example, the device can include pump, e.g., and, optionally, associated electronics.

Tissue, e.g., cells or organs can be treated with a formulation, ex vivo and then administered or implanted in a subject. The tissue can be autologous, allogeneic, or xenogeneic tissue. E.g., tissue can be treated to reduce graft v. host disease. In other embodiments, the tissue is allogeneic and the tissue is treated to treat a disorder characterized by unwanted gene expression in that tissue. E.g., tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, oligonucleotide treated cells are insulated from other cells, e.g., by a semi-permeable porous barrier that prevents the cells from leaving the implant, but enables molecules from the body to reach the cells and molecules produced by the cells to enter the body. In one embodiment, the porous barrier is formed from alginate.

In one embodiment, a contraceptive device is coated with or contains a formulation comprising a stabilizing oligonucleotide and a particle. Exemplary devices include condoms, diaphragms, IUD (implantable uterine devices, sponges, vaginal sheaths, and birth control devices.

### Dosage

In one aspect, the disclosure features a method of administering a formulation comprising a stabilizing oligonucleotide and a particle to a subject (e.g., a human subject). In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50 or 100 mg per kg of bodyweight.

The defined amount can be an amount effective to treat or prevent a disease or disorder, e.g., a disease or disorder associated with low levels of an RNA or protein. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of a formulation comprising a stabilizing oligonucleotide and a particle. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the formulation may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

In some cases, a patient is treated with a formulation comprising a stabilizing oligonucleotide and a particle in conjunction with other therapeutic modalities.

Following successful treatment, it may be desirable to have the patient undergo maintenance therapy to prevent the recurrence of the disease state, wherein the formulation of the disclosure is administered in maintenance doses, ranging from 0.0001 mg to 100 mg per kg of body weight.

The concentration of an oligonucleotide formulation is an amount sufficient to be effective in treating or preventing a disorder or to regulate a physiological condition in humans. The concentration or amount of formulation administered will depend on the parameters determined for the agent and the method of administration, e.g. nasal, buccal, pulmonary. For example, nasal formulations may tend to require much lower concentrations of some ingredients in order to avoid irritation or burning of the nasal passages. It is sometimes desirable to dilute an oral formulation up to 10-100 times in order to provide a suitable nasal formulation.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide in a formulation can include a single treatment or, preferably, can include a series of treatments. It will also be appreciated that the effective dosage of an oligonucleotide in a formulation used for treatment may increase or decrease over the course of a particular treatment. For example, the subject can be monitored after administering a formulation comprising a stabilizing oligonucleotide and a particle. Based on information from the monitoring, an additional amount of a formulation comprising a stabilizing oligonucleotide and a particle can be administered.

Dosing is dependent on severity and responsiveness of the disease condition to be treated, with the course of treatment lasting from several days to several months, or until a cure is effected or a diminution of disease state is achieved. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models.

In one embodiment, the administration of a formulation comprising a stabilizing oligonucleotide and a particle is parenteral, e.g. intravenous (e.g., as a bolus or as a diffusible infusion), intradermal, intraperitoneal, intramuscular, intrathecal, intraventricular, intracranial, subcutaneous, transmucosal, buccal, sublingual, endoscopic, rectal, oral, vaginal, topical, pulmonary, intranasal, urethral or ocular. Administration can be provided by the subject or by another person, e.g., a health care provider. The composition can be provided in measured doses or in a dispenser which delivers a metered dose. Selected modes of delivery are discussed in more detail below.

### Kits

In certain aspects of the disclosure, kits are provided, comprising a container housing a formulation comprising a stabilizing oligonucleotide and a particle. In some embodiments, the formulation comprises a stabilizing oligonucleotide and a nanoparticle, e.g., lipid nanoparticle. In some embodiments, the formulation further comprises a synthetic RNA. In some embodiments, the individual components of the formulation may be provided in one container. Alternatively, it may be desirable to provide the components of the formulation separately in two or more containers, e.g., one container for stabilizing oligonucleotide, and at least another for the particle. In some embodiments, it is desirable to provide a further container for a synthetic RNA. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1. Oligonucleotide for Targeting 5′ and 3′ Ends of RNAs

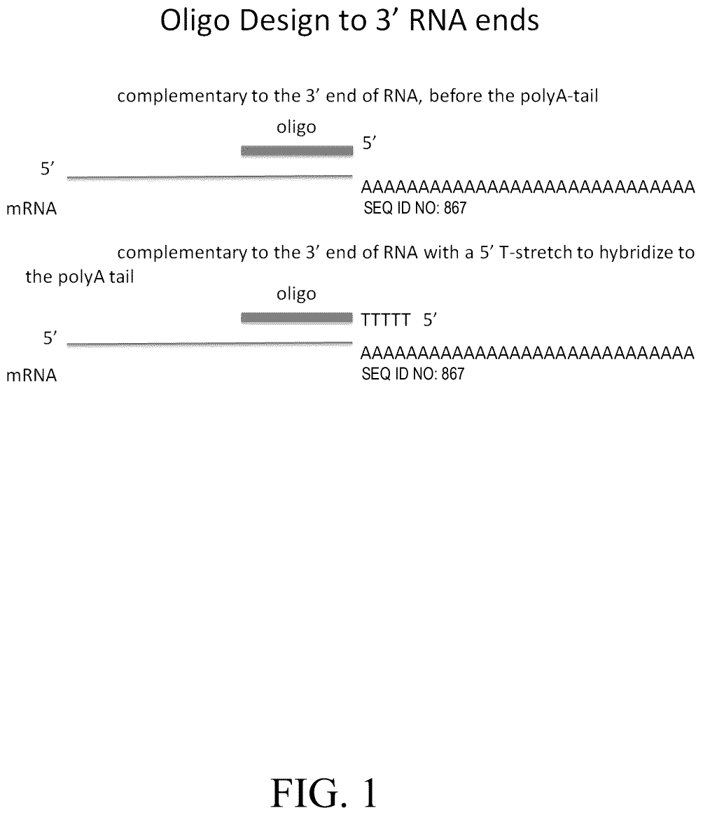

Several exemplary oligonucleotide design schemes are provided herein for increasing mRNA stability. With regard to oligonucleotides targeting the 3′ end of an RNA, at least two exemplary design schemes are provided. As a first scheme, an oligo nucleotide is designed to be complementary to the 3′ end of an RNA, before the poly-A tail (FIG. 1). As a second scheme, an oligonucleotide is designed to be complementary to the 3′ end of RNA with a 5′ poly-T region that hybridizes to a poly-A tail (FIG. 1).

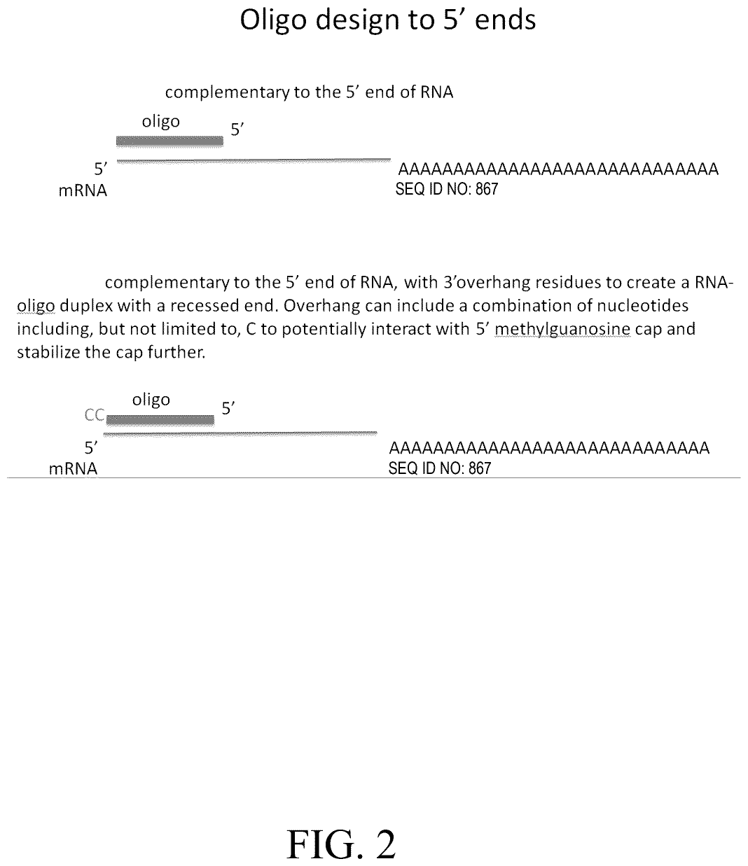

With regard to oligonucleotides targeting the 5′ end of an RNA, at least three exemplary design schemes are provided. For scheme one, an oligonucleotide is designed to be complementary to the 5′ end of RNA (FIG. 2). For scheme two, an oligonucleotide is designed to be complementary to the 5′ end of RNA and has a 3′ overhang to create a RNA-oligo duplex with a recessed end. In this example, the overhang is one or more C nucleotides, e.g., two Cs, which can potentially interact with a 5′ methylguanosine cap and stabilize the cap further (FIG. 2). The overhang could also potentially be another type of nucleotide, and is not limited to C. For scheme 3, an oligonucleotide is designed to include a loop region to stabilize 5′ RNA cap (FIG. 3A). FIG. 3A also shows an exemplary oligo for targeting 5′ and 3′ RNA ends. The example shows oligos that bind to a 5′ and 3′ RNA end to create a pseudo-circularized RNA (FIG. 3B).

An oligonucleotide designed as described in Example 1 may be used or evaluated for its ability to upregulate RNA by increasing mRNA stability using the methods outlined in Example 2.

### Example 2: Oligos for Targeting the 5′ and 3′ End of Frataxin

### Oligonucleotide Design

Oligonucleotides were designed to target the 5′ and 3′ ends of coding or non-coding mRNA. The 3′ end oligonucleotides were designed by identifying putative mRNA 3′ ends using quantitative end analysis of poly-A tails as described previously (see, e.g., Ozsolak et al. Comprehensive Polyadenylation Site Maps in Yeast and Human Reveal Pervasive Alternative Polyadenylation. Cell. Volume 143, Issue 6, 2010, Pages 1018-1029). The 5′ end oligonucleotides were designed by identifying potential 5′ start sites using Cap analysis gene expression (CAGE) as previously described (see, e.g., Cap analysis gene expression for high-throughput analysis of transcriptional starting point and identification of promoter usage. Proc Natl Acad Sci USA. 100 (26): 15776-81. 2003 Dec. 23 and Zhao, Xiaobei (2011). “Systematic Clustering of Transcription Start Site Landscapes”. PLOS ONE (Public Library of Science) 6 (8): e23409).

The sequence and structure of each oligonucleotide is shown in Tables 3-13. Table 14 provides a description of the nucleotide analogs, modifications and intranucleotide linkages used for certain oligonucleotides tested and described in the Tables. Certain oligos in Table 3 and Table 4 have two oligo names the “Oligo Name” and the “Alternative Oligo Name”, which are used interchangeably herein and are to be understood to refer to the same oligo.

TABLE 3
Oligonucleotides targeting 5′ and 3′ ends of FXN

| SEQ | | Alternative | | | | | |
|---|---|---|---|---|---|---|---|
| ID | Oligo | Oligo | Base | Targeting | Gene | | |
| NO | Name | Name | Sequence | Region | Name | Organism | Formatted Sequence |
| 1 | Oligo48 | FXN-371 | TGACCCA | 5′-End | FXN | human | dTs; InaGs; dAs; InaCs; dCs; |
| | | | AGGGAGA | | | | InaCs; dAs; InaAs; dGs; InaGs; |
| | | | C | | | | dGs; InaAs; dGs; InaAs; dC-Sup |
| 2 | Oligo49 | FXN-372 | TGGCCAC | 5′-End | FXN | human | dTs; InaGs; dGs; InaCs; dCs; |
| | | | TGGCCGC | | | | InaAs; dCs; InaTs; dGs; InaGs; |
| | | | A | | | | dCs; InaCs; dGs; InaCs; dA-Sup |
| 3 | Oligo50 | FXN-373 | CGGCGAC | 5′-End | FXN | human | dCs; InaGs; dGs; InaCs; dGs; |
| | | | CCCTGGT | | | | InaAs; dCs; InaCs; dCs; InaCs; |
| | | | G | | | | dTs; InaGs; dGs; InaTs; dG-Sup |
| 4 | Oligo51 | FXN-374 | CGCCCTCC | 5′-End | FXN | human | dCs; InaGs; dCs; InaCs; dCs; |
| | | | AGCGCTG | | | | InaTs; dCs; InaCs; dAs; InaGs; |
| | | | | | | | dCs; InaGs; dCs; InaTs; dG-Sup |
| 5 | Oligo52 | FXN-375 | CGCTCCG | 5′-End | FXN | human | dCs; InaGs; dCs; InaTs; dCs; |
| | | | CCCTCCA | | | | InaCs; dGs; InaCs; dCs; InaCs; |
| | | | G | | | | dTs; InaCs; dCs; InaAs; dG-Sup |
| 6 | Oligo53 | FXN-376 | TGACCCA | 5′-End | FXN | human | dTs; InaGs; dAs; InaCs; dCs; |
| | | | AGGGAGA | | | | InaCs; dAs; InaAs; dGs; InaGs; |
| | | | CCC | | | | dGs; InaAs; dGs; InaAs; dCs; |
| | | | | | | | InaCs; dC-Sup |
| 7 | Oligo54 | FXN-377 | TGGCCAC | 5′-End | FXN | human | dTs; InaGs; dGs; InaCs; dCs; |
| | | | TGGCCGC | | | | InaAs; dCs; InaTs; dGs; InaGs; |
| | | | ACC | | | | dCs; InaCs; dGs; InaCs; dAs; |
| | | | | | | | InaCs; dC-Sup |
| 8 | Oligo55 | FXN-378 | CGGCGAC | 5′-End | FXN | human | dCs; InaGs; dGs; InaCs; dGs; |
| | | | CCCTGGT | | | | InaAs; dCs; InaCs; dCs; InaCs; |
| | | | GCC | | | | dTs; InaGs; dGs; InaTs; dGs; |
| | | | | | | | InaCs; dC-Sup |
| 9 | Oligo56 | FXN-379 | CGCCCTCC | 5′-End | FXN | human | dCs; InaGs; dCs; InaCs; dCs; |
| | | | AGCGCTG | | | | InaTs; dCs; InaCs; dAs; InaGs; |
| | | | CC | | | | dCs; InaGs; dCs; InaTs; dGs; |
| | | | | | | | InaCs; dC-Sup |
| 10 | Oligo57 | FXN-380 | CGCTCCG | 5′-End | FXN | human | dCs; InaGs; dCs; InaTs; dCs; |
| | | | CCCTCCA | | | | InaCs; dGs; InaCs; dCs; InaCs; |
| | | | GCC | | | | dTs; InaCs; dCs; InaAs; dGs; |
| | | | | | | | InaCs; dC-Sup |
| 11 | Oligo58 | FXN-381 | TGACCCA | 5′-End | FXN | human | dTs; InaGs; dAs; InaCs; dCs; |
| | | | AGGGAGA | | | | InaCs; dAs; InaAs; dGs; InaGs; |
| | | | CGGAAAC | | | | dGs; InaAs; dGs; InaAs; dCs; |
| | | | CAC | | | | InaGs; dGs; dAs; dAs; dAs; dCs; |
| | | | | | | | InaCs; dAs; InaC-Sup |
| 12 | Oligo59 | FXN-382 | TGGCCAC | 5′-End | FXN | human | dTs; InaGs; dGs; InaCs; dCs; |
| | | | TGGCCGC | | | | InaAs; dCs; InaTs; dGs; InaGs; |
| | | | AGGAAAC | | | | dCs; InaCs; dGs; InaCs; dAs; |
| | | | CAC | | | | InaGs; dGs; dAs; dAs; dAs; dCs; |
| | | | | | | | InaCs; dAs; InaC-Sup |
| 13 | Oligo60 | FXN-383 | CGGCGAC | 5′-End | FXN | human | dCs; InaGs; dGs; InaCs; dGs; |
| | | | CCCTGGT | | | | InaAs; dCs; InaCs; dCs; InaCs; |
| | | | GGGAAAC | | | | dTs; InaGs; dGs; InaTs; dGs; |
| | | | CTC | | | | InaGs; dGs; dAs; dAs; dAs; dCs; |
| | | | | | | | InaCs; dTs; InaC-Sup |
| 14 | Oligo61 | FXN-384 | CGCCCTCC | 5′-End | FXN | human | dCs; InaGs; dCs; InaCs; dCs; |
| | | | AGCGCTG | | | | InaTs; dCs; InaCs; dAs; InaGs; |
| | | | GGAAACC | | | | dCs; InaGs; dCs; InaTs; dGs; |
| | | | TC | | | | InaGs; dGs; dAs; dAs; dAs; dCs; |
| | | | | | | | InaCs; dTs; InaC-Sup |
| 15 | Oligo62 | FXN-385 | CGCTCCG | 5′-End | FXN | human | dCs; InaGs; dCs; InaTs; dCs; |
| | | | CCCTCCA | | | | InaCs; dGs; InaCs; dCs; InaCs; |
| | | | GCCAAAG | | | | dTs; InaCs; dCs; InaAs; dGs; |
| | | | GTC | | | | InaCs; dCs; dAs; dAs; dAs; dGs; |
| | | | | | | | InaGs; dTs; InaC-Sup |
| 16 | Oligo63 | FXN-386 | GGTTTTTA | 3′-End | FXN | human | dGs; InaGs; dTs; InaTs; dTs; |
| | | | AGGCTTT | | | | InaTs; dTs; InaAs; dAs; InaGs; |
| | | | | | | | dGs; InaCs; dTs; InaTs; dT-Sup |
| 17 | Oligo64 | FXN-387 | GGGGTCT | 3′-End | FXN | human | dGs; InaGs; dGs; InaGs; dTs; |
| | | | TGGCCTG | | | | InaCs; dTs; InaTs; dGs; InaGs; |
| | | | A | | | | dCs; InaCs; dTs; InaGs; dA- |
| | | | | | | | Sup |
| 18 | Oligo65 | FXN-388 | CATAATG | 3′-End | FXN | human | dCs; InaAs; dTs; InaAs; dAs; |
| | | | AAGCTGG | | | | InaTs; dGs; InaAs; dAs; InaGs; |
| | | | G | | | | dCs; InaTs; dGs; InaGs; dG-Sup |
| 19 | Oligo66 | FXN-389 | AGGAGGC | 3′-End | FXN | human | dAs; InaGs; dGs; InaAs; dGs; |
| | | | AACACAT | | | | InaGs; dCs; InaAs; dAs; InaCs; |
| | | | T | | | | dAs; InaCs; dAs; InaTs; dT- |
| | | | | | | | Sup |
| 20 | Oligo67 | FXN-390 | ATTATTTT | 3′-End | FXN | human | dAs; InaTs; dTs; InaAs; dTs; |
| | | | GCTTTTT | | | | InaTs; dTs; InaTs; dGs; InaCs; |
| | | | | | | | dTs; InaTs; dTs; InaTs; dT-Sup |
| 21 | Oligo68 | FXN-391 | CATTTTCC | 3′-End | FXN | human | dCs; InaAs; dTs; InaTs; dTs; |
| | | | CTCCTGG | | | | InaTs; dCs; InaCs; dCs; InaTs; |
| | | | | | | | dCs; InaCs; dTs; InaGs; dG-Sup |
| 22 | Oligo69 | FXN-392 | GTAGGCT | 3′-End | FXN | human | dGs; InaTs; dAs; InaGs; dGs; |
| | | | ACCCTTTA | | | | InaCs; dTs; InaAs; dCs; InaCs; |
| | | | | | | | dCs; InaTs; dTs; InaTs; dA-Sup |
| 23 | Oligo70 | FXN-393 | GAGGCTT | 3′-End | FXN | human | dGs; InaAs; dGs; InaGs; dCs; |
| | | | GTTGCTTT | | | | InaTs; dTs; InaGs; dTs; InaTs; |
| | | | | | | | dGs; InaCs; dTs; InaTs; dT-Sup |
| 24 | Oligo71 | FXN-394 | CATGTAT | 3′-End | FXN | human | dCs; InaAs; dTs; InaGs; dTs; |
| | | | GATGTTA | | | | InaAs; dTs; InaGs; dAs; InaTs; |
| | | | T | | | | dGs; InaTs; dTs; InaAs; dT-Sup |
| 25 | Oligo72 | FXN-395 | TTTTTGGT | 3′-End | FXN | human | dTs; InaTs; dTs; InaTs; dTs; |
| | | | TTTTAAG | | | | InaGs; dGs; InaTs; dTs; InaTs; |
| | | | GCTTT | | | | dTs; InaTs; dAs; InaAs; dGs; |
| | | | | | | | InaGs; dCs; InaTs; dTs; InaT-Sup |
| 26 | Oligo73 | FXN-396 | TTTTTGG | 3′-End | FXN | human | dTs; InaTs; dTs; InaTs; dTs; |
| | | | GGTCTTG | | | | InaGs; dGs; InaGs; dGs; InaTs; |
| | | | GCCTGA | | | | dCs; InaTs; dTs; InaGs; dGs; |
| | | | | | | | InaCs; dCs; InaTs; dGs; InaA-Sup |
| 27 | Oligo74 | FXN-397 | TTTTTCAT | 3′-End | FXN | human | dTs; InaTs; dTs; InaTs; dTs; |
| | | | AATGAAG | | | | InaCs; dAs; InaTs; dAs; InaAs; |
| | | | CTGGG | | | | dTs; InaGs; dAs; InaAs; dGs; |
| | | | | | | | InaCs; dTs; InaGs; dGs; InaG-Sup |
| 28 | Oligo75 | FXN-398 | TTTTTAGG | 3′-End | FXN | human | dTs; InaTs; dTs; InaTs; dTs; |
| | | | AGGCAAC | | | | InaAs; dGs; InaGs; dAs; InaGs; |
| | | | ACATT | | | | dGs; InaCs; dAs; InaAs; dCs; |
| | | | | | | | InaAs; dCs; InaAs; dTs; InaT-Sup |
| 29 | Oligo76 | FXN-399 | TTTTTATT | 3′-End | FXN | human | dTs; InaTs; dTs; InaTs; dTs; |
| | | | ATTTTGCT | | | | InaAs; dTs; InaTs; dAs; InaTs; |
| | | | TTTT | | | | dTs; InaTs; dTs; InaGs; dCs; |
| | | | | | | | InaTs; dTs; InaTs; dTs; InaT-Sup |
| 30 | Oligo77 | FXN-400 | TTTTTCAT | 3′-End | FXN | human | dTs; InaTs; dTs; InaTs; dTs; |
| | | | TTTCCCTC | | | | InaCs; dAs; InaTs; dTs; InaTs; |
| | | | CTGG | | | | dTs; InaCs; dCs; InaCs; dTs; |
| | | | | | | | InaCs; dCs; InaTs; dGs; InaG-Sup |
| 31 | Oligo78 | FXN-401 | TTTTTGTA | 3′-End | FXN | human | dTs; InaTs; dTs; InaTs; dTs; |
| | | | GGCTACC | | | | InaGs; dTs; InaAs; dGs; InaGs; |
| | | | CTTTA | | | | dCs; InaTs; dAs; InaCs; dCs; |
| | | | | | | | InaCs; dTs; InaTs; dTs; InaA-Sup |
| 32 | Oligo79 | FXN-402 | TTTTTGAG | 3′-End | FXN | human | dTs; InaTs; dTs; InaTs; dTs; |
| | | | GCTTGTT | | | | InaGs; dAs; InaGs; dGs; InaCs; |
| | | | GCTTT | | | | dTs; InaTs; dGs; InaTs; dTs; |
| | | | | | | | InaGs; dCs; InaTs; dTs; InaT-Sup |
| 33 | Oligo80 | FXN-403 | TTTTTCAT | 3′-End | FXN | human | dTs; InaTs; dTs; InaTs; dTs; |
| | | | GTATGAT | | | | InaCs; dAs; InaTs; dGs; InaTs; |
| | | | GTTAT | | | | dAs; InaTs; dGs; InaAs; dTs; |
| | | | | | | | InaGs; dTs; InaTs; dAs; InaT-Sup |

TABLE 4
Other oligonucleotides targeting FXN

| SEQ | | Alternative | | | | | |
|---|---|---|---|---|---|---|---|
| ID | Oligo | Oligo | Base | Targeting | Gene | | Formatted |
| NO | Name | Name | Sequence | Region | Name | Organism | Sequence |
| 34 | Oligo1 | FXN-324 | CGGCGCC | Internal | FXN | human | dCs; InaGs; dGs; InaCs; |
| | | | CGAGAGT | | | | dGs; InaCs; dCs; InaCs; |
| | | | CCACAT | | | | dGs; InaAs; dGs; |
| | | | | | | | InaAs; dGs; InaTs; dCs; |
| | | | | | | | InaCs; dAs; InaCs; |
| | | | | | | | dAs; InaT-Sup |
| 35 | Oligo2 | FXN-325 | CCAGGAG | Internal | FXN | human | dCs; InaCs; dAs; InaGs; |
| | | | GCCGGCT | | | | dGs; InaAs; dGs; InaGs; |
| | | | ACTGCG | | | | dCs; InaCs; dGs; InaGs; |
| | | | | | | | dCs; InaTs; dAs; |
| | | | | | | | InaCs; dTs; InaGs; dCs; |
| | | | | | | | InaG-Sup |
| 36 | Oligo3 | FXN-326 | CTGGGCT | Internal | FXN | human | dCs; InaTs; dGs; InaGs; |
| | | | GGGCTGG | | | | dGs; InaCs; dTs; InaGs; |
| | | | GTGACG | | | | dGs; InaGs; dCs; |
| | | | | | | | InaTs; dGs; InaGs; dGs; |
| | | | | | | | InaTs; dGs; InaAs; |
| | | | | | | | dCs; InaG-Sup |
| 37 | Oligo4 | FXN-327 | ACCCGGG | Internal | FXN | human | dAs; InaCs; dCs; InaCs; |
| | | | TGAGGGT | | | | dGs; InaGs; dGs; InaTs; |
| | | | CTGGGC | | | | dGs; InaAs; dGs; |
| | | | | | | | InaGs; dGs; InaTs; dCs; |
| | | | | | | | InaTs; dGs; InaGs; |
| | | | | | | | dGs; InaC-Sup |
| 38 | Oligo5 | FXN-328 | CCAACTCT | Internal | FXN | human | dCs; InaCs; dAs; InaAs; |
| | | | GCCGGCC | | | | dCs; InaTs; dCs; InaTs; |
| | | | GCGGG | | | | dGs; InaCs; dCs; InaGs; |
| | | | | | | | dGs; InaCs; dCs; |
| | | | | | | | InaGs; dCs; InaGs; dGs; |
| | | | | | | | InaG-Sup |
| 39 | Oligo6 | FXN-329 | ACGGCGG | Internal | FXN | human | dAs; InaCs; dGs; InaGs; |
| | | | CCGCAGA | | | | dCs; InaGs; dGs; InaCs; |
| | | | GTGGGG | | | | dCs; InaGs; dCs; |
| | | | | | | | InaAs; dGs; InaAs; dGs; |
| | | | | | | | InaTs; dGs; InaGs; dGs; |
| | | | | | | | InaG-Sup |
| 40 | Oligo7 | FXN-330 | TCGATGT | Internal | FXN | human | dTs; InaCs; dGs; InaAs; |
| | | | CGGTGCG | | | | dTs; InaGs; dTs; InaCs; |
| | | | CAGGCC | | | | dGs; InaGs; dTs; |
| | | | | | | | InaGs; dCs; InaGs; dCs; |
| | | | | | | | InaAs; dGs; InaGs; dCs; |
| | | | | | | | InaC-Sup |
| 41 | Oligo8 | FXN-331 | GGCGGGG | Internal | FXN | human | dGs; InaGs; dCs; InaGs; |
| | | | CGTGCAG | | | | dGs; InaGs; dGs; InaCs; |
| | | | GTCGCA | | | | dGs; InaTs; dGs; |
| | | | | | | | InaCs; dAs; InaGs; dGs; |
| | | | | | | | InaTs; dCs; InaGs; |
| | | | | | | | dCs; InaA-Sup |
| 42 | Oligo9 | FXN-332 | ACGTTGG | Internal | FXN | human | dAs; InaCs; dGs; InaTs; |
| | | | TTCGAACT | | | | dTs; InaGs; dGs; InaTs; |
| | | | TGCGC | | | | dTs; InaCs; dGs; |
| | | | | | | | InaAs; dAs; InaCs; dTs; |
| | | | | | | | InaTs; dGs; InaCs; dGs; |
| | | | | | | | InaC-Sup |
| 43 | Oligo10 | FXN-333 | TTCCAAAT | Internal | FXN | human | dTs; InaTs; dCs; InaCs; |
| | | | CTGGTTG | | | | dAs; InaAs; dAs; InaTs; |
| | | | AGGCC | | | | dCs; InaTs; dGs; InaGs; |
| | | | | | | | dTs; InaTs; dGs; InaAs; |
| | | | | | | | dGs; InaGs; dCs; |
| | | | | | | | InaC-Sup |
| 44 | Oligo1l | FXN-334 | AGACACT | Internal | FXN | human | dAs; InaGs; dAs; InaCs; |
| | | | CTGCTTTT | | | | dAs; InaCs; dTs; InaCs; |
| | | | TGACA | | | | dTs; InaGs; dCs; InaTs; |
| | | | | | | | dTs; InaTs; dTs; |
| | | | | | | | InaTs; dGs; InaAs; dCs; |
| | | | | | | | InaA-Sup |
| 45 | Oligo12 | FXN-335 | TTTCCTCA | Internal | FXN | human | dTs; InaTs; dTs; InaCs; |
| | | | AATTCATC | | | | dCs; InaTs; dCs; InaAs; |
| | | | AAAT | | | | dAs; InaAs; dTs; InaTs; |
| | | | | | | | dCs; InaAs; dTs; |
| | | | | | | | InaCs; dAs; InaAs; dAs; |
| | | | | | | | InaT-Sup |
| 46 | Oligo13 | FXN-336 | GGGTGGC | Internal | FXN | human | dGs; InaGs; dGs; InaTs; |
| | | | CCAAAGT | | | | dGs; InaGs; dCs; InaCs; |
| | | | TCCAGA | | | | dCs; InaAs; dAs; |
| | | | | | | | InaAs; dGs; InaTs; dTs; |
| | | | | | | | InaCs; dCs; InaAs; dGs; |
| | | | | | | | InaA-Sup |
| 47 | Oligo14 | FXN-337 | TGGTCTC | Internal | FXN | human | dTs; InaGs; dGs; InaTs; |
| | | | ATCTAGA | | | | dCs; InaTs; dCs; InaAs; |
| | | | GAGCCT | | | | dTs; InaCs; dTs; |
| | | | | | | | InaAs; dGs; InaAs; dGs; |
| | | | | | | | InaAs; dGs; InaCs; dCs; |
| | | | | | | | InaT-Sup |
| 48 | Oligo15 | FXN-338 | CTCTGCTA | Internal | FXN | human | dCs; InaTs; dCs; InaTs; |
| | | | GTCTTTCA | | | | dGs; InaCs; dTs; InaAs; |
| | | | TAGG | | | | dGs; InaTs; dCs; InaTs; |
| | | | | | | | dTs; InaTs; dCs; |
| | | | | | | | InaAs; dTs; InaAs; dGs; |
| | | | | | | | InaG-Sup |
| 49 | Oligo16 | FXN-339 | GCTAAAG | Internal | FXN | human | dGs; InaCs; dTs; InaAs; |
| | | | AGTCCAG | | | | dAs; InaAs; dGs; InaAs; |
| | | | CGTTTC | | | | dGs; InaTs; dCs; |
| | | | | | | | InaCs; dAs; InaGs; dCs; |
| | | | | | | | InaGs; dTs; InaTs; dTs; |
| | | | | | | | InaC-Sup |
| 50 | Oligo17 | FXN-340 | GCAAGGT | Internal | FXN | human | dGs; InaCs; dAs; InaAs; |
| | | | CTTCAAA | | | | dGs; InaGs; dTs; InaCs; |
| | | | AAACTCT | | | | dTs; InaTs; dCs; |
| | | | | | | | InaAs; dAs; InaAs; dAs; |
| | | | | | | | InaAs; dAs; InaCs; dTs; |
| | | | | | | | InaCs; dT-Sup |
| 51 | Oligo18 | FXN-341 | CTCAAAC | Internal | FXN | human | dCs; InaTs; dCs; InaAs; |
| | | | GTGTATG | | | | dAs; InaAs; dCs; InaGs; |
| | | | GCTTGTCT | | | | dTs; InaGs; dTs; |
| | | | | | | | InaAs; dTs; InaGs; dGs; |
| | | | | | | | InaCs; dTs; InaTs; dGs; |
| | | | | | | | InaTs; dCs; InaT-Sup |
| 52 | Oligo19 | FXN-342 | CCCAAAG | Internal | FXN | human | dCs; InaCs; dCs; InaAs; |
| | | | GAGACAT | | | | dAs; InaAs; dGs; InaGs; |
| | | | CATAGTC | | | | dAs; InaGs; dAs; |
| | | | | | | | InaCs; dAs; InaTs; dCs; |
| | | | | | | | InaAs; dTs; InaAs; dGs; |
| | | | | | | | InaTs; dC-Sup |
| 53 | Oligo20 | FXN-343 | CAGTTTG | Internal | FXN | human | dCs; InaAs; dGs; InaTs; |
| | | | ACAGTTA | | | | dTs; InaTs; dGs; InaAs; |
| | | | AGACACC | | | | dCs; InaAs; dGs; |
| | | | ACT | | | | InaTs; dTs; InaAs; dAs; |
| | | | | | | | InaGs; dAs; InaCs; dAs; |
| | | | | | | | InaCs; dCs; InaAs; dCs; |
| | | | | | | | InaT-Sup |
| 54 | Oligo21 | FXN-344 | ATAGGTT | Internal | FXN | human | dAs; InaTs; dAs; InaGs; |
| | | | CCTAGAT | | | | dGs; InaTs; dTs; InaCs; |
| | | | CTCCACC | | | | dCs; InaTs; dAs; |
| | | | | | | | InaGs; dAs; InaTs; dCs; |
| | | | | | | | InaTs; dCs; InaCs; dAs; |
| | | | | | | | InaCs; dC-Sup |
| 55 | Oligo22 | FXN-345 | GGCGTCT | Internal | FXN | human | dGs; InaGs; dCs; InaGs; |
| | | | GCTTGTT | | | | dTs; InaCs; dTs; InaGs; |
| | | | GATCAC | | | | dCs; InaTs; dTs; |
| | | | | | | | InaGs; dTs; InaTs; dGs; |
| | | | | | | | InaAs; dTs; InaCs; dAs; |
| | | | | | | | InaC-Sup |
| 56 | Oligo23 | FXN-346 | AAGATAG | Internal | FXN | human | dAs; InaAs; dGs; InaAs; |
| | | | CCAGATTT | | | | dTs; InaAs; dGs; InaCs; |
| | | | GCTTGTTT | | | | dCs; InaAs; dGs; |
| | | | | | | | InaAs; dTs; InaTs; dTs; |
| | | | | | | | InaGs; dCs; InaTs; dTs; |
| | | | | | | | InaGs; dTs; InaTs; dT- |
| | | | | | | | Sup |
| 57 | Oligo24 | FXN-347 | GGTCCAC | Internal | FXN | human | dGs; InaGs; dTs; InaCs; |
| | | | TACATACC | | | | dCs; InaAs; dCs; InaTs; |
| | | | TGGATGG | | | | dAs; InaCs; dAs; |
| | | | AG | | | | InaTs; dAs; InaCs; dCs; |
| | | | | | | | InaTs; dGs; InaGs; dAs; |
| | | | | | | | InaTs; dGs; InaGs; dAs; |
| | | | | | | | InaG-Sup |
| 58 | Oligo25 | FXN-348 | CCCAGTC | Internal | FXN | human | dCs; InaCs; dCs; InaAs; |
| | | | CAGTCAT | | | | dGs; InaTs; dCs; InaCs; |
| | | | AACGCTT | | | | dAs; InaGs; dTs; |
| | | | | | | | InaCs; dAs; InaTs; dAs; |
| | | | | | | | InaAs; dCs; InaGs; dCs; |
| | | | | | | | InaTs; dT-Sup |
| 59 | Oligo26 | FXN-349 | CGTGGGA | Internal | FXN | human | dCs; InaGs; dTs; InaGs; |
| | | | GTACACC | | | | dGs; InaGs; dAs; InaGs; |
| | | | CAGTTTTT | | | | dTs; InaAs; dCs; |
| | | | | | | | InaAs; dCs; InaCs; dCs; |
| | | | | | | | InaAs; dGs; InaTs; dTs; |
| | | | | | | | InaTs; dTs; InaT-Sup |
| 60 | Oligo27 | FXN-350 | CATGGAG | Internal | FXN | human | dCs; InaAs; dTs; InaGs; |
| | | | GGACACG | | | | dGs; InaAs; dGs; |
| | | | CCGT | | | | InaGs; dGs; InaAs; dCs; |
| | | | | | | | InaAs; dCs; InaGs; dCs; |
| | | | | | | | InaCs; dGs; InaT- |
| | | | | | | | Sup |
| 61 | Oligo28 | FXN-351 | GTGAGCT | Internal | FXN | human | dGs; InaTs; dGs; InaAs; |
| | | | CTGCGGC | | | | dGs; InaCs; dTs; |
| | | | CAGCAGC | | | | InaCs; dTs; InaGs; dCs; |
| | | | T | | | | InaGs; dGs; InaCs; dCs; |
| | | | | | | | InaAs; dGs; InaCs; dAs; |
| | | | | | | | InaGs; dCs; InaT- |
| | | | | | | | Sup |
| 62 | Oligo29 | FXN-352 | AGTTTGG | Internal | FXN | human | dAs; InaGs; dTs; InaTs; |
| | | | TTTTTAAG | | | | dTs; InaGs; dGs; |
| | | | GCTTTA | | | | InaTs; dTs; InaTs; dTs; |
| | | | | | | | InaTs; dAs; InaAs; dGs; |
| | | | | | | | InaGs; dCs; InaTs; dTs; |
| | | | | | | | InaTs; dA-Sup |
| 63 | Oligo30 | FXN-353 | TAGGCCA | Internal | FXN | human | dTs; InaAs; dGs; InaGs; |
| | | | AGGAAGA | | | | dCs; InaCs; dAs; InaAs; |
| | | | CAAGTCC | | | | dGs; InaGs; dAs; |
| | | | | | | | InaAs; dGs; InaAs; dCs; |
| | | | | | | | InaAs; dAs; InaGs; |
| | | | | | | | dTs; InaCs; dC-Sup |
| 64 | Oligo31 | FXN-354 | TCAAGCA | Internal | FXN | human | dTs; InaCs; dAs; InaAs; |
| | | | TCTTTTCC | | | | dGs; InaCs; dAs; InaTs; |
| | | | GGAA | | | | dCs; InaTs; dTs; |
| | | | | | | | InaTs; dTs; InaCs; dCs; |
| | | | | | | | naGs; dGs; InaAs; dA- |
| | | | | | | | Sup |
| 65 | Oligo32 | FXN-355 | TCCTTAAA | Internal | FXN | human | dTs; InaCs; dCs; InaTs; |
| | | | ACGGGGC | | | | dTs; InaAs; dAs; InaAs; |
| | | | TGGGCA | | | | dAs; InaCs; dGs; InaGs; |
| | | | | | | | dGs; InaGs; dCs; |
| | | | | | | | InaTs; dGs; InaGs; dGs; |
| | | | | | | | InaCs; dA-Sup |
| 66 | Oligo33 | FXN-356 | TTGGCCT | Internal | FXN | human | dTs; InaTs; dGs; InaGs; |
| | | | GATAGCT | | | | dCs; InaCs; dTs; InaGs; |
| | | | TTTAATG | | | | dAs; InaTs; dAs; |
| | | | | | | | InaGs; dCs; InaTs; dTs; |
| | | | | | | | InaTs; dTs; InaAs; dAs; |
| | | | | | | | InaTs; dG-Sup |
| 67 | Oligo34 | FXN-357 | CCTCAGCT | Internal | FXN | human | dCs; InaCs; dTs; InaCs; |
| | | | GCATAAT | | | | dAs; InaGs; dCs; InaTs; |
| | | | GAAGCTG | | | | dGs; InaCs; dAs; InaTs; |
| | | | GGGTC | | | | dAs; InaAs; dTs; |
| | | | | | | | InaGs; dAs; InaAs; dGs; |
| | | | | | | | InaCs; dTs; InaGs; dGs; |
| | | | | | | | InaGs; dGs; InaTs; |
| | | | | | | | dC-Sup |
| 68 | Oligo35 | FXN-358 | AACAACA | Internal | FXN | human | dAs; InaAs; dCs; InaAs; |
| | | | ACAACAA | | | | dAs; InaCs; dAs; InaAs; |
| | | | CAAAAAA | | | | dCs; InaAs; dAs; |
| | | | CAGA | | | | InaCs; dAs; InaAs; dCs; |
| | | | | | | | InaAs; dAs; InaAs; dAs; |
| | | | | | | | InaAs; dAs; InaCs; |
| | | | | | | | dAs; InaGs; dA-Sup |
| 69 | Oligo36 | FXN-359 | CCTCAAA | Internal | FXN | human | dCs; InaCs; dTs; InaCs; |
| | | | AGCAGGA | | | | dAs; InaAs; dAs; InaAs; |
| | | | ATAAAAA | | | | dGs; InaCs; dAs; |
| | | | AAATA | | | | InaGs; dGs; InaAs; dAs; |
| | | | | | | | InaTs; dAs; InaAs; dAs; |
| | | | | | | | InaAs; dAs; InaAs; |
| | | | | | | | dAs; InaAs; dTs; InaA- |
| | | | | | | | Sup |
| 70 | Oligo37 | FXN-360 | GCTGTGA | Internal | FXN | human | dGs; InaCs; dTs; InaGs; |
| | | | CACATAG | | | | dTs; InaGs; dAs; InaCs; |
| | | | CCCAACT | | | | dAs; InaCs; dAs; |
| | | | GT | | | | InaTs; dAs; InaGs; dCs; |
| | | | | | | | InaCs; dCs; InaAs; dAs; |
| | | | | | | | InaCs; dTs; InaGs; dT- |
| | | | | | | | Sup |
| 71 | Oligo38 | FXN-361 | GGAGGCA | Internal | FXN | human | dGs; InaGs; dAs; InaGs; |
| | | | ACACATTC | | | | dGs; InaCs; dAs; InaAs |
| | | | TTTCTACA | | | | dCs; InaAs; dCs; |
| | | | GA | | | | InaAs; dTs; InaTs; dCs; |
| | | | | | | | InaTs; dTs; InaTs; dCs; |
| | | | | | | | InaTs; dAs; InaCs; dAs; |
| | | | | | | | InaGs; dA-Sup |
| 72 | Oligo39 | FXN-362 | CTATTAAT | Intron | FXN | human | dCs; InaTs; dAs; InaTs; |
| | | | ATTACTG | | | | dTs; InaAs; dAs; InaTs; |
| | | | | | | | dAs; InaTs; dTs; InaAs; |
| | | | | | | | dCs; InaTs; dG- |
| | | | | | | | Sup |
| 73 | Oligo40 | FXN-363 | CATTATGT | Intron | FXN | human | dCs; InaAs; dTs; InaTs; |
| | | | GTATGTA | | | | dAs; InaTs; dGs; InaTs; |
| | | | T | | | | dGs; InaTs; dAs; InaTs; |
| | | | | | | | dGs; InaTs; dAs; InaT- |
| | | | | | | | Sup |
| 74 | Oligo41 | FXN-364 | TTTATCTA | Intron | FXN | human | dTs; InaTs; dTs; InaAs; |
| | | | TGTTATT | | | | dTs; InaCs; dTs; InaAs; |
| | | | | | | | dTs; InaGs; dTs; InaTs; |
| | | | | | | | dAs; InaTs; dT- |
| | | | | | | | Sup |
| 75 | Oligo42 | FXN-365 | CTAATTTG | Intron | FXN | human | dCs; InaTs; dAs; InaAs; |
| | | | AAGTTCT | | | | dTs; InaTs; dTs; InaGs; |
| | | | | | | | dAs; InaAs; dGs; |
| | | | | | | | InaTs; dTs; InaCs; dT- |
| | | | | | | | Sup |
| 76 | Oligo43 | FXN-366 | TTCGAACT | Exon | FXN | human | dTs; InaTs; dCs; InaGs; |
| | | | TGCGCGG | Spanning | | | dAs; InaAs; dCs; InaTs; |
| | | | | | | | dTs; InaGs; dCs; |
| | | | | | | | InaGs; dCs; InaGs; dG- |
| | | | | | | | Sup |
| 77 | Oligo44 | FXN-367 | TAGAGAG | Exon | FXN | human | dTs; InaAs; dGs; InaAs; |
| | | | CCTGGGT | Spanning | | | dGs; InaAs; dGs; InaCs; |
| | | | | | | | dCs; InaTs; dGs; |
| | | | | | | | InaGs; dGs; InaT-Sup |
| 78 | Oligo45 | FXN-368 | ACACCAC | Exon | FXN | human | dAs; InaCs; dAs; InaCs; |
| | | | TCCCAAA | Spanning | | | dCs; InaAs; dCs; InaTs; |
| | | | G | | | | dCs; InaCs; dCs; |
| | | | | | | | InaAs; dAs; InaAs; dG- |
| | | | | | | | Sup |
| 79 | Oligo46 | FXN-369 | AGGTCCA | Exon | FXN | human | dAs; InaGs; dGs; InaTs; |
| | | | CTACATAC | Spanning | | | dCs; InaCs; dAs; InaCs; |
| | | | | | | | dTs; InaAs; dCs; |
| | | | | | | | InaAs; dTs; InaAs; dC- |
| | | | | | | | Sup |
| 80 | Oligo47 | FXN-370 | CGTTAAC | Exon | FXN | human | dCs; InaGs; dTs; InaTs; |
| | | | CTGGATG | Spanning | | | dAs; InaAs; dCs; InaCs; |
| | | | G | | | | dTs; InaGs; dGs; |
| | | | | | | | InaAs; dTs; InaGs; dG- |
| | | | | | | | Sup |
| 81 | Oligo81 | FXN-404 | AAAGCCT | Antisense | FXN | human | dAs; InaAs; dAs; InaGs; |
| | | | TAAAAAC | | | | dCs; InaCs; dTs; InaTs; |
| | | | C | | | | dAs; InaAs; dAs; |
| | | | | | | | InaAs; dAs; InaCs; dC- |
| | | | | | | | Sup |
| 82 | Oligo82 | FXN-405 | TCAGGCC | Antisense | FXN | human | dTs; InaCs; dAs; InaGs; |
| | | | AAGACCC | | | | dGs; InaCs; dCs; InaAs; |
| | | | C | | | | dAs; InaGs; dAs; |
| | | | | | | | InaCs; dCs; InaCs; dC- |
| | | | | | | | Sup |
| 83 | Oligo83 | FXN-406 | CCCAGCTT | Antisense | FXN | human | dCs; InaCs; dCs; InaAs; |
| | | | CATTATG | | | | dGs; InaCs; dTs; InaTs; |
| | | | | | | | dCs; InaAs; dTs; |
| | | | | | | | InaTs; dAs; InaTs; dG- |
| | | | | | | | Sup |
| 84 | Oligo84 | FXN-407 | AATGTGT | Antisense | FXN | human | dAs; InaAs; dTs; InaGs; |
| | | | TGCCTCCT | | | | dTs; InaGs; dTs; InaTs; |
| | | | | | | | dGs; InaCs; dCs; |
| | | | | | | | InaTs; dCs; InaCs; dT- |
| | | | | | | | Sup |
| 85 | Oligo85 | FXN-408 | AAAAAGC | Antisense | FXN | human | dAs; InaAs; dAs; InaAs; |
| | | | AAAATAA | | | | dAs; InaGs; dCs; InaAs; |
| | | | T | | | | dAs; InaAs; dAs; |
| | | | | | | | InaTs; dAs; InaAs; dT- |
| | | | | | | | Sup |
| 86 | Oligo86 | FXN-409 | CCAGGAG | Antisense | FXN | human | dCs; InaCs; dAs; InaGs; |
| | | | GGAAAAT | | | | dGs; InaAs; dGs; InaGs; |
| | | | G | | | | dGs; InaAs; dAs; |
| | | | | | | | InaAs; dAs; InaTs; dG- |
| | | | | | | | Sup |
| 87 | Oligo87 | FXN-410 | TAAAGGG | Antisense | FXN | human | dTs; InaAs; dAs; InaAs; |
| | | | TAGCCTA | | | | dGs; InaGs; dGs; |
| | | | C | | | | InaTs; dAs; InaGs; dCs; |
| | | | | | | | InaCs; dTs; InaAs; dC- |
| | | | | | | | Sup |
| 88 | Oligo88 | FXN-411 | AAAGCAA | Antisense | FXN | human | dAs; InaAs; dAs; InaGs; |
| | | | CAAGCCT | | | | dCs; InaAs; dAs; InaCs; |
| | | | C | | | | dAs; InaAs; dGs; |
| | | | | | | | InaCs; dCs; InaTs; dC- |
| | | | | | | | Sup |
| 89 | Oligo89 | FXN-412 | ATAACAT | Antisense | FXN | human | dAs; InaTs; dAs; InaAs; |
| | | | CATACAT | | | | dCs; InaAs; dTs; InaCs; |
| | | | G | | | | dAs; InaTs; dAs; |
| | | | | | | | InaCs; dAs; InaTs; dG- |
| | | | | | | | Sup |
| 90 | Oligo90 | FXN-413 | GATACTA | Antisense | FXN | human | dGs; InaAs; dTs; InaAs; |
| | | | TCTTCCTC | | | | dCs; InaTs; dAs; InaTs; |
| | | | | | | | dCs; InaTs; dTs; |
| | | | | | | | InaCs; dCs; InaTs; dC- |
| | | | | | | | Sup |
| 91 | Oligo91 | FXN-414 | ATGGGGG | Antisense | FXN | human | dAs; InaTs; dGs; InaGs; |
| | | | ACGGGGC | | | | dGs; InaGs; dGs; |
| | | | A | | | | InaAs; dCs; InaGs; dGs; |
| | | | | | | | InaGs; dGs; InaCs; dA- |
| | | | | | | | Sup |
| 92 | Oligo92 | FXN-415 | GGTTGAG | Antisense | FXN | human | dGs; InaGs; dTs; InaTs; |
| | | | ACTGGGT | | | | dGs; InaAs; dGs; InaAs; |
| | | | G | | | | dCs; InaTs; dGs; |
| | | | | | | | InaGs; dGs; InaTs; dG- |
| | | | | | | | Sup |
| 93 | Oligo93 | FXN-416 | AGACTGA | Antisense | FXN | human | dAs; InaGs; dAs; InaCs; |
| | | | AGAGGTG | | | | dTs; InaGs; dAs; InaAs; |
| | | | C | | | | dGs; InaAs; dGs; |
| | | | | | | | InaGs; dTs; InaGs; dC- |
| | | | | | | | Sup |
| 94 | Oligo94 | FXN-417 | CGGGACG | Antisense | FXN | human | dCs; InaGs; dGs; InaGs; |
| | | | GCTGTGT | | | | dAs; InaCs; dGs; InaGs; |
| | | | T | | | | dCs; InaTs; dGs; |
| | | | | | | | InaTs; dGs; InaTs; dT- |
| | | | | | | | Sup |
| 95 | Oligo95 | FXN-418 | TCTGTGT | Antisense | FXN | human | dTs; InaCs; dTs; InaGs; |
| | | | GGGCAGC | | | | dTs; InaGs; dTs; InaGs; |
| | | | A | | | | dGs; InaGs; dCs; |
| | | | | | | | InaAs; dGs; InaCs; dA- |
| | | | | | | | Sup |
| 96 | Oligo96 | FXN-419 | AAAGCCT | Antisense | FXN | human | InaAs; InaAs; InaAs; |
| | | | TAAAAAC | | | | dGs; dCs; dCs; dTs; dTs; |
| | | | C | | | | dAs; dAs; dAs; dAs; |
| | | | | | | | InaAs; InaCs; InaC- |
| | | | | | | | Sup |
| 97 | Oligo97 | FXN-420 | TCAGGCC | Antisense | FXN | human | InaTs; InaCs; InaAs; |
| | | | AAGACCC | | | | dGs; dGs; dCs; dCs; dAs; |
| | | | C | | | | dAs; dGs; dAs; dCs; |
| | | | | | | | InaCs; InaCs; InaC- |
| | | | | | | | Sup |
| 98 | Oligo98 | FXN-421 | CCCAGCTT | Antisense | FXN | human | InaCs; InaCs; InaCs; |
| | | | CATTATG | | | | dAs; dGs; dCs; dTs; dTs; |
| | | | | | | | dCs; dAs; dTs; dTs; |
| | | | | | | | InaAs; InaTs; InaG-Sup |
| 99 | Oligo99 | FXN-422 | AATGTGT | Antisense | FXN | human | InaAs; InaAs; InaTs; |
| | | | TGCCTCCT | | | | dGs; dTs; dGs; dTs; dTs; |
| | | | | | | | dGs; dCs; dCs; dTs; |
| | | | | | | | InaCs; InaCs; InaT-Sup |
| 100 | Oligo100 | FXN-423 | AAAAAGC | Antisense | FXN | human | InaAs; InaAs; InaAs; |
| | | | AAAATAA | | | | dAs; dAs; dGs; dCs; dAs; |
| | | | T | | | | dAs; dAs; dAs; dTs; |
| | | | | | | | InaAs; InaAs; InaT- |
| | | | | | | | Sup |
| 101 | Oligo101 | FXN-424 | CCAGGAG | Antisense | FXN | human | InaCs; InaCs; InaAs; |
| | | | GGAAAAT | | | | dGs; dGs; dAs; dGs; dGs; |
| | | | G | | | | dGs; dAs; dAs; dAs; |
| | | | | | | | InaAs; InaTs; InaG- |
| | | | | | | | Sup |
| 102 | Oligo102 | FXN-425 | TAAAGGG | Antisense | FXN | human | InaTs; InaAs; InaAs; |
| | | | TAGCCTA | | | | dAs; dGs; dGs; dGs; dTs; |
| | | | C | | | | dAs; dGs; dCs; dCs; |
| | | | | | | | InaTs; InaAs; InaC- |
| | | | | | | | Sup |
| 103 | Oligo103 | FXN-426 | AAAGCAA | Antisense | FXN | human | InaAs; InaAs; InaAs; |
| | | | CAAGCCT | | | | dGs; dCs; dAs; dAs; dCs; |
| | | | C | | | | dAs; dAs; dGs; dCs; |
| | | | | | | | InaCs; InaTs; InaC- |
| | | | | | | | Sup |
| 104 | Oligo104 | FXN-427 | ATAACAT | Antisense | FXN | human | InaAs; InaTs; InaAs; |
| | | | CATACAT | | | | dAs; dCs; dAs; dTs; dCs; |
| | | | G | | | | dAs; dTs; dAs; dCs; |
| | | | | | | | InaAs; InaTs; InaG-Sup |
| 105 | Oligo105 | FXN-428 | GATACTA | Antisense | FXN | human | InaGs; InaAs; InaTs; |
| | | | TCTTCCTC | | | | dAs; dCs; dTs; dAs; dTs; |
| | | | | | | | dCs; dTs; dTs; dCs; |
| | | | | | | | InaCs; InaTs; InaC-Sup |
| 106 | Oligo106 | FXN-429 | ATGGGGG | Antisense | FXN | human | InaAs; InaTs; InaGs; |
| | | | ACGGGGC | | | | dGs; dGs; dGs; dGs; dAs; |
| | | | A | | | | dCs; dGs; dGs; dGs; |
| | | | | | | | InaGs; InaCs; InaA- |
| | | | | | | | Sup |
| 107 | Oligo107 | FXN-430 | GGTTGAG | Antisense | FXN | human | InaGs; InaGs; InaTs; |
| | | | ACTGGGT | | | | dTs; dGs; dAs; dGs; dAs; |
| | | | G | | | | dCs; dTs; dGs; dGs; |
| | | | | | | | InaGs; InaTs; InaG- |
| | | | | | | | Sup |
| 108 | Oligo108 | FXN-431 | AGACTGA | Antisense | FXN | human | InaAs; InaGs; InaAs; |
| | | | AGAGGTG | | | | dCs; dTs; dGs; dAs; dAs; |
| | | | C | | | | dGs; dAs; dGs; dGs; |
| | | | | | | | InaTs; InaGs; InaC- |
| | | | | | | | Sup |
| 109 | Oligo109 | FXN-432 | CGGGACG | Antisense | FXN | human | InaCs; InaGs; InaGs; |
| | | | GCTGTGT | | | | dGs; dAs; dCs; dGs; dGs; |
| | | | T | | | | dCs; dTs; dGs; dTs; |
| | | | | | | | InaGs; InaTs; InaT- |
| | | | | | | | Sup |
| 110 | Oligo110 | FXN-433 | TCTGTGT | Antisense | FXN | human | InaTs; InaCs; InaTs; |
| | | | GGGCAGC | | | | dGs; dTs; dGs; dTs; dGs; |
| | | | A | | | | dGs; dGs; dCs; dAs; |
| | | | | | | | InaGs; InaCs; InaA- |
| | | | | | | | Sup |
| 111 | Oligo111 | FXN-115 | GAAGAAG | Antisense | FXN | human | InaGs; InaAs; InaAs; |
| | | | AAGAAGA | | | | dGs; dAs; dAs; dGs; dAs; |
| | | | A | | | | dAs; dGs; dAs; dAs; |
| | | | | | | | InaGs; InaAs; InaA- |
| | | | | | | | Sup |
| 112 | Oligo112 | FXN-117 | TTCTTCTT | Antisense | FXN | human | InaTs; InaTs; InaCs; |
| | | | CTTCTTC | | | | dTs; dTs; dCs; dTs; dTs; |
| | | | | | | | dCs; dTs; dTs; dCs; |
| | | | | | | | InaTs; InaTs; InaC-Sup |

TABLE 5
Other targeting FXN

| SEQ | | | | | |
|---|---|---|---|---|---|
| ID | Oligo | | Gene | | Formatted |
| NO | Name | Base Sequence | Name | Organism | Sequence |
| 113 | 324 | CGGCGCCCGAGAG | FXN | human | dCs; InaGs; dGs; InaCs; dGs; |
| | | TCCACAT | | | InaCs; dCs; InaCs; dGs; InaAs; |
| | | | | | dGs; InaAs; dGs; InaTs; dCs; |
| | | | | | InaCs; dAs; InaCs; dAs; InaT-Sup |
| 114 | 329 | ACGGCGGCCGCAG | FXN | human | dAs; InaCs; dGs; InaGs; dCs; |
| | | AGTGGGG | | | InaGs; dGs; InaCs; dCs; InaGs; |
| | | | | | dCs; InaAs; dGs; InaAs; dGs; |
| | | | | | InaTs; dGs; InaGs; dGs; InaG-Sup |
| 115 | 359 | CCTCAAAAGCAGGA | FXN | human | dCs; InaCs; dTs; InaCs; dAs; |
| | | ATAAAAAAAATA | | | InaAs; dAs; InaAs; dGs; InaCs; |
| | | | | | dAs; InaGs; dGs; InaAs; dAs; InaTs; |
| | | | | | dAs; InaAs; dAs; InaAs; dAs; |
| | | | | | InaAs; dAs; InaAs; dTs; InaA-Sup |
| 116 | 414 | ATGGGGGACGGGG | FXN | human | dAs; InaTs; dGs; InaGs; dGs; |
| | | CA | | | InaGs; dGs; InaAs; dCs; InaGs; |
| | | | | | dGs; InaGs; dGs; InaCs; dA-Sup |
| 117 | 415 | GGTTGAGACTGGG | FXN | human | dGs; InaGs; dTs; InaTs; dGs; |
| | | TG | | | InaAs; dGs; InaAs; dCs; InaTs; |
| | | | | | dGs; InaGs; dGs; InaTs; dG-Sup |
| 118 | 429 | ATGGGGGACGGGG | FXN | human | dAs; InaTs; dGs; InaGs; dGs; |
| | | CA | | | InaGs; dGs; InaAs; dCs; InaGs; |
| | | | | | dGs; InaGs; dGs; InaCs; dA-Sup |

Several additional oligonucleotides were designed to target the 5′ end of an RNA, the 3′ end of an RNA, or target both the 5′ end and 3′ end of an RNA (“bridging oligos”). These oligos are shown in Table 6.

TABLE 6
Oligonucleotides designed to target 5′ and 3′ ends of RNAs

| SEQ | Oligo | | Gene | Target | | |
|---|---|---|---|---|---|---|
| ID NO | Name | Base Sequence | Name | Region | Organism | Formatted Sequence |
| 119 | FXN-437 | TGACCCAAGGGAGACTT | FXN | 5′ and 3′ | human | dTs; InaGs; dAs; InaCs; dCs; |
| | m02 | TTTGGTTTTTAAGGCTTT | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dGs; InaGs; dTs; InaTs; |
| | | | | | | dTs; InaTs; dTs; InaAs; dAs; |
| | | | | | | InaGs; dGs; InaCs; dTs; |
| | | | | | | InaTs; dT-Sup |
| 120 | FXN-438 | TGGCCACTGGCCGCATT | FXN | 5′ and 3′ | human | dTs; InaGs; dGs; InaCs; dCs; |
| | m02 | TTTGGTTTTTAAGGCTTT | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dGs; InaGs; dTs; InaTs; |
| | | | | | | dTs; InaTs; dTs; InaAs; dAs; |
| | | | | | | InaGs; dGs; InaCs; dTs; InaTs; |
| | | | | | | dT-Sup |
| 121 | FXN-439 | CGGCGACCCCTGGTGTT | FXN | 5′ and 3′ | human | dCs; InaGs; dGs; InaCs; dGs; |
| | m02 | TTTGGTTTTTAAGGCTTT | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dGs; InaGs; dTs; InaTs; |
| | | | | | | dTs; InaTs; dTs; InaAs; dAs; |
| | | | | | | InaGs; dGs; InaCs; dTs; InaTs; |
| | | | | | | dT-Sup |
| 122 | FXN-440 | CGCCCTCCAGCGCTGTT | FXN | 5′ and 3′ | human | dCs; InaGs; dCs; InaCs; dCs; |
| | m02 | TTTGGTTTTTAAGGCTTT | | | | InaTs; dCs; InaCs; dAs; InaGs; |
| | | | | | | dCs; InaGs; dCs; InaTs; dGs; |
| | | | | | | InaTs; dTs; InaTs; dTs; InaTs; |
| | | | | | | dGs; InaGs; dTs; InaTs; |
| | | | | | | dTs; InaTs; dTs; InaAs; dAs; |
| | | | | | | InaGs; dGs; InaCs; dTs; InaTs; |
| | | | | | | dT-Sup |
| 123 | FXN-441 | CGCTCCGCCCTCCAGTTT | FXN | 5′ and 3′ | human | dCs; InaGs; dCs; InaTs; dCs; |
| | m02 | TTGGTTTTTAAGGCTTT | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dGs; InaGs; dTs; InaTs; |
| | | | | | | dTs; InaTs; dTs; InaAs; dAs; |
| | | | | | | InaGs; dGs; InaCs; dTs; InaTs; |
| | | | | | | dT-Sup |
| 124 | FXN-442 | TGACCCAAGGGAGACTT | FXN | 5′ and 3′ | human | dTs; InaGs; dAs; InaCs; dCs; |
| | m02 | TTTGGGGTCTTGGCCTG | | | | InaCs; dAs; InaAs; dGs; InaGs; |
| | | A | | | | dGs; InaAs; dGs; InaAs; |
| | | | | | | dCs; InaTs; dTs; InaTs; dTs; |
| | | | | | | InaTs; dGs; InaGs; dGs; InaGs; |
| | | | | | | dTs; InaCs; dTs; InaTs; dGs; |
| | | | | | | InaGs; dCs; InaCs; dTs; InaGs; |
| | | | | | | dA-Sup |
| 125 | FXN-443 | TGGCCACTGGCCGCATT | FXN | 5′ and 3′ | human | dTs; InaGs; dGs; InaCs; dCs; |
| | m02 | TTTGGGGTCTTGGCCTG | | | | InaAs; dCs; InaTs; dGs; InaGs; |
| | | A | | | | dCs; InaCs; dGs; InaCs; |
| | | | | | | dAs; InaTs; dTs; InaTs; dTs; |
| | | | | | | InaTs; dGs; InaGs; dGs; InaGs; |
| | | | | | | dTs; InaCs; dTs; InaTs; dGs; |
| | | | | | | InaGs; dCs; InaCs; dTs; InaGs; |
| | | | | | | dA-Sup |
| 126 | FXN-444 | CGGCGACCCCTGGTGTT | FXN | 5′ and 3′ | human | dCs; InaGs; dGs; InaCs; dGs; |
| | m02 | TTTGGGGTCTTGGCCTG | | | | InaAs; dCs; InaCs; dCs; InaCs; |
| | | A | | | | dTs; InaGs; dGs; InaTs; |
| | | | | | | dGs; InaTs; dTs; InaTs; dTs; |
| | | | | | | InaTs; dGs; InaGs; dGs; InaGs; |
| | | | | | | dTs; InaCs; dTs; InaTs; dGs; |
| | | | | | | InaGs; dCs; InaCs; dTs; InaGs; |
| | | | | | | dA-Sup |
| 127 | FXN-445 | CGCCCTCCAGCGCTGTT | FXN | 5′ and 3′ | human | dCs; InaGs; dCs; InaCs; dCs; |
| | m02 | TTTGGGGTCTTGGCCTG | | | | InaTs; dCs; InaCs; dAs; InaGs; |
| | | A | | | | dCs; InaGs; dCs; InaTs; dGs; |
| | | | | | | InaTs; dTs; InaTs; dTs; InaTs; |
| | | | | | | ; dGs; InaGs; dGs; InaGs; |
| | | | | | | dTs; InaCs; dTs; InaTs; dGs; |
| | | | | | | InaGs; dCs; InaCs; dTs; InaGs; |
| | | | | | | dA-Sup |
| 128 | FXN-446 | CGCTCCGCCCTCCAGTTT | FXN | 5′ and 3′ | human | dCs; InaGs; dCs; InaTs; dCs; |
| | m02 | TTGGGGTCTTGGCCTGA | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dGs; InaGs; dGs; InaGs; |
| | | | | | | dTs; InaCs; dTs; InaTs; dGs; |
| | | | | | | InaGs; dCs; InaCs; dTs; InaGs; |
| | | | | | | dA-Sup |
| 129 | FXN-447 | TGACCCAAGGGAGACTT | FXN | 5′ and 3′ | human | dTs; InaGs; dAs; InaCs; dCs; |
| | m02 | TTTCATAATGAAGCTGG | | | | InaCs; dAs; InaAs; dGs; InaGs; |
| | | G | | | | dGs; InaAs; dGs; InaAs; |
| | | | | | | dCs; InaTs; dTs; InaTs; dTs; |
| | | | | | | InaTs; dCs; InaAs; dTs; InaAs; |
| | | | | | | dAs; InaTs; dGs; InaAs; |
| | | | | | | dAs; InaGs; dCs; InaTs; dGs; |
| | | | | | | InaGs; dG-Sup |
| 130 | FXN-448 | TGGCCACTGGCCGCATT | FXN | 5′ and 3′ | human | dTs; InaGs; dGs; InaCs; dCs; |
| | m02 | TTTCATAATGAAGCTGG | | | | InaAs; dCs; InaTs; dGs; InaGs; |
| | | G | | | | dCs; InaCs; dGs; InaCs; |
| | | | | | | dAs; InaTs; dTs; InaTs; dTs; |
| | | | | | | InaTs; dCs; InaAs; dTs; InaAs; |
| | | | | | | dAs; InaTs; dGs; InaAs; |
| | | | | | | dAs; InaGs; dCs; InaTs; dGs; |
| | | | | | | InaGs; dG-Sup |
| 131 | FXN-449 | CGGCGACCCCTGGTGTT | FXN | 5′ and 3′ | human | dCs; InaGs; dGs; InaCs; dGs; |
| | m02 | TTTCATAATGAAGCTGG | | | | InaAs; dCs; InaCs; dCs; InaCs; |
| | | G | | | | dTs; InaGs; dGs; InaTs; |
| | | | | | | dGs; InaTs; dTs; InaTs; dTs; |
| | | | | | | InaTs; dCs; InaAs; dTs; InaAs; |
| | | | | | | dAs; InaTs; dGs; InaAs; dAs; |
| | | | | | | InaGs; dCs; InaTs; dGs; InaGs; |
| | | | | | | dG-Sup |
| 132 | FXN-450 | CGCCCTCCAGCGCTGTT | FXN | 5′ and 3′ | human | dCs; InaGs; dCs; InaCs; dCs; |
| | m02 | TTTCATAATGAAGCTGG | | | | InaTs; dCs; InaCs; dAs; InaGs; |
| | | G | | | | dCs; InaGs; dCs; InaTs; dGs; |
| | | | | | | InaTs; dTs; InaTs; dTs; InaTs; |
| | | | | | | dCs; InaAs; dTs; InaAs; dAs; |
| | | | | | | InaTs; dGs; InaAs; dAs; |
| | | | | | | InaGs; dCs; InaTs; dGs; InaGs; |
| | | | | | | dG-Sup |
| 133 | FXN-451 | CGCTCCGCCCTCCAGTTT | FXN | 5′ and 3′ | human | dCs; InaGs; dCs; InaTs; dCs; |
| | m02 | TTCATAATGAAGCTGGG | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dCs; InaAs; dTs; InaAs; |
| | | | | | | dAs; InaTs; dGs; InaAs; dAs; |
| | | | | | | InaGs; dCs; InaTs; dGs; InaGs; |
| | | | | | | dG-Sup |
| 134 | FXN-452 | TGACCCAAGGGAGACTT | FXN | 5′ and 3′ | human | dTs; InaGs; dAs; InaCs; dCs; |
| | m02 | TTTAGGAGGCAACACAT | | | | InaCs; dAs; InaAs; dGs; InaGs; |
| | | T | | | | dGs; InaAs; dGs; InaAs; |
| | | | | | | dCs; InaTs; dTs; InaTs; dTs; |
| | | | | | | InaTs; dAs; InaGs; dGs; InaAs; |
| | | | | | | dGs; InaGs; dCs; InaAs; |
| | | | | | | dAs; InaCs; dAs; InaCs; dAs; |
| | | | | | | InaTs; dT-Sup |
| 135 | FXN-453 | TGGCCACTGGCCGCATT | FXN | 5′ and 3′ | human | dTs; InaGs; dGs; InaCs; dCs; |
| | m02 | TTTAGGAGGCAACACAT | | | | InaAs; dCs; InaTs; dGs; InaGs; |
| | | T | | | | dCs; InaCs; dGs; InaCs; |
| | | | | | | dAs; InaTs; dTs; InaTs; dTs; |
| | | | | | | InaTs; dAs; InaGs; dGs; InaAs; |
| | | | | | | dGs; InaGs; dCs; InaAs; |
| | | | | | | dAs; InaCs; dAs; InaCs; dAs; |
| | | | | | | InaTs; dT-Sup |
| 136 | FXN-454 | CGGCGACCCCTGGTGTT | FXN | 5′ and 3′ | human | dCs; InaGs; dGs; InaCs; dGs; |
| | m02 | TTTAGGAGGCAACACAT | | | | InaAs; dCs; InaCs; dCs; InaCs; |
| | | T | | | | dTs; InaGs; dGs; InaTs; |
| | | | | | | dGs; InaTs; dTs; InaTs; dTs; |
| | | | | | | InaTs; dAs; InaGs; dGs; InaAs; |
| | | | | | | dGs; InaGs; dCs; InaAs; dAs; |
| | | | | | | InaCs; dAs; InaCs; dAs; |
| | | | | | | InaTs; dT-Sup |
| 137 | FXN-455 | CGCCCTCCAGCGCTGTT | FXN | 5′ and 3′ | human | dCs; InaGs; dCs; InaCs; dCs; |
| | m02 | TTTAGGAGGCAACACAT | | | | InaTs; dCs; InaCs; dAs; InaGs; |
| | | T | | | | dCs; InaGs; dCs; InaTs; dGs; |
| | | | | | | InaTs; dTs; InaTs; dTs; InaTs; |
| | | | | | | dAs; InaGs; dGs; InaAs; |
| | | | | | | dGs; InaGs; dCs; InaAs; dAs; |
| | | | | | | InaCs; dAs; InaCs; dAs; InaTs; |
| | | | | | | dT-Sup |
| 138 | FXN-456 | CGCTCCGCCCTCCAGTTT | FXN | 5′ and 3′ | human | dCs; InaGs; dCs; InaTs; dCs; |
| | m02 | TTAGGAGGCAACACATT | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dAs; InaGs; dGs; InaAs; |
| | | | | | | dGs; InaGs; dCs; InaAs; dAs; |
| | | | | | | InaCs; dAs; InaCs; dAs; InaTs; |
| | | | | | | dT-Sup |
| 139 | FXN-457 | TGACCCAAGGGAGACTT | FXN | 5′ and 3′ | human | dTs; InaGs; dAs; InaCs; dCs; |
| | m02 | TTTATTATTTTGCTTTTT | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dAs; InaTs; dTs; InaAs; |
| | | | | | | dTs; InaTs; dTs; InaTs; dGs; |
| | | | | | | InaCs; dTs; InaTs; dTs; InaTs; |
| | | | | | | dT-Sup |
| 140 | FXN-458 | TGGCCACTGGCCGCATT | FXN | 5′ and 3′ | human | dTs; InaGs; dGs; InaCs; dCs; |
| | m02 | TTTATTATTTTGCTTTTT | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dAs; InaTs; dTs; InaAs; |
| | | | | | | dTs; InaTs; dTs; InaTs; dGs; |
| | | | | | | InaCs; dTs; InaTs; dTs; InaTs; |
| | | | | | | dT-Sup |
| 141 | FXN-459 | CGGCGACCCCTGGTGTT | FXN | 5′ and 3′ | human | dCs; InaGs; dGs; InaCs; dGs; |
| | m02 | TTTATTATTTTGCTTTTT | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dAs; InaTs; dTs; InaAs; |
| | | | | | | dTs; InaTs; dTs; InaTs; dGs; |
| | | | | | | InaCs; dTs; InaTs; dTs; InaTs; |
| | | | | | | dT-Sup |
| 142 | FXN-460 | CGCCCTCCAGCGCTGTT | FXN | 5′ and 3′ | human | dCs; InaGs; dCs; InaCs; dCs; |
| | m02 | TTTATTATTTTGCTTTTT | | | | InaTs; dCs; InaCs; dAs; InaGs; |
| | | | | | | dCs; InaGs; dCs; InaTs; dGs; |
| | | | | | | InaTs; dTs; InaTs; dTs; InaTs; |
| | | | | | | dAs; InaTs; dTs; InaAs; |
| | | | | | | dTs; InaTs; dTs; InaTs; dGs; |
| | | | | | | InaCs; dTs; InaTs; dTs; InaTs; |
| | | | | | | dT-Sup |
| 143 | FXN-461 | CGCTCCGCCCTCCAGTTT | FXN | 5′ and 3′ | human | dCs; InaGs; dCs; InaTs; dCs; |
| | m02 | TTATTATTTTGCTTTTT | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dAs; InaTs; dTs; InaAs; |
| | | | | | | dTs; InaTs; dTs; InaTs; dGs; |
| | | | | | | InaCs; dTs; InaTs; dTs; InaTs; |
| | | | | | | dT-Sup |
| 144 | FXN-462 | TGACCCAAGGGAGACTT | FXN | 5′ and 3′ | human | dTs; InaGs; dAs; InaCs; dCs; |
| | m02 | TTTCATTTTCCCTCCTGG | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dCs; InaAs; dTs; InaTs; |
| | | | | | | dTs; InaTs; dCs; InaCs; dCs; |
| | | | | | | InaTs; dCs; InaCs; dTs; InaGs; |
| | | | | | | dG-Sup |
| 145 | FXN-463 | TGGCCACTGGCCGCATT | FXN | 5′ and 3′ | human | dTs; InaGs; dGs; InaCs; dCs; |
| | m02 | TTTCATTTTCCCTCCTGG | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dCs; InaAs; dTs; InaTs; |
| | | | | | | dTs; InaTs; dCs; InaCs; dCs; |
| | | | | | | InaTs; dCs; InaCs; dTs; InaGs; |
| | | | | | | dG-Sup |
| 146 | FXN-464 | CGGCGACCCCTGGTGTT | FXN | 5′ and 3′ | human | dCs; InaGs; dGs; InaCs; dGs; |
| | m02 | TTTCATTTTCCCTCCTGG | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dCs; InaAs; dTs; InaTs; |
| | | | | | | dTs; InaTs; dCs; InaCs; dCs; |
| | | | | | | InaTs; dCs; InaCs; dTs; InaGs; |
| | | | | | | dG-Sup |
| 147 | FXN-465 | CGCCCTCCAGCGCTGTT | FXN | 5′ and 3′ | human | dCs; InaGs; dCs; InaCs; dCs; |
| | m02 | TTTCATTTTCCCTCCTGG | | | | InaTs; dCs; InaCs; dAs; InaGs; |
| | | | | | | dCs; InaGs; dCs; InaTs; dGs; |
| | | | | | | InaTs; dTs; InaTs; dTs; InaTs; |
| | | | | | | dCs; InaAs; dTs; InaTs; |
| | | | | | | dTs; InaTs; dCs; InaCs; dCs; |
| | | | | | | InaTs; dCs; InaCs; dTs; InaGs; |
| | | | | | | dG-Sup |
| 148 | FXN-466 | CGCTCCGCCCTCCAGTTT | FXN | 5′ and 3′ | human | dCs; InaGs; dCs; InaTs; dCs; |
| | m02 | TTCATTTTCCCTCCTGG | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dCs; InaAs; dTs; InaTs; |
| | | | | | | dTs; InaTs; dCs; InaCs; dCs; |
| | | | | | | InaTs; dCs; InaCs; dTs; InaGs; |
| | | | | | | dG-Sup |
| 149 | FXN-467 | TGACCCAAGGGAGACTT | FXN | 5′ and 3′ | human | dTs; InaGs; dAs; InaCs; dCs; |
| | m02 | TTTGTAGGCTACCCTTTA | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dGs; InaTs; dAs; InaGs; |
| | | | | | | dGs; InaCs; dTs; InaAs; dCs; |
| | | | | | | InaCs; dCs; InaTs; dTs; InaTs; |
| | | | | | | dA-Sup |
| 150 | FXN-468 | TGGCCACTGGCCGCATT | FXN | 5′ and 3′ | human | dTs; InaGs; dGs; InaCs; dCs; |
| | m02 | TTTGTAGGCTACCCTTTA | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dGs; InaTs; dAs; InaGs; |
| | | | | | | dGs; InaCs; dTs; InaAs; dCs; |
| | | | | | | InaCs; dCs; InaTs; dTs; InaTs; |
| | | | | | | dA-Sup |
| 151 | FXN-469 | CGGCGACCCCTGGTGTT | FXN | 5′ and 3′ | human | dCs; InaGs; dGs; InaCs; dGs; |
| | m02 | TTTGTAGGCTACCCTTTA | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dGs; InaTs; dAs; InaGs; |
| | | | | | | dGs; InaCs; dTs; InaAs; dCs; |
| | | | | | | InaCs; dCs; InaTs; dTs; InaTs; |
| | | | | | | s; dA-Sup |
| 152 | FXN-470 | CGCCCTCCAGCGCTGTT | FXN | 5′ and 3′ | human | dCs; InaGs; dCs; InaCs; dCs; |
| | m02 | TTTGTAGGCTACCCTTTA | | | | InaTs; dCs; InaCs; dAs; InaG |
| | | | | | | s; dCs; InaGs; dCs; InaTs; dG |
| | | | | | | InaTs; dTs; InaTs; dTs; InaTs; |
| | | | | | | dGs; InaTs; dAs; InaGs; |
| | | | | | | dGs; InaCs; dTs; InaAs; dCs; |
| | | | | | | InaCs; dCs; InaTs; dTs; InaTs; |
| | | | | | | dA-Sup |
| 153 | FXN-471 | CGCTCCGCCCTCCAGTTT | FXN | 5′ and 3′ | human | dCs; InaGs; dCs; InaTs; dCs; |
| | m02 | TTGTAGGCTACCCTTTA | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dGs; InaTs; dAs; InaGs; |
| | | | | | | dGs; InaCs; dTs; InaAs; dCs; |
| | | | | | | InaCs; dCs; InaTs; dTs; InaTs; |
| | | | | | | dA-Sup |
| 154 | FXN-472 | TGACCCAAGGGAGACTT | FXN | 5′ and 3′ | human | dTs; InaGs; dAs; InaCs; dCs; |
| | m02 | TTTGAGGCTTGTTGCTTT | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dGs; InaAs; dGs; InaGs; |
| | | | | | | dCs; InaTs; dTs; InaGs; dTs; |
| | | | | | | InaTs; dGs; InaCs; dTs; InaTs; |
| | | | | | | dT-Sup |
| 155 | FXN-473 | TGGCCACTGGCCGCATT | FXN | 5′ and 3′ | human | dTs; InaGs; dGs; InaCs; dCs; |
| | m02 | TTTGAGGCTTGTTGCTTT | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dGs; InaAs; dGs; InaGs; |
| | | | | | | dCs; InaTs; dTs; InaGs; dTs; |
| | | | | | | InaTs; dGs; InaCs; dTs; |
| | | | | | | InaTs; dT-Sup |
| 156 | FXN-474 | CGGCGACCCCTGGTGTT | FXN | 5′ and 3′ | human | dCs; InaGs; dGs; InaCs; dGs; |
| | m02 | TTTGAGGCTTGTTGCTTT | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dGs; InaAs; dGs; InaGs; |
| | | | | | | dCs; InaTs; dTs; InaGs; dTs; |
| | | | | | | InaTs; dGs; InaCs; dTs; InaTs; |
| | | | | | | dT-Sup |
| 157 | FXN-475 | CGCCCTCCAGCGCTGTT | FXN | 5′ and 3′ | human | dCs; InaGs; dCs; InaCs; dCs; |
| | m02 | TTTGAGGCTTGTTGCTTT | | | | InaTs; dCs; InaCs; dAs; InaGs; |
| | | | | | | dCs; InaGs; dCs; InaTs; dGs; |
| | | | | | | InaTs; dTs; InaTs; dTs; InaTs; |
| | | | | | | dGs; InaAs; dGs; InaGs; |
| | | | | | | dCs; InaTs; dTs; InaGs; dTs; |
| | | | | | | InaTs; dGs; InaCs; dTs; InaTs; |
| | | | | | | dT-Sup |
| 158 | FXN-476 | CGCTCCGCCCTCCAGTTT | FXN | 5′ and 3′ | human | dCs; InaGs; dCs; InaTs; dCs; |
| | m02 | TTGAGGCTTGTTGCTTT | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | ; dGs; InaAs; dGs; InaGs; |
| | | | | | | dCs; InaTs; dTs; InaGs; dTs; |
| | | | | | | InaTs; dGs; InaCs; dTs; InaTs; |
| | | | | | | dT-Sup |
| 159 | FXN-477 | TGACCCAAGGGAGACTT | FXN | 5′ and 3′ | human | dTs; InaGs; dAs; InaCs; dCs; |
| | m02 | TTTCATGTATGATGTTAT | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dCs; InaAs; dTs; InaGs; |
| | | | | | | dTs; InaAs; dTs; InaGs; dAs; |
| | | | | | | InaTs; dGs; InaTs; dTs; InaAs; |
| | | | | | | dT-Sup |
| 160 | FXN-478 | TGGCCACTGGCCGCATT | FXN | 5′ and 3′ | human | dTs; InaGs; dGs; InaCs; dCs; |
| | m02 | TTTCATGTATGATGTTAT | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dCs; InaAs; dTs; InaGs; |
| | | | | | | dTs; InaAs; dTs; InaGs; dAs; |
| | | | | | | InaTs; dGs; InaTs; dTs; InaAs; |
| | | | | | | dT-Sup |
| 161 | FXN-479 | CGGCGACCCCTGGTGTT | FXN | 5′ and 3′ | human | dCs; InaGs; dGs; InaCs; dGs; |
| | m02 | TTTCATGTATGATGTTAT | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dCs; InaAs; dTs; InaGs; |
| | | | | | | dTs; InaAs; dTs; InaGs; dAs; |
| | | | | | | InaTs; dGs; InaTs; dTs; InaAs; |
| | | | | | | dT-Sup |
| 162 | FXN-480 | CGCCCTCCAGCGCTGTT | FXN | 5′ and 3′ | human | dCs; InaGs; dCs; InaCs; dCs; |
| | m02 | TTTCATGTATGATGTTAT | | | | InaTs; dCs; InaCs; dAs; InaGs; |
| | | | | | | dCs; InaGs; dCs; InaTs; dGs; |
| | | | | | | InaTs; dTs; InaTs; dTs; InaTs; |
| | | | | | | dCs; InaAs; dTs; InaGs; |
| | | | | | | dTs; InaAs; dTs; InaGs; dAs; |
| | | | | | | InaTs; dGs; InaTs; dTs; InaAs; |
| | | | | | | dT-Sup |
| 163 | FXN-481 | CGCTCCGCCCTCCAGTTT | FXN | 5′ and 3′ | human | dCs; InaGs; dCs; InaTs; dCs; |
| | m02 | TTCATGTATGATGTTAT | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dCs; InaAs; dTs; InaGs; dTs; |
| | | | | | | InaAs; dTs; InaGs; dAs; |
| | | | | | | InaTs; dGs; InaTs; dTs; InaAs; |
| | | | | | | dT-Sup |
| 164 | FXN-482 | CGCCCTCCAGTTTTTGGT | FXN | 5′ and 3′ | human | dCs; InaGs; dCs; InaCs; dCs; |
| | m02 | TTTTAAG | | | | InaTs; dCs; InaCs; dAs; InaGs; |
| | | | | | | dTs; InaTs; dTs; InaTs; dTs; |
| | | | | | | naGs; dGs; InaTs; dTs; InaTs; |
| | | | | | | dTs; InaTs; dAs; InaAs; |
| | | | | | | dG-Sup |
| 165 | FXN-483 | CGCCCTCCAGTTTTTGG | FXN | 5′ and 3′ | human | dCs; InaGs; dCs; InaCs; dCs; |
| | m02 | GGTCTTGG | | | | InaTs; dCs; InaCs; dAs; InaGs; |
| | | | | | | dTs; InaTs; dTs; InaTs; dTs; |
| | | | | | | InaGs; dGs; InaGs; dGs; |
| | | | | | | InaTs; dCs; InaTs; dTs; InaGs; |
| | | | | | | dG-Sup |
| 166 | FXN-484 | CGCCCTCCAGTTTTTCAT | FXN | 5′ and 3′ | human | dCs; InaGs; dCs; InaCs; dCs; |
| | m02 | AATGAAG | | | | InaTs; dCs; InaCs; dAs; InaGs; |
| | | | | | | dTs; InaTs; dTs; InaTs; dTs; |
| | | | | | | InaCs; dAs; InaTs; dAs; InaAs; |
| | | | | | | dTs; InaGs; dAs; InaAs; |
| | | | | | | dG-Sup |
| 167 | FXN-485 | CGCCCTCCAGTTTTTAG | FXN | 5′ and 3′ | human | dCs; InaGs; dCs; InaCs; dCs; |
| | m02 | GAGGCAAC | | | | InaTs; dCs; InaCs; dAs; InaGs; |
| | | | | | | dTs; InaTs; dTs; InaTs; dTs; |
| | | | | | | InaAs; dGs; InaGs; dAs; InaGs; |
| | | | | | | dGs; InaCs; dAs; InaAs; |
| | | | | | | dC-Sup |
| 168 | FXN-486 | CGCCCTCCAGTTTTTATT | FXN | 5′ and 3′ | human | dCs; InaGs; dCs; InaCs; dCs; |
| | m02 | ATTTTGC | | | | InaTs; dCs; InaCs; dAs; InaGs; |
| | | | | | | dTs; InaTs; dTs; InaTs; dTs; |
| | | | | | | InaAs; dTs; InaTs; dAs; InaTs; |
| | | | | | | dTs; InaTs; dTs; InaGs; |
| | | | | | | dC-Sup |
| 169 | FXN-487 | CGCCCTCCAGTTTTTCAT | FXN | 5′ and 3′ | human | dCs; InaGs; dCs; InaCs; dCs; |
| | m02 | TTTCCCT | | | | InaTs; dCs; InaCs; dAs; InaGs; |
| | | | | | | dTs; InaTs; dTs; InaTs; dTs; |
| | | | | | | InaCs; dAs; InaTs; dTs; InaTs; |
| | | | | | | dTs; InaCs; dCs; InaCs; |
| | | | | | | dT-Sup |
| 170 | FXN-488 | CGCCCTCCAGTTTTTGTA | FXN | 5′ and 3′ | human | dCs; InaGs; dCs; InaCs; dCs; |
| | m02 | GGCTACC | | | | InaTs; dCs; InaCs; dAs; InaGs; |
| | | | | | | dTs; InaTs; dTs; InaTs; dTs; |
| | | | | | | InaGs; dTs; InaAs; dGs; InaGs; |
| | | | | | | dCs; InaTs; dAs; InaCs; |
| | | | | | | dC-Sup |
| 171 | FXN-489 | CGCCCTCCAGTTTTTGA | FXN | 5′ and 3′ | human | dCs; InaGs; dCs; InaCs; dCs; |
| | m02 | GGCTTGTT | | | | InaTs; dCs; InaCs; dAs; InaGs; |
| | | | | | | dTs; InaTs; dTs; InaTs; dTs; |
| | | | | | | InaGs; dAs; InaGs; dGs; InaCs; |
| | | | | | | dTs; InaTs; dGs; InaTs; |
| | | | | | | dT-Sup |
| 172 | FXN-490 | CGCCCTCCAGTTTTTCAT | FXN | 5′ and 3′ | human | dCs; InaGs; dCs; InaCs; dCs; |
| | m02 | GTATGAT | | | | InaTs; dCs; InaCs; dAs; InaGs; |
| | | | | | | dTs; InaTs; dTs; InaTs; dTs; |
| | | | | | | InaCs; dAs; InaTs; dGs; InaTs; |
| | | | | | | dAs; InaTs; dGs; InaAs; |
| | | | | | | dT-Sup |
| 173 | FXN-491 | TGACCCAAGGGAGACTT | FXN | 5′ and 3′ | human | dTs; InaGs; dAs; InaCs; dCs; |
| | m02 | TTTTTTTTTT | | | | InaCs; dAs; InaAs; dGs; InaGs; |
| | | | | | | dGs; InaAs; dGs; InaAs; |
| | | | | | | dCs; InaTs; dTs; InaTs; dTs; |
| | | | | | | InaTs; dTs; InaTs; dTs; InaTs; |
| | | | | | | dTs; InaTs; dT-Sup |
| 174 | FXN-492 | TGGCCACTGGCCGCATT | FXN | 5′ and 3′ | human | dTs; InaGs; dGs; InaCs; dCs; |
| | m02 | TTTTTTTTTT | | | | InaAs; dCs; InaTs; dGs; InaGs; |
| | | | | | | dCs; InaCs; dGs; InaCs; |
| | | | | | | dAs; InaTs; dTs; InaTs; dTs; |
| | | | | | | InaTs; dTs; InaTs; dTs; InaTs; |
| | | | | | | dTs; InaTs; dT-Sup |
| 175 | FXN-493 | CGGCGACCCCTGGTGTT | FXN | 5′ and 3′ | human | dCs; InaGs; dGs; InaCs; dGs; |
| | m02 | TTTTTTTTTT | | | | InaAs; dCs; InaCs; dCs; InaCs; |
| | | | | | | dTs; InaGs; dGs; InaTs; |
| | | | | | | dGs; InaTs; dTs; InaTs; dTs; |
| | | | | | | InaTs; dTs; InaTs; dTs; InaTs; |
| | | | | | | dTs; InaTs; dT-Sup |
| 176 | FXN-494 | CGCCCTCCAGCGCTGTT | FXN | 5′ and 3′ | human | dCs; InaGs; dCs; InaCs; dCs; |
| | m02 | TTTTTTTTTT | | | | InaTs; dCs; InaCs; dAs; InaGs; |
| | | | | | | dCs; InaGs; dCs; InaTs; dGs; |
| | | | | | | InaTs; dTs; InaTs; dTs; InaTs; |
| | | | | | | dTs; InaTs; dTs; InaTs; |
| | | | | | | dTs; InaTs; dT-Sup |
| 177 | FXN-495 | CGCTCCGCCCTCCAGTTT | FXN | 5′ and 3′ | human | dCs; InaGs; dCs; InaTs; dCs; |
| | m02 | TTTTTTTTT | | | | InaCs; dGs; InaCs; dCs; InaCs; |
| | | | | | | dTs; InaCs; dCs; InaAs; dGs; |
| | | | | | | InaTs; dTs; InaTs; dTs; InaTs; |
| | | | | | | dTs; InaTs; dTs; InaTs; |
| | | | | | | dTs; InaTs; dT-Sup |
| 178 | FXN-496 | AAAATAAACAACAAC | FXN | UTR | human | dAs; InaAs; dAs; InaAs; dTs; |
| | m02 | | | | | InaAs; dAs; InaAs; dCs; InaAs; |
| | | | | | | dAs; InaCs; dAs; InaAs; |
| | | | | | | dC-Sup |
| 179 | FXN-497 | AGGAATAAAAAAAATA | FXN | UTR | human | dAs; InaGs; dGs; InaAs; dAs; |
| | m02 | | | | | InaTs; dAs; InaAs; dAs; InaAs; |
| | | | | | | dAs; InaAs; dAs; InaAs; |
| | | | | | | dTs; InaA-Sup |
| 180 | FXN-498 | TCAAAAGCAGGAATA | FXN | UTR | human | dTs; InaCs; dAs; InaAs; dAs; |
| | m02 | | | | | InaAs; dGs; InaCs; dAs; InaGs; |
| | | | | | | dGs; InaAs; dAs; InaTs; |
| | | | | | | dA-Sup |
| 181 | FXN-499 | ACTGTCCTCAAAAGC | FXN | UTR | human | dAs; InaCs; dTs; InaGs; dTs; |
| | m02 | | | | | InaCs; dCs; InaTs; dCs; InaAs; |
| | | | | | | dAs; InaAs; dAs; InaGs; |
| | | | | | | dC-Sup |
| 182 | FXN-500 | AGCCCAACTGTCCTC | FXN | UTR | human | dAs; InaGs; dCs; InaCs; dCs; |
| | m02 | | | | | InaAs; dAs; InaCs; dTs; InaGs; |
| | | | | | | dTs; InaCs; dCs; InaTs; |
| | | | | | | dC-Sup |
| 183 | FXN-501 | TGACACATAGCCCAA | FXN | UTR | human | dTs; InaGs; dAs; InaCs; dAs; |
| | m02 | | | | | InaCs; dAs; InaTs; dAs; InaGs; |
| | | | | | | dCs; InaCs; dCs; InaAs; |
| | | | | | | dA-Sup |
| 184 | FXN-502 | GAGCTGTGACACATA | FXN | UTR | human | dGs; InaAs; dGs; InaCs; dTs; |
| | m02 | | | | | InaGs; dTs; InaGs; dAs; InaCs; |
| | | | | | | dAs; InaCs; dAs; InaTs; |
| | | | | | | dA-Sup |
| 185 | FXN-503 | TCTGGGCCTGGGCTG | FXN | UTR/internal | human | dTs; InaCs; dTs; InaGs; dGs; |
| | m02 | | | | | InaGs; dCs; InaCs; dTs; InaGs; |
| | | | | | | dGs; InaGs; dCs; InaTs; |
| | | | | | | dG-Sup |
| 186 | FXN-504 | GGTGAGGGTCTGGGC | FXN | UTR/internal | human | dGs; InaGs; dTs; InaGs; dAs; |
| | m02 | | | | | InaGs; dGs; InaGs; dTs; InaCs; |
| | | | | | | dTs; InaGs; dGs; InaGs; |
| | | | | | | dC-Sup |
| 187 | FXN-505 | GGGACCCGGGTGAGG | FXN | UTR/internal | human | dGs; InaGs; dGs; InaAs; dCs; |
| | m02 | | | | | InaCs; dCs; InaGs; dGs; InaGs; |
| | | | | | | dTs; InaGs; dAs; InaGs; |
| | | | | | | dG-Sup |
| 188 | FXN-506 | CCGGCCGCGGGACCC | FXN | UTR/internal | human | dCs; InaCs; dGs; InaGs; dCs; |
| | m02 | | | | | InaCs; dGs; InaCs; dGs; InaGs; |
| | | | | | | dGs; InaAs; dCs; InaCs; |
| | | | | | | dC-Sup |
| 189 | FXN-507 | CAACTCTGCCGGCCG | FXN | UTR/internal | human | dCs; InaAs; dAs; InaCs; dTs; |
| | m02 | | | | | InaCs; dTs; InaGs; dCs; InaCs; |
| | | | | | | dGs; InaGs; dCs; InaCs; |
| | | | | | | dG-Sup |
| 190 | FXN-508 | AGTGGGGCCAACTCT | FXN | UTR/internal | human | dAs; InaGs; dTs; InaGs; dGs; |
| | m02 | | | | | InaGs; dGs; InaCs; dCs; InaAs; |
| | | | | | | dAs; InaCs; dTs; InaCs; |
| | | | | | | dT-Sup |
| 191 | FXN-509 | GGCCGCAGAGTGGGG | FXN | UTR/internal | human | dGs; InaGs; dCs; InaCs; dGs; |
| | m02 | | | | | InaCs; dAs; InaGs; dAs; InaGs; |
| | | | | | | dTs; InaGs; dGs; InaGs; |
| | | | | | | dG-Sup |
| 192 | FXN-510 | GCCACGGCGGCCGCA | FXN | UTR/internal | human | dGs; InaCs; dCs; InaAs; dCs; |
| | m02 | | | | | InaGs; dGs; InaCs; dGs; InaGs; |
| | | | | | | dCs; InaCs; dGs; InaCs; |
| | | | | | | dA-Sup |
| 193 | FXN-511 | GTGCGCAGGCCACGG | FXN | UTR/internal | human | dGs; InaTs; dGs; InaCs; dGs; |
| | m02 | | | | | InaCs; dAs; InaGs; dGs; InaCs; |
| | | | | | | dCs; InaAs; dCs; InaGs; |
| | | | | | | dG-Sup |
| 194 | FXN-512 | GGGGGACGGGGCAGG | FXN | intron | human | dGs; InaGs; dGs; InaGs; dGs; |
| | m02 | | | | | InaAs; dCs; InaGs; dGs; InaGs; |
| | | | | | | dGs; InaCs; dAs; InaGs; |
| | | | | | | dG-Sup |
| 195 | FXN-513 | GGGACGGGGCAGGTT | FXN | intron | human | dGs; InaGs; dGs; InaAs; dCs; |
| | m02 | | | | | InaGs; dGs; InaGs; dGs; InaCs; |
| | | | | | | dAs; InaGs; dGs; InaTs; |
| | | | | | | dT-Sup |
| 196 | FXN-514 | GACGGGGCAGGTTGA | FXN | intron | human | dGs; InaAs; dCs; InaGs; dGs; |
| | m02 | | | | | InaGs; dGs; InaCs; dAs; InaGs; |
| | | | | | | dGs; InaTs; dTs; InaGs; |
| | | | | | | dA-Sup |
| 197 | FXN-515 | CGGGGCAGGTTGAGA | FXN | intron | human | dCs; InaGs; dGs; InaGs; dGs; |
| | m02 | | | | | InaCs; dAs; InaGs; dGs; InaTs; |
| | | | | | | dTs; InaGs; dAs; InaGs; |
| | | | | | | dA-Sup |
| 198 | FXN-516 | GGGCAGGTTGAGACT | FXN | intron | human | dGs; InaGs; dGs; InaCs; dAs; |
| | m02 | | | | | InaGs; dGs; InaTs; dTs; InaGs; |
| | | | | | | dAs; InaGs; dAs; InaCs; |
| | | | | | | dT-Sup |
| 199 | FXN-517 | GCAGGTTGAGACTGG | FXN | intron | human | dGs; InaCs; dAs; InaGs; dGs; |
| | m02 | | | | | InaTs; dTs; InaGs; dAs; InaGs; |
| | | | | | | dAs; InaCs; dTs; InaGs; |
| | | | | | | dG-Sup |
| 200 | FXN-518 | AGGTTGAGACTGGGT | FXN | intron | human | dAs; InaGs; dGs; InaTs; dTs; |
| | m02 | | | | | InaGs; dAs; InaGs; dAs; InaCs; |
| | | | | | | dTs; In aGs; dGs; InaGs; |
| | | | | | | dT-Sup |
| 201 | FXN-519 | GGAAAAATTCCAGGA | FXN | Antisense/ | human | dGs; InaGs; dAs; InaAs; dAs; |
| | m02 | | | UTR | | InaAs; dAs; InaTs; dTs; InaCs; |
| | | | | | | dCs; InaAs; dGs; InaGs; |
| | | | | | | dA-Sup |
| 202 | FXN-520 | AATTCCAGGAGGGAA | FXN | Antisense/ | human | dAs; InaAs; dTs; InaTs; dCs; |
| | m02 | | | UTR | | InaCs; dAs; InaGs; dGs; InaAs; |
| | | | | | | dGs; InaGs; dGs; InaAs; |
| | | | | | | dA-Sup |
| 203 | FXN-521 | GAGGGAAAATGAATT | FXN | Antisense/ | human | dGs; InaAs; dGs; InaGs; dGs; |
| | m02 | | | UTR | | InaAs; dAs; InaAs; dAs; InaTs; |
| | | | | | | dGs; InaAs; dAs; InaTs; |
| | | | | | | dT-Sup |
| 204 | FXN-522 | GAAAATGAATTGTCTTC | FXN | Antisense/ | human | dGs; InaAs; dAs; InaAs; dAs; |
| | m02 | | | UTR | | InaTs; dGs; InaAs; dAs; InaTs; |
| | | | | | | dTs; InaGs; dTs; InaCs; |
| | | | | | | dTs; InaTs; dC-Sup |
| 205 | FXN-512 | GGGGGACGGGGCAGG | FXN | intron | human | InaGs; InaGs; InaGs; dGs; |
| | m08 | | | | | dGs; dAs; dCs; dGs; dGs; dGs; |
| | | | | | | dGs; dCs; InaAs; InaGs; InaG- |
| | | | | | | Sup |
| 206 | FXN-513 | GGGACGGGGCAGGTT | FXN | intron | human | InaGs; InaGs; InaGs; dAs; |
| | m08 | | | | | dCs; dGs; dGs; dGs; dGs; dCs; |
| | | | | | | dAs; dGs; InaGs; InaTs; InaT- |
| | | | | | | Sup |
| 207 | FXN-514 | GACGGGGCAGGTTGA | FXN | intron | human | InaGs; InaAs; InaCs; dGs; |
| | m08 | | | | | dGs; dGs; dGs; dCs; dAs; dGs; |
| | | | | | | dGs; dTs; InaTs; InaGs; InaA- |
| | | | | | | Sup |
| 208 | FXN-515 | CGGGGCAGGTTGAGA | FXN | intron | human | InaCs; InaGs; InaGs; dGs; |
| | m08 | | | | | dGs; dCs; dAs; dGs; dGs; dTs; |
| | | | | | | dTs; dGs; InaAs; InaGs; InaA- |
| | | | | | | Sup |
| 209 | FXN-516 | GGGCAGGTTGAGACT | FXN | intron | human | InaGs; InaGs; InaGs; dCs; |
| | m08 | | | | | dAs; dGs; dGs; dTs; dTs; dGs; |
| | | | | | | dAs; dGs; InaAs; InaCs; InaT- |
| | | | | | | Sup |
| 210 | FXN-517 | GCAGGTTGAGACTGG | FXN | intron | human | InaGs; InaCs; InaAs; dGs; |
| | m08 | | | | | dGs; dTs; dTs; dGs; dAs; dGs; |
| | | | | | | dAs; dCs; InaTs; InaGs; InaG- |
| | | | | | | Sup |
| 211 | FXN-518 | AGGTTGAGACTGGGT | FXN | intron | human | InaAs; InaGs; InaGs; dTs; |
| | m08 | | | | | dTs; dGs; dAs; dGs; dAs; dCs; |
| | | | | | | dTs; dGs; InaGs; InaGs; InaT- |
| | | | | | | Sup |
| 212 | FXN-519 | GGAAAAATTCCAGGA | FXN | Antisense/ | human | InaGs; InaGs; InaAs; dAs; |
| | m08 | | | UTR | | dAs; dAs; dAs; dTs; dTs; dCs; |
| | | | | | | dCs; dAs; InaGs; InaGs; InaA- |
| | | | | | | Sup |
| 213 | FXN-520 | AATTCCAGGAGGGAA | FXN | Antisense/ | human | InaAs; InaAs; InaTs; dTs; dCs; |
| | m08 | | | UTR | | dCs; dAs; dGs; dGs; dAs; dGs; |
| | | | | | | dGs; InaGs; InaAs; InaA- |
| | | | | | | Sup |
| 214 | FXN-521 | GAGGGAAAATGAATT | FXN | Antisense/ | human | InaGs; InaAs; InaGs; dGs; |
| | m08 | | | UTR | | dGs; dAs; dAs; dAs; dAs; dTs; |
| | | | | | | dGs; dAs; InaAs; InaTs; InaT- |
| | | | | | | Sup |
| 215 | FXN-522 | GAAAATGAATTGTCTTC | FXN | Antisense/ | human | InaGs; InaAs; InaAs; dAs; |
| | m08 | | | UTR | | dAs; dTs; dGs; dAs; dAs; dTs; |
| | | | | | | dTs; dGs; dTs; dCs; InaTs; |
| | | | | | | InaTs; InaC-Sup |
| 216 | EPO-37 | GGTGGTTTCAGTTCT | EPO | 3′ | human | dGs; InaGs; dTs; InaGs; dGs; |
| | m02 | | | | | InaTs; dTs; InaTs; dCs; InaAs; |
| | | | | | | dGs; InaTs; dTs; InaCs; |
| | | | | | | dT-Sup |
| 217 | EPO-38 | TTTTTGGTGGTTTCAGTT | EPO | 3′ | human | dTs; InaTs; dTs; InaTs; dTs; |
| | m02 | CT | | | | InaGs; dGs; InaTs; dGs; InaGs; |
| | | | | | | dTs; InaTs; dTs; InaCs; dAs; |
| | | | | | | InaGs; dTs; InaTs; dCs; |
| | | | | | | InaT-Sup |
| 218 | EPO-39 | AGCGTGCTATCTGGG | EPO | 5′ | human | dAs; InaGs; dCs; InaGs; dTs; |
| | m02 | | | | | InaGs; dCs; InaTs; dAs; InaTs; |
| | | | | | | dCs; InaTs; dGs; InaGs; |
| | | | | | | dG-Sup |
| 219 | EPO-40 | TGGCCCAGGGACTCT | EPO | 5′ | human | dTs; InaGs; dGs; InaCs; dCs; |
| | m02 | | | | | InaCs; dAs; InaGs; dGs; InaGs; |
| | | | | | | dAs; InaCs; dTs; InaCs; |
| | | | | | | dT-Sup |
| 220 | EPO-41 | TCTGCGGCTCTGGC | EPO | 5′ | human | dTs; InaCs; dTs; InaGs; dCs; |
| | m02 | | | | | InaGs; dGs; InaCs; dTs; InaCs; |
| | | | | | | dTs; InaGs; dGs; InaC- |
| | | | | | | Sup |
| 221 | EPO-42 | CGGTCCGGCTCTGGG | EPO | 5′ | human | dCs; InaGs; dGs; InaTs; dCs; |
| | m02 | | | | | InaCs; dGs; InaGs; dCs; InaTs; |
| | | | | | | dCs; InaTs; dGs; InaGs; |
| | | | | | | dG-Sup |
| 222 | EPO-43 | TCATCCCGGGAAGCT | EPO | 5′ | human | dTs; InaCs; dAs; InaTs; dCs; |
| | m02 | | | | | InaCs; dCs; InaGs; dGs; InaGs; |
| | | | | | | dAs; InaAs; dGs; InaCs; |
| | | | | | | dT-Sup |
| 223 | EPO-44 | CCCCAAGTCCCCGCT | EPO | 5′ | human | dCs; InaCs; dCs; InaCs; dAs; |
| | m02 | | | | | InaAs; dGs; InaTs; dCs; InaCs; |
| | | | | | | s; dCs; InaCs; dGs; InaCs; dT- |
| | | | | | | Sup |
| 224 | EPO-45 | CCAACCATGCAAGCA | EPO | 5′ | human | dCs; InaCs; dAs; InaAs; dCs; |
| | m02 | | | | | InaCs; dAs; InaTs; dGs; InaCs; |
| | | | | | | dAs; InaAs; dGs; InaCs; |
| | | | | | | dA-Sup |
| 225 | EPO-46 | TGGCCCAGGGACTCTTC | EPO | 5′ | human | dTs; InaGs; dGs; InaCs; dCs; |
| | m02 | | | | | InaCs; dAs; InaGs; dGs; InaGs; |
| | | | | | | dAs; InaCs; dTs; InaCs; |
| | | | | | | dTs; InaTs; dC-Sup |
| 226 | EPO-47 | CGGTCCGGCTCTGGGTT | EPO | 5′ | human | dCs; InaGs; dGs; InaTs; dCs; |
| | m02 | C | | | | InaCs; dGs; InaGs; dCs; InaTs; |
| | | | | | | dCs; InaTs; dGs; InaGs; |
| | | | | | | dGs; InaTs; dTs; InaC-Sup |
| 227 | EPO-48 | CCAACCATGCAAGCACC | EPO | 5′ | human | dCs; InaCs; dAs; InaAs; dCs; |
| | m02 | | | | | InaCs; dAs; InaTs; dGs; InaCs; |
| | | | | | | dAs; InaAs; dGs; InaCs; |
| | | | | | | dAs; InaCs; dC-Sup |
| 228 | EPO-49 | TGGCCCAGGGACTCTCA | EPO | 5′ | human | dTs; InaGs; dGs; InaCs; dCs; |
| | m02 | CAAAGTGAC | | | | InaCs; dAs; InaGs; dGs; InaGs; |
| | | | | | | Gs; dAs; InaCs; dTs; InaCs; |
| | | | | | | dTs; InaCs; dAs; dCs; dAs; dAs; |
| | | | | | | dAs; dGs; dTs; InaGs; dAs; |
| | | | | | | InaC-Sup |
| 229 | EPO-50 | CGGTCCGGCTCTGGGAA | EPO | 5′ | human | dCs; InaGs; dGs; InaTs; dCs; |
| | m02 | GAAACTTTC | | | | InaCs; dGs; InaGs; dCs; InaTs; |
| | | | | | | dCs; InaTs; dGs; InaGs; dGs; |
| | | | | | | InaAs; dAs; dGs; dAs; |
| | | | | | | dAs; dAs; dCs; dTs; InaTs; dTs; |
| | | | | | | InaC-Sup |
| 230 | EPO-51 | CCAACCATGCAAGCACT | EPO | 5′ | human | dCs; InaCs; dAs; InaAs; dCs; |
| | m02 | CAAAGAGTC | | | | InaCs; dAs; InaTs; dGs; InaCs; |
| | | | | | | dAs; InaAs; dGs; InaCs; |
| | | | | | | dAs; InaCs; dTs; dCs; dAs; dAs; |
| | | | | | | dAs; dGs; dAs; InaGs; dTs; |
| | | | | | | InaC-Sup |
| 231 | EPO-52 | TGGCCCAGGGACTCTTT | EPO | 5′ and 3′ | human | dTs; InaGs; dGs; InaCs; dCs; |
| | m02 | TTGGTGGTTTCAGTTCT | | | | InaCs; dAs; InaGs; dGs; InaGs; |
| | | | | | | dAs; InaCs; dTs; InaCs; |
| | | | | | | dTs; InaTs; dTs; InaTs; dTs; |
| | | | | | | InaGs; dGs; InaTs; dGs; InaGs; |
| | | | | | | dTs; InaTs; dTs; InaCs; dAs; |
| | | | | | | InaGs; dTs; InaTs; dCs; InaT- |
| | | | | | | Sup |
| 232 | EPO-53 | CGGTCCGGCTCTGGGTT | EPO | 5′ and 3′ | human | dCs; InaGs; dGs; InaTs; dCs; |
| | m02 | TTTGGTGGTTTCAGTTCT | | | | InaCs; dGs; InaGs; dCs; InaTs; |
| | | | | | | dCs; InaTs; dGs; InaGs; |
| | | | | | | dGs; InaTs; dTs; InaTs; dTs; |
| | | | | | | InaTs; dGs; InaGs; dTs; InaGs; |
| | | | | | | dGs; InaTs; dTs; InaTs; dCs; |
| | | | | | | InaAs; dGs; InaTs; dTs; InaCs; |
| | | | | | | dT-Sup |
| 233 | EPO-54 | CCAACCATGCAAGCATT | EPO | 5′ and 3′ | human | dCs; InaCs; dAs; InaAs; dCs; |
| | m02 | TTTGGTGGTTTCAGTTCT | | | | InaCs; dAs; InaTs; dGs; InaCs; |
| | | | | | | dAs; InaAs; dGs; InaCs; dAs; |
| | | | | | | InaTs; dTs; InaTs; dTs; |
| | | | | | | InaTs; dGs; InaGs; dTs; InaGs; |
| | | | | | | dGs; InaTs; dTs; InaTs; dCs; |
| | | | | | | InaAs; dGs; InaTs; dTs; InaCs; |
| | | | | | | dT-Sup |
| 234 | EPO-55 | CAGGGACTCTTTTTGGT | EPO | 5′ and 3′ | human | dCs; InaAs; dGs; InaGs; dGs; |
| | m02 | GGTTTCA | | | | InaAs; dCs; InaTs; dCs; InaTs; |
| | | | | | | dTs; InaTs; dTs; InaTs; |
| | | | | | | dGs; InaGs; dTs; InaGs; dGs; |
| | | | | | | InaTs; dTs; InaTs; dCs; InaA- |
| | | | | | | Sup |
| 235 | EPO-56 | CGGCTCTGGGTTTTTGG | EPO | 5′ and 3′ | human | dCs; InaGs; dGs; InaCs; dTs; |
| | m02 | TGGTTTCA | | | | InaCs; dTs; InaGs; dGs; InaGs; |
| | | | | | | dTs; InaTs; dTs; InaTs; |
| | | | | | | dTs; InaGs; dGs; InaTs; dGs; |
| | | | | | | InaGs; dTs; InaTs; dTs; InaCs; |
| | | | | | | dA-Sup |
| 236 | EPO-57 | CATGCAAGCATTTTTGG | EPO | 5′ and 3′ | human | dCs; InaAs; dTs; InaGs; dCs; |
| | m02 | TGGTTTCA | | | | InaAs; dAs; InaGs; dCs; InaAs; |
| | | | | | | dTs; InaTs; dTs; InaTs; |
| | | | | | | dTs; InaGs; dGs; InaTs; dGs; |
| | | | | | | InaGs; dTs; InaTs; dTs; InaCs; |
| | | | | | | dA-Sup |
| 237 | EPO-58 | TGGCCCAGGGACTCGGT | EPO | 5′ and 3′ | human | dTs; InaGs; dGs; InaCs; dCs; |
| | m02 | GGTTTCAGTTCT | | | | InaCs; dAs; InaGs; dGs; InaGs; |
| | | | | | | dAs; InaCs; dTs; InaCs; |
| | | | | | | dGs; InaGs; dTs; InaGs; dGs; |
| | | | | | | InaTs; dTs; InaTs; dCs; InaAs; |
| | | | | | | dGs; InaTs; dTs; InaCs; dT- |
| | | | | | | Sup |
| 238 | EPO-59 | CGGTCCGGCTCTGGTGG | EPO | 5′ and 3′ | human | dCs; InaGs; dGs; InaTs; dCs; |
| | m02 | TGGTTTCAGTTCT | | | | InaCs; dGs; InaGs; dCs; InaTs; |
| | | | | | | dCs; InaTs; dGs; InaGs; |
| | | | | | | dTs; InaGs; dGs; InaTs; dGs; |
| | | | | | | InaGs; dTs; InaTs; dTs; InaCs; |
| | | | | | | dAs; InaGs; dTs; InaTs; dCs; |
| | | | | | | InaT-Sup |
| 239 | EPO-60 | CCAACCATGCAAGCAGG | EPO | 5′ and 3′ | human | dCs; InaCs; dAs; InaAs; dCs; |
| | m02 | TGGTTTCAGTTCT | | | | InaCs; dAs; InaTs; dGs; InaCs; |
| | | | | | | dAs; InaAs; dGs; InaCs; |
| | | | | | | dAs; InaGs; dGs; InaTs; dGs; |
| | | | | | | InaGs; dTs; InaTs; dTs; InaCs; |
| | | | | | | dAs; InaGs; dTs; InaTs; dCs; |
| | | | | | | InaT-Sup |
| 240 | KLF4-31 | TTTTTAGATAAAATATTA | KLF4 | 3′ | human | dTs; InaTs; dTs; InaTs; dTs; |
| | m02 | TA | | | | InaAs; dGs; InaAs; dTs; InaAs; |
| | | | | | | dAs; InaAs; dAs; InaTs; dAs; |
| | | | | | | InaTs; dTs; InaAs; dTs; InaA- |
| | | | | | | Sup |
| 241 | KLF4-32 | TTTTTATTCAGATAAAAT | KLF4 | 3′ | human | dTs; InaTs; dTs; InaTs; dTs; |
| | m02 | A | | | | InaAs; dTs; InaTs; dCs; InaAs; |
| | | | | | | dGs; InaAs; dTs; InaAs; |
| | | | | | | dAs; InaAs; dAs; InaTs; dA- |
| | | | | | | Sup |
| 242 | KLF4-33 | TTTTTGGTTTATTTAAAA | KLF4 | 3′ | human | dTs; InaTs; dTs; InaTs; dTs; |
| | m02 | CT | | | | InaGs; dGs; InaTs; dTs; InaTs; |
| | | | | | | dAs; InaTs; dTs; InaTs; dAs; |
| | | | | | | InaAs; dAs; InaAs; dCs; InaT- |
| | | | | | | Sup |
| 243 | KLF4-34 | TTTTTAAATTTATATTAC | KLF4 | 3′ | human | dTs; InaTs; dTs; InaTs; dTs; |
| | m02 | AT | | | | InaAs; dAs; InaAs; dTs; InaTs; |
| | | | | | | dTs; InaAs; dTs; InaAs; dTs; |
| | | | | | | InaTs; dAs; InaCs; dAs; |
| | | | | | | InaT-Sup |
| 244 | KLF4-35 | TTTTTCTTAAATTTATAT | KLF4 | 3′ | human | dTs; InaTs; dTs; InaTs; dTs; |
| | m02 | TA | | | | InaCs; dTs; InaTs; dAs; InaAs; |
| | | | | | | dAs; InaTs; dTs; InaTs; dAs; |
| | | | | | | InaTs; dAs; InaTs; dTs; InaA- |
| | | | | | | Sup |
| 245 | KLF4-36 | TTTTTCACAAAATGTTCA | KLF4 | 3′ | human | dTs; InaTs; dTs; InaTs; dTs; |
| | m02 | TT | | | | InaCs; dAs; InaCs; dAs; InaAs; |
| | | | | | | dAs; InaAs; dTs; InaGs; dTs; |
| | | | | | | InaTs; dCs; InaAs; dTs; InaT- |
| | | | | | | Sup |
| 246 | KLF4-37 | CCTCCGCCTTCTCCC | KLF4 | 5′ | human | dCs; InaCs; dTs; InaCs; dCs; |
| | m02 | | | | | InaGs; dCs; InaCs; dTs; InaTs; |
| | | | | | | dCs; InaTs; dCs; InaCs; dC- |
| | | | | | | Sup |
| 247 | KLF4-38 | TCTGGTCGGGAAACT | KLF4 | 5′ | human | dTs; InaCs; dTs; InaGs; dGs; |
| | m02 | | | | | InaTs; dCs; InaGs; dGs; InaGs; |
| | | | | | | dAs; InaAs; dAs; InaCs; |
| | | | | | | dT-Sup |
| 248 | KLF4-39 | GCTACAGCCTTTTCC | KLF4 | 5′ | human | dGs; InaCs; dTs; InaAs; dCs; |
| | m02 | | | | | InaAs; dGs; InaCs; dCs; InaTs; |
| | | | | | | dTs; InaTs; dTs; InaCs; dC- |
| | | | | | | Sup |
| 249 | KLF4-40 | CCTCCGCCTTCTCCCC | KLF4 | 5′ | human | dCs; InaCs; dTs; InaCs; dCs; |
| | m02 | | | | | InaGs; dCs; InaCs; dTs; InaTs; |
| | | | | | | dCs; InaTs; dCs; InaCs; dCs; |
| | | | | | | InaC-Sup |
| 250 | KLF4-41 | TCTGGTCGGGAAACTCC | KLF4 | 5′ | human | dTs; InaCs; dTs; InaGs; dGs; |
| | m02 | | | | | InaTs; dCs; InaGs; dGs; InaGs; |
| | | | | | | dAs; InaAs; dAs; InaCs; |
| | | | | | | dTs; InaCs; dC-Sup |
| 251 | KLF4-42 | GCTACAGCCTTTTCCC | KLF4 | 5′ | human | dGs; InaCs; dTs; InaAs; dCs; |
| | m02 | | | | | InaAs; dGs; InaCs; dCs; InaTs; |
| | | | | | | dTs; InaTs; dTs; InaCs; dCs; |
| | | | | | | InaC-Sup |
| 252 | KLF4-43 | CCTCCGCCTTCTCCCTCT | KLF4 | 5′ | human | dCs; InaCs; dTs; InaCs; dCs; |
| | m02 | TTGATC | | | | InaGs; dCs; InaCs; dTs; InaTs; |
| | | | | | | dCs; InaTs; dCs; InaCs; dCs; |
| | | | | | | InaTs; dCs; dTs; dTs; dTs; |
| | | | | | | dGs; InaAs; dTs; InaC-Sup |
| 253 | KLF4-44 | TCTGGTCGGGAAACTCA | KLF4 | 5′ | human | dTs; InaCs; dTs; InaGs; dGs; |
| | m02 | ATTATTGTC | | | | InaTs; dCs; InaGs; dGs; InaGs; |
| | | | | | | dAs; InaAs; dAs; InaCs; |
| | | | | | | dTs; InaCs; dAs; dAs; dTs; |
| | | | | | | dTs; dAs; dTs; dTs; InaGs; dTs; |
| | | | | | | InaC-Sup |
| 254 | KLF4-45 | GCTACAGCCTTTTCCACT | KLF4 | 5′ | human | dGs; InaCs; dTs; InaAs; dCs; |
| | m02 | TTGTTC | | | | InaAs; dGs; InaCs; dCs; InaTs; |
| | | | | | | dTs; InaTs; dTs; InaCs; dCs; |
| | | | | | | InaAs; dCs; dTs; dTs; dTs; |
| | | | | | | dGs; InaTs; dTs; InaC-Sup |
| 255 | KLF4-46 | CCTCCGCCTTCTCCCTTT | KLF4 | 5′ and 3′ | human | dCs; InaCs; dTs; InaCs; dCs; |
| | m02 | TTAGATAAAATATTATA | | | | InaGs; dCs; InaCs; dTs; InaTs; |
| | | | | | | dCs; InaTs; dCs; InaCs; dCs; |
| | | | | | | InaTs; dTs; InaTs; dTs; InaTs; |
| | | | | | | dAs; InaGs; dAs; InaTs; |
| | | | | | | dAs; InaAs; dAs; InaAs; dTs; |
| | | | | | | InaAs; dTs; InaTs; dAs; InaTs; |
| | | | | | | dA-Sup |
| 256 | KLF4-47 | TCTGGTCGGGAAACTTT | KLF4 | 5′ and 3′ | human | dTs; InaCs; dTs; InaGs; dGs; |
| | m02 | TTAGATAAAATATTATA | | | | InaTs; dCs; InaGs; dGs; InaGs; |
| | | | | | | dAs; InaAs; dAs; InaCs; |
| | | | | | | dTs; InaTs; dTs; InaTs; dTs; |
| | | | | | | InaAs; dGs; InaAs; dTs; InaAs; |
| | | | | | | dAs; InaAs; dAs; InaTs; dAs; |
| | | | | | | InaTs; dTs; InaAs; dTs; InaA- |
| | | | | | | Sup |
| 257 | KLF4-48 | GCTACAGCCTTTTCCTTT | KLF4 | 5′ and 3′ | human | dGs; InaCs; dTs; InaAs; dCs; |
| | m02 | TTAGATAAAATATTATA | | | | InaAs; dGs; InaCs; dCs; InaTs; |
| | | | | | | dTs; InaTs; dTs; InaCs; dCs; |
| | | | | | | InaTs; dTs; InaTs; dTs; InaTs; |
| | | | | | | dAs; InaGs; dAs; InaTs; |
| | | | | | | dAs; InaAs; dAs; InaAs; dTs; |
| | | | | | | InaAs; dTs; InaTs; dAs; InaTs; |
| | | | | | | dA-Sup |
| 258 | KLF4-49 | CCTCCGCCTTCTCCCTTT | KLF4 | 5′ and 3′ | human | dCs; InaCs; dTs; InaCs; dCs; |
| | m02 | TTGGTTTATTTAAAACT | | | | InaGs; dCs; InaCs; dTs; InaTs; |
| | | | | | | dCs; InaTs; dCs; InaCs; dCs; |
| | | | | | | InaTs; dTs; InaTs; dTs; InaTs; |
| | | | | | | dGs; InaGs; dTs; InaTs; |
| | | | | | | dTs; InaAs; dTs; InaTs; dTs; |
| | | | | | | InaAs; dAs; InaAs; dAs; InaCs; |
| | | | | | | dT-Sup |
| 259 | KLF4-50 | TCTGGTCGGGAAACTTT | KLF4 | 5′ and 3′ | human | dTs; InaCs; dTs; InaGs; dGs; |
| | m02 | TTGGTTTATTTAAAACT | | | | InaTs; dCs; InaGs; dGs; InaGs; |
| | | | | | | dAs; InaAs; dAs; InaCs; |
| | | | | | | dTs; InaTs; dTs; InaTs; dTs; |
| | | | | | | InaGs; dGs; InaTs; dTs; InaTs; |
| | | | | | | dAs; InaTs; dTs; InaTs; dAs; |
| | | | | | | InaAs; dAs; InaAs; dCs; InaT- |
| | | | | | | Sup |
| 260 | KLF4-51 | GCTACAGCCTTTTCCTTT | KLF4 | 5′ and 3′ | human | dGs; InaCs; dTs; InaAs; dCs; |
| | m02 | TTGGTTTATTTAAAACT | | | | InaAs; dGs; InaCs; dCs; InaTs; |
| | | | | | | dTs; InaTs; dTs; InaCs; dCs; |
| | | | | | | InaTs; dTs; InaTs; dTs; InaTs; |
| | | | | | | dGs; InaGs; dTs; InaTs; |
| | | | | | | dTs; InaAs; dTs; InaTs; dTs; |
| | | | | | | InaAs; dAs; InaAs; dAs; InaCs; |
| | | | | | | dT-Sup |
| 261 | KLF4-52 | CCTCCGCCTTCTCCCTTT | KLF4 | 5′ and 3′ | human | dCs; InaCs; dTs; InaCs; dCs; |
| | m02 | TTAAATTTATATTACAT | | | | InaGs; dCs; InaCs; dTs; InaTs; |
| | | | | | | dCs; InaTs; dCs; InaCs; dCs; |
| | | | | | | InaTs; dTs; InaTs; dTs; InaTs; |
| | | | | | | dAs; InaAs; dAs; InaTs; |
| | | | | | | dTs; InaTs; dAs; InaTs; dAs; |
| | | | | | | InaTs; dTs; InaAs; dCs; InaAs; |
| | | | | | | dT |
| 262 | KLF4-53 | TCTGGTCGGGAAACTTT | KLF4 | 5′ and 3′ | human | dTs; InaCs; dTs; InaGs; dGs; |
| | m02 | TTAAATTTATATTACAT | | | | InaTs; dCs; InaGs; dGs; InaGs; |
| | | | | | | dAs; InaAs; dAs; InaCs; |
| | | | | | | dTs; InaTs; dTs; InaTs; dTs; |
| | | | | | | InaAs; dAs; InaAs; dTs; InaTs; |
| | | | | | | dTs; InaAs; dTs; InaAs; dTs; |
| | | | | | | InaTs; dAs; InaCs; dAs; InaT- |
| | | | | | | Sup |
| 263 | KLF4-54 | GCTACAGCCTTTTCCTTT | KLF4 | 5′ and 3′ | human | dGs; InaCs; dTs; InaAs; dCs; |
| | m02 | TTAAATTTATATTACAT | | | | InaAs; dGs; InaCs; dCs; InaTs; |
| | | | | | | dTs; InaTs; dTs; InaCs; dCs; |
| | | | | | | InaTs; dTs; InaTs; dTs; InaTs; |
| | | | | | | dAs; InaAs; dAs; InaTs; |
| | | | | | | dTs; InaTs; dAs; InaTs; dAs; |
| | | | | | | InaTs; dTs; InaAs; dCs; InaAs; |
| | | | | | | dT-Sup |
| 264 | KLF4-55 | GCCTTCTCCCTTTTTAGA | KLF4 | 5′ and 3′ | human | dGs; InaCs; dCs; InaTs; dTs; |
| | m02 | TAAAATA | | | | InaCs; dTs; InaCs; dCs; InaCs; |
| | | | | | | dTs; InaTs; dTs; InaTs; dTs; |
| | | | | | | InaAs; dGs; InaAs; dTs; InaAs; |
| | | | | | | dAs; InaAs; dAs; InaTs; |
| | | | | | | dA-Sup |
| | | | | | | dTs; InaCs; dGs; InaGs; dGs; |
| 265 | KLF4-56 | TCGGGAAACTTTTTAGA | KLF4 | 5′ and 3′ | human | InaAs; dAs; InaAs; dCs; InaTs; |
| | m02 | TAAAATA | | | | dTs; InaTs; dTs; InaTs; |
| | | | | | | dAs; InaGs; dAs; InaTs; dAs; |
| | | | | | | InaAs; dAs; InaAs; dTs; InaA- |
| | | | | | | Sup |
| 266 | KLF4-57 | AGCCTTTTCCTTTTTAGA | KLF4 | 5′ and 3′ | human | dAs; InaGs; dCs; InaCs; dTs; |
| | m02 | TAAAATA | | | | InaTs; dTs; InaTs; dCs; InaCs; |
| | | | | | | dTs; InaTs; dTs; InaTs; dTs; |
| | | | | | | InaAs; dGs; InaAs; dTs; InaAs; |
| | | | | | | dAs; InaAs; dAs; InaTs; |
| | | | | | | dA-Sup |
| 267 | KLF4-58 | GCCTTCTCCCTTTTTGGT | KLF4 | 5′ and 3′ | human | dGs; InaCs; dCs; InaTs; dTs; |
| | m02 | TTATTTA | | | | InaCs; dTs; InaCs; dCs; InaCs; |
| | | | | | | dTs; InaTs; dTs; InaTs; dTs; |
| | | | | | | InaGs; dGs; InaTs; dTs; InaTs; |
| | | | | | | dAs; InaTs; dTs; InaTs; |
| | | | | | | dA-Sup |
| 268 | KLF4-59 | TCGGGAAACTTTTTGGT | KLF4 | 5′ and 3′ | human | dTs; InaCs; dGs; InaGs; dGs; |
| | m02 | TTATTTA | | | | InaAs; dAs; InaAs; dCs; InaTs; |
| | | | | | | dTs; InaTs; dTs; InaTs; |
| | | | | | | dGs; InaGs; dTs; InaTs; dTs; |
| | | | | | | InaAs; dTs; InaTs; dTs; InaA- |
| | | | | | | Sup |
| 269 | KLF4-60 | AGCCTTTTCCTTTTTGGT | KLF4 | 5′ and 3′ | human | dAs; InaGs; dCs; InaCs; dTs; |
| | m02 | TTATTTA | | | | InaTs; dTs; InaTs; dCs; InaCs; |
| | | | | | | dTs; InaTs; dTs; InaTs; dTs; |
| | | | | | | InaGs; dGs; InaTs; dTs; InaTs; |
| | | | | | | dAs; InaTs; dTs; InaTs; |
| | | | | | | dA-Sup |
| 270 | KLF4-61 | GCCTTCTCCCTTTTTAAA | KLF4 | 5′ and 3′ | human | dGs; InaCs; dCs; InaTs; dTs; |
| | m02 | TTTATAT | | | | InaCs; dTs; InaCs; dCs; InaCs; |
| | | | | | | dTs; InaTs; dTs; InaTs; dTs; |
| | | | | | | InaAs; dAs; InaAs; dTs; InaTs; |
| | | | | | | dTs; InaAs; dTs; InaAs; |
| | | | | | | dT-Sup |
| 271 | KLF4-62 | TCGGGAAACTTTTTAAA | KLF4 | 5′ and 3′ | human | dTs; InaCs; dGs; InaGs; dGs; |
| | m02 | TTTATAT | | | | InaAs; dAs; InaAs; dCs; InaTs; |
| | | | | | | dTs; InaTs; dTs; InaTs; |
| | | | | | | dAs; InaAs; dAs; InaTs; dTs; |
| | | | | | | InaTs; dAs; InaTs; dAs; InaT- |
| | | | | | | Sup |
| 272 | KLF4-63 | AGCCTTTTCCTTTTTAAA | KLF4 | 5′ and 3′ | human | dAs; InaGs; dCs; InaCs; dTs; |
| | m02 | TTTATAT | | | | InaTs; dTs; InaTs; dCs; InaCs; |
| | | | | | | dTs; InaTs; dTs; InaTs; dTs; |
| | | | | | | InaAs; dAs; InaAs; dTs; InaTs; |
| | | | | | | dTs; InaAs; dTs; InaAs; dT- |
| | | | | | | Sup |
| 273 | ACTB-01 | AGGTGTGCACTTTTA | ACTB | 3′ | human | dAs; InaGs; dGs; InaTs; dGs; |
| | m02 | | | | | InaTs; dGs; InaCs; dAs; InaCs; |
| | | | | | | dTs; InaTs; dTs; InaTs; |
| | | | | | | dA-Sup |
| 274 | ACTB-02 | TCATTTTTAAGGTGT | ACTB | 3′ | human | dTs; InaCs; dAs; InaTs; dTs; |
| | m02 | | | | | InaTs; dTs; InaTs; dAs; InaAs; |
| | | | | | | dGs; InaGs; dTs; InaGs; dT- |
| | | | | | | Sup |
| 275 | ACTB-03 | TTTTTAGGTGTGCACTTT | ACTB | 3′ | human | dTs; InaTs; dTs; InaTs; dTs; |
| | m02 | TA | | | | InaAs; dGs; InaGs; dTs; InaG; |
| | | | | | | s; dTs; InaGs; dCs; InaAs; dCs; |
| | | | | | | InaTs; dTs; InaTs; dTs; InaA- |
| | | | | | | Sup |
| 276 | ACTB-04 | TTTTTCATTTTTAAGGTG | ACTB | 3′ | human | dTs; InaTs; dTs; InaTs; dTs; |
| | m02 | T | | | | InaCs; dAs; InaTs; dTs; InaTs; |
| | | | | | | dTs; InaTs; dAs; InaAs; dGs; |
| | | | | | | InaGs; dTs; InaGs; dT-Sup |
| 277 | ACTB-05 | CGCGGTCTCGGCGGT | ACTB | 5′ | human | dCs; InaGs; dCs; InaGs; dGs; |
| | m02 | | | | | InaTs; dCs; InaTs; dCs; InaGs; |
| | | | | | | dGs; InaCs; dGs; InaGs; |
| | | | | | | dT-Sup |
| 278 | ACTB-06 | ATCATCCATGGTGAG | ACTB | 5′ | human | dAs; InaTs; dCs; InaAs; dTs; |
| | m02 | | | | | InaCs; dCs; InaAs; dTs; InaGs; |
| | | | | | | dGs; InaTs; dGs; InaAs; |
| | | | | | | dG-Sup |
| 279 | ACTB-07 | CGCGGTCTCGGCGGTTT | ACTB | 5′ and 3′ | human | dCs; InaGs; dCs; InaGs; dGs; |
| | m02 | TTAGGTGTGCACTTTTA | | | | InaTs; dCs; InaTs; dCs; InaGs; |
| | | | | | | dGs; InaCs; dGs; InaGs; |
| | | | | | | dTs; InaTs; dTs; InaTs; dTs; |
| | | | | | | InaAs; dGs; InaGs; dTs; InaGs; |
| | | | | | | dTs; InaGs; dCs; InaAs; dCs; |
| | | | | | | InaTs; dTs; InaTs; dTs; InaA- |
| | | | | | | Sup |
| 280 | ACTB-08 | ATCATCCATGGTGAGTT | ACTB | 5′ and 3′ | human | dAs; InaTs; dCs; InaAs; dTs; |
| | m02 | TTTAGGTGTGCACTTTTA | | | | InaCs; dCs; InaAs; dTs; InaGs; |
| | | | | | | dGs; InaTs; dGs; InaAs; |
| | | | | | | dGs; InaTs; dTs; InaTs; dTs; |
| | | | | | | InaTs; dAs; InaGs; dGs; InaTs; |
| | | | | | | dGs; InaTs; dGs; InaCs; dAs; |
| | | | | | | InaCs; dTs; InaTs; dTs; InaTs; |
| | | | | | | dA-Sup |
| 281 | ACTB-09 | CGCGGTCTCGGCGGTTT | ACTB | 5′ and 3′ | human | dCs; InaGs; dCs; InaGs; dGs; |
| | m02 | TTCATTTTTAAGGTGT | | | | InaTs; dCs; InaTs; dCs; InaGs; |
| | | | | | | dGs; InaCs; dGs; InaGs; |
| | | | | | | dTs; InaTs; dTs; InaTs; dTs; |
| | | | | | | InaCs; dAs; InaTs; dTs; InaTs; |
| | | | | | | dTs; InaTs; dAs; InaAs; dGs; |
| | | | | | | InaGs; dTs; InaGs; dT-Sup |
| 282 | ACTB-10 | ATCATCCATGGTGAGTT | ACTB | 5′ and 3′ | human | dAs; InaTs; dCs; InaAs; dTs; |
| | m02 | TTTCATTTTTAAGGTGT | | | | InaCs; dCs; InaAs; dTs; InaGs; |
| | | | | | | dGs; InaTs; dGs; InaAs; |
| | | | | | | dGs; InaTs; dTs; InaTs; dTs; |
| | | | | | | InaTs; dCs; InaAs; dTs; InaTs; |
| | | | | | | dTs; InaTs; dTs; InaAs; dAs; |
| | | | | | | InaGs; dGs; InaTs; dGs; InaT- |
| | | | | | | Sup |
| 283 | ACTB-11 | TCTCGGCGGTTTTTAGG | ACTB | 5′ and 3′ | human | dTs; InaCs; dTs; InaCs; dGs; |
| | m02 | TGTGCAC | | | | InaGs; dCs; InaGs; dGs; InaTs; |
| | | | | | | dTs; InaTs; dTs; InaTs; |
| | | | | | | dAs; InaGs; dGs; InaTs; dGs; |
| | | | | | | InaTs; dGs; InaCs; dAs; InaC- |
| | | | | | | Sup |
| 284 | ACTB-12 | CCATGGTGAGTTTTTAG | ACTB | 5′ and 3′ | human | dCs; InaCs; dAs; InaTs; dGs; |
| | m02 | GTGTGCAC | | | | InaGs; dTs; InaGs; dAs; InaGs; |
| | | | | | | dTs; InaTs; dTs; InaTs; |
| | | | | | | dTs; InaAs; dGs; InaGs; dTs; |
| | | | | | | InaGs; dTs; InaGs; dCs; InaAs; |
| | | | | | | dC-Sup |
| 285 | ACTB-13 | TCTCGGCGGTTTTTCATT | ACTB | 5′ and 3′ | human | dTs; InaCs; dTs; InaCs; dGs; |
| | m02 | TTTAA | | | | InaGs; dCs; InaGs; dGs; InaTs; |
| | | | | | | dTs; InaTs; dTs; InaTs; |
| | | | | | | dCs; InaAs; dTs; InaTs; dTs; |
| | | | | | | InaTs; dTs; InaAs; dA-Sup |
| 286 | ACTB-14 | CCATGGTGAGTTTTTCA | ACTB | 5′ and 3′ | human | dCs; InaCs; dAs; InaTs; dGs; |
| | m02 | TTTTTAA | | | | InaGs; dTs; InaGs; dAs; InaGs; |
| | | | | | | dTs; InaTs; dTs; InaTs; |
| | | | | | | dTs; InaCs; dAs; InaTs; dTs; |
| | | | | | | InaTs; dTs; InaTs; dAs; InaA- |
| | | | | | | Sup |
| 287 | ACTB-15 | CGCGGTCTCGGCGGTA | ACTB | 5′ and 3′ | human | dCs; InaGs; dCs; InaGs; dGs; |
| | m02 | GGTGTGCACTTTTA | | | | InaTs; dCs; InaTs; dCs; InaGs; |
| | | | | | | dGs; InaCs; dGs; InaGs; |
| | | | | | | dTs; InaAs; dGs; InaGs; dTs; |
| | | | | | | InaGs; dTs; InaGs; dCs; InaAs; |
| | | | | | | dCs; InaTs; dTs; InaTs; |
| | | | | | | dTs; InaA-Sup |
| 288 | ACTB-16 | ATCATCCATGGTGAGAG | ACTB | 5′ and 3′ | human | dAs; InaTs; dCs; InaAs; dTs; |
| | m02 | GTGTGCACTTTTA | | | | InaCs; dCs; InaAs; dTs; InaGs; |
| | | | | | | dGs; InaTs; dGs; InaAs; |
| | | | | | | dGs; InaAs; dGs; InaGs; dTs; |
| | | | | | | InaGs; dTs; InaGs; dCs; InaAs; |
| | | | | | | dCs; InaTs; dTs; InaTs; dTs; |
| | | | | | | InaA-Sup |
| 289 | ACTB-17 | CGCGGTCTCGGCGGTTC | ACTB | 5′ and 3′ | human | dCs; InaGs; dCs; InaGs; dGs; |
| | m02 | ATTTTTAAGGTGT | | | | InaTs; dCs; InaTs; dCs; InaGs; |
| | | | | | | dGs; InaCs; dGs; InaGs; |
| | | | | | | dTs; InaTs; dCs; InaAs; dTs; |
| | | | | | | InaTs; dTs; InaTs; dTs; InaAs; |
| | | | | | | dAs; InaGs; dGs; InaTs; dGs; |
| | | | | | | InaT-Sup |
| | | | | | | dAs; InaTs; dCs; InaAs; dTs; |
| 290 | ACTB-18 | ATCATCCATGGTGAGTC | ACTB | 5′ and 3′ | human | InaCs; dCs; InaAs; dTs; InaGs; |
| | m02 | ATTTTTAAGGTGT | | | | dGs; InaTs; dGs; InaAs; |
| | | | | | | dGs; InaTs; dCs; InaAs; dTs; |
| | | | | | | InaTs; dTs; InaTs; dTs; InaAs; |
| | | | | | | dAs; InaGs; dGs; InaTs; dGs; |
| | | | | | | InaT-Sup |
| 291 | UTRN- | TGGAGCCGAGCGCTG | UTRN | 5′ | human | dTs; InaGs; dGs; InaAs; dGs; |
| | 192 m02 | | | | | InaCs; dCs; InaGs; dAs; InaGs; |
| | | | | | | dCs; InaGs; dCs; InaTs; |
| | | | | | | dG-Sup |
| 292 | UTRN- | GGGCCTGCCCCTTTG | UTRN | 5′ | human | dGs; InaGs; dGs; InaCs; dCs; |
| | 193 m02 | | | | | InaTs; dGs; InaCs; dCs; InaCs; |
| | | | | | | dCs; InaTs; dTs; InaTs; |
| | | | | | | dG-Sup |
| 293 | UTRN- | CCCCAAGTCACCTGA | UTRN | 5′ | human | dCs; InaCs; dCs; InaCs; dAs; |
| | 194 m02 | | | | | InaAs; dGs; InaTs; dCs; InaAs; |
| | | | | | | dCs; InaCs; dTs; InaGs; |
| | | | | | | dA-Sup |
| 294 | UTRN- | GACATCAATACCTAA | UTRN | 5′ | human | dGs; InaAs; dCs; InaAs; dTs; |
| | 195 m02 | | | | | InaCs; dAs; InaAs; dTs; InaAs; |
| | | | | | | dCs; InaCs; dTs; InaAs; dA- |
| | | | | | | Sup |
| 295 | UTRN- | AAACTTTACCAAGTC | UTRN | 5′ | human | dAs; InaAs; dAs; InaCs; dTs; |
| | 196 m02 | | | | | InaTs; dTs; InaAs; dCs; InaCs; |
| | | | | | | dAs; InaAs; dGs; InaTs; dC- |
| | | | | | | Sup |
| 296 | UTRN- | TGGAGCCGAGCGCTGC | UTRN | 5′ | human | dTs; InaGs; dGs; InaAs; dGs; |
| | 197 m02 | C | | | | InaCs; dCs; InaGs; dAs; InaGs; |
| | | | | | | dCs; InaGs; dCs; InaTs; dGs; |
| | | | | | | InaCs; dC-Sup |
| 297 | UTRN- | GGGCCTGCCCCTTTGCC | UTRN | 5′ | human | dGs; InaGs; dGs; InaCs; dCs; |
| | 198 m02 | | | | | InaTs; dGs; InaCs; dCs; InaCs; |
| | | | | | | dCs; InaTs; dTs; InaTs; |
| | | | | | | dGs; InaCs; dC-Sup |
| 298 | UTRN- | CCCCAAGTCACCTGACC | UTRN | 5′ | human | dCs; InaCs; dCs; InaCs; dAs; |
| | 199 m02 | | | | | InaAs; dGs; InaTs; dCs; InaAs; |
| | | | | | | dCs; InaCs; dTs; InaGs; |
| | | | | | | dAs; InaCs; dC-Sup |
| 299 | UTRN- | GACATCAATACCTAACC | UTRN | 5′ | human | dGs; InaAs; dCs; InaAs; dTs; |
| | 200 m02 | | | | | InaCs; dAs; InaAs; dTs; InaAs; |
| | | | | | | dCs; InaCs; dTs; InaAs; dAs; |
| | | | | | | InaCs; dC-Sup |
| 300 | UTRN- | AAACTTTACCAAGTCCC | UTRN | 5′ | human | dAs; InaAs; dAs; InaCs; dTs; |
| | 201 m02 | | | | | InaTs; dTs; InaAs; dCs; InaCs; |
| | | | | | | dAs; InaAs; dGs; InaTs; dCs; |
| | | | | | | InaCs; dC-Sup |
| 301 | UTRN- | TGGAGCCGAGCGCTGG | UTRN | 5′ | human | dTs; InaGs; dGs; InaAs; dGs; |
| | 202 | GAAACCAC | | | | InaCs; dCs; InaGs; dAs; InaGs; |
| | m1000 | | | | | dCs; InaGs; dCs; InaTs; |
| | | | | | | dGs; InaGs; dGs; dAs; dAs; |
| | | | | | | dAs; dCs; InaCs; dAs; InaC- |
| | | | | | | Sup |
| 302 | UTRN- | GGGCCTGCCCCTTTGGG | UTRN | 5′ | human | dGs; InaGs; dGs; InaCs; dCs; |
| | 203 | AAACCAC | | | | InaTs; dGs; InaCs; dCs; InaCs; |
| | m1000 | | | | | dCs; InaTs; dTs; InaTs; |
| | | | | | | dGs; InaGs; dGs; dAs; dAs; |
| | | | | | | dAs; dCs; InaCs; dAs; InaC- |
| | | | | | | Sup |
| 303 | UTRN- | CCCCAAGTCACCTGAGG | UTRN | 5′ | human | dCs; InaCs; dCs; InaCs; dAs; |
| | 204 | AAACCAC | | | | InaAs; dGs; InaTs; dCs; InaAs; |
| | m1000 | | | | | dCs; InaCs; dTs; InaGs; |
| | | | | | | dAs; InaGs; dGs; dAs; dAs; |
| | | | | | | dAs; dCs; InaCs; dAs; InaC- |
| | | | | | | Sup |
| 304 | UTRN- | GACATCAATACCTAAGG | UTRN | 5′ | human | dGs; InaAs; dCs; InaAs; dTs; |
| | 205 | AAACCAC | | | | InaCs; dAs; InaAs; dTs; InaAs; |
| | m1000 | | | | | dCs; InaCs; dTs; InaAs; dAs; |
| | | | | | | InaGs; dGs; dAs; dAs; dAs; |
| | | | | | | dCs; InaCs; dAs; InaC-Sup |
| 305 | UTRN- | AAACTTTACCAAGTCGG | UTRN | 5′ | human | dAs; InaAs; dAs; InaCs; dTs; |
| | 206 | AAACCAC | | | | InaTs; dTs; InaAs; dCs; InaCs; |
| | m1000 | | | | | dAs; InaAs; dGs; InaTs; dCs; |
| | | | | | | InaGs; dGs; dAs; dAs; dAs; |
| | | | | | | dCs; InaCs; dAs; InaC-Sup |
| 306 | UTRN- | ACTGCAATATATTTC | UTRN | 3′ | human | dAs; InaCs; dTs; InaGs; dCs; |
| | 207 m02 | | | | | InaAs; dAs; InaTs; dAs; InaTs; |
| | | | | | | dAs; InaTs; dTs; InaTs; dC- |
| | | | | | | Sup |
| 307 | UTRN- | GTGTTAAAATTACTT | UTRN | 3′ | human | dGs; InaTs; dGs; InaTs; dTs; |
| | 208 m02 | | | | | InaAs; dAs; InaAs; dAs; InaTs; |
| | | | | | | dTs; InaAs; dCs; InaTs; |
| | | | | | | dT-Sup |
| 308 | UTRN- | TTTTTACTGCAATATATT | UTRN | 3′ | human | dTs; InaTs; dTs; InaTs; dTs; |
| | 209 m02 | TC | | | | InaAs; dCs; InaTs; dGs; InaCs; |
| | | | | | | dAs; InaAs; dTs; InaAs; dTs; |
| | | | | | | InaAs; dTs; InaTs; dTs; InaC- |
| | | | | | | Sup |
| 309 | UTRN- | TTTTTGTGTTAAAATTAC | UTRN | 3′ | human | dTs; InaTs; dTs; InaTs; dTs; |
| | 210 m02 | TT | | | | InaGs; dTs; InaGs; dTs; InaTs; |
| | | | | | | dAs; InaAs; dAs; InaAs; dTs; |
| | | | | | | InaTs; dAs; InaCs; dTs; InaT- |
| | | | | | | Sup |
| 310 | UTRN- | CCGAGCGCTGTTTTTAC | UTRN | 5′ and 3′ | human | dCs; InaCs; dGs; InaAs; dGs; |
| | 211 m02 | TGCAATAT | | | | InaCs; dGs; InaCs; dTs; InaGs; |
| | | | | | | dTs; InaTs; dTs; InaTs; |
| | | | | | | dTs; InaAs; dCs; InaTs; dGs; |
| | | | | | | InaCs; dAs; InaAs; dTs; InaAs; |
| | | | | | | dT-Sup |
| 311 | UTRN- | TGCCCCTTTGTTTTTACT | UTRN | 5′ and 3′ | human | dTs; InaGs; dCs; InaCs; dCs; |
| | 212 m02 | GCAATAT | | | | InaCs; dTs; InaTs; dTs; InaGs; |
| | | | | | | dTs; InaTs; dTs; InaTs; dTs; |
| | | | | | | InaAs; dCs; InaTs; dGs; InaCs; |
| | | | | | | dAs; InaAs; dTs; InaAs; dT- |
| | | | | | | Sup |
| 312 | UTRN- | AGTCACCTGATTTTTACT | UTRN | 5′ and 3′ | human | dAs; InaGs; dTs; InaCs; dAs; |
| | 213 m02 | GCAATAT | | | | InaCs; dCs; InaTs; dGs; InaAs; |
| | | | | | | dTs; InaTs; dTs; InaTs; dTs; |
| | | | | | | InaAs; dCs; InaTs; dGs; InaCs; |
| | | | | | | dAs; InaAs; dTs; InaAs; |
| | | | | | | dT-Sup |
| 313 | UTRN- | CAATACCTAATTTTTACT | UTRN | 5′ and 3′ | human | dCs; InaAs; dAs; InaTs; dAs; |
| | 214 m02 | GCAATAT | | | | InaCs; dCs; InaTs; dAs; InaAs; |
| | | | | | | dTs; InaTs; dTs; InaTs; dTs; |
| | | | | | | InaAs; dCs; InaTs; dGs; InaCs; |
| | | | | | | dAs; InaAs; dTs; InaAs; dT- |
| | | | | | | Sup |
| 314 | UTRN- | TTACCAAGTCTTTTTACT | UTRN | 5′ and 3′ | human | dTs; InaTs; dAs; InaCs; dCs; |
| | 215 m02 | GCAATAT | | | | InaAs; dAs; InaGs; dTs; InaCs; |
| | | | | | | dTs; InaTs; dTs; InaTs; |
| | | | | | | dTs; InaAs; dCs; InaTs; dGs; |
| | | | | | | InaCs; dAs; InaAs; dTs; InaAs; |
| | | | | | | dT-Sup |
| 315 | UTRN- | CCGAGCGCTGTTTTTGT | UTRN | 5′ and 3′ | human | dCs; InaCs; dGs; InaAs; dGs; |
| | 216 m02 | GTTAAAAT | | | | InaCs; dGs; InaCs; dTs; InaGs; |
| | | | | | | dTs; InaTs; dTs; InaTs; |
| | | | | | | dTs; InaGs; dTs; InaGs; dTs; |
| | | | | | | InaTs; dAs; InaAs; dAs; InaAs; |
| | | | | | | dT-Sup |
| 316 | UTRN- | TGCCCCTTTGTTTTTGTG | UTRN | 5′ and 3′ | human | dTs; InaGs; dCs; InaCs; dCs; |
| | 217 m02 | TTAAAAT | | | | InaCs; dTs; InaTs; dTs; InaGs; |
| | | | | | | dTs; InaTs; dTs; InaTs; dTs; |
| | | | | | | InaGs; dTs; InaGs; dTs; InaTs; |
| | | | | | | dAs; InaAs; dAs; InaAs; |
| | | | | | | dT-Sup |
| 317 | UTRN- | AGTCACCTGATTTTTGT | UTRN | 5′ and 3′ | human | dAs; InaGs; dTs; InaCs; dAs; |
| | 218 m02 | GTTAAAAT | | | | InaCs; dCs; InaTs; dGs; InaAs; |
| | | | | | | dTs; InaTs; dTs; InaTs; dTs; |
| | | | | | | InaGs; dTs; InaGs; dTs; InaTs; |
| | | | | | | dAs; InaAs; dAs; InaAs; dT- |
| | | | | | | Sup |
| 318 | UTRN- | CAATACCTAATTTTTGTG | UTRN | 5′ and 3′ | human | dCs; InaAs; dAs; InaTs; dAs; |
| | 219 m02 | TTAAAAT | | | | InaCs; dCs; InaTs; dAs; InaAs; |
| | | | | | | dTs; InaTs; dTs; InaTs; dTs; |
| | | | | | | InaGs; dTs; InaGs; dTs; InaTs; |
| | | | | | | dAs; InaAs; dAs; InaAs; |
| | | | | | | dT-Sup |
| 319 | UTRN- | TTACCAAGTCTTTTTGTG | UTRN | 5′ and 3′ | human | dTs; InaTs; dAs; InaCs; dCs; |
| | 220 m02 | TTAAAAT | | | | InaAs; dAs; InaGs; dTs; InaCs; |
| | | | | | | dTs; InaTs; dTs; InaTs; |
| | | | | | | dTs; InaGs; dTs; InaGs; dTs; |
| | | | | | | InaTs; dAs; InaAs; dAs; InaAs; |
| | | | | | | dT-Sup |
| 320 | HBF-XXX | TGTCTGTAGCTCCAG | HBF | 5′ | human | dTs; InaGs; dTs; InaCs; |
| | m02 | | | | | dTs; InaG; dTs; InaA; dGs; |
| | | | | | | InaC; dTs; InaC; dCs; InaA; |
| | | | | | | dGs-Sup |
| 321 | HBF-XXX | TAGCTCCAGTGAGGC | HBF | 5′ | human | dTs; InaAs; dGs; InaCs; |
| | m02 | | | | | dTs; InaCs; dCs; InaAs; dGs; |
| | | | | | | InaTs; dGs; InaAs; dGs; |
| | | | | | | InaGs; dC-Sup |
| 322 | HBF-XXX | TTTCTTCTCCCACCA | HBF | 5′ | human | dTs; InaTs; dTs; InaCs; dTs; |
| | m02 | | | | | InaTs; dCs; InaTs; dCs; |
| | | | | | | InaCs; dCs; InaAs; dCs; |
| | | | | | | InaCs; dA-Sup |
| 323 | HBF-XXX | TGTCTGTAGCTCCAGC | HBF | 5′ | human | dTs; InaGs; dTs; InaCs; |
| | m02 | C | | | | dTs; InaG; dTs; InaA; dGs; |
| | | | | | | InaC; dTs; InaC; dCs; InaA; |
| | | | | | | dGs; InaCs; dC-Sup |
| 324 | HBF-XXX | TAGCTCCAGTGAGGC | HBF | 5′ | human | dTs; InaAs; dGs; InaCs; |
| | m02 | CC | | | | dTs; InaCs; dCs; InaAs; dGs; |
| | | | | | | InaTs; dGs; InaAs; dGs; |
| | | | | | | InaGs; dC; InaCs; dC-Sup |
| 325 | HBF-XXX | TTTCTTCTCCCACCAC | HBF | 5′ | human | dTs; InaTs; dTs; InaCs; dTs; |
| | m02 | C | | | | InaTs; dCs; InaTs; dCs; |
| | | | | | | InaCs; dCs; InaAs; dCs; |
| | | | | | | InaCs; dA; InaCs; dC-Sup |
| 326 | HBF-XXX | TGTCTGTAGCTCCAG | HBF | 5′ | human | dTs; InaGs; dTs; InaCs; |
| | m03 | GGAAACCAC | | | | dTs; InaG; dTs; InaA; dGs; |
| | | | | | | InaC; dTs; InaC; dCs; InaA; |
| | | | | | | dGs; InaGs; dGs; dAs; |
| | | | | | | dAs; dAs; dCs; InaCs; dAs; |
| | | | | | | InaC-Sup |
| 327 | HBF-XXX | TAGCTCCAGTGAGGC | HBF | 5′ | human | dTs; InaAs; dGs; InaCs; |
| | m04 | GGAAACCAC | | | | dTs; InaCs; dCs; InaAs; dGs; |
| | | | | | | InaTs; dGs; InaAs; dGs; |
| | | | | | | InaGs; dC; InaGs; dGs; |
| | | | | | | dAs; dAs; dAs; dCs; InaCs; |
| | | | | | | dAs; InaC-Sup |
| 328 | HBF-XXX | TTTCTTCTCCCACCAG | HBF | 5′ | human | dTs; InaTs; dTs; InaCs; dTs; |
| | m05 | GAAACCAC | | | | InaTs; dCs; InaTs; dCs; |
| | | | | | | InaCs; dCs; InaAs; dCs; |
| | | | | | | InaCs; dA; InaGs; dGs; dAs; |
| | | | | | | dAs; dAs; dCs; InaCs; dAs; |
| | | | | | | InaC-Sup |
| 329 | HBF-XXX | TTTTTGTGTGATCTCT | HBF | 3′ | human | dTs; InaTs; dTs; InaTs; dTs; |
| | m06 | TAGC | | | | InaGs; dATs; InaGs; dTs; |
| | | | | | | InaGs; dAs; InaTs; dCs; |
| | | | | | | InaTs; dCs; InaTs; dTs; |
| | | | | | | InaAs; dGs; InaC-Sup |
| 330 | HBF-XXX | TTTTTGTGATCTCTTA | HBF | 3′ | human | dTs; InaTs; dTs; InaTs; dTs; |
| | m07 | GCAG | | | | InaGs; dTs; InaGs; dAs; |
| | | | | | | InaTs; dCs; InaTs; dCs; |
| | | | | | | InaTs; dTs; InaAs; dGs; |
| | | | | | | InaCs; dAs; InaG-Sup |
| 331 | HBF-XXX | TTTTTTGATCTCTTAG | HBF | 3′ | human | dTs; InaTs; dTs; InaTs; dTs; |
| | m08 | CAGA | | | | InaTs; dGs; InaAs; dTs; |
| | | | | | | InaCs; dTs; InaCs; dTs; |
| | | | | | | InaTs; dAs; InaGs; dCs; |
| | | | | | | InaAs; dGs; InaA-Sup |
| 332 | SMN-XXX | ATTTCTCTCAATCCT | SMN | 5′ | human | dAs; InaTs; dTs; InaTs; |
| | m02 | | | | | dCs; InaT; dCs; InaT; dCs; |
| | | | | | | InaA; dAs; InaT; dCs; InaC; |
| | | | | | | dTs-Sup |
| 333 | SMN- | GGCGTGTATATTTTT | SMN | 5′ | human | dGs; InaGs; dCs; InaGs; |
| | XXX | | | | | dTs; InaGs; dTs; InaAs; dTs; |
| | m03 | | | | | InaAs; dTs; InaTs; dTs; |
| | | | | | | InaTs; dT-Sup |
| 334 | SMN- | GGTTATCGCCCTCCC | SMN | 5′ | human | dGs; InaGs; dTs; InaTs; |
| | XXX | | | | | dAs; InaTs; dCs; InaGs; dCs; |
| | m04 | | | | | InaCs; dCs; InaTs; dCs; |
| | | | | | | InaCs; dC-Sup |
| 335 | SMN- | ACGACTTCCGCCGCC | SMN | 5′ | human | dAs; InaCs; dGs; InaAs; |
| | XXX | | | | | dCs; InaTs; dTs; InaCs; dCs; |
| | m05 | | | | | InaGs; dCs; InaCs; dGs; |
| | | | | | | InaCs; dC-Sup |
| 336 | SMN- | ATTTCTCTCAATCCTC | SMN | 5′ | human | dAs; InaTs; dTs; InaTs; |
| | XXX | C | | | | dCs; InaT; dCs; InaT; dCs; |
| | m06 | | | | | InaA; dAs; InaT; dCs; InaC; |
| | | | | | | dTs; InaCs; dC-Sup |
| 337 | SMN- | GGCGTGTATATTTTTC | SMN | 5′ | human | dGs; InaGs; dCs; InaGs; |
| | XXX | C | | | | dTs; InaGs; dTs; InaAs; dTs; |
| | m07 | | | | | InaAs; dTs; InaTs; dTs; |
| | | | | | | InaTs; dT; InaCs; dC-Sup |
| 338 | SMN- | GGTTATCGCCCTCCCC | SMN | 5′ | human | dGs; InaGs; dTs; InaTs; |
| | XXX | C | | | | dAs; InaTs; dCs; InaGs; dCs; |
| | m08 | | | | | InaCs; dCs; InaTs; dCs; |
| | | | | | | InaCs; dC; InaCs; dC-Sup |
| 339 | SMN- | ACGACTTCCGCCGCCC | SMN | 5′ | human | dAs; InaCs; dGs; InaAs; |
| | XXX | C | | | | dCs; InaTs; dTs; InaCs; dCs; |
| | m09 | | | | | InaGs; dCs; InaCs; dGs; |
| | | | | | | InaCs; dC; InaCs; dC-Sup |
| 340 | SMN- | ATTTCTCTCAATCCTG | SMN | 5′ | human | dAs; InaTs; dTs; InaTs; |
| | XXX | GAAACCAC | | | | dCs; InaT; dCs; InaT; dCs; |
| | m10 | | | | | InaA; dAs; InaT; dCs; InaC; |
| | | | | | | dTs; InaGs; dGs; dAs; dAs; |
| | | | | | | dAs; dCs; InaCs; dAs; |
| | | | | | | InaC-Sup |
| 341 | SMN- | GGCGTGTATATTTTTG | SMN | 5′ | human | dGs; InaGs; dCs; InaGs; |
| | XXX | GAAACCAC | | | | dTs; InaGs; dTs; InaAs; dTs; |
| | m11 | | | | | InaAs; dTs; InaTs; dTs; |
| | | | | | | InaTs; dT; InaGs; dGs; dAs; |
| | | | | | | dAs; dAs; dCs; InaCs; dAs; |
| | | | | | | InaC-Sup |
| 342 | SMN- | GGTTATCGCCCTCCCG | SMN | 5′ | human | dGs; InaGs; dTs; InaTs; |
| | XXX | GAAACCAC | | | | dAs; InaTs; dCs; InaGs; dCs; |
| | m12 | | | | | InaCs; dCs; InaTs; dCs; |
| | | | | | | InaCs; dC; InaGs; dGs; dAs; |
| | | | | | | dAs; dAs; dCs; InaCs; |
| | | | | | | dAs; InaC-Sup |
| 343 | SMN-XXX | ACGACTTCCGCCGCC | SMN | 5′ | human | dAs; InaCs; dGs; InaAs; |
| | m13 | GGAAACCAC | | | | dCs; InaTs; dTs; InaCs; dCs; |
| | | | | | | InaGs; dCs; InaCs; dGs; |
| | | | | | | InaCs; dC; InaGs; dGs; dAs; |
| | | | | | | dAs; dAs; dCs; InaCs; |
| | | | | | | dAs; InaC-Sup |
| 344 | SMN- | TTTTTTAATTTTTTTTT | SMN | 3′ | human | dTs; InaTs; dTs; InaTs; dTs; |
| | XXX | AAA | | | | InaTs; dAs; InaAs; dTs; |
| | m14 | | | | | InaTs; dTs; InaTs; dTs; InaTs; |
| | | | | | | dTs; InaTs; dTs; InaAs; |
| | | | | | | dAs; InaA-Sup |
| 345 | SMN- | TTTTTATATGCAAAAA | SMN | 3′ | human | dTs; InaTs; dTs; InaTs; dTs; |
| | XXX | AGAA | | | | InaAs; dTs; InaAs; dTs; |
| | m15 | | | | | InaGs; dCs; InaAs; dAs; |
| | | | | | | InaAs; dAs; InaAs; dAs; |
| | | | | | | InaGs; dAs; InaA-Sup |
| 346 | SMN- | TTTTTCAAAATATGGG | SMN | 3′ | human | dTs; InaTs; dTs; InaTs; dTs; |
| | XXX | CCAA | | | | InaCs; dAs; InaAs; dAs; |
| | m16 | | | | | InaAs; dTs; InaAs; dTs; |
| | | | | | | InaGs; dGs; InaGs; dCs; |
| | | | | | | InaCs; dAs; InaA-Sup |

Table 7 provides exemplary oligonucleotides for targeting the 5′ and 3′ ends of noncoding RNAs HOTAIR and ANRIL.

TABLE 7
Oligos targeting non-coding RNAs

| | | | | Target | | |
|---|---|---|---|---|---|---|
| SEQ | Oligo | | Gene | Region (5′ | | Formatted |
| ID NO | Name | Base Sequence | Name | or 3′ End) | Organism | Sequence |
| 347 | HOTAIR- | TTCACCACATGTAAA | HOTAIR | 3′ | Human | dTs; InaTs; dCs; InaAs; |
| | 1 | | | | | dCs; InaCs; dAs; |
| | | | | | | InaCs; dAs; InaTs; |
| | | | | | | dGs; InaTs; dAs; |
| | | | | | | InaAs; dA-Sup |
| 348 | HOTAIR- | TTTTTTCACCACATGTAA | HOTAIR | 3′ | Human | dTs; InaTs; dTs; InaTs; |
| | 2 | A | | | | dTs; InaTs; dCs; |
| | | | | | | InaAs; dCs; InaCs; |
| | | | | | | dAs; InaCs; dAs; |
| | | | | | | InaTs; dGs; InaTs; |
| | | | | | | dAs; InaAs; dA- |
| | | | | | | Sup |
| 349 | HOTAIR- | AAATCAGGGCAGAATG | HOTAIR | 5′ | Human | dAs; InaAs; dAs; InaTs; |
| | 3 | T | | | | dCs; InaAs; dGs; |
| | | | | | | InaGs; dGs; InaCs; |
| | | | | | | dAs; InaGs; dAs; |
| | | | | | | InaAs; dTs; InaGs; |
| | | | | | | dT-Sup |
| 350 | HOTAIR- | AAATCAGGGCAGAATG | HOTAIR | 5′ | Human | dAs; InaAs; dAs; InaTs; |
| | 4 | TCC | | | | dCs; InaAs; dGs; |
| | | | | | | InaGs; dGs; InaCs; |
| | | | | | | dAs; InaGs; dAs; |
| | | | | | | InaAs; dTs; InaGs; |
| | | | | | | dTs; InaCs; dC- |
| | | | | | | Sup |
| 351 | HOTAIR- | AAATCAGGGCAGAATG | HOTAIR | 5′ | Human | dAs; InaAs; dAs; InaTs; |
| | 5 | TCCAAAGGTC | | | | dCs; InaAs; dGs; |
| | | | | | | InaGs; dGs; InaCs; |
| | | | | | | dAs; InaGs; dAs; |
| | | | | | | InaAs; dTs; InaGs; |
| | | | | | | dTs; InaCs; dCs; |
| | | | | | | InaAs; dAs; InaAs; |
| | | | | | | dGs; InaGs; dTs; |
| | | | | | | dC-Sup |
| 352 | HOTAIR- | AAATCAGGGCAGAATG | HOTAIR | 5′ and 3′ | Human | dAs; InaAs; dAs; InaTs; |
| | 6 | TTTTTTTCACCACATGTA | | | | dCs; InaAs; dGs; |
| | | AA | | | | InaGs; dGs; InaCs; |
| | | | | | | dAs; InaGs; dAs; |
| | | | | | | InaAs; dTs; InaGs; |
| | | | | | | dTs; InaTs; dTs; |
| | | | | | | InaTs; dTs; InaTs; |
| | | | | | | dTs; InaCs; dAs; InaCs; |
| | | | | | | dCs; InaAs; dCs; |
| | | | | | | InaAs; dTs; InaGs; |
| | | | | | | dTs; InaAs; dAs; |
| | | | | | | dA-Sup |
| 353 | ANRIL-1 | TTATTGTCTGAGCCC | ANRIL | 3′ | Human | dTs; InaTs; dAs; InaTs; |
| | | | | | | dTs; InaGs; dTs; |
| | | | | | | InaCs; dTs; InaGs; |
| | | | | | | dAs; InaGs; dCs; |
| | | | | | | InaCs; dC-Sup |
| 354 | ANRIL-2 | TTTTTATTGTCTGAGCCC | ANRIL | 3′ | Human | dTs; InaTs; dTs; InaTs; |
| | | | | | | dTs; InaAs; dTs; |
| | | | | | | InaTs; dGs; InaTs; |
| | | | | | | dCs; InaTs; dGs; |
| | | | | | | InaAs; dGs; InaCs; |
| | | | | | | dCs; dC-Sup |
| 355 | ANRIL-3 | TCAGGTGACGGATGT | ANRIL | 5′ | Human | dTs; InaCs; dAs; InaGs; |
| | | | | | | dGs; InaTs; dGs; |
| | | | | | | InaAs; dCs; InaGs; |
| | | | | | | dGs; InaAs; dTs; |
| | | | | | | InaGs; dT-Sup |
| 356 | ANRIL-4 | TCAGGTGACGGATGTCC | ANRIL | 5′ | Human | dTs; InaCs; dAs; InaGs; |
| | | | | | | dGs; InaTs; dGs; |
| | | | | | | InaAs; dCs; InaGs; |
| | | | | | | dGs; InaAs; dTs; |
| | | | | | | InaGs; dTs; InaCs; |
| | | | | | | dC-Sup |
| 357 | ANRIL-5 | TCAGGTGACGGATGTCC | ANRIL | 5′ | Human | dTs; InaCs; dAs; InaGs; |
| | | AAAGGTC | | | | dGs; InaTs; dGs; |
| | | | | | | InaAs; dCs; InaGs; |
| | | | | | | dGs; InaAs; dTs; |
| | | | | | | InaGs; dTs; InaCs; |
| | | | | | | dCs; InaAs; dAs; |
| | | | | | | InaAs; dGs; InaGs; |
| | | | | | | dTs; dC-Sup |
| 358 | ANRIL-6 | TCAGGTGACGGATGTTT | ANRIL | 5′ and 3′ | Human | dTs; InaCs; dAs; InaGs; |
| | | TTTATTGTCTGAGCCC | | | | dGs; InaTs; dGs; |
| | | | | | | InaAs; dCs; InaGs; |
| | | | | | | dGs; InaAs; dTs; |
| | | | | | | InaGs; dTs; InaTs; |
| | | | | | | dTs; InaTs; dTs; |
| | | | | | | InaTs; dAs; InaTs; |
| | | | | | | dTs; InaGs; dTs; |
| | | | | | | InaCs; dTs; InaGs; |
| | | | | | | dAs; InaGs; dCs; |
| | | | | | | InaCs; dC-Sup |

Table 8 provides further exemplary RNA stability oligos for multiple human and mouse genes.

TABLE 8
Oligonucleotides designed to target 5′ and 3′ ends of RNAs

| SEQ | Oligo | | | Target | | Formatted |
|---|---|---|---|---|---|---|
| ID NO | Name | Base Sequence | Gene Name | Region | Organism | Sequence |
| 359 | FOXP3- | TGTGGGGAGCTCGGC | FOXP3 | 3′ | human | dTs; InaGs; dTs; InaGs; |
| | 61 m02 | | | | | dGs; InaGs; dGs; |
| | | | | | | InaAs; dGs; InaCs; |
| | | | | | | dTs; InaCs; dGs; InaGs; |
| | | | | | | dC-Sup |
| 360 | FOXP3- | GGGGAGCTCGGCTGC | FOXP3 | 3′ | human | dGs; InaGs; dGs; InaGs; |
| | 62 m02 | | | | | dAs; InaGs; dCs; |
| | | | | | | InaTs; dCs; InaGs; |
| | | | | | | dGs; InaCs; dTs; InaGs; |
| | | | | | | dC-Sup |
| 361 | FOXP3- | TTTTTGTGGGGAGCTC | FOXP3 | 3′ | human | dTs; InaTs; dTs; InaTs; |
| | 63 m02 | GGC | | | | dTs; InaGs; dTs; InaGs; |
| | | | | | | dGs; InaGs; dGs; |
| | | | | | | InaAs; dGs; InaCs; |
| | | | | | | dTs; InaCs; dGs; InaGs; |
| | | | | | | dC-Sup |
| 362 | FOXP3- | TTTTGGGGAGCTCGGC | FOXP3 | 3′ | human | dTs; InaTs; dTs; InaTs; |
| | 64 m02 | TGC | | | | dGs; InaGs; dGs; InaGs; |
| | | | | | | dAs; InaGs; dCs; |
| | | | | | | InaTs; dCs; InaGs; |
| | | | | | | dGs; InaCs; dTs; |
| | | | | | | InaGs; dC-Sup |
| 363 | FOXP3- | TTGTCCAAGGGCAGG | FOXP3 | 5′ | human | dTs; InaTs; dGs; InaTs; |
| | 65 m02 | | | | | dCs; InaCs; dAs; |
| | | | | | | InaAs; dGs; InaGs; |
| | | | | | | dGs; InaCs; dAs; |
| | | | | | | InaGs; dG-Sup |
| 364 | FOXP3- | TCGATGAGTGTGTGC | FOXP3 | 5′ | human | dTs; InaCs; dGs; InaAs; |
| | 66 m02 | | | | | dTs; InaGs; dAs; |
| | | | | | | InaGs; dTs; InaGs; |
| | | | | | | dTs; InaGs; dTs; |
| | | | | | | InaGs; dC-Sup |
| 365 | FOXP3- | AGAAGAAAAACCACG | FOXP3 | 5′ | human | dAs; InaGs; dAs; InaAs; |
| | 67 m02 | | | | | dGs; InaAs; dAs; |
| | | | | | | InaAs; dAs; InaAs; |
| | | | | | | dCs; InaCs; dAs; InaCs; |
| | | | | | | dG-Sup |
| 366 | FOXP3- | AATATGATTTCTTCC | FOXP3 | 5′ | human | dAs; InaAs; dTs; InaAs; |
| | 68 m02 | | | | | dTs; InaGs; dAs; |
| | | | | | | InaTs; dTs; InaTs; dCs; |
| | | | | | | InaTs; dTs; InaCs; |
| | | | | | | dC-Sup |
| 367 | FOXP3- | GAGATGGGGGACATG | FOXP3 | 5′ | human | dGs; InaAs; dGs; InaAs; |
| | 69 m02 | | | | | dTs; InaGs; dGs; |
| | | | | | | InaGs; dGs; InaGs; |
| | | | | | | dAs; InaCs; dAs; InaTs; |
| | | | | | | dG-Sup |
| 368 | PTEN- | TTCAGTTTATTCAAG | PTEN | 3′ | human | dTs; InaTs; dCs; InaAs; |
| | 101 m02 | | | | | dGs; InaTs; dTs; |
| | | | | | | InaTs; dAs; InaTs; dTs; |
| | | | | | | InaCs; dAs; InaAs; |
| | | | | | | dG-Sup |
| 369 | PTEN- | CTGTCTCCACTTTTT | PTEN | 3′ | human | dCs; InaTs; dGs; InaTs; |
| | 102 m02 | | | | | dCs; InaTs; dCs; |
| | | | | | | InaCs; dAs; InaCs; dTs; |
| | | | | | | InaTs; dTs; InaTs; |
| | | | | | | dT-Sup |
| 370 | PTEN- | TGGAATAAAACGGG | PTEN | 3′ | human | dTs; InaGs; dGs; InaAs; |
| | 103 m02 | | | | | dAs; InaTs; dAs; |
| | | | | | | InaAs; dAs; InaAs; |
| | | | | | | dCs; InaGs; dGs; InaG- |
| | | | | | | Sup |
| 371 | PTEN- | ACAATTGAGAAAACA | PTEN | 3′ | human | dAs; InaCs; dAs; InaAs; |
| | 104 m02 | | | | | dTs; InaTs; dGs; |
| | | | | | | InaAs; dGs; InaAs; |
| | | | | | | dAs; InaAs; dAs; InaCs; |
| | | | | | | dA-Sup |
| 372 | PTEN- | CAGTTTTAAGTGGAG | PTEN | 3′ | human | dCs; InaAs; dGs; InaTs; |
| | 105 m02 | | | | | dTs; InaTs; dTs; |
| | | | | | | InaAs; dAs; InaGs; |
| | | | | | | dTs; InaGs; dGs; InaAs; |
| | | | | | | dG-Sup |
| 373 | PTEN- | TGACAAGAATGAGAC | PTEN | 3′ | human | dTs; InaGs; dAs; InaCs; |
| | 106 m02 | | | | | dAs; InaAs; dGs; |
| | | | | | | InaAs; dAs; InaTs; |
| | | | | | | dGs; InaAs; dGs; InaAs; |
| | | | | | | dC-Sup |
| 374 | PTEN- | CCGGGCGAGGGGAGG | PTEN | 5′ | human | dCs; InaCs; dGs; InaGs; |
| | 107 m02 | | | | | dGs; InaCs; dGs; |
| | | | | | | InaAs; dGs; InaGs; |
| | | | | | | dGs; InaGs; dAs; InaGs; |
| | | | | | | dG-Sup |
| 375 | PTEN- | CCGCCGGCCTGCCCG | PTEN | 5′ | human | dCs; InaCs; dGs; InaCs; |
| | 108 m02 | | | | | dCs; InaGs; dGs; |
| | | | | | | InaCs; dCs; InaTs; |
| | | | | | | dGs; InaCs; dCs; InaCs; |
| | | | | | | dG-Sup |
| 376 | PTEN- | CGAGCGCGTATCCTG | PTEN | 5′ | human | dCs; InaGs; dAs; InaGs; |
| | 109 m02 | | | | | dCs; InaGs; dCs; |
| | | | | | | InaGs; dTs; InaAs; |
| | | | | | | dTs; InaCs; dCs; InaTs; |
| | | | | | | dG-Sup |
| 377 | PTEN- | CTGCTTCTCCTCAGC | PTEN | 5′ | human | dCs; InaTs; dGs; InaCs; |
| | 110 m02 | | | | | dTs; InaTs; dCs; |
| | | | | | | InaTs; dCs; InaCs; dTs; |
| | | | | | | InaCs; dAs; InaGs; |
| | | | | | | dC-Sup |
| 378 | PTEN- | TTTTCAGTTTATTCAAG | PTEN | 3′ | human | dTs; InaTs; dTs; InaTs; |
| | 111 m02 | | | | | dCs; InaAs; dGs; InaTs; |
| | | | | | | dTs; InaTs; dAs; |
| | | | | | | InaTs; dTs; InaCs; |
| | | | | | | dAs; InaAs; dG-Sup |
| 379 | PTEN- | TTTTCTGTCTCCACTTTT | PTEN | 3′ | human | dTs; InaTs; dTs; InaTs; |
| | 112 m02 | T | | | | dCs; InaTs; dGs; |
| | | | | | | InaTs; dCs; InaTs; dCs; |
| | | | | | | InaCs; dAs; InaCs; |
| | | | | | | dTs; InaTs; dTs; InaTs; |
| | | | | | | dT-Sup |
| 380 | PTEN- | TTTTTGGAATAAAACG | PTEN | 3′ | human | dTs; InaTs; dTs; InaTs; |
| | 113 m02 | GG | | | | dTs; InaGs; dGs; InaAs; |
| | | | | | | dAs; InaTs; dAs; |
| | | | | | | InaAs; dAs; InaAs; |
| | | | | | | dCs; InaGs; dGs; InaG- |
| | | | | | | Sup |
| 381 | PTEN- | TTTTACAATTGAGAAAA | PTEN | 3′ | human | dTs; InaTs; dTs; InaTs; |
| | 114 m02 | CA | | | | dAs; InaCs; dAs; InaAs; |
| | | | | | | dTs; InaTs; dGs; |
| | | | | | | InaAs; dGs; InaAs; |
| | | | | | | dAs; InaAs; dAs; InaCs; |
| | | | | | | dA-Sup |
| 382 | PTEN- | TTTTCAGTTTTAAGTGG | PTEN | 3′ | human | dTs; InaTs; dTs; InaTs; |
| | 115 m02 | AG | | | | dCs; InaAs; dGs; InaTs; |
| | | | | | | dTs; InaTs; dTs; |
| | | | | | | InaAs; dAs; InaGs; |
| | | | | | | dTs; InaGs; dGs; InaAs; |
| | | | | | | dG-Sup |
| 383 | PTEN- | TTTTTGACAAGAATGA | PTEN | 3′ | human | dTs; InaTs; dTs; InaTs; |
| | 116 m02 | GAC | | | | dTs; InaGs; dAs; InaCs; |
| | | | | | | dAs; InaAs; dGs; |
| | | | | | | InaAs; dAs; InaTs; |
| | | | | | | dGs; InaAs; dGs; |
| | | | | | | InaAs; dC-Sup |
| 384 | NFE2L2- | AACAGTCATAATAAT | NFE2L2 | 3′ | human | dAs; InaAs; dCs; InaAs; |
| | 01 m02 | | | | | dGs; InaTs; dCs; |
| | | | | | | InaAs; dTs; InaAs; |
| | | | | | | dAs; InaTs; dAs; |
| | | | | | | InaAs; dT-Sup |
| 385 | NFE2L2- | TAATTTAACAGTCAT | NFE2L2 | 3′ | human | dTs; InaAs; dAs; InaTs; |
| | 02 m02 | | | | | dTs; InaTs; dAs; |
| | | | | | | InaAs; dCs; InaAs; |
| | | | | | | dGs; InaTs; dCs; |
| | | | | | | InaAs; dT-Sup |
| 386 | NFE2L2- | GCACGCTATAAAGCA | NFE2L2 | 5′ | human | dGs; InaCs; dAs; InaCs; |
| | 03 m02 | | | | | dGs; InaCs; dTs; |
| | | | | | | InaAs; dTs; InaAs; |
| | | | | | | dAs; InaAs; dGs; InaCs; |
| | | | | | | dA-Sup |
| 387 | NFE2L2- | CCCGGGGCTGGGCTT | NFE2L2 | 5′ | human | dCs; InaCs; dCs; InaGs; |
| | 04 m02 | | | | | dGs; InaGs; dGs; |
| | | | | | | InaCs; dTs; InaGs; |
| | | | | | | dGs; InaGs; dCs; InaTs; |
| | | | | | | dT-Sup |
| 388 | NFE2L2- | CCCCGCTCCGCCTCC | NFE2L2 | 5′ | human | dCs; InaCs; dCs; InaCs; |
| | 05 m02 | | | | | dGs; InaCs; dTs; |
| | | | | | | InaCs; dCs; InaGs; |
| | | | | | | dCs; InaCs; dTs; InaCs; |
| | | | | | | dC-Sup |
| 389 | NFE2L2- | GCGCCTCCCTGATTT | NFE2L2 | 5′ | human | dGs; InaCs; dGs; InaCs; |
| | 06 m02 | | | | | dCs; InaTs; dCs; |
| | | | | | | InaCs; dCs; InaTs; |
| | | | | | | sdG; InaAs; dTs; InaTs; |
| | | | | | | dT-Sup |
| 390 | NFE2L2- | TCGCCGCGGTGGCTG | NFE2L2 | 5′ | human | dTs; InaCs; dGs; InaCs; |
| | 07 m02 | | | | | dCs; InaGs; dCs; |
| | | | | | | InaGs; dGs; InaTs; |
| | | | | | | dGs; InaGs; dCs; InaTs; |
| | | | | | | dG-Sup |
| 391 | NFE2L2- | CAGCGAATGGTCGCG | NFE2L2 | 5′ | human | dCs; InaAs; dGs; InaCs; |
| | 08 m02 | | | | | dGs; InaAs; dAs; |
| | | | | | | InaTs; dGs; InaGs; |
| | | | | | | dTs; InaCs; dGs; InaCs; |
| | | | | | | dG-Sup |
| 392 | NFE2L2- | TTTTTAACAGTCATAAT | NFE2L2 | 3′ | human | dTs; InaTs; dTs; InaTs; |
| | 09 m02 | AAT | | | | dTs; InaAs; dAs; |
| | | | | | | InaCs; dAs; InaGs; dTs; |
| | | | | | | InaCs; dAs; InaTs; |
| | | | | | | dAs; InaAs; dTs; InaAs; |
| | | | | | | dAs; InaT-Sup |
| 393 | NFE2L2- | TTTTTAATTTAACAGTC | NFE2L2 | 3′ | human | dTs; InaTs; dTs; InaTs; |
| | 10 m02 | AT | | | | dTs; InaAs; dAs; |
| | | | | | | InaTs; dTs; InaTs; dAs; |
| | | | | | | InaAs; dCs; InaAs; |
| | | | | | | dGs; InaTs; dCs; InaAs; |
| | | | | | | dT-Sup |
| 394 | ATP2A2- | GCGGCGGCTGCTCTA | ATP2A2 | 5′ | human | dGs; InaCs; dGs; InaGs; |
| | 56 m02 | | | | | dCs; InaGs; dGs; |
| | | | | | | InaCs; dTs; InaGs; |
| | | | | | | dCs; InaTs; dCs; InaTs; |
| | | | | | | dA-Sup |
| 395 | ATP2A2- | TTATCGGCCGCTGCC | ATP2A2 | 5′ | human | dTs; InaTs; dAs; InaTs; |
| | 34 m02 | | | | | dCs; InaGs; dGs; |
| | | | | | | InaCs; dCs; InaGs; |
| | | | | | | dCs; InaTs; dGs; InaCs; |
| | | | | | | dC-Sup |
| 396 | ATP2A2- | GCGTCGGGGACGGCT | ATP2A2 | 5′ | human | dGs; InaCs; dGs; InaTs; |
| | 57 m02 | | | | | dCs; InaGs; dGs; |
| | | | | | | InaGs; dGs; InaAs; |
| | | | | | | dCs; InaGs; dGs; InaCs; |
| | | | | | | dT-Sup |
| 397 | ATP2A2- | GCGGAGGAAACTGCG | ATP2A2 | 5′ | human | dGs; InaCs; dGs; InaGs; |
| | 58 m02 | | | | | dAs; InaGs; dGs; |
| | | | | | | InaAs; dAs; InaAs; |
| | | | | | | dCs; InaTs; dGs; InaCs; |
| | | | | | | dG-Sup |
| 398 | ATP2A2- | GCCGCACGCCCGACA | ATP2A2 | 5′ | human | dGs; InaCs; dCs; InaGs; |
| | 59 m02 | | | | | dCs; InaAs; dCs; |
| | | | | | | InaGs; dCs; InaCs; |
| | | | | | | dCs; InaGs; dAs; InaCs; |
| | | | | | | dA-Sup |
| 399 | ATP2A2- | CCTGACCCACCCTCC | ATP2A2 | 5′ | human | dCs; InaCs; dTs; InaGs; |
| | 60 m02 | | | | | dAs; InaCs; dCs; |
| | | | | | | InaCs; dAs; InaCs; dCs; |
| | | | | | | InaCs; dTs; InaCs; |
| | | | | | | dC-Sup |
| 400 | ATP2A2- | AGGGCAGGCCGCGGC | ATP2A2 | 5′ | human | dAs; InaGs; dGs; InaGs; |
| | 61 m02 | | | | | dCs; InaAs; dGs; |
| | | | | | | InaGs; dCs; InaCs; |
| | | | | | | dGs; InaCs; dGs; InaGs; |
| | | | | | | dC-Sup |
| 401 | ATP2A2- | CTGAATCACCCCGCG | ATP2A2 | 5′ | human | dCs; InaTs; dGs; InaAs; |
| | 62 m02 | | | | | dAs; InaTs; dCs; |
| | | | | | | InaAs; dCs; InaCs; dCs; |
| | | | | | | InaCs; dGs; InaCs; |
| | | | | | | dG-Sup |
| 402 | ATP2A2- | GGCCCCGAGCTCCGC | ATP2A2 | 5′ | human | dGs; InaGs; dCs; InaCs; |
| | 63 m02 | | | | | dCs; InaCs; dGs; |
| | | | | | | InaAs; dGs; InaCs; |
| | | | | | | dTs; InaCs; dCs; InaGs; |
| | | | | | | dC-Sup |
| 403 | ATP2A2- | GCGGCTGCTCTAATA | ATP2A2 | 5′ | human | dGs; InaCs; dGs; InaGs; |
| | 64 m02 | | | | | dCs; InaTs; dGs; |
| | | | | | | InaCs; dTs; InaCs; dTs; |
| | | | | | | InaAs; dAs; InaTs; |
| | | | | | | dA-Sup |
| 404 | ATP2A2- | CGCCGCGGCATGTGG | ATP2A2 | 5′ | human | dCs; InaGs; dCs; InaCs; |
| | 65 m02 | | | | | dGs; InaCs; dGs; |
| | | | | | | InaGs; dCs; InaAs; |
| | | | | | | dTs; InaGs; dTs; InaGs; |
| | | | | | | dG-Sup |
| 405 | ATP2A2- | CCCTCCTCCTCTTGC | ATP2A2 | 5′ | human | dCs; InaCs; dCs; InaTs; |
| | 66 m02 | | | | | dCs; InaCs; dTs; |
| | | | | | | InaCs; dCs; InaTs; dCs; |
| | | | | | | InaTs; dTs; InaGs; |
| | | | | | | dC-Sup |
| 406 | ATP2A2- | GGCCGCGGGCTCGTG | ATP2A2 | 5′ | human | dGs; InaGs; dCs; InaCs; |
| | 67 m02 | | | | | dGs; InaCs; dGs; |
| | | | | | | InaGs; dGs; InaCs; |
| | | | | | | dTs; InaCs; dGs; InaTs; |
| | | | | | | dG-Sup |
| 407 | ATP2A2- | GTTATTTTTCTCTGT | ATP2A2 | 3′ | human | dGs; InaTs; dTs; InaAs; |
| | 68 m02 | | | | | dTs; InaTs; dTs; |
| | | | | | | InaTs; dTs; InaCs; dTs; |
| | | | | | | InaCs; dTs; InaGs; |
| | | | | | | dT-Sup |
| 408 | ATP2A2- | ATTTAAAATGTTTTA | ATP2A2 | 3′ | human | dAs; InaTs; dTs; InaTs; |
| | 69 m02 | | | | | dAs; InaAs; dAs; |
| | | | | | | InaAs; dTs; InaGs; dTs; |
| | | | | | | InaTs; dTs; InaTs; |
| | | | | | | dA-Sup |
| 409 | ATP2A2- | TCTCTGTCCATTTAA | ATP2A2 | 3′ | human | dTs; InaCs; dTs; InaCs; |
| | 70 m02 | | | | | dTs; InaGs; dTs; |
| | | | | | | InaCs; dCs; InaAs; |
| | | | | | | dTs; InaTs; dTs; InaAs; |
| | | | | | | dA-Sup |
| 410 | ATP2A2- | TCATTTGGTCATGTG | ATP2A2 | 3′ | human | dTs; InaCs; dAs; InaTs; |
| | 71 m02 | | | | | dTs; InaTs; dGs; |
| | | | | | | InaGs; dTs; InaCs; |
| | | | | | | dAs; InaTs; dGs; InaTs; |
| | | | | | | dG-Sup |
| 411 | ATP2A2- | TAGTTCTCTGTACAT | ATP2A2 | 3′ | human | dTs; InaAs; dGs; InaTs; |
| | 72 m02 | | | | | dTs; InaCs; dTs; |
| | | | | | | InaCs; dTs; InaGs; dTs; |
| | | | | | | InaAs; dCs; InaAs; |
| | | | | | | dT-Sup |
| 412 | ATP2A2- | TCTGCTGGCTCAACT | ATP2A2 | 3′ | human | dTs; InaCs; dTs; InaGs; |
| | 73 m02 | | | | | dCs; InaTs; dGs; |
| | | | | | | InaGs; dCs; InaTs; dCs; |
| | | | | | | InaAs; dAs; InaCs; |
| | | | | | | dT-Sup |
| 413 | ATP2A2- | ATCATAGAATAGATT | ATP2A2 | 3′ | human | dAs; InaTs; dCs; InaAs; |
| | 74 m02 | | | | | dTs; InaAs; dGs; |
| | | | | | | InaAs; dAs; InaTs; |
| | | | | | | dAs; InaGs; dAs; InaTs; |
| | | | | | | dT-Sup |
| 414 | ATP2A2- | TTATCATAGAATAGA | ATP2A2 | 3′ | human | dTs; InaTs; dAs; InaTs; |
| | 75 m02 | | | | | dCs; InaAs; dTs; |
| | | | | | | InaAs; dGs; InaAs; |
| | | | | | | dAs; InaTs; dAs; InaGs; |
| | | | | | | dA-Sup |
| 415 | ATP2A2- | AATTGACATTTAGCA | ATP2A2 | 3′ | human | dAs; InaAs; dTs; InaTs; |
| | 76 m02 | | | | | dGs; InaAs; dCs; |
| | | | | | | InaAs; dTs; InaTs; dTs; |
| | | | | | | InaAs; dGs; InaCs; |
| | | | | | | dA-Sup |
| 416 | ATP2A2- | GACATTTAGCATTTT | ATP2A2 | 3′ | human | dGs; InaAs; dCs; InaAs; |
| | 77 m02 | | | | | dTs; InaTs; dTs; |
| | | | | | | InaAs; dGs; InaCs; |
| | | | | | | dAs; InaTs; dTs; InaTs; |
| | | | | | | dT-Sup |
| 417 | ATP2A2- | TTAACCATTCAACAC | ATP2A2 | 3′ | human | dTs; InaTs; dAs; InaAs; |
| | 78 m02 | | | | | dCs; InaCs; dAs; |
| | | | | | | InaTs; dTs; InaCs; dAs; |
| | | | | | | InaAs; dCs; InaAs; |
| | | | | | | dC-Sup |
| 418 | mKLF4- | CTTGGCCGGGGAAC | KLF4 | 5′ | mouse | dCs; InaTs; dTs; InaGs; |
| | 01 m02 | T | | | | dGs; InaCs; dCs; |
| | | | | | | InaGs; dGs; InaGs; |
| | | | | | | dGs; InaAs; dAs; |
| | | | | | | InaCs; dT-Sup |
| 419 | mKLF4- | GCCGGGGAACTGCC | KLF4 | 5′ | mouse | dGs; InaCs; dCs; InaGs; |
| | 02 m02 | G | | | | dGs; InaGs; dGs; |
| | | | | | | InaAs; dAs; InaCs; |
| | | | | | | dTs; InaGs; dCs; |
| | | | | | | InaCs; dG-Sup |
| 420 | mKLF4- | CGCCCGGAGCCGCG | KLF4 | 5′ | mouse | dCs; InaGs; dCs; InaCs; |
| | 03 m02 | C | | | | dCs; InaGs; dGs; |
| | | | | | | InaAs; dGs; InaCs; |
| | | | | | | dCs; InaGs; dCs; |
| | | | | | | InaGs; dC-Sup |
| 421 | mKLF4- | CTTGGCCGGGGAAC | KLF4 | 5′ | mouse | dCs; InaTs; dTs; InaGs; |
| | 04 m02 | TCC | | | | dGs; InaCs; dCs; |
| | | | | | | InaGs; dGs; InaGs; |
| | | | | | | dGs; InaAs; dAs; |
| | | | | | | InaCs; dTs; InaCs; |
| | | | | | | dC-Sup |
| 422 | mKLF4- | GCCGGGGAACTGCC | KLF4 | 5′ | mouse | dGs; InaCs; dCs; InaGs; |
| | 05 m02 | GC | | | | dGs; InaGs; dGs; |
| | | | | | | InaAs; dAs; InaCs; |
| | | | | | | dTs; InaGs; dCs; |
| | | | | | | InaCs; dGs; InaC- |
| | | | | | | Sup |
| 423 | mKLF4- | CGCCCGGAGCCGCG | KLF4 | 5′ | mouse | dCs; InaGs; dCs; InaCs; |
| | 06 m02 | CC | | | | dCs; InaGs; dGs; |
| | | | | | | InaAs; dGs; InaCs; |
| | | | | | | dCs; InaGs; dCs; |
| | | | | | | InaGs; dCs; InaC- |
| | | | | | | Sup |
| 424 | mKLF4- | CTTGGCCGGGGAAC | KLF4 | 5′ and | mouse | dCs; InaTs; dTs; InaGs; |
| | 07 m02 | TATAAAATTC | | 3′ | | dGs; InaCs; dCs; |
| | | | | | | InaGs; dGs; InaGs; |
| | | | | | | dGs; InaAs; dAs; |
| | | | | | | InaCs; dTs; InaAs; |
| | | | | | | dTs; dAs; dAs; dAs; |
| | | | | | | dAs; InaTs; dTs; |
| | | | | | | InaC-Sup |
| 425 | mKLF4- | CTTGGCCGGGGAAC | KLF4 | 5′ and | mouse | dCs; InaTs; dTs; InaGs; |
| | 08 m02 | TTTTTGTCGTTCAGAT | | 3′ | | dGs; InaCs; dCs; |
| | | AAAA | | | | InaGs; dGs; InaGs; |
| | | | | | | dGs; InaAs; dAs; |
| | | | | | | InaCs; dTs; InaTs; |
| | | | | | | dTs; InaTs; dTs; |
| | | | | | | InaGs; dTs; InaCs; |
| | | | | | | dGs; InaTs; dTs; |
| | | | | | | InaCs; dAs; InaGs; |
| | | | | | | dAs; InaTs; dAs; InaAs; |
| | | | | | | dAs; InaA-Sup |
| 426 | mKLF4- | CTTGGCCGGGGAAC | KLF4 | 5′ and | mouse | dCs; InaTs; dTs; InaGs; |
| | 09 m02 | TTTTTCAGATAAAAT | | 3′ | | dGs; InaCs; dCs; |
| | | ATT | | | | InaGs; dGs; InaGs; |
| | | | | | | dGs; InaAs; dAs; |
| | | | | | | InaCs; dTs; InaTs; |
| | | | | | | dTs; InaTs; dTs; |
| | | | | | | InaCs; dAs; InaGs; |
| | | | | | | dAs; InaTs; dAs; |
| | | | | | | InaAs; dAs; InaAs; |
| | | | | | | dTs; InaAs; dTs; |
| | | | | | | InaT-Sup |
| 427 | mKLF4- | CTTGGCCGGGGAAC | KLF4 | 5′ and | mouse | dCs; InaTs; dTs; InaGs; |
| | 10 m02 | TGTCGTTCAGATAAA | | 3′ | | dGs; InaCs; dCs; |
| | | A | | | | InaGs; dGs; InaGs; |
| | | | | | | dGs; InaAs; dAs; |
| | | | | | | InaCs; dTs; InaGs; |
| | | | | | | dTs; InaCs; dGs; |
| | | | | | | InaTs; dTs; InaCs; |
| | | | | | | dAs; InaGs; dAs; |
| | | | | | | InaTs; dAs; InaAs; |
| | | | | | | dAs; InaA-Sup |
| 428 | mKLF4- | CTTGGCCGGGGAAC | KLF4 | 5′ and | mouse | dCs; InaTs; dTs; InaGs; |
| | 11 m02 | TTTCAGATAAAATAT | | 3′ | | dGs; InaCs; dCs; |
| | | T | | | | InaGs; dGs; InaGs; |
| | | | | | | dGs; InaAs; dAs; |
| | | | | | | InaCs; dTs; InaTs; |
| | | | | | | dTs; InaCs; dAs; |
| | | | | | | InaGs; dAs; InaTs; |
| | | | | | | dAs; InaAs; dAs; |
| | | | | | | InaAs; dTs; InaAs; |
| | | | | | | dTs; InaT-Sup |
| 429 | mKLF4- | CCGGGGAACTTTTTG | KLF4 | 5′ and | mouse | dCs; InaCs; dGs; InaGs; |
| | 12 m02 | TCGTTCAGA | | 3′ | | dGs; InaGs; dAs; |
| | | | | | | InaAs; dCs; InaTs; |
| | | | | | | dTs; InaTs; dTs; |
| | | | | | | InaTs; dGs; InaTs; |
| | | | | | | dCs; InaGs; dTs; |
| | | | | | | InaTs; dCs; InaAs; |
| | | | | | | dGs; InaA-Sup |
| 430 | mKLF4- | CGGGGAACTTTTTCA | KLF4 | 5′ and | mouse | dCs; InaGs; dGs; |
| | 13 m02 | GATAAA | | 3′ | | InaGs; dGs; InaAs; |
| | | | | | | dAs; InaCs; dTs; |
| | | | | | | InaTs; dTs; InaTs; |
| | | | | | | dTs; InaCs; dAs; InaGs; |
| | | | | | | dAs; InaTs; dAs; |
| | | | | | | InaAs; dA-Sup |
| 431 | mKLF4- | CGGGGAACTGTCGTT | KLF4 | 5′ and | mouse | dCs; InaGs; dGs; |
| | 14 m02 | CAGA | | 3′ | | InaGs; dGs; InaAs; |
| | | | | | | dAs; InaCs; dTs; |
| | | | | | | InaGs; dTs; InaCs; |
| | | | | | | dGs; InaTs; dTs; InaCs; |
| | | | | | | dAs; InaGs; dA- |
| | | | | | | Sup |
| 432 | mKLF4- | CCGGGGAACTTTCAG | KLF4 | 5′ and | mouse | dCs; InaCs; dGs; InaGs; |
| | 15 m02 | ATAAA | | 3′ | | dGs; InaGs; dAs; |
| | | | | | | InaAs; dCs; InaTs; |
| | | | | | | dTs; InaTs; dCs; |
| | | | | | | InaAs; dGs; InaAs; |
| | | | | | | dTs; InaAs; dAs; |
| | | | | | | InaA-Sup |
| 433 | mKLF4- | GTCGTTCAGATAAAA | KLF4 | 3′ | mouse | dGs; InaTs; dCs; InaGs; |
| | 16 m02 | | | | | dTs; InaTs; dCs; |
| | | | | | | InaAs; dGs; InaAs; |
| | | | | | | dTs; InaAs; dAs; |
| | | | | | | InaAs; dA-Sup |
| 434 | mKLF4- | TTCAGATAAAATATT | KLF4 | 3′ | mouse | dTs; InaTs; dCs; InaAs; |
| | 17 m02 | | | | | dGs; InaAs; dTs; |
| | | | | | | InaAs; dAs; InaAs; |
| | | | | | | dAs; InaTs; dAs; |
| | | | | | | InaTs; dT-Sup |
| 435 | mKLF4- | TTTTTGTCGTTCAGAT | KLF4 | 3′ | mouse | dTs; InaTs; dTs; InaTs; |
| | 18 m02 | AAAA | | | | dTs; InaGs; dTs; |
| | | | | | | InaCs; dGs; InaTs; |
| | | | | | | dTs; InaCs; dAs; |
| | | | | | | InaGs; dAs; InaTs; |
| | | | | | | dAs; InaAs; dAs; |
| | | | | | | InaA-Sup |
| 436 | mKLF4- | TTTTTCAGATAAAAT | KLF4 | 3′ | mouse | dTs; InaTs; dTs; InaTs; |
| | 19 m02 | ATT | | | | dTs; InaCs; dAs; |
| | | | | | | InaGs; dAs; InaTs; |
| | | | | | | dAs; InaAs; dAs; |
| | | | | | | InaAs; dTs; InaAs; |
| | | | | | | dTs; InaT-Sup |
| 437 | mFXN- | CTCCGCGGCCGCTCC | FXN | 5′ | mouse | dCs; InaTs; dCs; InaCs; |
| | 01 m02 | | | | | dGs; InaCs; dGs; |
| | | | | | | InaGs; dCs; InaCs; |
| | | | | | | dGs; InaCs; dTs; |
| | | | | | | InaCs; dC-Sup |
| 438 | mFXN- | GCCCACATGCTACTC | FXN | 5′ | mouse | dGs; InaCs; dCs; InaCs; |
| | 02 m02 | | | | | dAs; InaCs; dAs; |
| | | | | | | InaTs; dGs; InaCs; |
| | | | | | | dTs; InaAs; dCs; |
| | | | | | | InaTs; dC-Sup |
| 439 | mFXN- | TCCGAACGCCCACAT | FXN | 5′ | mouse | dTs; InaCs; dCs; InaGs; |
| | 03 m02 | | | | | dAs; InaAs; dCs; |
| | | | | | | InaGs; dCs; InaCs; |
| | | | | | | dCs; InaAs; dCs; |
| | | | | | | InaAs; dT-Sup |
| 440 | mFXN- | CGAGGACTCGGTGG | FXN | 5′ | mouse | dCs; InaGs; dAs; InaGs; |
| | 04 m02 | T | | | | dGs; InaAs; dCs; |
| | | | | | | InaTs; dCs; InaGs; |
| | | | | | | dGs; InaTs; dGs; |
| | | | | | | InaGs; dT-Sup |
| 441 | mFXN- | CCAGCTCCGCGGCCG | FXN | 5′ | mouse | dCs; InaCs; dAs; InaGs; |
| | 05 m02 | | | | | dCs; InaTs; dCs; |
| | | | | | | InaCs; dGs; InaCs; |
| | | | | | | dGs; InaGs; dCs; |
| | | | | | | InaCs; dG-Sup |
| 442 | mFXN- | CTCCGCGGCCGCTCC | FXN | 5′ | mouse | dCs; InaTs; dCs; InaCs; |
| | 06 m02 | C | | | | dGs; InaCs; dGs; |
| | | | | | | InaGs; dCs; InaCs; |
| | | | | | | dGs; InaCs; dTs; |
| | | | | | | InaCs; dCs; InaC- |
| | | | | | | Sup |
| 443 | mFXN- | GCCCACATGCTACTC | FXN | 5′ | mouse | dGs; InaCs; dCs; InaCs; |
| | 07 m02 | C | | | | dAs; InaCs; dAs; |
| | | | | | | InaTs; dGs; InaCs; |
| | | | | | | dTs; InaAs; dCs; |
| | | | | | | InaTs; dCs; InaC- |
| | | | | | | Sup |
| 444 | mFXN- | CTCCGCGGCCGCTCC | FXN | 5′ | mouse | dCs; InaTs; dCs; InaCs; |
| | 08 m02 | TCAAAGATC | | | | dGs; InaCs; dGs; |
| | | | | | | InaGs; dCs; InaCs; |
| | | | | | | dGs; InaCs; dTs; |
| | | | | | | InaCs; dCs; InaTs; |
| | | | | | | dCs; dAs; dAs; dAs; |
| | | | | | | dGs; InaAs; dTs; |
| | | | | | | InaC-Sup |
| 445 | mFXN- | GCCCACATGCTACTC | FXN | 5′ | mouse | dGs; InaCs; dCs; InaCs; |
| | 09 m02 | CCAAAGGTC | | | | dAs; InaCs; dAs; |
| | | | | | | InaTs; dGs; InaCs; |
| | | | | | | dTs; InaAs; dCs; |
| | | | | | | InaTs; dCs; InaCs; |
| | | | | | | dCs; dAs; dAs; dAs; |
| | | | | | | dGs; InaGs; dTs; |
| | | | | | | InaC-Sup |
| 446 | mFXN- | CTCCGCGGCCGCTCC | FXN | 5′ and | mouse | dCs; InaTs; dCs; InaCs; |
| | 10 m02 | TTTTTGGGAGGGAAC | | 3′ | | dGs; InaCs; dGs; |
| | | ACACT | | | | InaGs; dCs; InaCs; |
| | | | | | | dGs; InaCs; dTs; |
| | | | | | | InaCs; dCs; InaTs; |
| | | | | | | dTs; InaTs; dTs; |
| | | | | | | InaTs; dGs; InaGs; |
| | | | | | | dGs; InaAs; dGs; |
| | | | | | | InaGs; dGs; InaAs; |
| | | | | | | dAs; InaCs; dAs; |
| | | | | | | InaCs; dAs; InaCs; |
| | | | | | | dT-Sup |
| 447 | mFXN- | GCCCACATGCTACTC | FXN | 5′ and | mouse | dGs; InaCs; dCs; |
| | 11 m02 | TTTTTGGGAGGGAAC | | 3′ | | InaCs; dAs; InaCs; |
| | | ACACT | | | | dAs; InaTs; dGs; |
| | | | | | | InaCs; dTs; InaAs; dCs; |
| | | | | | | InaTs; dCs; InaTs; |
| | | | | | | dTs; InaTs; dTs; |
| | | | | | | InaTs; dGs; InaGs; |
| | | | | | | dGs; InaAs; dGs; |
| | | | | | | InaGs; dGs; InaAs; |
| | | | | | | dAs; InaCs; dAs; |
| | | | | | | InaCs; dAs; InaCs; dT- |
| | | | | | | Sup |
| 448 | mFXN- | CTCCGCGGCCGCTCC | FXN | 5′ and | mouse | dCs; InaTs; dCs; InaCs; |
| | 12 m02 | GGGAGGGAACACAC | | 3′ | | dGs; InaCs; dGs; |
| | | T | | | | InaGs; dCs; InaCs; |
| | | | | | | dGs; InaCs; dTs; |
| | | | | | | InaCs; dCs; InaGs; |
| | | | | | | dGs; InaGs; dAs; |
| | | | | | | InaGs; dGs; InaGs; |
| | | | | | | dAs; InaAs; dCs; |
| | | | | | | InaAs; dCs; InaAs; |
| | | | | | | dCs; InaT-Sup |
| 449 | mFXN- | GCCCACATGCTACTC | FXN | 5′ and | mouse | dGs; InaCs; dCs; InaCs; |
| | 13 m02 | GGGAGGGAACACAC | | 3′ | | dAs; InaCs; dAs; |
| | | T | | | | InaTs; dGs; InaCs; |
| | | | | | | dTs; InaAs; dCs; |
| | | | | | | InaTs; dCs; InaGs; |
| | | | | | | dGs; InaGs; dAs; |
| | | | | | | InaGs; dGs; InaGs; |
| | | | | | | dAs; InaAs; dCs; |
| | | | | | | InaAs; dCs; InaAs; |
| | | | | | | dCs; InaT-Sup |
| 450 | mFXN- | CGGCCGCTCCGGGA | FXN | 5′ and | mouse | dCs; InaGs; dGs; |
| | 14 m02 | GGGAAC | | 3′ | | InaCs; dCs; InaGs; |
| | | | | | | dCs; InaTs; dCs; |
| | | | | | | InaCs; dGs; InaGs; |
| | | | | | | dGs; InaAs; dGs; |
| | | | | | | InaGs; dGs; InaAs; |
| | | | | | | dAs; InaC-Sup |
| 451 | mFXN- | CATGCTACTCGGGAG | FXN | 5′ and | mouse | dCs; InaAs; dTs; InaGs; |
| | 15 m02 | GGAAC | | 3′ | | dCs; InaTs; dAs; |
| | | | | | | InaCs; dTs; InaCs; |
| | | | | | | dGs; InaGs; dGs; |
| | | | | | | InaAs; dGs; InaGs; |
| | | | | | | dGs; InaAs; dAs; |
| | | | | | | InaC-Sup |
| 452 | mFXN- | GGGAGGGAACACAC | FXN | 3′ | mouse | dGs; InaGs; dGs; |
| | 16 m02 | T | | | | InaAs; dGs; InaGs; |
| | | | | | | dGs; InaAs; dAs; |
| | | | | | | InaCs; dAs; InaCs; |
| | | | | | | dAs; InaCs; dT- |
| | | | | | | Sup |
| 453 | mFXN- | GGGGTCTTCACCTGA | FXN | 3′ | mouse | dGs; InaGs; dGs; |
| | 17 m02 | | | | | InaGs; dTs; InaCs; |
| | | | | | | dTs; InaTs; dCs; |
| | | | | | | InaAs; dCs; InaCs; |
| | | | | | | dTs; InaGs; dA-Sup |
| 454 | mFXN- | GGCTGTTATATCATG | FXN | 3′ | mouse | dGs; InaGs; dCs; |
| | 18 m02 | | | | | InaTs; dGs; InaTs; |
| | | | | | | dTs; InaAs; dTs; |
| | | | | | | InaAs; dTs; InaCs; |
| | | | | | | dAs; InaTs; dG-Sup |
| 455 | mFXN- | GGCATTTTAAGATGG | FXN | 3′ | mouse | dGs; InaGs; dCs; |
| | 19 m02 | | | | | InaAs; dTs; InaTs; |
| | | | | | | dTs; InaTs; dAs; |
| | | | | | | InaAs; dGs; InaAs; |
| | | | | | | dTs; InaGs; dG-Sup |
| 456 | mFXN- | TTTTTGGGAGGGAAC | FXN | 3′ | mouse | dTs; InaTs; dTs; |
| | 20 m02 | ACACT | | | | InaTs; dTs; InaGs; |
| | | | | | | dGs; InaGs; dAs; |
| | | | | | | InaGs; dGs; InaGs; |
| | | | | | | Ads; InaAs; dCs; |
| | | | | | | InaAs; dCs; InaAs; |
| | | | | | | dCs; InaT-Sup |
| 457 | mFXN- | TTTTTGGCTGTTATAT | FXN | 3′ | mouse | dTs; InaTs; dTs; |
| | 21 m02 | CATG | | | | InaTs; dTs; InaGs; |
| | | | | | | dGs; InaCs; dTs; |
| | | | | | | InaGs; dTs; InaTs; dAs; |
| | | | | | | InaTs; dAs; InaTs; |
| | | | | | | dCs; InaAs; dTs; |
| | | | | | | InaG-Sup |

Table 9 provides further exemplary RNA 5′ and 3′ end targeting oligos for multiple human and mouse genes.

TABLE 9
Oligonucleotides designed to target 5′ and 3′ ends of RNAs

| SEQ | Oligo | | Gene | Target | | Formatted |
|---|---|---|---|---|---|---|
| ID NO | Name | Base Sequence | Name | Region | Organism | Sequence |
| 459 | FXN-654 | TGTCTCATTTGGAGA | FXN | 3′ | human | dTs; InaGs; dTs; InaCs; |
| | m02 | | | | | dTs; InaCs; dAs; InaTs; |
| | | | | | | dTs; InaTs; dGs; InaGs; |
| | | | | | | dAs; InaGs; dA-Sup |
| 460 | FXN-655 | ATAATGAAGCTGGG | FXN | 3′ | human | dAs; InaTs; dAs; InaAs; |
| | m02 | | | | | dTs; InaGs; dAs; InaAs; |
| | | | | | | dGs; InaCs; dTs; InaGs; |
| | | | | | | dGs; InaG-Sup |
| 461 | FXN-656 | TTTTCCCTCCTGGAA | FXN | 3′ | human | dTs; InaTs; dTs; InaTs; |
| | m02 | | | | | dCs; InaCs; dCs; InaTs; |
| | | | | | | dCs; InaCs; dTs; InaGs; |
| | | | | | | dGs; InaAs; dA-Sup |
| 462 | FXN-657 | TGCATAATGAAGCTG | FXN | 3′ | human | dTs; InaGs; dCs; InaAs; |
| | m02 | | | | | dTs; InaAs; dAs; InaTs; |
| | | | | | | dGs; InaAs; dAs; |
| | | | | | | InaGs; dCs; InaTs; dG- |
| | | | | | | Sup |
| 463 | FXN-658 | AAATCCTTCAAAGAA | FXN | 3′ | human | dAs; InaAs; dAs; InaTs; |
| | m02 | | | | | dCs; InaCs; dTs; InaTs; |
| | | | | | | dCs; InaAs; dAs; InaAs; |
| | | | | | | dGs; InaAs; dA-Sup |
| 464 | FXN-659 | TTGGAAGATTTTTTG | FXN | 3′ | human | dTs; InaTs; dGs; InaGs; |
| | m02 | | | | | dAs; InaAs; dGs; InaAs; |
| | | | | | | dTs; InaTs; dTs; |
| | | | | | | InaTs; dTs; InaTs; dG- |
| | | | | | | Sup |
| 465 | FXN-660 | GCATTCTTGTAGCAG | FXN | 3′ | human | dGs; InaCs; dAs; InaTs; |
| | m02 | | | | | dTs; InaCs; dTs; InaTs; |
| | | | | | | dGs; InaTs; dAs; InaGs; |
| | | | | | | dCs; InaAs; dG-Sup |
| 466 | FXN-557 | ACAACAAAAAACAGA | FXN | 3′ | human | dAs; InaCs; dAs; InaAs; |
| | m02 | | | | | dCs; InaAs; dAs; InaAs; |
| | | | | | | dAs; InaAs; dAs; |
| | | | | | | InaCs; dAs; InaGs; dA- |
| | | | | | | Sup |
| 467 | FXN-662 | TGAAGCTGGGGTCTT | FXN | 3′ | human | dTs; InaGs; dAs; InaAs; |
| | m02 | | | | | dGs; InaCs; dTs; InaGs; |
| | | | | | | dGs; InaGs; dGs; |
| | | | | | | InaTs; dCs; InaTs; dT- |
| | | | | | | Sup |
| 468 | FXN-663 | CCTGAAAACATTTGT | FXN | 3′ | human | dCs; InaCs; dTs; InaGs; |
| | m02 | | | | | dAs; InaAs; dAs; InaAs; |
| | | | | | | dCs; InaAs; dTs; InaTs; |
| | | | | | | dTs; InaGs; dT-Sup |
| 469 | FXN-664 | TTCATTTTCCCTCCT | FXN | 3′ | human | dTs; InaTs; dCs; InaAs; |
| | m02 | | | | | dTs; InaTs; dTs; InaTs; |
| | | | | | | dCs; InaCs; dCs; InaTs; |
| | | | | | | dCs; InaCs; dT-Sup |
| 470 | FXN-665 | TTATTATTATTATAT | FXN | 3′ | human | dTs; InaTs; dAs; InaTs; |
| | m02 | | | | | dTs; InaAs; dTs; InaTs; |
| | | | | | | dAs; InaTs; dTs; InaAs; |
| | | | | | | dTs; InaAs; dT-Sup |
| 471 | FXN-666 | TAACTTTGCATGAAT | FXN | 3′ | human | dTs; InaAs; dAs; InaCs; |
| | m02 | | | | | dTs; InaTs; dTs; InaGs; |
| | | | | | | dCs; InaAs; dTs; InaGs; |
| | | | | | | dAs; InaAs; dT-Sup |
| 472 | FXN-667 | ATACAAACATGTATG | FXN | 3′ | human | dAs; InaTs; dAs; InaCs; |
| | m02 | | | | | dAs; InaAs; dAs; InaCs; |
| | | | | | | dAs; InaTs; dGs; InaTs; |
| | | | | | | dAs; InaTs; dG-Sup |
| 473 | FXN-668 | ATTGTAAACCTATAA | FXN | 3′ | human | dAs; InaTs; dTs; InaGs; |
| | m02 | | | | | dTs; InaAs; dAs; InaAs; |
| | | | | | | dCs; InaCs; dTs; InaAs; |
| | | | | | | dTs; InaAs; dA-Sup |
| 474 | FXN-669 | TGGAGTTGGGGTTAT | FXN | 3′ | human | dTs; InaGs; dGs; InaAs; |
| | m02 | | | | | dGs; InaTs; dTs; InaGs; |
| | | | | | | dGs; InaGs; dGs; InaTs; |
| | | | | | | dTs; InaAs; dT-Sup |
| 475 | FXN-670 | GTTGGGGTTATTTAG | FXN | 3′ | human | dGs; InaTs; dTs; InaGs; |
| | m02 | | | | | dGs; InaGs; dGs; InaTs; |
| | | | | | | dTs; InaAs; dTs; |
| | | | | | | InaTs; dTs; InaAs; dG- |
| | | | | | | Sup |
| 476 | FXN-671 | CTCCGCCCTCCAG | FXN | 5′ | human | dCs; InaTs; dCs; InaCs; |
| | m02 | | | | | dGs; InaCs; dCs; InaCs; |
| | | | | | | dTs; InaCs; dCs; InaAs; |
| | | | | | | dG-Sup |
| 477 | FXN-672 | CCGCCCTCCAG | FXN | 5′ | human | dCs; InaCs; dGs; InaCs; |
| | m02 | | | | | dCs; InaCs; dTs; InaCs; |
| | | | | | | dCs; InaAs; dG-Sup |
| 478 | FXN-673 | GCCCTCCAG | FXN | 5′ | human | dGs; InaCs; dCs; InaCs; |
| | m02 | | | | | dTs; InaCs; dCs; InaAs; |
| | | | | | | dG-Sup |
| 479 | FXN-674 | CCCGCTCCGCCCTCC | FXN | 5′ | human | dCs; InaCs; dCs; InaGs; |
| | m02 | | | | | dCs; InaTs; dCs; InaCs; |
| | | | | | | dGs; InaCs; dCs; InaCs; |
| | | | | | | dTs; InaCs; dC-Sup |
| 480 | FXN-675 | CGCTCCGCCCTCC | FXN | 5′ | human | dCs; InaGs; dCs; InaTs; |
| | m02 | | | | | dCs; InaCs; dGs; InaCs; |
| | | | | | | dCs; InaCs; dTs; InaCs; |
| | | | | | | dC-Sup |
| 481 | FXN-676 | CTCCGCCCTCC | FXN | 5′ | human | dCs; InaTs; dCs; InaCs; |
| | m02 | | | | | dGs; InaCs; dCs; InaCs; |
| | | | | | | dTs; InaCs; dC-Sup |
| 482 | FXN-677 | CCGCCCTCC | FXN | 5′ | human | dCs; InaCs; dGs; InaCs; |
| | m02 | | | | | dCs; InaCs; dTs; InaCs; |
| | | | | | | dC-Sup |
| 483 | FXN-678 | GCCACTGGCCGCA | FXN | 5′ | human | dGs; InaCs; dCs; InaAs; |
| | m02 | | | | | dCs; InaTs; dGs; InaGs; |
| | | | | | | dCs; InaCs; dGs; |
| | | | | | | InaCs; dA-Sup |
| 484 | FXN-679 | CACTGGCCGCA | FXN | 5′ | human | dCs; InaAs; dCs; InaTs; |
| | m02 | | | | | dGs; InaGs; dCs; InaCs; |
| | | | | | | dGs; InaCs; dA-Sup |
| 485 | FXN-680 | GCGACCCCTGGTG | FXN | 5′ | human | dGs; InaCs; dGs; InaAs; |
| | m02 | | | | | dCs; InaCs; dCs; InaCs; |
| | | | | | | dTs; InaGs; dGs; |
| | | | | | | InaTs; dG-Sup |
| 486 | FXN-681 | GACCCCTGGTG | FXN | 5′ | human | dGs; InaAs; dCs; InaCs; |
| | m02 | | | | | dCs; InaCs; dTs; InaGs; |
| | | | | | | dGs; InaTs; dG-Sup |
| 487 | FXN-682 | CTGGCCGCAGGCA | FXN | 5′ | human | dCs; InaTs; dGs; InaGs; |
| | m02 | | | | | dCs; InaCs; dGs; InaCs; |
| | | | | | | dAs; InaGs; dGs; |
| | | | | | | InaCs; dA-Sup |
| 488 | FXN-683 | GGCCACTGGCCGC | FXN | 5′ | human | dGs; InaGs; dCs; InaCs; |
| | m02 | | | | | dAs; InaCs; dTs; InaGs; |
| | | | | | | dGs; InaCs; dCs; |
| | | | | | | InaGs; dC-Sup |
| 489 | FXN-684 | CTGGTGGCCACTG | FXN | 5′ | human | dCs; InaTs; dGs; InaGs; |
| | m02 | | | | | dTs; InaGs; dGs; InaCs; |
| | | | | | | dCs; InaAs; dCs; InaTs; |
| | | | | | | dG-Sup |
| 490 | FXN-685 | GACCCCTGGTGGC | FXN | 5′ | human | dGs; InaAs; dCs; InaCs; |
| | m02 | | | | | dCs; InaCs; dTs; InaGs; |
| | | | | | | dGs; InaTs; dGs; |
| | | | | | | InaGs; dC-Sup |
| 491 | FXN-686 | GCGGCGACCCCTG | FXN | 5′ | human | dGs; InaCs; dGs; InaGs; |
| | m02 | | | | | dCs; InaGs; dAs; |
| | | | | | | InaCs; dCs; InaCs; dCs; |
| | | | | | | InaTs; dG-Sup |
| 492 | FXN-687 | GTGCTGCGGCGAC | FXN | 5′ | human | dGs; InaTs; dGs; InaCs; |
| | m02 | | | | | dTs; InaGs; dCs; InaGs; |
| | | | | | | dGs; InaCs; dGs; |
| | | | | | | InaAs; dC-Sup |
| 493 | FXN-688 | GCTGGGTGCTGCG | FXN | 5′ | human | dGs; InaCs; dTs; InaGs; |
| | m02 | | | | | dGs; InaGs; dTs; InaGs; |
| | | | | | | dCs; InaTs; dGs; |
| | | | | | | InaCs; dG-Sup |
| 494 | FXN-689 | CCAGCGCTGGGTG | FXN | 5′ | human | dCs; InaCs; dAs; InaGs; |
| | m02 | | | | | dCs; InaGs; dCs; InaTs; |
| | | | | | | dGs; InaGs; dGs; |
| | | | | | | InaTs; dG-Sup |
| 495 | FXN-690 | GCCCTCCAGCGCT | FXN | 5′ | human | dGs; InaCs; dCs; InaCs; |
| | m02 | | | | | dTs; InaCs; dCs; InaAs; |
| | | | | | | dGs; InaCs; dGs; |
| | | | | | | InaCs; dT-Sup |
| 496 | FXN-691 | CGCCCGCTCCGCC | FXN | 5′ | human | dCs; InaGs; dCs; InaCs; |
| | m02 | | | | | dCs; InaGs; dCs; InaTs; |
| | | | | | | dCs; InaCs; dGs; InaCs; |
| | | | | | | dC-Sup |
| 497 | FXN-460 | CGCCCTCCAGCGCTGTT | FXN | 5′ and 3′ | human | dCs; InaGs; dCs; InaCs; |
| | m1000 | TTTATTATTTTGCTTTTT | | | | dCs; InaTs; dCs; InaCs; |
| | | | | | | dAs; InaGs; dCs; |
| | | | | | | InaGs; dCs; InaTs; dGs; |
| | | | | | | dT; dT; dT; dT; dT; dAs; |
| | | | | | | InaTs; dTs; InaAs; |
| | | | | | | dTs; InaTs; dTs; InaTs; |
| | | | | | | dGs; InaCs; dTs; InaTs; |
| | | | | | | dTs; InaTs; dT-Sup |
| 498 | FXN-461 | CGCTCCGCCCTCCAGTTT | FXN | 5′ and 3′ | human | dCs; InaGs; dCs; InaTs; |
| | m1000 | TTATTATTTTGCTTTTT | | | | dCs; InaCs; dGs; InaCs; |
| | | | | | | dCs; InaCs; dTs; |
| | | | | | | InaCs; dCs; InaAs; dGs; |
| | | | | | | dT; dT; dT; dT; dT; dAs; |
| | | | | | | InaTs; dTs; InaAs; dTs; |
| | | | | | | InaTs; dTs; InaTs; dGs; |
| | | | | | | InaCs; dTs; InaTs; |
| | | | | | | dTs; InaTs; dT-Sup |
| 499 | FXN-523 | CAAGTCCAGTTTGGTTT | FXN | 3′ | human | InaCs; omeAs; InaAs; |
| | m01 | | | | | omeGs; InaTs; omeCs; |
| | | | | | | InaCs; omeAs; InaGs; |
| | | | | | | omeUs; InaTs; |
| | | | | | | omeUs; InaGs; omeGs; |
| | | | | | | InaTs; omeUs; InaT-Sup |
| 500 | FXN-524 | GAATAGGCCAAGGAAG | FXN | 3′ | human | InaGs; omeAs; InaAs; |
| | m01 | A | | | | omeUs; InaAs; omeGs; |
| | | | | | | InaGs; omeCs; InaCs; |
| | | | | | | omeAs; InaAs; |
| | | | | | | omeGs; InaGs; omeAs; |
| | | | | | | InaAs; omeGs; InaA-Sup |
| 501 | FXN-525 | ATCAAGCATCTTTTCCG | FXN | 3′ | human | InaAs; omeUs; InaCs; |
| | m01 | | | | | omeAs; InaAs; omeGs; |
| | | | | | | InaCs; omeAs; InaTs; |
| | | | | | | omeCs; InaTs; |
| | | | | | | omeUs; InaTs; omeUs; |
| | | | | | | InaCs; omeCs; InaG-Sup |
| 502 | FXN-526 | TTAAAACGGGGCTGGG | FXN | 3′ | human | InaTs; omeUs; InaAs; |
| | m01 | C | | | | omeAs; InaAs; omeAs; |
| | | | | | | InaCs; omeGs; |
| | | | | | | InaGs; deaGs; InaGs; |
| | | | | | | omeCs; InaTs; omeGs; |
| | | | | | | InaGs; omeGs; InaC-Sup |
| 503 | FXN-527 | GATAGCTTTTAATGTCC | FXN | 3′ | human | InaGs; omeAs; InaTs; |
| | m01 | | | | | omeAs; InaGs; omeCs; |
| | | | | | | InaTs; omeUs; |
| | | | | | | InaTs; omeUs; InaAs; |
| | | | | | | omeAs; InaTs; omeGs; |
| | | | | | | InaTs; omeCs; InaC-Sup |
| 504 | FXN-528 | AGCTGGGGTCTTGGCCT | FXN | 3′ | human | InaAs; omeGs; InaCs; |
| | m01 | | | | | omeUs; InaGs; deaGs; |
| | | | | | | InaGs; omeGs; |
| | | | | | | InaTs; omeCs; InaTs; |
| | | | | | | omeUs; InaGs; omeGs; |
| | | | | | | InaCs; omeCs; InaT-Sup |
| 505 | FXN-529 | CCTCAGCTGCATAATGA | FXN | 3′ | human | InaCs; omeCs; InaTs; |
| | m01 | | | | | omeCs; InaAs; omeGs; |
| | | | | | | InaCs; omeUs; |
| | | | | | | InaGs; omeCs; InaAs; |
| | | | | | | omeUs; InaAs; omeAs; |
| | | | | | | InaTs; omeGs; InaA-Sup |
| 506 | FXN-530 | CAACAACAAAAAACAGA | FXN | 3′ | human | InaCs; omeAs; InaAs; |
| | m01 | | | | | omeCs; InaAs; omeAs; |
| | | | | | | InaCs; omeAs; |
| | | | | | | InaAs; omeAs; InaAs; |
| | | | | | | omeAs; InaAs; omeCs; |
| | | | | | | InaAs; omeGs; InaA-Sup |
| 507 | FXN-531 | AAAAAAATAAACAACAA | FXN | 3′ | human | InaAs; omeAs; InaAs; |
| | m01 | | | | | omeAs; InaAs; omeAs; |
| | | | | | | InaAs; omeUs; |
| | | | | | | InaAs; omeAs; InaAs; |
| | | | | | | omeCs; InaAs; omeAs; |
| | | | | | | InaCs; omeAs; InaA-Sup |
| 508 | FXN-532 | CCTCAAAAGCAGGAATA | FXN | 3′ | human | InaCs; omeCs; InaTs; |
| | m01 | | | | | omeCs; InaAs; omeAs; |
| | | | | | | InaAs; omeAs; InaGs; |
| | | | | | | omeCs; InaAs; |
| | | | | | | omeGs; InaGs; omeAs; |
| | | | | | | InaAs; omeUs; InaA-Sup |
| 509 | FXN-533 | ACACATAGCCCAACTGT | FXN | 3′ | human | InaAs; omeCs; InaAs; |
| | m01 | | | | | omeCs; InaAs; omeUs; |
| | | | | | | InaAs; omeGs; |
| | | | | | | InaCs; omeCs; InaCs; |
| | | | | | | omeAs; InaAs; omeCs; |
| | | | | | | InaTs; omeGs; InaT-Sup |
| 510 | FXN-534 | CTTTCTACAGAGCTGTG | FXN | 3′ | human | InaCs; omeUs; InaTs; |
| | m01 | | | | | omeUs; InaCs; omeUs; |
| | | | | | | InaAs; omeCs; |
| | | | | | | InaAs; omeGs; InaAs; |
| | | | | | | omeGs; InaCs; omeUs; |
| | | | | | | InaGs; omeUs; InaG-Sup |
| 511 | FXN-535 | GTAGGAGGCAACACATT | FXN | 3′ | human | InaGs; omeUs; InaAs; |
| | m01 | | | | | omeGs; InaGs; omeAs; |
| | | | | | | InaGs; omeGs; |
| | | | | | | InaCs; omeAs; InaAs; |
| | | | | | | omeCs; InaAs; omeCs; |
| | | | | | | InaAs; omeUs; InaT-Sup |
| 512 | FXN-536 | CAGAACTTGGGGGCAA | FXN | 3′ | human | InaCs; omeAs; InaGs; |
| | m01 | G | | | | omeAs; InaAs; omeCs; |
| | | | | | | InaTs; omeUs; |
| | | | | | | InaGs; deaGs; InaGs; |
| | | | | | | deaGs; InaGs; omeCs; |
| | | | | | | InaAs; omeAs; InaG-Sup |
| 513 | FXN-537 | CCATAGAAATTAAAAAT | FXN | 3′ | human | InaCs; omeCs; InaAs; |
| | m01 | | | | | omeUs; InaAs; omeGs; |
| | | | | | | InaAs; omeAs; |
| | | | | | | InaAs; omeUs; InaTs; |
| | | | | | | omeAs; InaAs; omeAs; |
| | | | | | | InaAs; omeAs; InaT-Sup |
| 514 | FXN-538 | ACAATCCAAAAAATCTT | FXN | 3′ | human | InaAs; omeCs; InaAs; |
| | m01 | | | | | omeAs; InaTs; omeCs; |
| | | | | | | InaCs; omeAs; InaAs; |
| | | | | | | omeAs; InaAs; |
| | | | | | | omeAs; InaAs; omeUs; |
| | | | | | | InaCs; omeUs; InaT-Sup |
| 515 | FXN-539 | GTGAGGGAGGAAATCC | FXN | 3′ | human | InaGs; omeUs; InaGs; |
| | m01 | G | | | | omeAs; InaGs; omeGs; |
| | | | | | | InaGs; omeAs; |
| | | | | | | InaGs; omeGs; InaAs; |
| | | | | | | omeAs; InaAs; omeUs; |
| | | | | | | InaCs; omeCs; InaG-Sup |
| 516 | FXN-540 | AAGATAAGGGGTATCAT | FXN | 3′ | human | InaAs; omeAs; InaGs; |
| | m01 | | | | | omeAs; InaTs; omeAs; |
| | | | | | | InaAs; omeGs; |
| | | | | | | InaGs; omeGs; InaGs; |
| | | | | | | omeUs; InaAs; omeUs; |
| | | | | | | InaCs; omeAs; InaT-Sup |
| 517 | FXN-541 | GGCATAAGACATTATAA | FXN | 3′ | human | InaGs; omeGs; InaCs; |
| | m01 | | | | | omeAs; InaTs; omeAs; |
| | | | | | | InaAs; omeGs; InaAs; |
| | | | | | | omeCs; InaAs; |
| | | | | | | omeUs; InaTs; omeAs; |
| | | | | | | InaTs; omeAs; InaA-Sup |
| 518 | FXN-542 | TGTTATATTCAGGTATA | FXN | 3′ | human | InaTs; omeGs; InaTs; |
| | m01 | | | | | omeUs; InaAs; omeUs; |
| | | | | | | InaAs; omeUs; |
| | | | | | | InaTs; omeCs; InaAs; |
| | | | | | | omeGs; InaGs; omeUs; |
| | | | | | | InaAs; omeUs; InaA-Sup |
| 519 | FXN-543 | TTTGCTTTTTTAAAGGT | FXN | 3′ | human | InaTs; omeUs; InaTs; |
| | m01 | | | | | omeGs; InaCs; omeUs; |
| | | | | | | InaTs; omeUs; InaTs; |
| | | | | | | omeUs; InaTs; |
| | | | | | | omeAs; InaAs; omeAs; |
| | | | | | | InaGs; omeGs; InaT-Sup |
| 520 | FXN-544 | TTTTTCCTTCTTATTAT | FXN | 3′ | human | InaTs; omeUs; InaTs; |
| | m01 | | | | | omeUs; InaTs; omeCs; |
| | | | | | | InaCs; omeUs; InaTs; |
| | | | | | | omeCs; InaTs; omeUs; |
| | | | | | | InaAs; omeUs; InaTs; |
| | | | | | | omeAs; InaT-Sup |
| 521 | FXN-545 | CATTTTCCCTCCTGGAA | FXN | 3′ | human | InaCs; omeAs; InaTs; |
| | m01 | | | | | omeUs; InaTs; omeUs; |
| | | | | | | InaCs; omeCs; InaCs; |
| | | | | | | omeUs; InaCs; omeCs; |
| | | | | | | InaTs; omeGs; |
| | | | | | | InaGs; omeAs; InaA-Sup |
| 522 | FXN-546 | GAAGAGTGAAGACAAT | FXN | 3′ | human | InaGs; omeAs; InaAs; |
| | m01 | T | | | | omeGs; InaAs; omeGs; |
| | | | | | | InaTs; omeGs; InaAs; |
| | | | | | | omeAs; InaGs; omeAs; |
| | | | | | | InaCs; omeAs; |
| | | | | | | InaAs; omeUs; InaT-Sup |
| 523 | FXN-547 | TAAATCCTTCAAAGAAT | FXN | 3′ | human | InaTs; omeAs; InaAs; |
| | m01 | | | | | omeAs; InaTs; omeCs; |
| | | | | | | InaCs; omeUs; InaTs; |
| | | | | | | omeCs; InaAs; |
| | | | | | | omeAs; InaAs; omeGs; |
| | | | | | | InaAs; omeAs; InaT-Sup |
| 524 | FXN-548 | TCATGTACTTCTTGCAG | FXN | 3′ | human | InaTs; omeCs; InaAs; |
| | m01 | | | | | omeUs; InaGs; omeUs; |
| | | | | | | InaAs; omeCs; InaTs; |
| | | | | | | omeUs; InaCs; omeUs; |
| | | | | | | InaTs; omeGs; |
| | | | | | | InaCs; omeAs; InaG-Sup |
| 525 | FXN-549 | GGTTGACCAGCTGCTCT | FXN | 3′ | human | InaGs; omeGs; InaTs; |
| | m01 | | | | | omeUs; InaGs; omeAs; |
| | | | | | | InaCs; omeCs; InaAs; |
| | | | | | | omeGs; InaCs; omeUs; |
| | | | | | | InaGs; omeCs; |
| | | | | | | InaTs; omeCs; InaT-Sup |
| 526 | FXN-550 | AGATAGAACAGTGAGC | FXN | 3′ | human | InaAs; omeGs; InaAs; |
| | m01 | A | | | | omeUs; InaAs; omeGs; |
| | | | | | | InaAs; omeAs; InaCs; |
| | | | | | | omeAs; InaGs; |
| | | | | | | omeUs; InaGs; omeAs; |
| | | | | | | InaGs; omeCs; InaA-Sup |
| 527 | FXN-551 | TAATGTGTCTCATTTGG | FXN | 3′ | human | InaTs; omeAs; InaAs; |
| | m01 | | | | | omeUs; InaGs; omeUs; |
| | | | | | | InaGs; omeUs; InaCs; |
| | | | | | | omeUs; InaCs; |
| | | | | | | omeAs; InaTs; omeUs; |
| | | | | | | InaTs; omeGs; InaG-Sup |
| 528 | FXN-552 | ATTTGTAGGCTACCCTT | FXN | 3′ | human | InaAs; omeUs; InaTs; |
| | m01 | | | | | omeUs; InaGs; omeUs; |
| | | | | | | InaAs; omeGs; InaGs; |
| | | | | | | omeCs; InaTs; |
| | | | | | | omeAs; InaCs; omeCs; |
| | | | | | | InaCs; omeUs; InaT-Sup |
| 529 | FXN-553 | GAAAGAAGCCTGAAAA | FXN | 3′ | human | InaGs; omeAs; InaAs; |
| | m01 | C | | | | omeAs; InaGs; omeAs; |
| | | | | | | InaAs; omeGs; InaCs; |
| | | | | | | omeCs; InaTs; |
| | | | | | | omeGs; InaAs; omeAs; |
| | | | | | | InaAs; omeAs; InaC-Sup |
| 530 | FXN-554 | AGAAGTGCTTACACTTT | FXN | 3′ | human | InaAs; omeGs; InaAs; |
| | m01 | | | | | omeAs; InaGs; omeUs; |
| | | | | | | InaGs; omeCs; InaTs; |
| | | | | | | omeUs; InaAs; |
| | | | | | | omeCs; InaAs; omeCs; |
| | | | | | | InaTs; omeUs; InaT-Sup |
| 531 | FXN-555 | TCAATGCTAAAGAGCTC | FXN | 3′ | human | InaTs; omeCs; InaAs; |
| | m01 | | | | | omeAs; InaTs; omeGs; |
| | | | | | | InaCs; omeUs; InaAs; |
| | | | | | | omeAs; InaAs; |
| | | | | | | omeGs; InaAs; omeGs; |
| | | | | | | InaCs; omeUs; InaC-Sup |
| 532 | Apoa1_ | AGTCTGGGTGTCC | Apoa1 | 5′ | mouse | InaAs; dGs; InaTs; |
| | mus-01 | | | | | dCs; InaTs; dGs; InaGs; |
| | m12 | | | | | dGs; InaTs; dGs; |
| | | | | | | InaTs; dCs; InaC-Sup |
| 533 | Apoa1_ | CCGACAGTCTGGG | Apoa1 | 5′ | mouse | InaCs; dCs; InaGs; |
| | mus-02 | | | | | dAs; InaCs; dAs; InaGs; |
| | m12 | | | | | dTs; InaCs; dTs; |
| | | | | | | InaGs; dGs; InaG- |
| 534 | Apoa1_ | CTCCGACAGTCTG | Apoa1 | 5′ | mouse | InaCs; dTs; InaCs; |
| | mus-03 | | | | | dCs; InaGs; dAs; InaCs; |
| | m12 | | | | | dAs; InaGs; dTs; |
| | | | | | | InaCs; dTs; InaG-Sup |
| 535 | Apoa1_ | GACAGTCTGGGTG | Apoa1 | 5′ | mouse | InaGs; dAs; InaCs; |
| | mus-04 | | | | | dAs; InaGs; dTs; InaCs; |
| | m12 | | | | | dTs; InaGs; dGs; |
| | | | | | | InaGs; dTs; InaG-Sup |
| 536 | Apoa1_ | CAGTCTGGGTG | Apoa1 | 5′ | mouse | InaCs; dAs; InaGs; |
| | mus-05 | | | | | dTs; InaCs; dTs; InaGs; |
| | m12 | | | | | dGs; InaGs; dTs; |
| | | | | | | InaG-Sup |
| 537 | Apoa1_ | CTCAGCCTGGCCCTG | Apoa1 | 5′ | mouse | InaCs; dTs; InaCs; |
| | mus-06 | | | | | dAs; InaGs; dCs; InaCs; |
| | m12 | | | | | dTs; InaGs; dGs; |
| | | | | | | InaCs; dCs; InaCs; |
| | | | | | | dTs; InaG-Sup |
| 538 | Apoa1_ | AGTTCAAGGATCAGC | Apoa1 | 5′ | mouse | InaAs; dGs; InaTs; |
| | mus-07 | | | | | dTs; InaCs; dAs; InaAs; |
| | m12 | | | | | dGs; InaGs; dAs; |
| | | | | | | InaTs; dCs; InaAs; |
| | | | | | | dGs; InaC-Sup |
| 539 | Apoa1_ | GCTCTCCGACAGTCT | Apoa1 | 5′ | mouse | InaGs; dCs; InaTs; |
| | mus-08 | | | | | dCs; InaTs; dCs; InaCs; |
| | m12 | | | | | dGs; InaAs; dCs; |
| | | | | | | InaAs; dGs; InaTs; |
| | | | | | | dCs; InaT-Sup |
| 540 | Apoa1_ | TCTCCGACAGTCT | Apoa1 | 5′ | mouse | InaTs; dCs; InaTs; |
| | mus-09 | | | | | dCs; InaCs; dGs; InaAs; |
| | m12 | | | | | dCs; InaAs; dGs; |
| | | | | | | InaTs; dCs; InaT-Sup |
| 541 | Apoa1_ | TCCGACAGTCT | Apoa1 | 5′ | mouse | InaTs; dCs; InaCs; |
| | mus-10 | | | | | dGs; InaAs; dCs; InaAs; |
| | m12 | | | | | dGs; InaTs; dCs; |
| | | | | | | InaT-Sup |
| 542 | Apoa1_ | CGGAGCTCTCCGACA | Apoa1 | 5′ | mouse | InaCs; dGs; InaGs; |
| | mus-11 | | | | | dAs; InaGs; dCs; |
| | m12 | | | | | InaTs; dCs; InaTs; dCs; |
| | | | | | | InaCs; dGs; InaAs; |
| | | | | | | dCs; InaA-Sup |
| 543 | Apoa1_ | GAGCTCTCCGACA | Apoa1 | 5′ | mouse | InaGs; dAs; InaGs; |
| | mus-12 | | | | | dCs; InaTs; dCs; InaTs; |
| | m12 | | | | | dCs; InaCs; dGs; |
| | | | | | | InaAs; dCs; InaA-Sup |
| 544 | Apoa1_ | GCTCTCCGACA | Apoa1 | 5′ | mouse | InaGs; dCs; InaTs; |
| | mus-13 | | | | | dCs; InaTs; dCs; InaCs; |
| | m12 | | | | | dGs; InaAs; dCs; |
| | | | | | | InaA-Sup |
| 545 | Apoa1_ | CTATTCCATTTTGGA | Apoa1 | 3′ | mouse | InaCs; dTs; InaAs; |
| | mus-14 | | | | | dTs; InaTs; dCs; InaCs; |
| | m12 | | | | | dAs; InaTs; dTs; |
| | | | | | | InaTs; dTs; InaGs; |
| | | | | | | dGs; InaA-Sup |
| 546 | Apoa1_ | CTATTCCATTTTG | Apoa1 | 3′ | mouse | InaCs; dTs; InaAs; |
| | mus-15 | | | | | dTs; InaTs; dCs; InaCs; |
| | m12 | | | | | dAs; InaTs; dTs; |
| | | | | | | InaTs; dTs; InaG-Sup |
| 547 | Apoa1_ | ATTCCATTTTGGAAA | Apoa1 | 3′ | mouse | InaAs; dTs; InaTs; |
| | mus-16 | | | | | dCs; InaCs; dAs; InaTs; |
| | m12 | | | | | dTs; InaTs; dTs; |
| | | | | | | InaGs; dGs; InaAs; |
| | | | | | | dAs; InaA-Sup |
| 548 | Apoa1_ | CCATTTTGGAAAGGT | Apoa1 | 3′ | mouse | InaCs; dCs; InaAs; |
| | mus-17 | | | | | dTs; InaTs; dTs; InaTs; |
| | m12 | | | | | dGs; InaGs; dAs; |
| | | | | | | InaAs; dAs; InaGs; |
| | | | | | | dGs; InaT-Sup |
| 549 | Apoa1_ | CCATTTTGGAAAG | Apoa1 | 3′ | mouse | InaCs; dCs; InaAs; |
| | mus-18 | | | | | dTs; InaTs; dTs; InaTs; |
| | m12 | | | | | dGs; InaGs; dAs; |
| | | | | | | InaAs; dAs; InaG-Sup |
| 550 | Apoa1_ | CATTTTGGAAAGGTT | Apoa1 | 3′ | mouse | InaCs; dAs; InaTs; |
| | mus-19 | | | | | dTs; InaTs; dTs; InaGs; |
| | m12 | | | | | dGs; InaAs; dAs; |
| | | | | | | InaAs; dGs; InaGs; |
| | | | | | | dTs; InaT-Sup |
| 551 | Apoa1_ | CATTTTGGAAAGG | Apoa1 | 3′ | mouse | InaCs; dAs; InaTs; |
| | mus-20 | | | | | dTs; InaTs; dTs; InaGs; |
| | m12 | | | | | dGs; InaAs; dAs; |
| | | | | | | InaAs; dGs; InaG-Sup |
| 552 | Apoa1_ | GGAAAGGTTTATTGT | Apoa1 | 3′ | mouse | InaGs; dGs; InaAs; |
| | mus-21 | | | | | dAs; InaAs; dGs; |
| | m12 | | | | | InaGs; dTs; InaTs; |
| | | | | | | dTs; InaAs; dTs; InaTs; |
| | | | | | | dGs; InaT-Sup |
| | | | | | | InaTs; dCs; dCs; InaGs; |
| 553 | Apoa1_ | TCCGACAGTCTCCATT | Apoa1 | 5′ and 3′ | mouse | dAs; dCs; InaAs; |
| | mus-22 | TTGGAA | | | | dGs; dTs; InaCs; dTs; |
| | m22 | | | | | dCs; InaCs; dAs; |
| | | | | | | dTs; InaTs; dTs; dTs; |
| | | | | | | InaGs; dGs; dAs; InaA- |
| | | | | | | Sup |
| 554 | Apoa1_ | GCTCTCCGACACCATT | Apoa1 | 5′ and 3′ | mouse | InaGs; dCs; dTs; InaCs; |
| | mus-23 | TTGGAA | | | | dTs; dCs; InaCs; |
| | m22 | | | | | dGs; dAs; InaCs; |
| | | | | | | dAs; dCs; InaCs; dAs; |
| | | | | | | dTs; InaTs; dTs; dTs; |
| | | | | | | InaGs; dGs; dAs; InaA- |
| | | | | | | Sup |
| 555 | Apoa1_ | TCCGACAGTCTCATTT | Apoa1 | 5′ and 3′ | mouse | InaTs; dCs; dCs; InaGs; |
| | mus-24 | TGGAAA | | | | dAs; dCs; InaAs; |
| | m22 | | | | | dGs; dTs; InaCs; |
| | | | | | | dTs; dCs; InaAs; dTs; |
| | | | | | | dTs; InaTs; dTs; dGs; |
| | | | | | | InaGs; dAs; dAs; InaA- |
| | | | | | | Sup |
| 556 | Apoa1_ | GCTCTCCGACACATTT | Apoa1 | 5′ and 3′ | mouse | InaGs; dCs; dTs; InaCs; |
| | mus-25 | TGGAAA | | | | dTs; dCs; InaCs; |
| | m22 | | | | | dGs; dAs; InaCs; dAs; |
| | | | | | | dCs; InaAs; dTs; |
| | | | | | | dTs; InaTs; dTs; dGs; |
| | | | | | | InaGs; dAs; dAs; |
| | | | | | | InaA-Sup |
| 557 | FXN-761 | CCTCAAAAGCAGGAA | FXN | 3′ | human | InaCs; omeCs; InaTs; |
| | m01 | | | | | omeCs; InaAs; |
| | | | | | | omeAs; InaAs; omeAs; |
| | | | | | | InaGs; omeCs; |
| | | | | | | InaAs; omeGs; InaGs; |
| | | | | | | omeAs; InaA- |
| | | | | | | Sup |
| 558 | FXN-762 | CCTCAAAAGCAGG | FXN | 3′ | human | InaCs; omeCs; InaTs; |
| | m01 | | | | | omeCs; InaAs; |
| | | | | | | omeAs; InaAs; omeAs; |
| | | | | | | InaGs; omeCs; |
| | | | | | | InaAs; omeGs; InaG-Sup |
| 559 | FXN-763 | CCTCAAAAGCA | FXN | 3′ | human | InaCs; omeCs; InaTs; |
| | m01 | | | | | omeCs; InaAs; |
| | | | | | | omeAs; InaAs; omeAs; |
| | | | | | | InaGs; omeCs; InaA-Sup |
| 560 | FXN-764 | TCAAAAGCAGGAA | FXN | 3′ | human | InaTs; omeCs; InaAs; |
| | m01 | | | | | omeAs; InaAs; |
| | | | | | | omeAs; InaGs; omeCs; |
| | | | | | | InaAs; omeGs; |
| | | | | | | InaGs; omeAs; InaA-Sup |
| 561 | FXN-765 | CAAAAGCAGGA | FXN | 3′ | human | InaCs; omeAs; InaAs; |
| | m01 | | | | | omeAs; InaAs; |
| | | | | | | omeGs; InaCs; omeAs; |
| | | | | | | InaGs; omeGs; |
| | | | | | | InaA-Sup |
| 562 | FXN-766 | CCGCCCTCCAGCCTCA | FXN | 5′ and 3′ | human | InaCs; omeCs; InaGs; |
| | m01 | AAAGCAGGAAT | | | | omeCs; InaCs; |
| | | | | | | omeCs; InaTs; |
| | | | | | | omeCs; InaCs; omeAs; |
| | | | | | | InaGs; omeCs; |
| | | | | | | InaCs; omeTs; InaCs; |
| | | | | | | omeAs; InaAs; omeAs; |
| | | | | | | InaAs; omeGs; |
| | | | | | | InaCs; omeAs; InaGs; |
| | | | | | | omeGs; InaAs; |
| | | | | | | omeAs; InaT-Sup |
| 563 | FXN-767 | CCGCCCTCCAGCCTCA | FXN | 5′ and 3′ | human | InaCs; omeCs; InaGs; |
| | m01 | AAAGCAGGA | | | | omeCs; InaCs; |
| | | | | | | omeCs; InaTs; omeCs; |
| | | | | | | InaCs; omeAs; |
| | | | | | | InaGs; omeCs; InaCs; |
| | | | | | | omeTs; InaCs; |
| | | | | | | omeAs; InaAs; omeAs; |
| | | | | | | InaAs; omeGs; |
| | | | | | | InaCs; omeAs; InaGs; |
| | | | | | | omeGs; InaA-Sup |
| 564 | FXN-768 | CCGCCCTCCAGCCTCA | FXN | 5′ and 3′ | human | InaCs; omeCs; InaGs; |
| | m01 | AAAGCAG | | | | omeCs; InaCs; |
| | | | | | | omeCs; InaTs; omeCs; |
| | | | | | | InaCs; omeAs; |
| | | | | | | InaGs; omeCs; InaCs; |
| | | | | | | omeTs; InaCs; |
| | | | | | | omeAs; InaAs; omeAs; |
| | | | | | | InaAs; omeGs; |
| | | | | | | InaCs; omeAs; InaG-Sup |
| 565 | FXN-769 | CCGCCCTCCAGCCTCA | FXN | 5′ and 3′ | human | InaCs; omeCs; InaGs; |
| | m01 | AAAGC | | | | omeCs; InaCs; |
| | | | | | | omeCs; InaTs; omeCs; |
| | | | | | | InaCs; omeAs; |
| | | | | | | InaGs; omeCs; InaCs; |
| | | | | | | omeTs; InaCs; |
| | | | | | | omeAs; InaAs; omeAs; |
| | | | | | | InaAs; omeGs; |
| | | | | | | InaC-Sup |
| 566 | FXN-770 | GCCCTCCAGCCTCAAA | FXN | 5′ and 3′ | human | InaGs; omeCs; InaCs; |
| | m01 | AGCAGGAAT | | | | omeCs; InaTs; |
| | | | | | | omeCs; InaCs; omeAs; |
| | | | | | | InaGs; omeCs; |
| | | | | | | InaCs; omeTs; InaCs; |
| | | | | | | omeAs; InaAs; |
| | | | | | | omeAs; InaAs; omeGs; |
| | | | | | | InaCs; omeAs; |
| | | | | | | InaGs; omeGs; InaAs; |
| | | | | | | omeAs; InaT-Sup |
| 567 | FXN-771 | GCCCTCCAGCCTCAAA | FXN | 5′ and 3′ | human | InaGs; omeCs; InaCs; |
| | m01 | AGCAGGA | | | | omeCs; InaTs; |
| | | | | | | omeCs; InaCs; omeAs; |
| | | | | | | InaGs; omeCs; |
| | | | | | | InaCs; omeTs; InaCs; |
| | | | | | | omeAs; InaAs; |
| | | | | | | omeAs; InaAs; omeGs; |
| | | | | | | InaCs; omeAs; |
| | | | | | | InaGs; omeGs; InaA-Sup |
| 568 | FXN-772 | GCCCTCCAGCCTCAAA | FXN | 5′ and 3′ | human | InaGs; omeCs; InaCs; |
| | m01 | AGCAG | | | | omeCs; InaTs; |
| | | | | | | omeCs; InaCs; omeAs; |
| | | | | | | InaGs; omeCs; |
| | | | | | | InaCs; omeTs; InaCs; |
| | | | | | | omeAs; InaAs; |
| | | | | | | omeAs; InaAs; omeGs; |
| | | | | | | InaCs; omeAs; |
| | | | | | | InaG-Sup |
| 569 | FXN-773 | GCCCTCCAGCCTCAAA | FXN | 5′ and 3′ | human | InaGs; omeCs; InaCs; |
| | m01 | AGC | | | | omeCs; InaTs; |
| | | | | | | omeCs; InaCs; omeAs; |
| | | | | | | InaGs; omeCs; |
| | | | | | | InaCs; omeTs; InaCs; |
| | | | | | | omeAs; InaAs; |
| | | | | | | omeAs; InaAs; omeGs; |
| | | | | | | InaC-Sup |
| 570 | FXN-774 | CCCTCCAGCCTCAAAA | FXN | 5′ and 3′ | human | InaCs; omeCs; InaCs; |
| | m01 | G | | | | omeTs; InaCs; |
| | | | | | | omeCs; InaAs; omeGs; |
| | | | | | | InaCs; omeCs; |
| | | | | | | InaTs; omeCs; InaAs; |
| | | | | | | omeAs; InaAs; |
| | | | | | | omeAs; InaG-Sup |
| 571 | FXN-776 | CCTCCAGCCTCAAAA | FXN | 5′ and 3′ | human | InaCs; omeCs; InaTs; |
| | m01 | | | | | omeCs; InaCs; |
| | | | | | | omeAs; InaGs; omeCs; |
| | | | | | | InaCs; omeTs; |
| | | | | | | InaCs; omeAs; InaAs; |
| | | | | | | omeAs; InaA-Sup |
| 572 | FXN-777 | GCCCTCCAGTCAAAA | FXN | 5′ and 3′ | human | InaGs; omeCs; InaCs; |
| | m01 | GCAGGA | | | | omeCs; InaTs; |
| | | | | | | omeCs; InaCs; omeAs; |
| | | | | | | InaGs; omeTs; |
| | | | | | | InaCs; omeAs; InaAs; |
| | | | | | | omeAs; InaAs; |
| | | | | | | omeGs; InaCs; omeAs; |
| | | | | | | InaGs; omeGs; |
| | | | | | | InaA-Sup |
| 573 | FXN-778 | GCCCTCCAGCAAAAG | FXN | 5′ and 3′ | human | InaGs; omeCs; InaCs; |
| | m01 | CAGG | | | | omeCs; InaTs; |
| | | | | | | omeCs; InaCs; omeAs; |
| | | | | | | InaGs; omeCs; |
| | | | | | | InaAs; omeAs; InaAs; |
| | | | | | | omeAs; InaGs; |
| | | | | | | omeCs; InaAs; omeGs; |
| | | | | | | InaG-Sup |
| 574 | FXN-779 | CCGCCCTCCAGTCAAA | FXN | 5′ and 3′ | human | InaCs; omeCs; InaGs; |
| | m01 | AGCAGGA | | | | omeCs; InaCs; |
| | | | | | | omeCs; InaTs; omeCs; |
| | | | | | | InaCs; omeAs; |
| | | | | | | InaGs; omeTs; InaCs; |
| | | | | | | omeAs; InaAs; |
| | | | | | | omeAs; InaAs; omeGs; |
| | | | | | | InaCs; omeAs; |
| | | | | | | InaGs; omeGs; InaA-Sup |
| 575 | FXN-780 | CCGCCCTCCAGCAAA | FXN | 5′ and 3′ | human | InaCs; omeCs; InaGs; |
| | m01 | AGCAGG | | | | omeCs; InaCs; |
| | | | | | | omeCs; InaTs; omeCs; |
| | | | | | | InaCs; omeAs; |
| | | | | | | InaGs; omeCs; InaAs; |
| | | | | | | omeAs; InaAs; |
| | | | | | | omeAs; InaGs; omeCs; |
| | | | | | | InaAs; omeGs; |
| | | | | | | InaG-Sup |
| 576 | FXN-671 | CTCCGCCCTCCAG | FXN | 5′ | human | InaCs; omeTs; InaCs; |
| | m01 | | | | | omeCs; InaGs; |
| | | | | | | omeCs; InaCs; omeCs; |
| | | | | | | InaTs; omeCs; |
| | | | | | | InaCs; omeAs; InaG-Sup |
| 577 | FXN-672 | CCGCCCTCCAG | FXN | 5′ | human | InaCs; omeCs; InaGs; |
| | m01 | | | | | omeCs; InaCs; |
| | | | | | | omeCs; InaTs; |
| | | | | | | omeCs; InaCs; omeAs; |
| | | | | | | InaG-Sup |
| 578 | FXN-673 | GCCCTCCAG | FXN | 5′ | human | InaGs; omeCs; InaCs; |
| | m01 | | | | | omeCs; InaTs; |
| | | | | | | omeCs; InaCs; omeAs; |
| | | | | | | InaG-Sup |
| 579 | FXN-674 | CCCGCTCCGCCCTCC | FXN | 5′ | human | InaCs; omeCs; InaCs; |
| | m01 | | | | | omeGs; InaCs; |
| | | | | | | omeTs; InaCs; omeCs; |
| | | | | | | InaGs; omeCs; |
| | | | | | | InaCs; omeCs; InaTs; |
| | | | | | | omeCs; InaC-Sup |
| 580 | FXN-675 | CGCTCCGCCCTCC | FXN | 5′ | human | InaCs; omeGs; InaCs; |
| | m01 | | | | | omeTs; InaCs; |
| | | | | | | omeCs; InaGs; omeCs; |
| | | | | | | InaCs; omeCs; |
| | | | | | | InaTs; omeCs; InaC-Sup |
| 581 | FXN-676 | CTCCGCCCTCC | FXN | 5′ | human | InaCs; omeTs; InaCs; |
| | m01 | | | | | omeCs; InaGs; |
| | | | | | | omeCs; InaCs; |
| | | | | | | omeCs; InaTs; omeCs; |
| | | | | | | InaC-Sup |
| 582 | FXN-677 | CCGCCCTCC | FXN | 5′ | human | InaCs; omeCs; InaGs; |
| | m01 | | | | | omeCs; InaCs; |
| | | | | | | omeCs; InaTs; |
| | | | | | | omeCs; InaC-Sup |
| 583 | CD247- | GCCTTTGAGAAAGCA | CD247 | 5′ | human | dGs; InaCs; dCs; InaTs; |
| | 90 m02 | | | | | dTs; InaTs; dGs; |
| | | | | | | InaAs; dGs; InaAs; |
| | | | | | | dAs; InaAs; dGs; |
| | | | | | | InaCs; dA-Sup |
| 584 | CD247- | GACTGTGGGGCCTTT | CD247 | 5′ | human | dGs; InaAs; dCs; |
| | 91 m02 | | | | | InaTs; dGs; InaTs; |
| | | | | | | dGs; InaGs; dGs; InaGs; |
| | | | | | | dCs; InaCs; dTs; |
| | | | | | | InaTs; dT-Sup |
| 585 | CD247- | AGGAAGTGGAGGACT | CD247 | 5′ | human | dAs; InaGs; dGs; InaAs; |
| | 92 m02 | | | | | dAs; InaGs; dTs; |
| | | | | | | InaGs; dGs; InaAs; |
| | | | | | | dGs; InaGs; dAs; |
| | | | | | | InaCs; dT-Sup |
| 586 | CD247- | TGCATTTTCACTGAA | CD247 | 3′ | human | dTs; InaGs; dCs; InaAs; |
| | 93 m02 | | | | | dTs; InaTs; dTs; |
| | | | | | | InaTs; dCs; InaAs; |
| | | | | | | dCs; InaTs; dGs; InaAs; |
| | | | | | | dA-Sup |
| 587 | CD247- | CATTTTCACTGAAGC | CD247 | 3′ | human | dCs; InaAs; dTs; InaTs; |
| | 94 m02 | | | | | dTs; InaTs; dCs; |
| | | | | | | InaAs; dCs; InaTs; |
| | | | | | | dGs; InaAs; dAs; |
| | | | | | | InaGs; dC-Sup |
| 588 | CD247- | ACTGAAGCATTTATT | CD247 | 3′ | human | dAs; InaCs; dTs; InaGs; |
| | 95 m02 | | | | | dAs; InaAs; dGs; |
| | | | | | | InaCs; dAs; InaTs; |
| | | | | | | dTs; InaTs; dAs; |
| | | | | | | InaTs; dT-Sup |
| 589 | CFTR-84 | CACACAAATGTATGG | CFTR | 3′ | human | dCs; InaAs; dCs; InaAs; |
| | m02 | | | | | dCs; InaAs; dAs; |
| | | | | | | InaAs; dTs; InaGs; |
| | | | | | | dTs; InaAs; dTs; |
| | | | | | | InaGs; dG-Sup |
| 590 | CFTR-85 | GGATTTTATTGACAA | CFTR | 3′ | human | dGs; InaGs; dAs; InaTs; |
| | m02 | | | | | dTs; InaTs; dTs; |
| | | | | | | InaAs; dTs; InaTs; |
| | | | | | | dGs; InaAs; dCs; |
| | | | | | | InaAs; dA-Sup |
| 591 | CFTR-86 | AAAACAACAAAGTTT | CFTR | 3′ | human | dAs; InaAs; dAs; InaAs; |
| | m02 | | | | | dCs; InaAs; dAs; |
| | | | | | | InaCs; dAs; InaAs; |
| | | | | | | dAs; InaGs; dTs; |
| | | | | | | InaTs; dT-Sup |
| 592 | CFTR-87 | AGTGCCATAAAAAGT | CFTR | 3′ | human | dAs; InaGs; dTs; InaGs; |
| | m02 | | | | | dCs; InaCs; dAs; |
| | | | | | | InaTs; dAs; InaAs; |
| | | | | | | dAs; InaAs; dAs; |
| | | | | | | InaGs; dT-Sup |
| 593 | CFTR-88 | TCAAATATAAAAATT | CFTR | 3′ | human | dTs; InaCs; dAs; InaAs; |
| | m02 | | | | | dAs; InaTs; dAs; |
| | | | | | | InaTs; dAs; InaAs; |
| | | | | | | dAs; InaAs; dAs; |
| | | | | | | InaTs; dT-Sup |
| 594 | CFTR-89 | TTCCCCCCACCCACC | CFTR | 3′ | human | dTs; InaTs; dCs; InaCs; |
| | m02 | | | | | dCs; InaCs; dCs; |
| | | | | | | InaCs; dAs; InaCs; |
| | | | | | | dCs; InaCs; dAs; |
| | | | | | | InaCs; dC-Sup |
| 595 | CFTR-90 | CATTTGCTTCCAATT | CFTR | 5′ | human | dCs; InaAs; dTs; InaTs; |
| | m02 | | | | | dTs; InaGs; dCs; |
| | | | | | | InaTs; dTs; InaCs; |
| | | | | | | dCs; InaAs; dAs; |
| | | | | | | InaTs; dT-Sup |
| 596 | CFTR-91 | GCTCAACCCTTTTTC | CFTR | 5′ | human | dGs; InaCs; dTs; InaCs; |
| | m02 | | | | | dAs; InaAs; dCs; |
| | | | | | | InaCs; dCs; InaTs; |
| | | | | | | dTs; InaTs; dTs; |
| | | | | | | InaTs; dC-Sup |
| 597 | CFTR-92 | AGACCTACTACTCTG | CFTR | 5′ | human | dAs; InaGs; dAs; InaCs; |
| | m02 | | | | | dCs; InaTs; dAs; |
| | | | | | | InaCs; dTs; InaAs; |
| | | | | | | dCs; InaTs; dCs; |
| | | | | | | InaTs; dG-Sup |
| 598 | FMR1- | CCCTCCACCGGAAGT | FMR1 | 5′ | human | dCs; InaCs; dCs; InaTs; |
| | 58 m02 | | | | | dCs; InaCs; dAs; |
| | | | | | | InaCs; dCs; InaGs; |
| | | | | | | dGs; InaAs; dAs; |
| | | | | | | InaGs; dT-Sup |
| 599 | FMR1- | GCCCGCGCTCGCCGT | FMR1 | 5′ | human | dGs; InaCs; dCs; InaCs; |
| | 59 m02 | | | | | dGs; InaCs; dGs; |
| | | | | | | InaCs; dTs; InaCs; |
| | | | | | | dGs; InaCs; dCs; |
| | | | | | | InaGs; dT-Sup |
| 600 | FMR1- | ACGCCCCCTGGCAGC | FMR1 | 5′ | human | dAs; InaCs; dGs; InaCs; |
| | 60 m02 | | | | | dCs; InaCs; dCs; |
| | | | | | | InaCs; dTs; InaGs; |
| | | | | | | dGs; InaCs; dAs; |
| | | | | | | InaGs; dC-Sup |
| 601 | FMR1- | GCTCAGCCCCTCGGC | FMR1 | 5′ | human | dGs; InaCs; dTs; InaCs; |
| | 61 m02 | | | | | dAs; InaGs; dCs; |
| | | | | | | InaCs; dCs; InaCs; dTs; |
| | | | | | | InaCs; dGs; InaGs; |
| | | | | | | dC-Sup |
| 602 | FMR1- | AGCAGAGGAAGATCA | FMR1 | 3′ | human | dAs; InaGs; dCs; InaAs; |
| | 62 m02 | | | | | dGs; InaAs; dGs; |
| | | | | | | InaGs; dAs; InaAs; |
| | | | | | | dGs; InaAs; dTs; |
| | | | | | | InaCs; dA-Sup |
| 603 | FMR1- | CAGAGGAAGATCAAA | FMR1 | 3′ | human | dCs; InaAs; dGs; InaAs; |
| | 63 m02 | | | | | dGs; InaGs; dAs; |
| | | | | | | InaAs; dGs; InaAs; |
| | | | | | | dTs; InaCs; dAs; |
| | | | | | | InaAs; dA-Sup |
| 604 | FMR1- | CAGATTTTTGAAACT | FMR1 | 3′ | human | dCs; InaAs; dGs; InaAs; |
| | 64 m02 | | | | | dTs; InaTs; dTs; |
| | | | | | | InaTs; dTs; InaGs; |
| | | | | | | dAs; InaAs; dAs; |
| | | | | | | InaCs; dT-Sup |
| 605 | FMR1- | CAGACTAATTTTTTG | FMR1 | 3′ | human | dCs; InaAs; dGs; InaAs; |
| | 65 m02 | | | | | dCs; InaTs; dAs; |
| | | | | | | InaAs; dTs; InaTs; |
| | | | | | | dTs; InaTs; dTs; |
| | | | | | | InaTs; dG-Sup |
| 606 | FMR1- | TTTTTGCTTTTTCAT | FMR1 | 3′ | human | dTs; InaTs; dTs; InaTs; |
| | 66 m02 | | | | | dTs; InaGs; dCs; |
| | | | | | | InaTs; dTs; InaTs; |
| | | | | | | dTs; InaTs; dCs; InaAs; |
| | | | | | | dT-Sup |
| 607 | FMR1- | AATTTTTTGCTTTTT | FMR1 | 3′ | human | dAs; InaAs; dTs; InaTs; |
| | 67 m02 | | | | | dTs; InaTs; dTs; |
| | | | | | | InaTs; dGs; InaCs; |
| | | | | | | dTs; InaTs; dTs; InaTs; |
| | | | | | | dT-Sup |
| 608 | FMR1- | ATGTTTGGCAATACT | FMR1 | 3′ | human | dAs; InaTs; dGs; InaTs; |
| | 68 m02 | | | | | dTs; InaTs; dGs; |
| | | | | | | InaGs; dCs; InaAs; |
| | | | | | | dAs; InaTs; dAs; |
| | | | | | | InaCs; dT-Sup |
| 609 | FMR1- | TTGGCAATACTTTTT | FMR1 | 3′ | human | dTs; InaTs; dGs; InaGs; |
| | 69 m02 | | | | | dCs; InaAs; dAs; |
| | | | | | | InaTs; dAs; InaCs; |
| | | | | | | dTs; InaTs; dTs; InaTs; |
| | | | | | | dT-Sup |
| 610 | LAMA1- | GCTGCCCTGGCCCCG | LAMA1 | 5′ | human | dGs; InaCs; dTs; InaGs; |
| | 105 | | | | | dCs; InaCs; dCs; |
| | m02 | | | | | InaTs; dGs; InaGs; |
| | | | | | | dCs; InaCs; dCs; InaCs; |
| | | | | | | dG-Sup |
| 611 | LAMA1- | CGGACACACCCCTCG | LAMA1 | 5′ | human | dCs; InaGs; dGs; InaAs; |
| | 106 | | | | | dCs; InaAs; dCs; |
| | m02 | | | | | InaAs; dCs; InaCs; |
| | | | | | | dCs; InaCs; dTs; |
| | | | | | | InaCs; dG-Sup |
| 612 | LAMA1- | ACGGGACGCGAGTCC | LAMA1 | 5′ | human | dAs; InaCs; dGs; InaGs; |
| | 107 | | | | | dGs; InaAs; dCs; |
| | m02 | | | | | InaGs; dCs; InaGs; |
| | | | | | | dAs; InaGs; dTs; |
| | | | | | | InaCs; dC-Sup |
| 613 | LAMA1- | GTCTGGGGAGAAAGC | LAMA1 | 5′ | human | dGs; InaTs; dCs; InaTs; |
| | 108 | | | | | dGs; InaGs; dGs; |
| | m02 | | | | | InaGs; dAs; InaGs; |
| | | | | | | dAs; InaAs; dAs; |
| | | | | | | InaGs; dC-Sup |
| 614 | LAMA1- | CCACTCGGTGGGTCT | LAMA1 | 5′ | human | dCs; InaCs; dAs; InaCs; |
| | 109 | | | | | dTs; InaCs; dGs; |
| | m02 | | | | | InaGs; dTs; InaGs; |
| | | | | | | dGs; InaGs; dTs; |
| | | | | | | InaCs; dT-Sup |
| 615 | LAMA1- | TGATCTGTTATCATC | LAMA1 | 5′ | human | dTs; InaGs; dAs; InaTs; |
| | 110 | | | | | dCs; InaTs; dGs; |
| | m02 | | | | | InaTs; dTs; InaAs; |
| | | | | | | dTs; InaCs; dAs; InaTs; |
| | | | | | | dC-Sup |
| 616 | LAMA1- | CTGTTATCATCTGTA | LAMA1 | 3′ | human | dCs; InaTs; dGs; InaTs; |
| | 111 | | | | | dTs; InaAs; dTs; |
| | m02 | | | | | nIaCs; dAs; InaTs; |
| | | | | | | dCs; InaTs; dGs; InaTs; |
| | | | | | | dA-Sup |
| 617 | LAMA1- | GTGTATAAAGATTTT | LAMA1 | 3′ | human | dGs; InaTs; dGs; InaTs; |
| | 112 | | | | | dAs; InaTs; dAs; |
| | m02 | | | | | InaAs; dAs; InaGs; |
| | | | | | | dAs; InaTs; dTs; |
| | | | | | | InaTs; dT-Sup |
| 618 | LAMA1- | CAATTTACATTTTAG | LAMA1 | 3′ | human | dCs; InaAs; dAs; InaTs; |
| | 113 | | | | | dTs; InaTs; dAs; |
| | m02 | | | | | InaCs; dAs; InaTs; |
| | | | | | | dTs; InaTs; dTs; InaAs; |
| | | | | | | dG-Sup |
| 619 | LAMA1- | TACATTTTAGACCAT | LAMA1 | 3′ | human | dTs; InaAs; dCs; InaAs; |
| | 114 | | | | | dTs; InaTs; dTs; |
| | m02 | | | | | InaTs; dAs; InaGs; |
| | | | | | | dAs; InaCs; dCs; InaAs; |
| | | | | | | dT-Sup |
| 620 | MBNL1- | TGCTATAAGATGTAA | MBNL1 | 5′ | human | dTs; InaGs; dCs; InaTs; |
| | 73 m02 | | | | | dAs; InaTs; dAs; |
| | | | | | | InaAs; dGs; InaAs; |
| | | | | | | dTs; InaGs; dTs; |
| | | | | | | InaAs; dA-Sup |
| 621 | MBNL1- | AAGGAAGCCGGCAA | MBNL1 | 5′ | human | dAs; InaAs; dGs; InaGs; |
| | 74 m02 | G | | | | dAs; InaAs; dGs; |
| | | | | | | InaCs; dCs; InaGs; |
| | | | | | | dGs; InaCs; dAs; |
| | | | | | | InaAs; dG-Sup |
| 622 | MBNL1- | CGCCACAACTCATTC | MBNL1 | 5′ | human | dCs; InaGs; dCs; InaCs; |
| | 75 m02 | | | | | dAs; InaCs; dAs; |
| | | | | | | InaAs; dCs; InaTs; |
| | | | | | | dCs; InaAs; dTs; InaTs; |
| | | | | | | dC-Sup |
| 623 | MBNL1- | ATGGGAGCATTGTGG | MBNL1 | 5′ | human | dAs; InaTs; dGs; InaGs; |
| | 76 m02 | | | | | dGs; InaAs; dGs; |
| | | | | | | InaCs; dAs; InaTs; |
| | | | | | | dTs; InaGs; dTs; InaGs; |
| | | | | | | dG-Sup |
| 624 | MBNL1- | CGCCCGCCCAGCCCC | MBNL1 | 5′ | human | dCs; InaGs; dCs; InaCs; |
| | 77 m02 | | | | | dCs; InaGs; dCs; |
| | | | | | | InaCs; dCs; InaAs; |
| | | | | | | dGs; InaCs; dCs; InaCs; |
| | | | | | | dC-Sup |
| 625 | MBNL1- | CCCCTCCCCCGCCCG | MBNL1 | 5′ | human | dCs; InaCs; dCs; InaCs; |
| | 78 m02 | | | | | dTs; InaCs; dCs; |
| | | | | | | InaCs; dCs; InaCs; |
| | | | | | | dGs; InaCs; dCs; InaCs; |
| | | | | | | dG-Sup |
| 626 | MBNL1- | CTTCCGCTGCTGCTG | MBNL1 | 5′ | human | dCs; InaTs; dTs; InaCs; |
| | 79 m02 | | | | | dCs; InaGs; dCs; |
| | | | | | | InaTs; dGs; InaCs; |
| | | | | | | dTs; InaGs; dCs; InaTs; |
| | | | | | | dG-Sup |
| 627 | MBNL1- | CTTCTTAGTACCAAC | MBNL1 | 5′ | human | dCs; InaTs; dTs; InaCs; |
| | 80 m02 | | | | | dTs; InaTs; dAs; |
| | | | | | | InaGs; dTs; InaAs; |
| | | | | | | dCs; InaCs; dAs; InaAs; |
| | | | | | | dC-Sup |
| 628 | MBNL1- | TTTAGAGCAAAATCG | MBNL1 | 5′ | human | dTs; InaTs; dTs; InaAs; |
| | 81 m02 | | | | | dGs; InaAs; dGs; |
| | | | | | | InaCs; dAs; InaAs; |
| | | | | | | dAs; InaAs; dTs; InaCs; |
| | | | | | | dG-Sup |
| 629 | MBNL1- | GGTAGTTAAATGTTT | MBNL1 | 5′ | human | dGs; InaGs; dTs; InaAs; |
| | 82 m02 | | | | | dGs; InaTs; dTs; |
| | | | | | | InaAs; dAs; InaAs; |
| | | | | | | dTs; InaGs; dTs; InaTs; |
| | | | | | | dT-Sup |
| 630 | MBNL1- | TACTTAAGAAAGAGA | MBNL1 | 3′ | human | dTs; InaAs; dCs; InaTs; |
| | 83 m02 | | | | | dTs; InaAs; dAs; |
| | | | | | | InaGs; dAs; InaAs; |
| | | | | | | dAs; InaGs; dAs; InaGs; |
| | | | | | | dA-Sup |
| 631 | MBNL1- | TATACTTAAGAAAGA | MBNL1 | 3′ | human | dTs; InaAs; dTs; InaAs; |
| | 84 m02 | | | | | dCs; InaTs; dTs; |
| | | | | | | InaAs; dAs; InaGs; |
| | | | | | | dAs; InaAs; dAs; InaGs; |
| | | | | | | dA-Sup |
| 632 | MECP2- | CGCCGCCGACGCCGG | MECP2 | 5′ | human | dCs; InaGs; dCs; InaCs; |
| | 61 m02 | | | | | dGs; InaCs; dCs; |
| | | | | | | InaGs; dAs; InaCs; |
| | | | | | | dGs; InaCs; dCs; InaGs; |
| | | | | | | dG-Sup |
| 633 | MECP2- | CTCTCTCCGAGAGGA | MECP2 | 5′ | human | dCs; InaTs; dCs; InaTs; |
| | 62 m02 | | | | | dCs; InaTs; dCs; |
| | | | | | | InaCs; dGs; InaAs; |
| | | | | | | dGs; InaAs; dGs; InaGs; |
| | | | | | | dA-Sup |
| 634 | MECP2- | CGCCCCGCCCTCTTG | MECP2 | 5′ | human | dCs; InaGs; dCs; InaCs; |
| | 63 m02 | | | | | dCs; InaCs; dGs; |
| | | | | | | InaCs; dCs; InaCs; |
| | | | | | | dTs; InaCs; dTs; InaTs; |
| | | | | | | dG-Sup |
| 635 | MECP2- | CCGCGCGCTGCTGCA | MECP2 | 5′ | human | dCs; InaCs; dGs; InaCs; |
| | 64 m02 | | | | | dGs; InaCs; dGs; |
| | | | | | | InaCs; dTs; InaGs; |
| | | | | | | dCs; InaTs; dGs; InaCs; |
| | | | | | | dA-Sup |
| 636 | MECP2- | CACTTTCACAGAGAG | MECP2 | 3′ | human | dCs; InaAs; dCs; InaTs; |
| | 65 m02 | | | | | dTs; InaTs; dCs; |
| | | | | | | InaAs; dCs; InaAs; |
| | | | | | | dGs; InaAs; dGs; InaAs; |
| | | | | | | dG-Sup |
| 637 | MECP2- | CTTTCACATGTATTAA | MECP2 | 3′ | human | dCs; InaTs; dTs; InaTs; |
| | 66 m02 | | | | | dCs; InaAs; dCs; |
| | | | | | | InaAs; dTs; InaGs; |
| | | | | | | dTs; InaAs; dTs; InaTs; |
| | | | | | | dAs; dA-Sup |
| 638 | MECP2- | ATGTATTAAAAAACT | MECP2 | 3′ | human | dAs; InaTs; dGs; InaTs; |
| | 67 m02 | | | | | dAs; InaTs; dTs; |
| | | | | | | InaAs; dAs; InaAs; |
| | | | | | | dAs; InaAs; dAs; InaCs; |
| | | | | | | dT-Sup |
| 639 | MECP2- | GACATTTTTATGTAA | MECP2 | 3′ | human | dGs; InaAs; dCs; InaAs; |
| | 68 m02 | | | | | dTs; InaTs; dTs; |
| | | | | | | InaTs; dTs; InaAs; |
| | | | | | | dTs; InaGs; dTs; |
| | | | | | | InaAs; dA-Sup |
| 640 | MECP2- | CATTTTTATGTAAAT | MECP2 | 3′ | human | dCs; InaAs; dTs; InaTs; |
| | 69 m02 | | | | | dTs; InaTs; dTs; |
| | | | | | | InaAs; dTs; InaGs; |
| | | | | | | dTs; InaAs; dAs; InaAs; |
| | | | | | | dT-Sup |
| 641 | MECP2- | AAATTTATAAGGCAA | MECP2 | 3′ | human | dAs; InaAs; dAs; InaTs; |
| | 70 m02 | | | | | dTs; InaTs; dAs; |
| | | | | | | InaTs; dAs; InaAs; |
| | | | | | | dGs; InaGs; dCs; |
| | | | | | | InaAs; dA-Sup |
| 642 | MECP2- | AGGCAAACTCTTTAT | MECP2 | 3′ | human | dAs; InaGs; dGs; InaCs; |
| | 71 m02 | | | | | dAs; InaAs; dAs; |
| | | | | | | InaCs; dTs; InaCs; |
| | | | | | | dTs; InaTs; dTs; |
| | | | | | | InaAs; dT-Sup |
| 643 | MECP2- | GTCTCTGGAACAATT | MECP2 | 3′ | human | dGs; InaTs; dCs; InaTs; |
| | 72 m02 | | | | | dCs; InaTs; dGs; |
| | | | | | | InaGs; dAs; InaAs; |
| | | | | | | dCs; InaAs; dAs; |
| | | | | | | InaTs; dT-Sup |
| 644 | MECP2- | CAGTTCAAACACAGA | MECP2 | 3′ | human | dCs; InaAs; dGs; InaTs; |
| | 73 m02 | | | | | dTs; InaCs; dAs; |
| | | | | | | InaAs; dAs; InaCs; |
| | | | | | | dAs; InaCs; dAs; InaGs; |
| | | | | | | dA-Sup |
| 645 | MECP2- | CAAACACAGAAGAGA | MECP2 | 3′ | human | dCs; InaAs; dAs; InaAs; |
| | 74 m02 | | | | | dCs; InaAs; dCs; |
| | | | | | | InaAs; dGs; InaAs; |
| | | | | | | dAs; InaGs; dAs; |
| | | | | | | InaGs; dA-Sup |
| 646 | MECP2- | AACACAGAAGAGATT | MECP2 | 3′ | human | dAs; InaAs; dCs; InaAs; |
| | 75 m02 | | | | | dCs; InaAs; dGs; |
| | | | | | | InaAs; dAs; InaGs; |
| | | | | | | dAs; InaGs; dAs; |
| | | | | | | InaTs; dT-Sup |
| 647 | MECP2- | GGGGGAGAAGAAAG | MECP2 | 3′ | human | dGs; InaGs; dGs; InaGs; |
| | 76 m02 | G | | | | dGs; InaAs; dGs; |
| | | | | | | InaAs; dAs; InaGs; |
| | | | | | | dAs; InaAs; dAs; |
| | | | | | | InaGs; dG-Sup |
| 648 | MECP2- | TCGTTTTTTTTTCTT | MECP2 | 3′ | human | dTs; InaCs; dGs; InaTs; |
| | 77 m02 | | | | | dTs; InaTs; dTs; |
| | | | | | | InaTs; dTs; InaTs; |
| | | | | | | dTs; InaTs; dCs; InaTs; |
| | | | | | | dT-Sup |
| 649 | MECP2- | CTTTTTTTTCTTTTT | MECP2 | 3′ | human | dCs; InaTs; dTs; InaTs; |
| | 78 m02 | | | | | dTs; InaTs; dTs; |
| | | | | | | InaTs; dTs; InaCs; |
| | | | | | | dTs; InaTs; dTs; InaTs; |
| | | | | | | dT-Sup |
| 650 | MECP2- | CCTATGCTATGGTTA | MECP2 | 3′ | human | dCs; InaCs; dTs; InaAs; |
| | 79 m02 | | | | | dTs; InaGs; dCs; |
| | | | | | | InaTs; dAs; InaTs; |
| | | | | | | dGs; InaGs; dTs; |
| | | | | | | InaTs; dA-Sup |
| 651 | MECP2- | AGTTTACTGAAAGAA | MECP2 | 3′ | human | dAs; InaGs; dTs; InaTs; |
| | 80 m02 | | | | | dTs; InaAs; dCs; |
| | | | | | | InaTs; dGs; InaAs; |
| | | | | | | dAs; InaAs; dGs; |
| | | | | | | InaAs; dA-Sup |
| 652 | MECP2- | ACTGAAAGAAAAAAA | MECP2 | 3′ | human | dAs; InaCs; dTs; InaGs; |
| | 81 m02 | | | | | dAs; InaAs; dAs; |
| | | | | | | InaGs; dAs; InaAs; |
| | | | | | | dAs; InaAs; dAs; |
| | | | | | | InaAs; dA-Sup |
| 653 | MERTK- | CCTTATTCATATTTT | MERTK | 3′ | human | dCs; InaCs; dTs; InaTs; |
| | 66 m02 | | | | | dAs; InaTs; dTs; |
| | | | | | | InaCs; dAs; InaTs; |
| | | | | | | dAs; InaTs; dTs; |
| | | | | | | InaTs; dT-Sup |
| 654 | MERTK- | CTTCCTTATTCATAT | MERTK | 3′ | human | dCs; InaTs; dTs; InaCs; |
| | 67 m02 | | | | | dCs; InaTs; dTs; |
| | | | | | | InaAs; dTs; InaTs; |
| | | | | | | dCs; InaAs; dTs; InaAs; |
| | | | | | | dT-Sup |
| 655 | MERTK- | CAATCCTTCAATATT | MERTK | 3′ | human | dCs; InaAs; dAs; InaTs; |
| | 68 m02 | | | | | dCs; InaCs; dTs; |
| | | | | | | InaTs; dCs; InaAs; |
| | | | | | | dAs; InaTs; dAs; |
| | | | | | | InaTs; dT-Sup |
| 656 | MERTK- | GGCATTTCATTTTAC | MERTK | 3′ | human | dGs; InaGs; dCs; InaAs; |
| | 69 m02 | | | | | dTs; InaTs; dTs; |
| | | | | | | InaCs; dAs; InaTs; |
| | | | | | | dTs; InaTs; dTs; |
| | | | | | | InaAs; dC-Sup |
| 657 | MERTK- | CATTTTACAAATATT | MERTK | 3′ | human | dCs; InaAs; dTs; InaTs; |
| | 70 m02 | | | | | dTs; InaTs; dAs; |
| | | | | | | InaCs; dAs; InaAs; |
| | | | | | | dAs; InaTs; dAs; |
| | | | | | | InaTs; dT-Sup |
| 658 | MERTK- | GAAATGAAATAAGTA | MERTK | 3′ | human | dGs; InaAs; dAs; InaAs; |
| | 71 m02 | | | | | dTs; InaGs; dAs; |
| | | | | | | InaAs; dAs; InaTs; |
| | | | | | | dAs; InaAs; dGs; |
| | | | | | | InaTs; dA-Sup |
| 659 | MERTK- | AGATATGCAAGATAA | MERTK | 3′ | human | dAs; InaGs; dAs; InaTs; |
| | 72 m02 | | | | | dAs; InaTs; dGs; |
| | | | | | | InaCs; dAs; InaAs; |
| | | | | | | dGs; InaAs; dTs; |
| | | | | | | InaAs; dA-Sup |
| 660 | MERTK- | GCGGGCCCAGCAGGT | MERTK | 5′ | human | dGs; InaCs; dGs; InaGs; |
| | 73 m02 | | | | | dGs; InaCs; dCs; |
| | | | | | | InaCs; dAs; InaGs; |
| | | | | | | dCs; InaAs; dGs; |
| | | | | | | InaGs; dT-Sup |
| 661 | MERTK- | CAGTGAGTGCCGAGT | MERTK | 5′ | human | dCs; InaAs; dGs; InaTs; |
| | 74 m02 | | | | | dGs; InaAs; dGs; |
| | | | | | | InaTs; dGs; InaCs; |
| | | | | | | dCs; InaGs; dAs; |
| | | | | | | InaGs; dT-Sup |
| 662 | MERTK- | GCCCGGGCAGTGAGT | MERTK | 5′ | human | dGs; InaCs; dCs; InaCs; |
| | 75 m02 | | | | | dGs; InaGs; dGs; |
| | | | | | | InaCs; dAs; InaGs; |
| | | | | | | dTs; InaGs; dAs; |
| | | | | | | InaGs; dT-Sup |
| 663 | MERTK- | TGTCCGGGCGGCCCG | MERTK | 5′ | human | dTs; InaGs; dTs; InaCs; |
| | 76 m02 | | | | | dCs; InaGs; dGs; |
| | | | | | | InaGs; dCs; InaGs; |
| | | | | | | dGs; InaCs; dCs; |
| | | | | | | InaCs; dG-Sup |
| 664 | SSPN-47 | CGCGCGTGTGCGAGT | SSPN | 5′ | human | dCs; InaGs; dCs; InaGs; |
| | m02 | | | | | dCs; InaGs; dTs; |
| | | | | | | InaGs; dTs; InaGs; |
| | | | | | | dCs; InaGs; dAs; |
| | | | | | | InaGs; dT-Sup |
| 665 | SSPN-48 | CTTCAGACAGGCTGC | SSPN | 5′ | human | dCs; InaTs; dTs; InaCs; |
| | m02 | | | | | dAs; InaGs; dAs; |
| | | | | | | InaCs; dAs; InaGs; |
| | | | | | | dGs; InaCs; dTs; |
| | | | | | | InaGs; dC-Sup |
| 666 | SSPN-49 | ACCTCTGCACTTCAG | SSPN | 5′ | human | dAs; InaCs; dCs; InaTs; |
| | m02 | | | | | dCs; InaTs; dGs; |
| | | | | | | InaCs; dAs; InaCs; |
| | | | | | | dTs; InaTs; dCs; |
| | | | | | | InaAs; dG-Sup |
| 667 | SSPN-50 | CGGCGCGGGTCCCTT | SSPN | 5′ | human | dCs; InaGs; dGs; InaCs; |
| | m02 | | | | | dGs; InaCs; dGs; |
| | | | | | | InaGs; dGs; InaTs; |
| | | | | | | dCs; InaCs; dCs; |
| | | | | | | InaTs; dT-Sup |
| 668 | SSPN-51 | TGGTATTCGAATTAT | SSPN | 5′ | human | dTs; InaGs; dGs; InaTs; |
| | m02 | | | | | dAs; InaTs; dTs; |
| | | | | | | InaCs; dGs; InaAs; |
| | | | | | | dAs; InaTs; dTs; |
| | | | | | | InaAs; dT-Sup |
| 669 | SSPN-52 | CGGCCTGCCCTGGTA | SSPN | 5′ | human | dCs; InaGs; dGs; InaCs; |
| | m02 | | | | | dCs; InaTs; dGs; |
| | | | | | | InaCs; dCs; InaCs; |
| | | | | | | dTs; InaGs; dGs; |
| | | | | | | InaTs; dA-Sup |
| 670 | SSPN-53 | TCAGAGATTATGAAA | SSPN | 3′ | human | dTs; InaCs; dAs; InaGs; |
| | m02 | | | | | dAs; InaGs; dAs; |
| | | | | | | InaTs; dTs; InaAs; |
| | | | | | | dTs; InaGs; dAs; |
| | | | | | | InaAs; dA-Sup |
| 671 | SSPN-54 | TGTTTTCAGAGATTA | SSPN | 3′ | human | dTs; InaGs; dTs; InaTs; |
| | m02 | | | | | dTs; InaTs; dCs; |
| | | | | | | InaAs; dGs; InaAs; |
| | | | | | | dGs; InaAs; dTs; |
| | | | | | | InaTs; dA-Sup |
| 672 | SSPN-55 | CATGTAGAAATGCTT | SSPN | 3′ | human | dCs; InaAs; dTs; InaGs; |
| | m02 | | | | | dTs; InaAs; dGs; |
| | | | | | | InaAs; dAs; InaAs; |
| | | | | | | dTs; InaGs; dCs; |
| | | | | | | InaTs; dT-Sup |
| 673 | SSPN-56 | AAACATGTAGAAATG | SSPN | 3′ | human | dAs; InaAs; dAs; InaCs; |
| | m02 | | | | | dAs; InaTs; dGs; |
| | | | | | | InaTs; dAs; InaGs; |
| | | | | | | dAs; InaAs; dAs; |
| | | | | | | InaTs; dG-Sup |
| 674 | SSPN-57 | TTGATACCATTTATG | SSPN | 3′ | human | dTs; InaTs; dGs; InaAs; |
| | m02 | | | | | dTs; InaAs; dCs; |
| | | | | | | InaCs; dAs; InaTs; |
| | | | | | | dTs; InaTs; dAs; InaTs; |
| | | | | | | dG-Sup |
| 675 | SSPN-58 | GAACTCAATTATTAT | SSPN | 3′ | human | dGs; InaAs; dAs; InaCs; |
| | m02 | | | | | dTs; InaCs; dAs; |
| | | | | | | InaAs; dTs; InaTs; |
| | | | | | | dAs; InaTs; dTs; |
| | | | | | | InaAs; dT-Sup |
| 676 | UTRN- | AAAACGACTCCACAA | UTRN | 5′ | human | dAs; InaAs; dAs; InaAs; |
| | 972 | | | | | dCs; InaGs; dAs; |
| | m02 | | | | | InaCs; dTs; InaCs; |
| | | | | | | dCs; InaAs; dCs; |
| | | | | | | InaAs; dA-Sup |
| 677 | UTRN- | CTCCGAGGAAAAACG | UTRN | 5′ | human | dCs; InaTs; dCs; InaCs; |
| | 312 | | | | | dGs; InaAs; dGs; |
| | m02 | | | | | InaGs; dAs; InaAs; |
| | | | | | | dAs; InaAs; dAs; |
| | | | | | | InaCs; dG-Sup |
| 678 | UTRN- | GCTCCGAGGAAAAAC | UTRN | 5′ | human | dGs; InaCs; dTs; InaCs; |
| | 313 | | | | | dCs; InaGs; dAs; |
| | m02 | | | | | InaGs; dGs; InaAs; |
| | | | | | | dAs; InaAs; dAs; |
| | | | | | | InaAs; dC-Sup |
| 679 | UTRN- | CTCGGCGGGAGAAAG | UTRN | 5′ | human | dCs; InaTs; dCs; InaGs; |
| | 975 | | | | | dGs; InaCs; dGs; |
| | m02 | | | | | InaGs; dGs; InaAs; |
| | | | | | | dGs; InaAs; dAs; |
| | | | | | | InaAs; dG-Sup |
| 680 | UTRN- | GAACCGAAATTTT | UTRN | 5′ | human | dGs; InaAs; dAs; InaCs; |
| | 976 | | | | | dCs; InaGs; dAs; |
| | m02 | | | | | InaAs; dAs; InaTs; |
| | | | | | | dTs; InaTs; dT-Sup |
| 681 | UTRN- | GAGAAGGGTGCAGAT | UTRN | 5′ | human | dGs; InaAs; dGs; InaAs; |
| | 977 | | | | | dAs; InaGs; dGs; |
| | m02 | | | | | InaGs; dTs; InaGs; |
| | | | | | | dCs; InaAs; dGs; |
| | | | | | | InaAs; dT-Sup |
| 682 | UTRN- | CTCTCCAGATGAGAA | UTRN | 5′ | human | dCs; InaTs; dCs; InaTs; |
| | 978 | | | | | dCs; InaCs; dAs; |
| | m02 | | | | | InaGs; dAs; InaTs; |
| | | | | | | dGs; InaAs; dGs; |
| | | | | | | InaAs; dA-Sup |
| 683 | UTRN- | CAGGGGTCCGCTCTC | UTRN | 5′ | human | dCs; InaAs; dGs; InaGs; |
| | 979 | | | | | dGs; InaGs; dTs; |
| | m02 | | | | | InaCs; dCs; InaGs; |
| | | | | | | dCs; InaTs; dCs; |
| | | | | | | InaTs; dC-Sup |
| 684 | UTRN- | TCCGGGCAGCCAGGG | UTRN | 5′ | human | dTs; InaCs; dCs; InaGs; |
| | 980 | | | | | dGs; InaGs; dCs; |
| | m02 | | | | | InaAs; dGs; InaCs; |
| | | | | | | dCs; InaAs; dGs; |
| | | | | | | InaGs; dG-Sup |
| 685 | UTRN- | GGGGCTCGCCTCCGG | UTRN | 5′ | human | dGs; InaGs; dGs; InaGs; |
| | 981 | | | | | dCs; InaTs; dCs; |
| | m02 | | | | | InaGs; dCs; InaCs; |
| | | | | | | dTs; InaCs; dCs; |
| | | | | | | InaGs; dG-Sup |
| 686 | UTRN- | CCCCCGGGAAGGGGC | UTRN | 5′ | human | dCs; InaCs; dCs; InaCs; |
| | 982 | | | | | dCs; InaGs; dGs; |
| | m02 | | | | | InaGs; dAs; InaAs; |
| | | | | | | dGs; InaGs; dGs; |
| | | | | | | InaGs; dC-Sup |
| 687 | UTRN- | CCCACCCCCCGGGAA | UTRN | 5′ | human | dCs; InaCs; dCs; InaAs; |
| | 983 | | | | | dCs; InaCs; dCs; |
| | m02 | | | | | InaCs; dCs; InaCs; |
| | | | | | | dGs; InaGs; dGs; |
| | | | | | | InaAs; dA-Sup |
| 688 | UTRN- | GCGTTGCCGCCCCCA | UTRN | 5′ | human | dGs; InaCs; dGs; InaTs; |
| | 984 | C | | | | dTs; InaGs; dCs; |
| | m02 | | | | | InaCs; dGs; InaCs; |
| | | | | | | dCs; InaCs; dCs; |
| | | | | | | InaCs; dAs; dC-Sup |
| 689 | UTRN- | GCTGGGTCGCGCGTT | UTRN | 5′ | human | dGs; InaCs; dTs; InaGs; |
| | 985 | | | | | dGs; InaGs; dTs; |
| | m02 | | | | | InaCs; dGs; InaCs; |
| | | | | | | dGs; InaCs; dGs; |
| | | | | | | InaTs; dT-Sup |
| 690 | UTRN- | GCGCAGGACCGCTGG | UTRN | 5′ | human | dGs; InaCs; dGs; InaCs; |
| | 986 | | | | | dAs; InaGs; dGs; |
| | m02 | | | | | InaAs; dCs; InaCs; |
| | | | | | | dGs; InaCs; dTs; |
| | | | | | | InaGs; dG-Sup |
| 691 | UTRN- | AGGAGGGAGGGTGG | UTRN | 5′ | human | dAs; InaGs; dGs; InaAs; |
| | 987 | G | | | | dGs; InaGs; dGs; |
| | m02 | | | | | InaAs; dGs; InaGs; |
| | | | | | | dGs; InaTs; dGs; |
| | | | | | | InaGs; dG-Sup |
| 692 | UTRN- | CGCTGGAGGCGGAG | UTRN | 5′ | human | dCs; InaGs; dCs; InaTs; |
| | 988 | G | | | | dGs; InaGs; dAs; |
| | m02 | | | | | InaGs; dGs; InaCs; |
| | | | | | | dGs; InaGs; dAs; |
| | | | | | | InaGs; dG-Sup |
| 693 | UTRN- | TGGAGCCGAGCGCTG | UTRN | 5′ | human | dTs; InaGs; dGs; InaAs; |
| | 192 | | | | | dGs; InaCs; dCs; |
| | m02 | | | | | InaGs; dAs; InaGs; |
| | | | | | | dCs; InaGs; dCs; |
| | | | | | | InaTs; dG-Sup |
| 694 | UTRN- | CTGCCCCTTTGTTGG | UTRN | 5′ | human | dCs; InaTs; dGs; InaCs; |
| | 303 | | | | | dCs; InaCs; dCs; |
| | m02 | | | | | InaTs; dTs; InaTs; |
| | | | | | | dGs; InaTs; dTs; |
| | | | | | | InaGs; dG-Sup |
| 695 | UTRN- | CTCCCCGCTGCGGGC | UTRN | 5′ | human | dCs; InaTs; dCs; InaCs; |
| | 991 | | | | | dCs; InaCs; dGs; |
| | m02 | | | | | InaCs; dTs; InaGs; |
| | | | | | | dCs; InaGs; dGs; |
| | | | | | | InaGs; dC-Sup |
| 696 | UTRN- | CGGCTCCTCCTCCTC | UTRN | 5′ | human | dCs; InaGs; dGs; InaCs; |
| | 992 | | | | | dTs; InaCs; dCs; |
| | m02 | | | | | InaTs; dCs; InaCs; |
| | | | | | | dTs; InaCs; dCs; |
| | | | | | | InaTs; dC-Sup |
| 697 | UTRN- | GGCTCGCTCCTTCGG | UTRN | 5′ | human | dGs; InaGs; dCs; InaTs; |
| | 993 | | | | | dCs; InaGs; dCs; |
| | m02 | | | | | InaTs; dCs; InaCs; |
| | | | | | | dTs; InaTs; dCs; |
| | | | | | | InaGs; dG-Sup |
| 698 | UTRN- | TTTGTGCGCGAGAGA | UTRN | 5′ | human | dTs; InaTs; dTs; InaGs; |
| | 994 | | | | | dTs; InaGs; dCs; |
| | m02 | | | | | InaGs; dCs; InaGs; |
| | | | | | | dAs; InaGs; dAs; |
| | | | | | | InaGs; dA-Sup |
| 699 | UTRN- | ACGACTCCACAACTT | UTRN | 5′ | human | dAs; InaCs; dGs; InaAs; |
| | 995 | | | | | dCs; InaTs; dCs; |
| | m02 | | | | | InaCs; dAs; InaCs; |
| | | | | | | dAs; InaAs; dCs; |
| | | | | | | InaTs; dT-Sup |
| 700 | UTRN- | GCCCGCTTCCCTGCT | UTRN | 5′ | human | dGs; InaCs; dCs; InaCs; |
| | 997 | | | | | dGs; InaCs; dTs; |
| | m02 | | | | | InaTs; dCs; InaCs; |
| | | | | | | dCs; InaTs; dGs; |
| | | | | | | InaCs; dT-Sup |
| 701 | UTRN- | CGGCCGGCTGCTGCT | UTRN | 5′ | human | dCs; InaGs; dGs; InaCs; |
| | 662 | | | | | dCs; InaGs; dGs; |
| | m02 | | | | | InaCs; dTs; InaGs; |
| | | | | | | dCs; InaTs; dGs; |
| | | | | | | InaCs; dT-Sup |
| 702 | UTRN- | GCGGGAGAAAGCCC | UTRN | 5′ | human | dGs; InaCs; dGs; InaGs; |
| | 999 | G | | | | dGs; InaAs; dGs; |
| | m02 | | | | | InaAs; dAs; InaAs; |
| | | | | | | dGs; InaCs; dCs; |
| | | | | | | InaCs; dG-Sup |
| 703 | UTRN- | CCTCCTCGCCCCTCG | UTRN | 5′ | human | dCs; InaCs; dTs; InaCs; |
| | 1000 | | | | | dCs; InaTs; dCs; |
| | m02 | | | | | InaGs; dCs; InaCs; |
| | | | | | | dCs; InaCs; dTs; |
| | | | | | | InaCs; dG-Sup |
| 704 | UTRN- | AGAGGCTCCTCCTCG | UTRN | 5′ | human | dAs; InaGs; dAs; InaGs; |
| | 1001 | | | | | dGs; InaCs; dTs; |
| | m02 | | | | | InaCs; dCs; InaTs; |
| | | | | | | dCs; InaCs; dTs; |
| | | | | | | InaCs; dG-Sup |
| 705 | UTRN- | TCGGCTTCTGGAGCC | UTRN | 5′ | human | dTs; InaCs; dGs; InaGs; |
| | 1002 | | | | | dCs; InaTs; dTs; |
| | m02 | | | | | InaCs; dTs; InaGs; |
| | | | | | | dGs; InaAs; dGs; |
| | | | | | | InaCs; dC-Sup |
| 706 | UTRN- | CCGTGATTCCCCAAT | UTRN | 5′ | human | dCs; InaCs; dGs; InaTs; |
| | 1003 | | | | | dGs; InaAs; dTs; |
| | m02 | | | | | InaTs; dCs; InaCs; |
| | | | | | | dCs; InaCs; dAs; |
| | | | | | | InaAs; dT-Sup |
| 707 | UTRN- | AGGGGGGCGCCGCTC | UTRN | 5′ | human | dAs; InaGs; dGs; InaGs; |
| | 1004 | | | | | dGs; InaGs; dGs; |
| | m02 | | | | | InaCs; dGs; InaCs; |
| | | | | | | dCs; InaGs; dCs; |
| | | | | | | InaTs; dC-Sup |
| 708 | UTRN- | AAATGACCCAAAAGA | UTRN | 5′ | human | dAs; InaAs; dAs; InaTs; |
| | 323 | | | | | dGs; InaAs; dCs; |
| | m02 | | | | | InaCs; dCs; InaAs; |
| | | | | | | dAs; InaAs; dAs; |
| | | | | | | InaGs; dA-Sup |
| 709 | UTRN- | GTTTTCCGTTTGCAG | UTRN | 5′ | human | dGs; InaTs; dTs; InaTs; |
| | 328 | | | | | dTs; InaCs; dCs; |
| | m02 | | | | | InaGs; dTs; InaTs; |
| | | | | | | dTs; InaGs; dCs; |
| | | | | | | InaAs; dG-Sup |
| 710 | UTRN- | CCAAACGCTACAGAG | UTRN | 5′ | human | dCs; InaCs; dAs; InaAs; |
| | 334 | | | | | dAs; InaCs; dGs; |
| | m02 | | | | | InaCs; dTs; InaAs; |
| | | | | | | dCs; InaAs; dGs; |
| | | | | | | InaAs; dG-Sup |
| 711 | UTRN- | CAGGCACCAACTTTG | UTRN | 5′ | human | dCs; InaAs; dGs; InaGs; |
| | 1008 | | | | | dCs; InaAs; dCs; |
| | m02 | | | | | InaCs; dAs; InaAs; |
| | | | | | | dCs; InaTs; dTs; |
| | | | | | | InaTs; dG-Sup |
| 712 | UTRN- | CCTGGAAGGGGCGCG | UTRN | 5′ | human | dCs; InaCs; dTs; InaGs; |
| | 1009 | | | | | dGs; InaAs; dAs; |
| | m02 | | | | | InaGs; dGs; InaGs; |
| | | | | | | dGs; InaCs; dGs; |
| | | | | | | InaCs; dG-Sup |
| 713 | UTRN- | CAGTCAAAGCGCAAA | UTRN | 5′ | human | dCs; InaAs; dGs; InaTs; |
| | 345 | | | | | dCs; InaAs; dAs; |
| | m02 | | | | | InaAs; dGs; InaCs; |
| | | | | | | dGs; InaCs; dAs; |
| | | | | | | InaAs; dA-Sup |
| 714 | UTRN- | CCAAAAACAAAACAG | UTRN | 5′ | human | dCs; InaCs; dAs; InaAs; |
| | 1011 | | | | | dAs; InaAs; dAs; |
| | m02 | | | | | InaCs; dAs; InaAs; |
| | | | | | | dAs; InaAs; dCs; |
| | | | | | | InaAs; dG-Sup |
| 715 | UTRN- | TTCCGCCAAAAACAA | UTRN | 5′ | human | dTs; InaTs; dCs; InaCs; |
| | 674 | | | | | dGs; InaCs; dCs; |
| | m02 | | | | | InaAs; dAs; InaAs; |
| | | | | | | dAs; InaAs; dCs; |
| | | | | | | InaAs; dA-Sup |
| 716 | UTRN- | GGAGGAGGGAGGGT | UTRN | 5′ | human | dGs; InaGs; dAs; InaGs; |
| | 1013 | G | | | | dGs; InaAs; dGs; |
| | m02 | | | | | InaGs; dGs; InaAs; |
| | | | | | | dGs; InaGs; dGs; |
| | | | | | | InaTs; dG-Sup |
| 717 | UTRN- | CGAGCGCTGGAGGCG | UTRN | 5′ | human | dCs; InaGs; dAs; InaGs; |
| | 1014 | | | | | dCs; InaGs; dCs; |
| | m02 | | | | | InaTs; dGs; InaGs; |
| | | | | | | dAs; InaGs; dGs; |
| | | | | | | InaCs; dG-Sup |
| 718 | UTRN- | CCTGCCCCTTTGTTG | UTRN | 5′ | human | dCs; InaCs; dTs; InaGs; |
| | 1015 | | | | | dCs; InaCs; dCs; |
| | m02 | | | | | InaCs; dTs; InaTs; |
| | | | | | | dTs; InaGs; dTs; |
| | | | | | | InaTs; dG-Sup |
| 719 | UTRN- | GGCGGCTCCTCCTCC | UTRN | 5′ | human | dGs; InaGs; dCs; InaGs; |
| | 1016 | | | | | dGs; InaCs; dTs; |
| | m02 | | | | | InaCs; dCs; InaTs; |
| | | | | | | dCs; InaCs; dTs; |
| | | | | | | InaCs; dC-Sup |

Table 10 provides further exemplary non-coding RNA 5′ and 3′ end targeting oligos.

TABLE 10
Oligonucleotides designed to target 5′ and 3′ ends of non-coding RNAs

| SEQ | Oligo | | Gene | Target | | Formatted |
|---|---|---|---|---|---|---|
| ID NO | Name | Base Sequence | Name | Region | Organism | Sequence |
| 720 | DINO-1 | TAGACACTTCCAGAA | DINO | 3′ | human | dTs; InaAs; dGs; InaAs; |
| | m02 | | | | | dCs; InaAs; dCs; InaTs; |
| | | | | | | dTs; InaCs; dCs; InaAs; |
| | | | | | | dGs; InaAs; dA- |
| | | | | | | Sup |
| 721 | DINO-2 | TTCCAGAATTGTCCT | DINO | 3′ | human | dTs; InaTs; dCs; InaCs; |
| | m02 | | | | | dAs; InaGs; dAs; InaAs; |
| | | | | | | dTs; InaTs; dGs; InaTs; |
| | | | | | | dCs; InaCs; dT- |
| | | | | | | Sup |
| 722 | DINO-3 | CAGAATTGTCCTTTA | DINO | 3′ | human | dCs; InaAs; dGs; InaAs; |
| | m02 | | | | | dAs; InaTs; dTs; InaGs; |
| | | | | | | dTs; InaCs; dCs; InaTs; |
| | | | | | | dTs; InaTs; dA- |
| | | | | | | Sup |
| 723 | DINO-4 | CTGCTGGAACTCGGC | DINO | 5′ | human | dCs; InaTs; dGs; InaCs; |
| | m02 | | | | | dTs; InaGs; dGs; InaAs; |
| | | | | | | dAs; InaCs; dTs; InaCs; |
| | | | | | | dGs; InaGs; dC- |
| | | | | | | Sup |
| 724 | DINO-5 | GGCCAGGCTCAGCTG | DINO | 5′ | human | dGs; InaGs; dCs; InaCs; |
| | m02 | | | | | dAs; InaGs; dGs; InaCs; |
| | | | | | | dTs; InaCs; dAs; InaGs; |
| | | | | | | dCs; InaTs; dG- |
| | | | | | | Sup |
| 725 | DINO-6 | GCAGCCAGGAGCCTG | DINO | 5′ | human | dGs; InaCs; dAs; InaGs; |
| | m02 | | | | | dCs; InaCs; dAs; InaGs; |
| | | | | | | dGs; InaAs; dGs; InaCs; |
| | | | | | | dCs; InaTs; dG- |
| | | | | | | Sup |
| 726 | DINO-7 | ACTCGGCCAGGCTCA | DINO | 5′ | human | dAs; InaCs; dTs; InaCs; |
| | m02 | | | | | dGs; InaGs; dCs; InaCs; |
| | | | | | | dAs; InaGs; dGs; |
| | | | | | | InaCs; dTs; InaCs; dA- |
| | | | | | | Sup |
| 727 | DINO-8 | GCTGGCCTGCTGGAA | DINO | 5′ | human | dGs; InaCs; dTs; InaGs; |
| | m02 | | | | | dGs; InaCs; dCs; InaTs; |
| | | | | | | dGs; InaCs; dTs; InaGs; |
| | | | | | | dGs; InaAs; dA- |
| | | | | | | Sup |
| 728 | HOTTIP-1 | TTTAAATTGTATCGG | HOTTIP | 3′ | human | dTs; InaTs; dTs; InaAs; |
| | m02 | | | | | dAs; InaAs; dTs; InaTs; |
| | | | | | | dGs; InaTs; dAs; InaTs; |
| | | | | | | dCs; InaGs; dG- |
| | | | | | | Sup |
| 729 | HOTTIP-2 | ATTGTATCGGGCAAA | HOTTIP | 3′ | human | dAs; InaTs; dTs; InaGs; |
| | m02 | | | | | dTs; InaAs; dTs; InaCs; |
| | | | | | | dGs; InaGs; dGs; InaCs; |
| | | | | | | dAs; InaAs; dA- |
| | | | | | | Sup |
| 730 | HOTTIP-3 | GATTAAAACAAAAGA | HOTTIP | 3′ | human | dGs; InaAs; dTs; InaTs; |
| | m02 | | | | | dAs; InaAs; dAs; InaAs; |
| | | | | | | dCs; InaAs; dAs; InaAs; |
| | | | | | | dAs; InaGs; dA- |
| | | | | | | Sup |
| 731 | HOTTIP-4 | AAAACAAAAGAAACC | HOTTIP | 3′ | human | dAs; InaAs; dAs; InaAs; |
| | m02 | | | | | dCs; InaAs; dAs; InaAs; |
| | | | | | | dAs; InaGs; dAs; InaAs; |
| | | | | | | dAs; InaCs; dC- |
| | | | | | | Sup |
| 732 | HOTTIP-5 | GGGATAAAGGAAGGG | HOTTIP | 5′ | human | dGs; InaGs; dGs; InaAs; |
| | m02 | | | | | dTs; InaAs; dAs; InaAs; |
| | | | | | | dGs; InaGs; dAs; |
| | | | | | | InaAs; dGs; InaGs; |
| | | | | | | dG-Sup |
| 733 | HOTTIP-6 | CACTGGGATAAAGGA | HOTTIP | 5′ | human | dCs; InaAs; dCs; InaTs; |
| | m02 | | | | | dGs; InaGs; dGs; InaAs; |
| | | | | | | dTs; InaAs; dAs; InaAs; |
| | | | | | | dGs; InaGs; dA- |
| | | | | | | Sup |
| 734 | HOTTIP-7 | GAGCCGCCCGCTTTG | HOTTIP | 5′ | human | dGs; InaAs; dGs; InaCs; |
| | m02 | | | | | dCs; InaGs; dCs; InaCs; |
| | | | | | | dCs; InaGs; dCs; InaTs; |
| | | | | | | dTs; InaTs; dG- |
| | | | | | | Sup |
| 735 | HOTTIP-8 | TCTGGGCCCCACTG | HOTTIP | 5′ | human | dTs; InaCs; dTs; InaGs; |
| | m02 | | | | | dGs; InaGs; dCs; InaCs; |
| | | | | | | dCs; InaCs; dAs; InaCs; |
| | | | | | | dTs; InaG-Sup |
| 736 | NEST-1 | CAAAAGGTCTTAGCT | NEST | 3′ | human | dCs; InaAs; dAs; InaAs; |
| | m02 | | | | | dAs; InaGs; dGs; InaTs; |
| | | | | | | dCs; InaTs; dTs; InaAs; |
| | | | | | | dGs; InaCs; dT- |
| | | | | | | Sup |
| 737 | NEST-2 | TAGCTATTATTACTG | NEST | 3′ | human | dTs; InaAs; dGs; InaCs; |
| | m02 | | | | | dTs; InaAs; dTs; InaTs; |
| | | | | | | dAs; InaTs; dTs; InaAs; |
| | | | | | | dCs; InaTs; dG- |
| | | | | | | Sup |
| 738 | NEST-3 | ACTGTTGTTGTTTTA | NEST | 3′ | human | dAs; InaCs; dTs; InaGs; |
| | m02 | | | | | dTs; InaTs; dGs; InaTs; |
| | | | | | | dTs; InaGs; dTs; InaTs; |
| | | | | | | dTs; InaTs; dA- |
| | | | | | | Sup |
| 739 | NEST-4 | ACCTTAGAGGTTGTA | NEST | 3′ | human | dAs; InaCs; dCs; InaTs; |
| | m02 | | | | | dTs; InaAs; dGs; InaAs; |
| | | | | | | dGs; InaGs; dTs; InaTs; |
| | | | | | | dGs; InaTs; dA- |
| | | | | | | Sup |
| 740 | NEST-5 | TACCTGAAATTGCAG | NEST | 5′ | human | dTs; InaAs; dCs; InaCs; |
| | m02 | | | | | dTs; InaGs; dAs; InaAs; |
| | | | | | | dAs; InaTs; dTs; InaGs; |
| | | | | | | dCs; InaAs; dG- |
| | | | | | | Sup |
| 741 | NEST-6 | GTCAGAAAAGCTACC | NEST | 5′ | human | dGs; InaTs; dCs; InaAs; |
| | m02 | | | | | dGs; InaAs; dAs; InaAs; |
| | | | | | | dAs; InaGs; dCs; |
| | | | | | | InaTs; dAs; InaCs; dC- |
| | | | | | | Sup |
| 742 | NEST-7 | CACGCTTGGTGTGCA | NEST | 5′ | human | dCs; InaAs; dCs; InaGs; |
| | m02 | | | | | dCs; InaTs; dTs; InaGs; |
| | | | | | | dGs; InaTs; dGs; InaTs; |
| | | | | | | dGs; InaCs; dA- |
| | | | | | | Sup |
| 743 | NEST-8 | CTGTGAATGTGTGAA | NEST | 5′ | human | dCs; InaTs; dGs; InaTs; |
| | m02 | | | | | dGs; InaAs; dAs; InaTs; |
| | | | | | | dGs; InaTs; dGs; InaTs; |
| | | | | | | dGs; InaAs; dA- |
| | | | | | | Sup |
| 744 | NEST-9 | AACAGGAAGCACCTG | NEST | 5′ | human | dAs; InaAs; dCs; InaAs; |
| | m02 | | | | | dGs; InaGs; dAs; InaAs; |
| | | | | | | dGs; InaCs; dAs; |
| | | | | | | InaCs; dCs; InaTs; dG- |
| | | | | | | Sup |

TABLE 11
Sequences for FXN-434 and FXN-436

| SEQ | Oligo | | Gene | Target | | Formatted |
|---|---|---|---|---|---|---|
| ID NO | Name | Base Sequence | Name | Region | Organism | Sequence |
| 745 | FXN-434 | CGCTCCGCCCTCCAGTTT | FXN | 5′ and 3′ | human | dCs; InaGs; dCs; InaTs; |
| | m02 | TTTTTTAGGAGGCAACA | | | | dCs; InaCs; dGs; InaCs; |
| | | CATT | | | | dCs; InaCs; dTs; InaCs; |
| | | | | | | dCs; InaAs; dG; dT; |
| | | | | | | dT; dT; dT; dTs; InaTs; |
| | | | | | | dTs; InaTs; dTs; InaAs; |
| | | | | | | dGs; InaGs; dAs; |
| | | | | | | InaGs; dGs; InaCs; dAs; |
| | | | | | | InaAs; dCs; InaAs; dCs; |
| | | | | | | InaAs; dTs; InaT- |
| | | | | | | Sup |
| 746 | FXN-436 | CGCTCCGCCCTCCAGCC | FXN | 5′ and 3′ | human | dCs; InaGs; dCs; InaTs; |
| | m02 | TTTTTTTTTAGGAGGCA | | | | dCs; InaCs; dGs; InaCs; |
| | | ACACATT | | | | dCs; InaCs; dTs; InaCs; |
| | | | | | | dCs; InaAs; dGs; InaCs; |
| | | | | | | dC; dT; dT; dT; dT; |
| | | | | | | dTs; InaTs; dTs; InaTs; |
| | | | | | | dTs; InaAs; dGs; InaGs; |
| | | | | | | dAs; InaGs; dGs; |
| | | | | | | InaCs; dAs; InaAs; dCs; |
| | | | | | | InaAs; dCs; InaAs; dTs; |
| | | | | | | InaT-Sup |

TABLE 12
Oligonucleotides targeting 5′ and 3′ ends of FXN, APOA1, THRB, HAMP
and NR1H4

| Oligo | | | SEQ ID | | |
|---|---|---|---|---|---|
| Name | Gene | Organism | NO. | Base Sequence | Formatted Sequence |
| THRB- | THRB | human | 747 | CTGTTATAAGCTTTT | InaCs; omeUs; InaGs; omeUs; InaTs; omeAs; |
| 67 | | | | | InaTs; omeAs; InaAs; omeGs; InaCs; |
| m01 | | | | | omeUs; InaTs; omeUs; InaT-Sup |
| THRB- | THRB | human | 748 | GTTATAAGCTTTTTC | InaGs; omeUs; InaTs; omeAs; InaTs; ome |
| 68 | | | | | As; InaAs; omeGs; InaCs; omeUs; InaTs; |
| m01 | | | | | omeUs; InaTs; omeUs; InaC-Sup |
| THRB- | THRB | human | 749 | TATCTGTTATAAGCT | InaTs; omeAs; InaTs; omeCs; InaTs; omeGs; |
| 69 | | | | | InaTs; omeUs; InaAs; omeUs; InaAs; omeAs; |
| m01 | | | | | InaGs; omeCs; InaT-Sup |
| THRB- | THRB | human | 750 | AGTAGATGTTTATTT | InaAs; omeGs; InaTs; omeAs; InaGs; omeAs; |
| 70 | | | | | InaTs; omeGs; InaTs; omeUs; InaTs; omeAs; |
| m01 | | | | | InaTs; omeUs; InaT-Sup |
| THRB- | THRB | human | 751 | TAGGCAAAGGAATAG | InaTs; omeAs; InaGs; omeGs; InaCs; omeAs; |
| 71 | | | | | InaAs; omeAs; InaGs; omeGs; InaAs; |
| m01 | | | | | omeAs; InaTs; omeAs; InaG-Sup |
| THRB- | THRB | human | 752 | GGTAGGCAAAGGAAT | InaGs; omeGs; InaTs; omeAs; InaGs; omeGs; |
| 72 | | | | | InaCs; omeAs; InaAs; omeAs; InaGs; |
| m01 | | | | | omeGs; InaAs; omeAs; InaT-Sup |
| THRB- | THRB | human | 753 | GGCAAAGGAATAGTT | InaGs; omeGs; InaCs; omeAs; InaAs; omeAs; |
| 73 | | | | | InaGs; omeGs; InaAs; omeAs; InaTs; |
| m01 | | | | | omeAs; InaGs; omeUs; InaT-Sup |
| THRB- | THRB | human | 754 | GAAATGACACCCAGT | InaGs; omeAs; InaAs; omeAs; InaTs; omeGs; |
| 74 | | | | | InaAs; omeCs; InaAs; omeCs; InaCs; |
| m01 | | | | | omeCs; InaAs; omeGs; InaT-Sup |
| THRB- | THRB | human | 755 | AAATGACACCCAGTA | InaAs; omeAs; InaAs; omeUs; InaGs; omeAs; |
| 75 | | | | | InaCs; omeAs; InaCs; omeCs; InaCs; |
| m01 | | | | | omeAs; InaGs; omeUs; InaA-Sup |
| THRB- | THRB | human | 756 | GGCAATGGAATGAAA | InaGs; omeGs; InaCs; omeAs; InaAs; omeUs; |
| 76 | | | | | InaGs; omeGs; InaAs; omeAs; InaTs; |
| m01 | | | | | omeGs; InaAs; omeAs; InaA-Sup |
| THRB- | THRB | human | 757 | CAATGGAATGAAATG | InaCs; omeAs; InaAs; omeUs; InaGs; omeGs; |
| 77 | | | | | InaAs; omeAs; InaTs; omeGs; InaAs; |
| m01 | | | | | omeAs; InaAs; omeUs; InaG-Sup |
| THRB- | THRB | human | 758 | ATGGAATGAAATGAC | InaAs; omeUs; InaGs; omeGs; InaAs; omeAs; |
| 78 | | | | | InaTs; omeGs; InaAs; omeAs; InaAs; |
| m01 | | | | | omeUs; InaGs; omeAs; InaC-Sup |
| THRB- | THRB | human | 759 | GTTTCAAGTACCCGC | InaGs; omeUs; InaTs; omeUs; InaCs; omeAs; |
| 79 | | | | | InaAs; omeGs; InaTs; omeAs; InaCs; |
| m01 | | | | | omeCs; InaCs; omeGs; InaC-Sup |
| THRB- | THRB | human | 760 | GACCGGAGAACGAAA | InaGs; omeAs; InaCs; omeCs; InaGs; omeGs; |
| 80 | | | | | InaAs; omeGs; InaAs; omeAs; InaCs; |
| m01 | | | | | omeGs; InaAs; omeAs; InaA-Sup |
| THRB- | THRB | human | 761 | CTTTGGAAGGTGTTT | InaCs; omeUs; InaTs; omeUs; InaGs; omeGs; |
| 81 | | | | | InaAs; omeAs; InaGs; omeGs; InaTs; |
| m01 | | | | | omeGs; InaTs; omeUs; InaT-Sup |
| THRB- | THRB | human | 762 | TTTCTTTGGAAGGTG | InaTs; omeUs; InaTs; omeCs; InaTs; omeUs; |
| 82 | | | | | InaTs; omeGs; InaGs; omeAs; InaAs; |
| m01 | | | | | omeGs; InaGs; omeUs; InaG-Sup |
| THRB- | THRB | human | 763 | AGTTAATCCCCGCCG | InaAs; omeGs; InaTs; omeUs; InaAs; omeAs; |
| 83 | | | | | InaTs; omeCs; InaCs; omeCs; InaCs; |
| m01 | | | | | omeGs; InaCs; omeCs; InaG-Sup |
| THRB- | THRB | human | 764 | TCCTGCAAAATGTCA | InaTs; omeCs; InaCs; omeUs; InaGs; omeCs; |
| 84 | | | | | InaAs; omeAs; InaAs; omeAs; InaTs; |
| m01 | | | | | omeGs; InaTs; omeCs; InaA-Sup |
| THRB- | THRB | human | 765 | CCCCGCAGTCTCCAC | InaCs; omeCs; InaCs; omeCs; InaGs; omeCs; |
| 85 | | | | | InaAs; omeGs; InaTs; omeCs; InaTs; |
| m01 | | | | | omeCs; InaCs; omeAs; InaC-Sup |
| THRB- | THRB | human | 766 | TCTCCACCCTCCTCC | InaTs; omeCs; InaTs; omeCs; InaCs; omeAs; |
| 86 | | | | | InaCs; omeCs; InaCs; omeUs; InaCs; |
| m01 | | | | | omeCs; InaTs; omeCs; InaC-Sup |
| THRB- | THRB | human | 767 | GAGCGCCGGCGACTG | InaGs; omeAs; InaGs; omeCs; InaGs; omeCs; |
| 87 | | | | | InaCs; omeGs; InaGs; omeCs; InaGs; |
| m01 | | | | | omeAs; InaCs; omeUs; InaG-Sup |
| THRB- | THRB | human | 768 | AGGATGTGCGCCTTC | InaAs; omeGs; InaGs; omeAs; InaTs; omeGs; |
| 88 | | | | | InaTs; omeGs; InaCs; omeGs; InaCs; |
| m01 | | | | | omeCs; InaTs; omeUs; InaC-Sup |
| THRB- | THRB | human | 769 | GGCGCAGCGAGGAA | InaGs; omeGs; InaCs; omeGs; InaCs; omeAs; |
| 89 | | | | | InaGs; omeCs; InaGs; omeAs; InaGs; |
| m01 | | | | | omeGs; InaAs; InaA-Sup |
| THRB- | THRB | human | 770 | TTTCACTGACATCTC | InaTs; omeUs; InaTs; omeCs; InaAs; omeCs; |
| 90 | | | | | InaTs; omeGs; InaAs; omeCs; InaAs; |
| m01 | | | | | omeUs; InaCs; omeUs; InaC-Sup |
| HAMP | HAMP | human | 771 | GGTGGTCTGAGCCCC | InaGs; omeGs; InaTs; omeGs; InaGs; omeUs; |
| 01 | | | | | InaCs; omeUs; InaGs; omeAs; InaGs; |
| m01 | | | | | omeCs; InaCs; omeCs; InaC-Sup |
| HAMP | HAMP | human | 772 | GGGCCTGCCAGGGGA | InaGs; omeGs; InaGs; omeCs; InaCs; omeUs; |
| 02 | | | | | InaGs; omeCs; InaCs; omeAs; InaGs; |
| m01 | | | | | omeGs; InaGs; omeGs; InaA-Sup |
| HAMP- | HAMP | human | 773 | ACCGAGTGACAGTCG | InaAs; omeCs; InaCs; omeGs; InaAs; omeGs; |
| 03 | | | | | InaTs; omeGs; InaAs; omeCs; InaAs; |
| m01 | | | | | omeGs; InaTs; omeCs; InaG-Sup |
| HAMP- | HAMP | human | 774 | GTCTGGGACCGAGTG | InaGs; omeUs; InaCs; omeUs; InaGs; omeGs; |
| 04 | | | | | InaGs; omeAs; InaCs; omeCs; InaGs; |
| m01 | | | | | omeAs; InaGs; omeUs; InaG-Sup |
| HAMP- | HAMP | human | 775 | TGGGACCGAGTGACA | InaTs; omeGs; InaGs; omeGs; InaAs; omeCs; |
| 05 | | | | | InaCs; omeGs; InaAs; omeGs; InaTs; |
| m01 | | | | | omeGs; InaAs; omeCs; InaA-Sup |
| HAMP- | HAMP | human | 776 | GCAGAGGTGTGTTCA | InaGs; omeCs; InaAs; omeGs; InaAs; omeGs; |
| 06 | | | | | InaGs; omeUs; InaGs; omeUs; InaGs; |
| m01 | | | | | omeUs; InaTs; omeCs; InaA-Sup |
| HAMP- | HAMP | human | 777 | CGGCAGAGGTGTGTT | InaCs; omeGs; InaGs; omeCs; InaAs; omeGs; |
| 07 | | | | | InaAs; omeGs; InaGs; omeUs; InaGs; |
| m01 | | | | | omeUs; InaGs; omeUs; InaT-Sup |
| HAMP- | HAMP | human | 778 | GGGCAGACGGGGTCA | InaGs; omeGs; InaGs; omeCs; InaAs; omeGs; |
| 08 | | | | | InaAs; omeCs; InaGs; omeGs; InaGs; |
| m01 | | | | | omeGs; InaTs; omeCs; InaA-Sup |
| HAMP- | HAMP | human | 779 | CTCTGGTTTGGAAAA | InaCs; omeUs; InaCs; omeUs; InaGs; omeGs; |
| 09 | | | | | InaTs; omeUs; InaTs; omeGs; InaGs; |
| m01 | | | | | omeAs; InaAs; omeAs; InaA-Sup |
| HAMP- | HAMP | human | 780 | CTGGTTTGGAAAACA | InaCs; omeUs; InaGs; omeGs; InaTs; omeUs; |
| 10 | | | | | InaTs; omeGs; InaGs; omeAs; InaAs; |
| m01 | | | | | omeAs; InaAs; omeCs; InaA-Sup |
| HAMP- | HAMP | human | 781 | GTTTGGAAAACAAAA | InaGs; omeUs; InaTs; omeUs; InaGs; omeGs; |
| 11 | | | | | InaAs; omeAs; InaAs; omeAs; InaCs; |
| m01 | | | | | omeAs; InaAs; omeAs; InaA-Sup |
| HAMP- | HAMP | human | 782 | GGAAAACAAAAGAAC | InaGs; omeGs; InaAs; omeAs; InaAs; omeAs; |
| 12 | | | | | InaCs; omeAs; InaAs; omeAs; InaAs; |
| m01 | | | | | omeGs; InaAs; omeAs; InaC-Sup |
| HAMP- | HAMP | human | 783 | GAAAACAAAAGAACC | InaGs; omeAs; InaAs; omeAs; InaAs; omeCs; |
| 13 | | | | | InaAs; omeAs; InaAs; omeAs; InaGs; |
| m01 | | | | | omeAs; InaAs; omeCs; InaC-Sup |
| HAMP- | HAMP | human | 784 | TTTGGAAAACAAAAG | InaTs; omeUs; InaTs; omeGs; InaGs; omeAs; |
| 14 | | | | | InaAs; omeAs; InaAs; omeCs; InaAs; |
| m01 | | | | | omeAs; InaAs; omeAs; InaG-Sup |
| HAMP- | HAMP | human | 785 | TGGAAAACAAAAGAA | InaTs; omeGs; InaGs; omeAs; InaAs; omeAs; |
| 15 | | | | | InaAs; omeCs; InaAs; omeAs; InaAs; |
| m01 | | | | | omeAs; InaGs; omeAs; InaA-Sup |
| HAMP- | HAMP | human | 786 | TCTGGGGCAGCAGGA | InaTs; omeCs; InaTs; omeGs; InaGs; omeGs; |
| 16 | | | | | InaGs; omeCs; InaAs; omeGs; InaCs; |
| m01 | | | | | omeAs; InaGs; omeGs; InaA-Sup |
| NR1H4- | NR1H4 | human | 787 | ATAGAAAGGAACCTT | InaAs; omeUs; InaAs; omeGs; InaAs; omeAs; |
| 01 | | | | | InaAs; omeGs; InaGs; omeAs; InaAs; |
| m01 | | | | | omeCs; InaCs; omeUs; InaT-Sup |
| NR1H4- | NR1H4 | human | 788 | TAGAAAGGAACCTTG | InaTs; omeAs; InaGs; omeAs; InaAs; omeAs; |
| 02 | | | | | InaGs; omeGs; InaAs; omeAs; InaCs; |
| m01 | | | | | omeCs; InaTs; omeUs; InaG-Sup |
| NR1H4- | NR1H4 | human | 789 | ATTAACAATCCTTCC | InaAs; omeUs; InaTs; omeAs; InaAs; omeCs; |
| 03 | | | | | InaAs; omeAs; InaTs; omeCs; InaCs; |
| m01 | | | | | omeUs; InaTs; omeCs; InaC-Sup |
| NR1H4- | NR1H4 | human | 790 | CTTCCCTGCTAAATG | InaCs; omeUs; InaTs; omeCs; InaCs; omeCs; |
| 04 | | | | | InaTs; omeGs; InaCs; omeUs; InaAs; |
| m01 | | | | | omeAs; InaAs; omeUs; InaG-Sup |
| NR1H4- | NR1H4 | human | 791 | CCCTGCTAAATGATA | InaCs; omeCs; InaCs; omeUs; InaGs; omeCs; |
| 05 | | | | | InaTs; omeAs; InaAs; omeAs; InaTs; |
| m01 | | | | | omeGs; InaAs; omeUs; InaA-Sup |
| NR1H4- | NR1H4 | human | 792 | TGATATAAACATAGA | InaTs; omeGs; InaAs; omeUs; InaAs; omeUs; |
| 06 | | | | | InaAs; omeAs; InaAs; omeCs; InaAs; |
| m01 | | | | | omeUs; InaAs; omeGs; InaA-Sup |
| NR1H4- | NR1H4 | human | 793 | TCCCCGGGACTGAAC | InaTs; omeCs; InaCs; omeCs; InaCs; omeGs; |
| 07 | | | | | InaGs; omeGs; InaAs; omeCs; InaTs; |
| m01 | | | | | omeGs; InaAs; omeAs; InaC-Sup |
| NR1H4- | NR1H4 | human | 794 | TACAACTTCTTTGAAT | InaTs; omeAs; InaCs; omeAs; InaAs; omeCs; |
| 08 | | | | | InaTs; omeUs; InaCs; omeUs; InaTs; |
| m01 | | | | | omeUs; InaGs; omeAs; InaAs; InaT-Sup |
| NR1H4- | NR1H4 | human | 795 | TCTATCACTTCCCCG | InaTs; omeCs; InaTs; omeAs; InaTs; omeCs; |
| 09 | | | | | InaAs; omeCs; InaTs; omeUs; InaCs; |
| m01 | | | | | omeCs; InaCs; omeCs; InaG-Sup |
| NR1H4- | NR1H4 | human | 796 | TCCTGGGCACCCGTA | InaTs; omeCs; InaCs; omeUs; InaGs; omeGs; |
| 10 | | | | | InaGs; omeCs; InaAs; omeCs; InaCs; |
| m01 | | | | | omeCs; InaGs; omeUs; InaA-Sup |
| NR1H4- | NR1H4 | human | 797 | TTTCTGTACACATCA | InaTs; omeUs; InaTs; omeCs; InaTs; omeGs; |
| 11 | | | | | InaTs; omeAs; InaCs; omeAs; InaCs; |
| m01 | | | | | omeAs; InaTs; omeCs; InaA-Sup |
| NR1H4- | NR1H4 | human | 798 | ACAGCCACTGAAAAT | InaAs; omeCs; InaAs; omeGs; InaCs; omeCs; |
| 12 | | | | | InaAs; omeCs; InaTs; omeGs; InaAs; |
| m01 | | | | | omeAs; InaAs; omeAs; InaT-Sup |
| NR1H4- | NR1H4 | human | 799 | TTCACAGCCACTGAA | InaTs; omeUs; InaCs; omeAs; InaCs; omeAs; |
| 13 | | | | | InaGs; omeCs; InaCs; omeAs; InaCs; |
| m01 | | | | | omeUs; InaGs; omeAs; InaA-Sup |
| NR1H4- | NR1H4 | human | 800 | TAAAGAAATGAGTTT | InaTs; omeAs; InaAs; omeAs; InaGs; omeAs; |
| 14 | | | | | InaAs; omeAs; InaTs; omeGs; InaAs; |
| m01 | | | | | omeGs; InaTs; omeUs; InaT-Sup |
| NR1H4- | NR1H4 | human | 801 | ACATTTAAAGAAATG | InaAs; omeCs; InaAs; omeUs; InaTs; omeUs; |
| 15 | | | | | InaAs; omeAs; InaAs; omeGs; InaAs; |
| m01 | | | | | omeAs; InaAs; omeUs; InaG-Sup |
| NR1H4- | NR1H4 | human | 802 | AAGAAATGAGTTTGT | InaAs; omeAs; InaGs; omeAs; InaAs; omeAs; |
| 16 | | | | | InaTs; omeGs; InaAs; omeGs; InaTs; |
| m01 | | | | | omeUs; InaTs; omeGs; InaT-Sup |
| NR1H4- | NR1H4 | human | 803 | TGCCATTATGTTTGC | InaTs; omeGs; InaCs; omeCs; InaAs; omeUs; |
| 17 | | | | | InaTs; omeAs; InaTs; omeGs; InaTs; |
| m01 | | | | | omeUs; InaTs; omeGs; InaC-Sup |
| NR1H4- | NR1H4 | human | 804 | TCCTGTTGCCATTAT | InaTs; omeCs; InaCs; omeUs; InaGs; omeUs; |
| 18 | | | | | InaTs; omeGs; InaCs; omeCs; InaAs; |
| m01 | | | | | omeUs; InaTs; omeAs; InaT-Sup |
| NR1H4- | NR1H4 | human | 805 | GAAAATCCTGTTGCC | InaGs; omeAs; InaAs; omeAs; InaAs; omeUs; |
| 19 | | | | | InaCs; omeCs; InaTs; omeGs; InaTs; |
| m01 | | | | | omeUs; InaGs; omeCs; InaC-Sup |
| NR1H4- | NR1H4 | human | 806 | CTGTTGCCATTATGT | InaCs; omeUs; InaGs; omeUs; InaTs; omeGs; |
| 20 | | | | | InaCs; omeCs; InaAs; omeUs; InaTs; |
| m01 | | | | | omeAs; InaTs; omeGs; InaT-Sup |
| NR1H4- | NR1H4 | human | 807 | TAGAATTGAAGTAAC | InaTs; omeAs; InaGs; omeAs; InaAs; omeUs; |
| 21 | | | | | InaTs; omeGs; InaAs; omeAs; InaGs; |
| m01 | | | | | omeUs; InaAs; omeAs; InaC-Sup |
| NR1H4- | NR1H4 | human | 808 | TGAAGTAACAATCAA | InaTs; omeGs; InaAs; omeAs; InaGs; omeUs; |
| 22 | | | | | InaAs; omeAs; InaCs; omeAs; InaAs; |
| m01 | | | | | omeUs; InaCs; omeAs; InaA-Sup |
| NR1H4- | NR1H4 | human | 809 | AAGTAACAATCAATT | InaAs; omeAs; InaGs; omeUs; InaAs; omeAs; |
| 23 | | | | | InaCs; omeAs; InaAs; omeUs; InaCs; |
| m01 | | | | | omeAs; InaAs; omeUs; InaT-Sup |
| NR1H4- | NR1H4 | human | 810 | TCATCAAGATTTCTT | InaTs; omeCs; InaAs; omeUs; InaCs; omeAs; |
| 24 | | | | | InaAs; omeGs; InaAs; omeUs; InaTs; |
| m01 | | | | | omeUs; InaCs; omeUs; InaT-Sup |
| NR1H4- | NR1H4 | human | 811 | TATCTAGCCCAATAT | InaTs; omeAs; InaTs; omeCs; InaTs; omeAs; |
| 25 | | | | | InaGs; omeCs; InaCs; omeCs; InaAs; |
| m01 | | | | | omeAs; InaTs; omeAs; InaT-Sup |
| NR1H4- | NR1H4 | human | 812 | TTCTATCTAGCCCAA | InaTs; omeUs; InaCs; omeUs; InaAs; omeUs; |
| 26 | | | | | InaCs; omeUs; InaAs; omeGs; InaCs; |
| m01 | | | | | omeCs; InaCs; omeAs; InaA-Sup |
| FXN- | FXN | human | 813 | CTCCGCCCTCCAGTTT | InaCs; omeUs; omeCs; omeCs; InaGs; omeCs; |
| 837 | | | | TTATTATTTTGCTTTTT | omeCs; omeCs; InaTs; omeCs; omeCs; |
| m1000 | | | | | omeAs; InaGs; omeUs; omeUs; omeUs; |
| | | | | | InaTs; omeUs; omeAs; omeUs; InaTs; omeAs; |
| | | | | | omeUs; omeUs; InaTs; omeUs; omeGs; |
| | | | | | omeCs; InaTs; omeUs; omeUs; omeUs; |
| | | | | | InaT-Sup |
| FXN- | FXN | human | 814 | CCGCCCTCCAGTTTTT | InaCs; omeCs; omeGs; omeCs; InaCs; omeCs; |
| 838 | | | | ATTATTTTGCTTTTT | omeUs; omeCs; InaCs; omeAs; omeGs; |
| m1000 | | | | | omeUs; InaTs; omeUs; omeUs; omeUs; |
| | | | | | InaAs; omeUs; omeUs; omeAs; InaTs; |
| | | | | | omeUs; omeUs; omeUs; InaGs; omeCs; |
| | | | | | omeUs; omeUs; InaTs; omeUs; InaT-Sup |
| FXN- | FXN | human | 815 | GCCCTCCAGTTTTTAT | InaGs; omeCs; omeCs; omeCs; InaTs; |
| 839 | | | | TATTTTGCTTTTT | omeCs; omeCs; omeAs; InaGs; omeUs; |
| m1000 | | | | | omeUs; omeUs; InaTs; omeUs; omeAs; |
| | | | | | omeUs; InaTs; omeAs; omeUs; omeUs; InaTs; |
| | | | | | omeUs; omeGs; omeCs; InaTs; omeUs; |
| | | | | | omeUs; omeUs; InaT-Sup |
| FXN- | FXN | human | 816 | CGCTCCGCCCTCCAGT | InaCs; omeGs; omeCs; omeUs; InaCs; |
| 840 | | | | TTTTATTATTTTGCTTT | omeCs; omeGs; omeCs; InaCs; omeCs; |
| m1000 | | | | | omeUs; omeCs; InaCs; omeAs; omeGs; |
| | | | | | omeUs; InaTs; omeUs; omeUs; omeUs; InaAs; |
| | | | | | omeUs; omeUs; omeAs; InaTs; omeUs; |
| | | | | | omeUs; omeUs; InaGs; omeCs; omeUs; |
| | | | | | omeUs; InaT-Sup |
| FXN- | FXN | human | 817 | CGCTCCGCCCTCCAGT | InaCs; omeGs; omeCs; omeUs; InaCs; |
| 841 | | | | TTTTATTATTTTGCT | omeCs; omeGs; omeCs; InaCs; omeCs; |
| m1000 | | | | | omeUs; omeCs; InaCs; omeAs; omeGs; |
| | | | | | omeUs; InaTs; omeUs; omeUs; omeUs; InaAs; |
| | | | | | omeUs; omeUs; omeAs; InaTs; omeUs; |
| | | | | | omeUs; omeUs; InaGs; omeCs; InaT-Sup |
| FXN- | FXN | human | 818 | CGCTCCGCCCTCCAGT | InaCs; omeGs; omeCs; omeUs; InaCs; |
| 842 | | | | TTTTATTATTTTG | omeCs; omeGs; omeCs; InaCs; omeCs; |
| m1000 | | | | | omeUs; omeCs; InaCs; omeAs; omeGs; |
| | | | | | omeUs; InaTs; omeUs; omeUs; omeUs; InaAs; |
| | | | | | omeUs; omeUs; omeAs; InaTs; omeUs; |
| | | | | | omeUs; omeUs; InaG-Sup |
| FXN- | FXN | human | 819 | CTCCGCCCTCCAGTTT | InaCs; omeUs; omeCs; omeCs; InaGs; |
| 843 | | | | TTATTATTTTGCTTT | omeCs; omeCs; omeCs; InaTs; omeCs; omeCs; |
| m1000 | | | | | omeAs; InaGs; omeUs; omeUs; omeUs; |
| | | | | | InaTs; omeUs; omeAs; omeUs; InaTs; |
| | | | | | omeAs; omeUs; omeUs; InaTs; omeUs; |
| | | | | | omeGs; omeCs; InaTs; omeUs; InaT-Sup |
| FXN- | FXN | human | 820 | CCGCCCTCCAGTTTTT | InaCs; omeCs; omeGs; InaCs; omeCs; |
| 844 | | | | ATTATTTTGCT | omeCs; InaTs; omeCs; omeCs; InaAs; omeGs; |
| m1000 | | | | | omeUs; InaTs; omeUs; omeUs; InaTs; |
| | | | | | omeAs; omeUs; InaTs; omeAs; omeUs; InaTs; |
| | | | | | omeUs; omeUs; InaGs; omeCs; InaT-Sup |
| FXN- | FXN | human | 821 | GCCCTCCAGTTTTTAT | InaGs; omeCs; omeCs; InaCs; omeUs; |
| 845 | | | | TATTTTGCT | omeCs; InaCs; omeAs; omeGs; InaTs; omeUs; |
| m1000 | | | | | omeUs; InaTs; omeUs; omeAs; InaTs; |
| | | | | | omeUs; omeAs; InaTs; omeUs; omeUs; InaTs; |
| | | | | | omeGs; omeCs; InaT-Sup |
| FXN- | FXN | human | 822 | CCCTCCAGTTTTTATT | InaCs; omeCs; omeCs; InaTs; omeCs; omeCs; |
| 846 | | | | ATTTTGC | InaAs; omeGs; omeUs; InaTs; omeUs; |
| m1000 | | | | | omeUs; InaTs; omeAs; omeUs; InaTs; omeAs; |
| | | | | | omeUs; InaTs; omeUs; omeUs; InaGs; |
| | | | | | InaC-Sup |
| FXN- | FXN | human | 823 | CCTCCAGTTTTTATTAT | InaCs; omeCs; omeUs; InaCs; omeCs; omeAs; |
| 847 | | | | TTTG | InaGs; omeUs; omeUs; InaTs; omeUs; |
| m1000 | | | | | omeUs; InaAs; omeUs; omeUs; InaAs; |
| | | | | | omeUs; omeUs; InaTs; omeUs; InaG-Sup |
| FXN- | FXN | human | 824 | GCTCCGCCCTCCAGAT | InaGs; omeCs; omeUs; omeCs; InaCs; omeGs; |
| 848 | | | | TATTTTGCTTTTT | omeCs; omeCs; InaCs; omeUs; omeCs; |
| m1000 | | | | | omeCs; InaAs; omeGs; omeAs; omeUs; |
| | | | | | InaTs; omeAs; omeUs; omeUs; InaTs; |
| | | | | | omeUs; omeGs; omeCs; InaTs; omeUs; |
| | | | | | omeUs; omeUs; InaT-Sup |
| FXN- | FXN | human | 825 | TCCGCCCTCCAGATTA | InaTs; omeCs; omeCs; InaGs; omeCs; omeCs; |
| 849 | | | | TTTTGCTTTTT | InaCs; omeUs; omeCs; InaCs; omeAs; |
| m1000 | | | | | omeGs; InaAs; omeUs; omeUs; InaAs; |
| | | | | | omeUs; omeUs; InaTs; omeUs; omeGs; |
| | | | | | InaCs; omeUs; omeUs; InaTs; omeUs; InaT- |
| | | | | | Sup |
| FXN- | FXN | human | 826 | CGCCCTCCAGATTATT | InaCs; omeGs; omeCs; InaCs; omeCs; omeUs; |
| 850 | | | | TTGCTTTTT | InaCs; omeCs; omeAs; InaGs; omeAs; |
| m1000 | | | | | omeUs; InaTs; omeAs; omeUs; InaTs; |
| | | | | | omeUs; omeUs; InaGs; omeCs; omeUs; InaTs; |
| | | | | | omeUs; omeUs; InaT-Sup |
| FXN- | FXN | human | 827 | CCCTCCAGATTATTTT | InaCs; omeCs; omeCs; InaTs; omeCs; omeCs; |
| 851 | | | | GCTTTTT | InaAs; omeGs; omeAs; InaTs; omeUs; |
| m1000 | | | | | omeAs; InaTs; omeUs; omeUs; InaTs; |
| | | | | | omeGs; omeCs; InaTs; omeUs; omeUs; InaTs; |
| | | | | | InaT-Sup |
| FXN- | FXN | human | 828 | GCTCCGCCCTCCAGAT | InaGs; omeCs; omeUs; InaCs; omeCs; omeGs; |
| 852 | | | | TATTTTGCTTT | InaCs; omeCs; omeCs; InaTs; omeCs; |
| m1000 | | | | | omeCs; InaAs; omeGs; omeAs; InaTs; omeUs; |
| | | | | | omeAs; InaTs; omeUs; omeUs; InaTs; |
| | | | | | omeGs; omeCs; InaTs; omeUs; InaT-Sup |
| FXN- | FXN | human | 829 | GCTCCGCCCTCCAGAT | InaGs; omeCs; omeUs; InaCs; omeCs; omeGs; |
| 853 | | | | TATTTTGCT | InaCs; omeCs; omeCs; InaTs; omeCs; |
| m1000 | | | | | omeCs; InaAs; omeGs; omeAs; InaTs; omeUs; |
| | | | | | omeAs; InaTs; omeUs; omeUs; InaTs; |
| | | | | | omeGs; omeCs; InaT-Sup |
| FXN- | FXN | human | 830 | GCTCCGCCCTCCAGAT | InaGs; omeCs; omeUs; InaCs; omeCs; omeGs; |
| 854 | | | | TATTTTG | InaCs; omeCs; omeCs; InaTs; omeCs; |
| m1000 | | | | | omeCs; InaAs; omeGs; omeAs; InaTs; omeUs; |
| | | | | | omeAs; InaTs; omeUs; omeUs; InaTs; |
| | | | | | InaG-Sup |
| FXN- | FXN | human | 831 | TCCGCCCTCCAGATTA | InaTs; omeCs; omeCs; InaGs; omeCs; omeCs; |
| 855 | | | | TTTTGCTTT | InaCs; omeUs; omeCs; InaCs; omeAs; |
| m1000 | | | | | omeGs; InaAs; omeUs; omeUs; InaAs; omeUs; |
| | | | | | omeUs; InaTs; omeUs; omeGs; InaCs; |
| | | | | | omeUs; omeUs; InaT-Sup |
| FXN- | FXN | human | 832 | CGCCCTCCAGATTATT | InaCs; omeGs; omeCs; InaCs; omeCs; omeUs; |
| 856 | | | | TTGCT | InaCs; omeCs; omeAs; InaGs; omeAs; |
| m1000 | | | | | omeUs; InaTs; omeAs; omeUs; InaTs; omeUs; |
| | | | | | omeUs; InaGs; omeCs; InaT-Sup |
| FXN- | FXN | human | 833 | GCCCTCCAGATTATTT | InaGs; omeCs; InaCs; omeCs; InaTs; omeCs; |
| 857 | | | | TGC | InaCs; omeAs; InaGs; omeAs; InaTs; omeUs; |
| m01 | | | | | InaAs; omeUs; InaTs; omeUs; InaTs; |
| | | | | | omeGs; InaC-Sup |
| FXN- | FXN | human | 834 | CCCTCCAGATTATTTT | InaCs; omeCs; InaCs; omeUs; InaCs; omeCs; |
| 858 | | | | G | InaAs; omeGs; InaAs; omeUs; InaTs; omeAs; |
| m01 | | | | | InaTs; omeUs; InaTs; omeUs; InaG- |
| | | | | | Sup |
| FXN- | FXN | human | 835 | CTCCAGATTATTTTG | InaCs; omeUs; InaCs; omeCs; InaAs; omeGs; |
| 859 | | | | | InaAs; omeUs; InaTs; omeAs; InaTs; omeUs; |
| m01 | | | | | InaTs; omeUs; InaG-Sup |
| FXN- | FXN | human | 836 | CGCTCCGCCCTCCAGT | dCs; InaGs; dCs; InaTs; dCs; InaCs; dGs; InaCs; |
| 461 | | | | TTTTATTATTTTGCTTT | dCs; InaCs; dTs; InaCs; dCs; InaAs; dGs; InaTs; |
| m02 | | | | TT | dTs; InaTs; dTs; InaTs; dAs; InaTs; dTs; |
| | | | | | InaAs; dTs; InaTs; dTs; InaTs; dGs; InaCs; |
| | | | | | dTs; InaTs; dTs; InaTs; dT-Sup |
| Apoa1_ | APOA1 | mouse | 837 | AGTTCAAGGATCAGC | InaAs; dGs; dTs; InaTs; dCs; dAs; InaAs; dGs; |
| mus- | | | | CATTTTGGAAAGG | dGs; InaAs; dTs; dCs; InaAs; dGs; dCs; InaCs; |
| 77 | | | | | dAs; dTs; InaTs; dTs; dTs; InaGs; dGs; dAs; |
| m1000 | | | | | InaAs; dAs; dGs; InaG-Sup |
| Apoa1_ | APOA1 | mouse | 838 | TCAAGGATCAGCCATT | InaTs; dCs; dAs; InaAs; dGs; dGs; InaAs; dTs; |
| mus- | | | | TTGGAAAGG | dCs; InaAs; dGs; dCs; InaCs; dAs; dTs; InaTs; |
| 78 | | | | | dTs; dTs; InaGs; dGs; dAs; InaAs; dAs; dGs; |
| m1000 | | | | | InaG-Sup |
| Apoa1_ | APOA1 | mouse | 839 | AAGGATCAGCCATTTT | InaAs; dAs; dGs; InaGs; dAs; dTs; InaCs; dAs; |
| mus- | | | | GGAAAGG | dGs; InaCs; dCs; dAs; InaTs; dTs; dTs; InaTs; |
| 79 | | | | | dGs; dGs; InaAs; dAs; dAs; InaGs; InaG- |
| m1000 | | | | | Sup |
| Apoa1_ | APOA1 | mouse | 840 | GGATCAGCCATTTTG | InaGs; dGs; dAs; InaTs; dCs; dAs; InaGs; dCs; |
| mus- | | | | GAAAGG | dCs; InaAs; dTs; dTs; InaTs; dTs; dGs; InaGs; |
| 80 | | | | | dAs; dAs; InaAs; dGs; InaG-Sup |
| m1000 | | | | | |
| Apoa1_ | APOA1 | mouse | 841 | AGTTCAAGGATCAGC | InaAs; dGs; dTs; InaTs; dCs; dAs; InaAs; dGs; |
| mus- | | | | CATTTTGGAA | dGs; InaAs; dTs; dCs; InaAs; dGs; dCs; InaCs; |
| 81 | | | | | dAs; dTs; InaTs; dTs; dTs; InaGs; dGs; dAs; |
| m1000 | | | | | InaA-Sup |
| Apoa1_ | APOA1 | mouse | 842 | AGTTCAAGGATCAGC | InaAs; dGs; dTs; InaTs; dCs; dAs; InaAs; dGs; |
| mus- | | | | CATTTTGG | dGs; InaAs; dTs; dCs; InaAs; dGs; dCs; InaCs; |
| 82 | | | | | dAs; dTs; InaTs; dTs; dTs; InaGs; InaG- |
| m1000 | | | | | Sup |
| Apoa1_ | APOA1 | mouse | 843 | GTTCAAGGATCAGCC | InaGs; dTs; dTs; InaCs; dAs; dAs; InaGs; dGs; |
| mus- | | | | ATTTTGGAAAGG | dAs; InaTs; dCs; dAs; InaGs; dCs; dCs; InaAs; |
| 83 | | | | | dTs; dTs; InaTs; dTs; dGs; InaGs; dAs; dAs; |
| m1000 | | | | | InaAs; dGs; InaG-Sup |
| Apoa1_ | APOA1 | mouse | 844 | TCAAGGATCAGCCATT | InaTs; dCs; dAs; InaAs; dGs; dGs; InaAs; dTs; |
| mus- | | | | TTGGAAA | dCs; InaAs; dGs; dCs; InaCs; dAs; dTs; InaTs; |
| 84 | | | | | dTs; dTs; InaGs; dGs; dAs; InaAs; InaA- |
| m1000 | | | | | Sup |
| Apoa1_ | APOA1 | mouse | 845 | AAGGATCAGCCATTTT | InaAs; dAs; dGs; InaGs; dAs; dTs; InaCs; dAs; |
| mus- | | | | GGAAA | dGs; InaCs; dCs; dAs; InaTs; dTs; dTs; InaTs; |
| 85 | | | | | dGs; dGs; InaAs; dAs; InaA-Sup |
| m1000 | | | | | |
| Apoa1_ | APOA1 | mouse | 846 | AGGATCAGCCATTTTG | InaAs; dGs; InaGs; dAs; InaTs; dCs; InaAs; dGs; |
| mus- | | | | GAA | InaCs; dCs; InaAs; dTs; InaTs; dTs; InaTs; |
| 86 | | | | | dGs; InaGs; dAs; InaA-Sup |
| m12 | | | | | |
| Apoa1_ | APOA1 | mouse | 847 | GGATCAGCCATTTTG | InaGs; dGs; InaAs; dTs; InaCs; dAs; InaGs; |
| mus- | | | | GA | dCs; InaCs; dAs; InaTs; dTs; InaTs; dTs; InaGs; |
| 87 | | | | | dGs; InaA-Sup |
| m12 | | | | | |
| Apoa1_ | APOA1 | mouse | 848 | CTCCGACAGTCTGCCA | InaCs; dTs; dCs; InaCs; dGs; dAs; InaCs; dAs; |
| mus- | | | | TTTTGGAAAGGT | dGs; InaTs; dCs; dTs; InaGs; dCs; dCs; InaAs; |
| 88 | | | | | dTs; dTs; InaTs; dTs; dGs; InaGs; dAs; dAs; |
| m1000 | | | | | InaAs; dGs; dGs; InaT-Sup |
| Apoa1_ | APOA1 | mouse | 849 | CGACAGTCTGCCATTT | InaCs; dGs; dAs; InaCs; dAs; dGs; InaTs; dCs; |
| mus- | | | | TGGAAAGGT | dTs; InaGs; dCs; dCs; InaAs; dTs; dTs; InaTs; |
| 89 | | | | | dTs; dGs; InaGs; dAs; dAs; InaAs; dGs; dGs; |
| m1000 | | | | | InaT-Sup |
| Apoa1_ | APOA1 | mouse | 850 | ACAGTCTGCCATTTTG | InaAs; dCs; dAs; InaGs; dTs; dCs; InaTs; dGs; |
| mus- | | | | GAAAGGT | dCs; InaCs; dAs; dTs; InaTs; dTs; dTs; InaGs; |
| 90 | | | | | dGs; dAs; InaAs; dAs; dGs; InaGs; InaT- |
| m1000 | | | | | Sup |
| Apoa1_ | APOA1 | mouse | 851 | CTCCGACAGTCTGCCA | InaCs; dTs; dCs; InaCs; dGs; dAs; InaCs; dAs; |
| mus- | | | | TTTTGGAAA | dGs; InaTs; dCs; dTs; InaGs; dCs; dCs; InaAs; |
| 91 | | | | | dTs; dTs; InaTs; dTs; dGs; InaGs; dAs; dAs; |
| m1000 | | | | | InaA-Sup |
| Apoa1_ | APOA1 | mouse | 852 | CTCCGACAGTCTGCCA | InaCs; dTs; dCs; InaCs; dGs; dAs; InaCs; dAs; |
| mus- | | | | TTTTGGA | dGs; InaTs; dCs; dTs; InaGs; dCs; dCs; InaAs; |
| 92 | | | | | dTs; dTs; InaTs; dTs; dGs; InaGs; InaA- |
| m1000 | | | | | Sup |
| Apoa1_ | APOA1 | mouse | 853 | CTCCGACAGTCTGCCA | InaCs; dTs; dCs; InaCs; dGs; dAs; InaCs; dAs; |
| mus- | | | | TTTTG | dGs; InaTs; dCs; dTs; InaGs; dCs; dCs; InaAs; |
| 93 | | | | | dTs; dTs; InaTs; dTs; InaG-Sup |
| m1000 | | | | | |
| Apoa1_ | APOA1 | mouse | 854 | CTCCGACAGTCTGCCA | InaCs; dTs; dCs; InaCs; dGs; dAs; InaCs; dAs; |
| mus- | | | | TTTTGGAAAGG | dGs; InaTs; dCs; dTs; InaGs; dCs; dCs; InaAs; |
| 94 | | | | | dTs; dTs; InaTs; dTs; dGs; InaGs; dAs; dAs; |
| m1000 | | | | | InaAs; dGs; InaG-Sup |
| Apoa1_ | APOA1 | mouse | 855 | CCGACAGTCTGCCATT | InaCs; dCs; dGs; InaAs; dCs; dAs; InaGs; dTs; |
| mus- | | | | TTGGAAA | dCs; InaTs; dGs; dCs; InaCs; dAs; dTs; InaTs; |
| 95 | | | | | dTs; dTs; InaGs; dGs; dAs; InaAs; InaA- |
| m1000 | | | | | Sup |
| Apoa1_ | APOA1 | mouse | 856 | GACAGTCTGCCATTTT | InaGs; dAs; InaCs; dAs; InaGs; dTs; InaCs; dTs; |
| mus- | | | | GGA | InaGs; dCs; InaCs; dAs; InaTs; dTs; InaTs; |
| 96 | | | | | dTs; InaGs; dGs; InaA-Sup |
| m12 | | | | | |
| Apoa1_ | APOA1 | mouse | 857 | ACAGTCTGCCATTTTG | InaAs; dCs; InaAs; dGs; InaTs; dCs; InaTs; dGs; |
| mus- | | | | G | InaCs; dCs; InaAs; dTs; InaTs; dTs; InaTs; |
| 97 | | | | | dGs; InaG-Sup |
| m12 | | | | | |
| Apoa1_ | APOA1 | mouse | 858 | CGGAGCTCTCCGACA | InaCs; dGs; dGs; InaAs; dGs; dCs; InaTs; dCs; |
| mus- | | | | CATTTTGGAAAGGTT | dTs; InaCs; dCs; dGs; InaAs; dCs; dAs; InaCs; |
| 98 | | | | | dAs; dTs; InaTs; dTs; dTs; InaGs; dGs; dAs; |
| m1000 | | | | | InaAs; dAs; dGs; InaGs; dTs; InaT-Sup |
| Apoa1_ | APOA1 | mouse | 859 | GGAGCTCTCCGACAC | InaGs; dGs; dAs; InaGs; dCs; dTs; InaCs; dTs; |
| mus- | | | | ATTTTGGAAAGGTT | dCs; InaCs; dGs; dAs; InaCs; dAs; dCs; InaAs; |
| 99 | | | | | dTs; dTs; InaTs; dTs; dGs; InaGs; dAs; dAs; |
| m1000 | | | | | InaAs; dGs; dGs; InaTs; InaT-Sup |
| Apoa1_ | APOA1 | mouse | 860 | AGCTCTCCGACACATT | InaAs; dGs; dCs; InaTs; dCs; dTs; InaCs; dCs; |
| mus- | | | | TTGGAAAGG | dGs; InaAs; dCs; dAs; InaCs; dAs; dTs; InaTs; |
| 100 | | | | | dTs; dTs; InaGs; dGs; dAs; InaAs; dAs; dGs; |
| m1000 | | | | | InaG-Sup |
| Apoa1_ | APOA1 | mouse | 861 | CTCTCCGACACATTTT | InaCs; dTs; dCs; InaTs; dCs; dCs; InaGs; dAs; |
| mus- | | | | GGAAA | dCs; InaAs; dCs; dAs; InaTs; dTs; dTs; InaTs; |
| 101 | | | | | dGs; dGs; InaAs; dAs; InaA-Sup |
| m1000 | | | | | |
| Apoa1_ | APOA1 | mouse | 862 | TCTCCGACACATTTTG | InaTs; dCs; InaTs; dCs; InaCs; dGs; InaAs; dCs; |
| mus- | | | | GAA | InaAs; dCs; InaAs; dTs; InaTs; dTs; InaTs; |
| 102 | | | | | dGs; InaGs; dAs; InaA-Sup |
| m12 | | | | | |
| Apoa1_ | APOA1 | mouse | 863 | CTCCGACACATTTTGG | InaCs; dTs; InaCs; dCs; InaGs; dAs; InaCs; dAs; |
| mus- | | | | A | InaCs; dAs; InaTs; dTs; InaTs; dTs; InaGs; |
| 103 | | | | | dGs; InaA-Sup |
| m12 | | | | | |
| Apoa1_ | APOA1 | mouse | 864 | TCCGACACATTTTGG | InaTs; dCs; InaCs; dGs; InaAs; dCs; InaAs; dCs; |
| mus- | | | | | InaAs; dTs; InaTs; dTs; InaTs; dGs; InaG- |
| 104 | | | | | Sup |
| m12 | | | | | |

TABLE 13
Other exemplary FXN oligos

| | | | SEQ ID | | |
|---|---|---|---|---|---|
| Oligo Name | Gene | Organism | NO. | Base Sequence | Formatted Sequence |
| FXN-375 | FXN | human | 865 | CGCTCCGCCCTCCAG | dCs; InaGs; dCs; InaTs; dCs; InaCs; |
| | | | | | dGs; InaCs; dCs; InaCs; dTs; InaCs; |
| | | | | | dCs; InaAs; dG-Sup |
| FXN-390 | FXN | human | 866 | ATTATTTTGCTTTTT | dAs; InaTs; dTs; InaAs; dTs; InaTs; |
| | | | | | dTs; InaTs; dGs; InaCs; dTs; InaTs; |
| | | | | | dTs; InaTs; dT-Sup |

TABLE 14
Oligonucleotide modifications

| Symbol | Feature Description |
|---|---|
| bio | 5′ biotin |
| dAs | DNA w/3′ thiophosphate |
| dCs | DNA w/3′ thiophosphate |
| dGs | DNA w/3′ thiophosphate |
| dTs | DNA w/3′ thiophosphate |
| dG | DNA |
| enaAs | ENA w/3′ thiophosphate |
| enaCs | ENA w/3′ thiophosphate |
| enaGs | ENA w/3′ thiophosphate |
| enaTs | ENA w/3′ thiophosphate |
| fluAs | 2′-fluoro w/3′ thiophosphate |
| fluCs | 2′-fluoro w/3′ thiophosphate |
| fluGs | 2′-fluoro w/3′ thiophosphate |
| fluUs | 2′-fluoro w/3′ thiophosphate |
| lnaAs | LNA w/3′ thiophosphate |
| lnaCs | LNA w/3′ thiophosphate |
| lnaGs | LNA w/3′ thiophosphate |
| lnaTs | LNA w/3′ thiophosphate |
| omeAs | 2′-OMe w/3′ thiophosphate |
| omeCs | 2′-OMe w/3′ thiophosphate |
| omeGs | 2′-OMe w/3′ thiophosphate |
| omeTs | 2′-OMe w/3′ thiophosphate |
| lnaAs-Sup | LNA w/3′ thiophosphate at 3′ terminus |
| lnaCs-Sup | LNA w/3′ thiophosphate at 3′ terminus |
| lnaGs-Sup | LNA w/3′ thiophosphate at 3′ terminus |
| lnaTs-Sup | LNA w/3′ thiophosphate at 3′ terminus |
| lnaA-Sup | LNA w/3′ OH at 3′ terminus |
| lnaC-Sup | LNA w/3′ OH at 3′ terminus |
| lnaG-Sup | LNA w/3′ OH at 3′ terminus |
| lnaT-Sup | LNA w/3′ OH at 3′ terminus |
| omeA-Sup | 2′-OMe w/3′ OH at 3′ terminus |
| omeC-Sup | 2′-OMe w/3′ OH at 3′ terminus |
| omeG-Sup | 2′-OMe w/3′ OH at 3′ terminus |
| omeU-Sup | 2′-OMe w/3′ OH at 3′ terminus |
| dAs-Sup | DNA w/3′ thiophosphate at 3′ terminus |
| dCs-Sup | DNA w/3′ thiophosphate at 3′ terminus |
| dGs-Sup | DNA w/3′ thiophosphate at 3′ terminus |
| dTs-Sup | DNA w/3′ thiophosphate at 3′ terminus |
| dA-Sup | DNA w/3′ OH at 3′ terminus |
| dC-Sup | DNA w/3′ OH at 3′ terminus |
| dG-Sup | DNA w/3′ OH at 3′ terminus |
| dT-Sup | DNA w/3′ OH at 3′ terminus |

The suffix “Sup” in Table 14 indicates that a 3′ end nucleotide may, for synthesis purposes, be conjugated to a solid support. It should be appreciated that in general when conjugated to a solid support for synthesis, the synthesized oligonucleotide is released such that the solid support is not part of the final oligonucleotide product.

Oligos targeting 3′ and 5′ ends of RNAs, as well as pseudocircularization oligos, can upregulate gene expression.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A formulation comprising:
a single stranded synthetic nucleic acid;
one or more stabilizing oligonucleotides complementary with the single stranded synthetic nucleic acid, and
a particle.

2. The formulation of claim 1, wherein the single stranded synthetic nucleic acid is a synthetic RNA.

3. The formulation of claim 1, wherein the particle is a nanoparticle.

4.-51. (canceled)

52. The formulation of claim 1, wherein:
(a) the particle is a microsphere; or
(b) the particle comprises a polymer; and/or
(c) wherein the particle comprises a lipidoid.

53. The formulation of claim 52, wherein:
(a) the microsphere comprises poly(lactic-co-glycolic acid) (PLGA);
(b) the polymer comprises a layer of a hydrogel or surgical sealant; and/or
(c) wherein the polymer is PLGA, ethylene vinyl acetate, poloxamer, GELSITE®, or a combination thereof.

54.-57. (canceled)

58. The formulation of claim 1, wherein:
(a) the formulation further comprises protamine or calcium phosphate;
(b) the formulation further comprises hyaluronic acid;
(c) the formulation further comprises polyglutamate;
(d) the particle comprises a lipoprotein or lipoprotein mimetic; and/or
(e) the lipidoid is penta[3-(1-laurylaminopropionyl)]-triethylenetetramine hydrochloride (TETA-5LAP; 98N12-5), C12-200, MD1, or a combination thereof.

59.-64. (canceled)

65. The formulation of claim 1, wherein the stabilizing oligonucleotide is a modified oligonucleotide.

66. The formulation of claim 65, wherein the modified oligonucleotide comprises a modified sugar moiety, a modified internucleoside linkage, or a modified nucleotide, or a combination thereof.

67. The formulation of claim 1, wherein the oligonucleotide is mixmer or a morpholino.

68. (canceled)

69. The formulation of claim 2, wherein:
(a) the synthetic RNA comprises a transcription start site; wherein
(i) the one or more stabilizing oligonucleotides comprises an oligonucleotide of 8 to 50 nucleotides in length and comprising a region of complementarity that is complementary with at least 5 contiguous nucleotides of the synthetic RNA, wherein the nucleotide at the 3′-end of the region of complementarity is complementary with a nucleotide within 10 nucleotides of the transcription start site of the synthetic RNA, wherein the oligonucleotide comprises nucleotides linked by at least one modified internucleoside linkage or at least one bridged nucleotide;
(ii) the one or more stabilizing oligonucleotides comprises an oligonucleotide of 8 to 50 nucleotides in length and comprising a region of complementarity that is complementary with at least 5 contiguous nucleotides of the synthetic RNA, wherein the nucleotide at the 3′-end of the region of complementarity is complementary with a nucleotide within 10 nucleotides of the transcription start site of the synthetic RNA, wherein the oligonucleotide comprises nucleotides linked by at least one modified internucleoside linkage or at least one bridged nucleotide;
(iii) the one or more stabilizing oligonucleotides comprises an oligonucleotide comprising two regions of complementarity each of which is complementary with at least 5 contiguous nucleotides of the synthetic RNA, wherein the nucleotide at the 3′-end of the first region of complementary is complementary with a nucleotide within 100 nucleotides of the transcription start site of the synthetic RNA and wherein the second region of complementarity is complementary with a region of the synthetic RNA that ends within 300 nucleotides of the 3′-end of the synthetic RNA; and/or
(b) wherein the synthetic RNA has a 7-methylguanosine cap at its 5′-end.

70.-85. (canceled)

86. The formulation of claim 69, wherein:
(a) the synthetic RNA is an mRNA, and wherein X₂ comprises a region of complementarity that is complementary with at least 5 contiguous nucleotides in the 3′-UTR of the transcript;
(b) the one or more stabilizing oligonucleotides comprises an oligonucleotide of 10 to 50 nucleotides in length having a first region complementary with at least 5 consecutive nucleotides of the 5′-UTR of the synthetic RNA, and a second region complementary with at least 5 consecutive nucleotides of the 3′-UTR, poly(A) tail, or overlapping the polyadenylation junction of the synthetic RNA;
(c) the one or more stabilizing oligonucleotides comprises an oligonucleotide comprising the general formula 5′-X₁—X₂-3′, wherein X₁ comprises 2 to 20 pyrimidine nucleotides that form base pairs with adenine; and X₂ comprises a region of complementarity that is complementary with at least 3 contiguous nucleotides of the synthetic RNA, wherein the synthetic RNA is a poly-adenylated, wherein the nucleotide at the 5′-end of the region of complementary of X₂ is complementary with the nucleotide of the synthetic RNA that is immediately internal to the poly-adenylation junction of the synthetic RNA; and/or
(d) the one or more stabilizing oligonucleotides comprises: a first oligonucleotide having 5 to 25 nucleotides linked through internucleoside linkages, and a second oligonucleotide having 5 to 25 nucleotides linked through internucleoside linkages, wherein the first oligonucleotide is complementary with at least 5 consecutive nucleotides within 100 nucleotides of the 5′-end of the synthetic RNA and wherein the second oligonucleotide is complementary with at least 5 consecutive nucleotides within 100 nucleotides of the 3′-end of the synthetic RNA.

87. (canceled)

88. The formulation of claim 86, wherein:
(a) the first of the at least 5 consecutive nucleotides of the 5′-UTR of the stabilizing oligonucleotide is within 10 nucleotides of the 5′-methylguanosine cap of the synthetic RNA;
(b) the second region of the stabilizing oligonucleotide is complementary with at least 5 consecutive nucleotides overlapping the polyadenylation junction;
(c) the stabilizing oligonucleotide further comprises 2-20 nucleotides that link the 5′ end of the first region with the 3′ end of the second region;
(d) wherein the stabilizing oligonucleotide further comprises 2-20 nucleotides that link the 3′ end of the first region with the 5′ end of the second region; and/or
(e) the oligonucleotide is 10 to 30 nucleotides in length.

89.-95. (canceled)

96. The formulation of claim 86, wherein the first oligonucleotide and second oligonucleotide are joined by a linker that is not an oligonucleotide having a sequence complementary with the synthetic RNA.

97. The formulation of claim 96, wherein:
(a) the linker is an oligonucleotide; or
(b) the linker is a polypeptide.

98.-99. (canceled)

100. The formulation of claim 1, wherein the synthetic RNA comprises one or more modified nucleotides.

101. The formulation of claim 100, wherein the formulation comprises a first and a second stabilizing oligonucleotide; wherein the first stabilizing oligonucleotide:
(a) comprises a region of complementarity to a 5′ region of the synthetic RNA and the second stabilizing oligonucleotide comprises a region of complementarity to a 3′ region of the synthetic RNA; and/or
(b) is covalently linked with the second stabilizing oligonucleotide through an internucleoside linkage, through an oligonucleotide, or through a linker.

102.-106. (canceled)

107. The formulation of claim 1, wherein the synthetic RNA is circularized.

108. (canceled)

109. The formulation of claim 101, wherein:
(a) the first stabilizing oligonucleotide comprises a region of complementarity that is complementary with the synthetic RNA at a position within 10 nucleotides of the first nucleotide at the 5′ end of the synthetic RNA;
(b) the synthetic RNA comprises a 5′-methylguanosine cap, and wherein the first stabilizing oligonucleotide comprises a region of complementarity that is complementary with the synthetic RNA at a position within 10 nucleotides of the nucleotide immediately internal to the 5′-methylguanosine cap;
(c) the second stabilizing oligonucleotide comprises a region of complementarity that is complementary with the synthetic RNA at a position within 250 nucleotides of the 3′ end of the synthetic RNA; and/or
(d) the synthetic RNA comprises a 3′-poly(A) tail, and wherein the second stabilizing oligonucleotide comprises a region of complementarity that is complementary with the synthetic RNA at a position within 100 nucleotides of the polyadenylation junction of the synthetic RNA.

110.-118. (canceled)

119. A method of delivering a synthetic RNA to a cell, the method comprising delivering to a cell the formulation of claim 1.

120.-123. (canceled)

124. A method of treating a condition or disease associated with decreased levels of an RNA transcript in a subject, the method comprising administering a formulation of claim 1 to the subject.

125.-127. (canceled)


SEQUENCE LISTING (868 sequences)

SEQ ID NO: 1 - DNA, 15 bp, synthetic construct
  misc_feature 1..15: note = Synthetic Oligonucleotide
  source 1..15: mol_type = other DNA
tgacccaagggagac

SEQ ID NO: 2 - DNA, 15 bp, synthetic construct
  misc_feature 1..15: note = Synthetic Oligonucleotide
  source 1..15: mol_type = other DNA
tggccactggccgca

SEQ ID NO: 3 - DNA, 15 bp, synthetic construct
  misc_feature 1..15: note = Synthetic Oligonucleotide
  source 1..15: mol_type = other DNA
cggcgacccctggtg

SEQ ID NO: 4 - DNA, 15 bp, synthetic construct
  misc_feature 1..15: note = Synthetic Oligonucleotide
  source 1..15: mol_type = other DNA
cgccctccagcgctg

SEQ ID NO: 5 - DNA, 15 bp, synthetic construct
  misc_feature 1..15: note = Synthetic Oligonucleotide
  source 1..15: mol_type = other DNA
cgctccgccctccag

SEQ ID NO: 6 - DNA, 17 bp, synthetic construct
  misc_feature 1..17: note = Synthetic Oligonucleotide
  source 1..17: mol_type = other DNA
tgacccaagggagaccc

SEQ ID NO: 7 - DNA, 17 bp, synthetic construct
  misc_feature 1..17: note = Synthetic Oligonucleotide
  source 1..17: mol_type = other DNA
tggccactggccgcacc

SEQ ID NO: 8 - DNA, 17 bp, synthetic construct
  misc_feature 1..17: note = Synthetic Oligonucleotide
  source 1..17: mol_type = other DNA
cggcgacccctggtgcc

SEQ ID NO: 9 - DNA, 17 bp, synthetic construct
  misc_feature 1..17: note = Synthetic Oligonucleotide
  source 1..17: mol_type = other DNA
cgccctccagcgctgcc

SEQ ID NO: 10 - DNA, 17 bp, synthetic construct
  misc_feature 1..17: note = Synthetic Oligonucleotide
  source 1..17: mol_type = other DNA
cgctccgccctccagcc

SEQ ID NO: 11 - DNA, 24 bp, synthetic construct
  misc_feature 1..24: note = Synthetic Oligonucleotide
  source 1..24: mol_type = other DNA
tgacccaagggagacggaaaccac

SEQ ID NO: 12 - DNA, 24 bp, synthetic construct
  misc_feature 1..24: note = Synthetic Oligonucleotide
  source 1..24: mol_type = other DNA
tggccactggccgcaggaaaccac

SEQ ID NO: 13 - DNA, 24 bp, synthetic construct
  misc_feature 1..24: note = Synthetic Oligonucleotide
  source 1..24: mol_type = other DNA
cggcgacccctggtgggaaacctc

SEQ ID NO: 14 - DNA, 24 bp, synthetic construct
  misc_feature 1..24: note = Synthetic Oligonucleotide
  source 1..24: mol_type = other DNA
cgccctccagcgctgggaaacctc

SEQ ID NO: 15 - DNA, 24 bp, synthetic construct
  misc_feature 1..24: note = Synthetic Oligonucleotide
  source 1..24: mol_type = other DNA
cgctccgccctccagccaaaggtc

SEQ ID NO: 16 - DNA, 15 bp, synthetic construct
  misc_feature 1..15: note = Synthetic Oligonucleotide
  source 1..15: mol_type = other DNA
ggtttttaaggcttt

SEQ ID NO: 17 - DNA, 15 bp, synthetic construct
  misc_feature 1..15: note = Synthetic Oligonucleotide
  source 1..15: mol_type = other DNA
ggggtcttggcctga

SEQ ID NO: 18 - DNA, 15 bp, synthetic construct
  misc_feature 1..15: note = Synthetic Oligonucleotide
  source 1..15: mol_type = other DNA
cataatgaagctggg

SEQ ID NO: 19 - DNA, 15 bp, synthetic construct
  misc_feature 1..15: note = Synthetic Oligonucleotide
  source 1..15: mol_type = other DNA
aggaggcaacacatt

SEQ ID NO: 20 - DNA, 15 bp, synthetic construct
  misc_feature 1..15: note = Synthetic Oligonucleotide
  source 1..15: mol_type = other DNA
attattttgcttttt

SEQ ID NO: 21 - DNA, 15 bp, synthetic construct
  misc_feature 1..15: note = Synthetic Oligonucleotide
  source 1..15: mol_type = other DNA
cattttccctcctgg

SEQ ID NO: 22 - DNA, 15 bp, synthetic construct
  misc_feature 1..15: note = Synthetic Oligonucleotide
  source 1..15: mol_type = other DNA
gtaggctacccttta

SEQ ID NO: 23 - DNA, 15 bp, synthetic construct
  misc_feature 1..15: note = Synthetic Oligonucleotide
  source 1..15: mol_type = other DNA
gaggcttgttgcttt

SEQ ID NO: 24 - DNA, 15 bp, synthetic construct
  misc_feature 1..15: note = Synthetic Oligonucleotide
  source 1..15: mol_type = other DNA
catgtatgatgttat

SEQ ID NO: 25 - DNA, 20 bp, synthetic construct
  misc_feature 1..20: note = Synthetic Oligonucleotide
  source 1..20: mol_type = other DNA
tttttggtttttaaggcttt

SEQ ID NO: 26 - DNA, 20 bp, synthetic construct
  misc_feature 1..20: note = Synthetic Oligonucleotide
  source 1..20: mol_type = other DNA
tttttggggtcttggcctga

SEQ ID NO: 27 - DNA, 20 bp, synthetic construct
  misc_feature 1..20: note = Synthetic Oligonucleotide
  source 1..20: mol_type = other DNA
tttttcataatgaagctggg

SEQ ID NO: 28 - DNA, 20 bp, synthetic construct
  misc_feature 1..20: note = Synthetic Oligonucleotide
  source 1..20: mol_type = other DNA
tttttaggaggcaacacatt

SEQ ID NO: 29 - DNA, 20 bp, synthetic construct
  misc_feature 1..20: note = Synthetic Oligonucleotide
  source 1..20: mol_type = other DNA
tttttattattttgcttttt

SEQ ID NO: 30 - DNA, 20 bp, synthetic construct
  misc_feature 1..20: note = Synthetic Oligonucleotide
  source 1..20: mol_type = other DNA
tttttcattttccctcctgg

SEQ ID NO: 31 - DNA, 20 bp, synthetic construct
  misc_feature 1..20: note = Synthetic Oligonucleotide
  source 1..20: mol_type = other DNA
tttttgtaggctacccttta

SEQ ID NO: 32 - DNA, 20 bp, synthetic construct
  misc_feature 1..20: note = Synthetic Oligonucleotide
  source 1..20: mol_type = other DNA
tttttgaggcttgttgcttt

SEQ ID NO: 33 - DNA, 20 bp, synthetic construct
  misc_feature 1..20: note = Synthetic Oligonucleotide
  source 1..20: mol_type = other DNA
tttttcatgtatgatgttat

SEQ ID NO: 34 - DNA, 20 bp, synthetic construct
  misc_feature 1..20: note = Synthetic Oligonucleotide
  source 1..20: mol_type = other DNA
cggcgcccgagagtccacat

SEQ ID NO: 35 - DNA, 20 bp, synthetic construct
  misc_feature 1..20: note = Synthetic Oligonucleotide
  source 1..20: mol_type = other DNA
ccaggaggccggctactgcg

SEQ ID NO: 36 - DNA, 20 bp, synthetic construct
  misc_feature 1..20: note = Synthetic Oligonucleotide
  source 1..20: mol_type = other DNA
ctgggctgggctgggtgacg

SEQ ID NO: 37 - DNA, 20 bp, synthetic construct
  misc_feature 1..20: note = Synthetic Oligonucleotide
  source 1..20: mol_type = other DNA
acccgggtgagggtctgggc

SEQ ID NO: 38 - DNA, 20 bp, synthetic construct
  misc_feature 1..20: note = Synthetic Oligonucleotide
  source 1..20: mol_type = other DNA
ccaactctgccggccgcggg

SEQ ID NO: 39 - DNA, 20 bp, synthetic construct
  misc_feature 1..20: note = Synthetic Oligonucleotide
  source 1..20: mol_type = other DNA
acggcggccgcagagtgggg

SEQ ID NO: 40 - DNA, 20 bp, synthetic construct
  misc_feature 1..20: note = Synthetic Oligonucleotide
  source 1..20: mol_type = other DNA
tcgatgtcggtgcgcaggcc

SEQ ID NO: 41 - DNA, 20 bp, synthetic construct
  misc_feature 1..20: note = Synthetic Oligonucleotide
  source 1..20: mol_type = other DNA
ggcggggcgtgcaggtcgca

SEQ ID NO: 42 - DNA, 20 bp, synthetic construct
  misc_feature 1..20: note = Synthetic Oligonucleotide
  source 1..20: mol_type = other DNA
acgttggttcgaacttgcgc

SEQ ID NO: 43 - DNA, 20 bp, synthetic construct
  misc_feature 1..20: note = Synthetic Oligonucleotide
  source 1..20: mol_type = other DNA
ttccaaatctggttgaggcc

SEQ ID NO: 44 - DNA, 20 bp, synthetic construct
  misc_feature 1..20: note = Synthetic Oligonucleotide
  source 1..20: mol_type = other DNA
agacactctgctttttgaca

SEQ ID NO: 45 - DNA, 20 bp, synthetic construct
  misc_feature 1..20: note = Synthetic Oligonucleotide
  source 1..20: mol_type = other DNA
tttcctcaaattcatcaaat

SEQ ID NO: 46 - DNA, 20 bp, synthetic construct
  misc_feature 1..20: note = Synthetic Oligonucleotide
  source 1..20: mol_type = other DNA
gggtggcccaaagttccaga

SEQ ID NO: 47 - DNA, 20 bp, synthetic construct
  misc_feature 1..20: note = Synthetic Oligonucleotide
  source 1..20: mol_type = other DNA
tggtctcatctagagagcct

SEQ ID NO: 48 - DNA, 20 bp, synthetic construct
  misc_feature 1..20: note = Synthetic Oligonucleotide
  source 1..20: mol_type = other DNA
ctctgctagtctttcatagg

SEQ ID NO: 49 - DNA, 20 bp, synthetic construct
  misc_feature 1..20: note = Synthetic Oligonucleotide
  source 1..20: mol_type = other DNA
gctaaagagtccagcgtttc

SEQ ID NO: 50 - DNA, 21 bp, synthetic construct
  misc_feature 1..21: note = Synthetic Oligonucleotide
  source 1..21: mol_type = other DNA
gcaaggtcttcaaaaaactct

SEQ ID NO: 51 - DNA, 22 bp, synthetic construct
  misc_feature 1..22: note = Synthetic Oligonucleotide
  source 1..22: mol_type = other DNA
ctcaaacgtgtatggcttgtct

SEQ ID NO: 52 - DNA, 21 bp, synthetic construct
  misc_feature 1..21: note = Synthetic Oligonucleotide
  source 1..21: mol_type = other DNA
cccaaaggagacatcatagtc

SEQ ID NO: 53 - DNA, 24 bp, synthetic construct
  misc_feature 1..24: note = Synthetic Oligonucleotide
  source 1..24: mol_type = other DNA
cagtttgacagttaagacaccact

SEQ ID NO: 54 - DNA, 21 bp, synthetic construct
  misc_feature 1..21: note = Synthetic Oligonucleotide
  source 1..21: mol_type = other DNA
ataggttcctagatctccacc

SEQ ID NO: 55 - DNA, 20 bp, synthetic construct
  misc_feature 1..20: note = Synthetic Oligonucleotide
  source 1..20: mol_type = other DNA
ggcgtctgcttgttgatcac

SEQ ID NO: 56 - DNA, 23 bp, synthetic construct
  misc_feature 1..23: note = Synthetic Oligonucleotide
  source 1..23: mol_type = other DNA
aagatagccagatttgcttgttt

SEQ ID NO: 57 - DNA, 24 bp, synthetic construct
  misc_feature 1..24: note = Synthetic Oligonucleotide
  source 1..24: mol_type = other DNA
ggtccactacatacctggatggag

SEQ ID NO: 58 - DNA, 21 bp, synthetic construct
  misc_feature 1..21: note = Synthetic Oligonucleotide
  source 1..21: mol_type = other DNA
cccagtccagtcataacgctt

SEQ ID NO: 59 - DNA, 22 bp, synthetic construct
  misc_feature 1..22: note = Synthetic Oligonucleotide
  source 1..22: mol_type = other DNA
cgtgggagtacacccagttttt

SEQ ID NO: 60 - DNA, 18 bp, synthetic construct
  misc_feature 1..18: note = Synthetic Oligonucleotide
  source 1..18: mol_type = other DNA
catggagggacacgccgt

SEQ ID NO: 61 - DNA, 22 bp, synthetic construct
  misc_feature 1..22: note = Synthetic Oligonucleotide
  source 1..22: mol_type = other DNA
gtgagctctgcggccagcagct

SEQ ID NO: 62 - DNA, 21 bp, synthetic construct
  misc_feature 1..21: note = Synthetic Oligonucleotide
  source 1..21: mol_type = other DNA
agtttggtttttaaggcttta

SEQ ID NO: 63 - DNA, 21 bp, synthetic construct
  misc_feature 1..21: note = Synthetic Oligonucleotide
  source 1..21: mol_type = other DNA
taggccaaggaagacaagtcc

SEQ ID NO: 64 - DNA, 19 bp, synthetic construct
  misc_feature 1..19: note = Synthetic Oligonucleotide
  source 1..19: mol_type = other DNA
tcaagcatcttttccggaa

SEQ ID NO: 65 - DNA, 21 bp, synthetic construct
  misc_feature 1..21: note = Synthetic Oligonucleotide
  source 1..21: mol_type = other DNA
tccttaaaacggggctgggca

SEQ ID NO: 66 - DNA, 21 bp, synthetic construct
  misc_feature 1..21: note = Synthetic Oligonucleotide
  source 1..21: mol_type = other DNA
ttggcctgatagcttttaatg

SEQ ID NO: 67 - DNA, 27 bp, synthetic construct
  misc_feature 1..27: note = Synthetic Oligonucleotide
  source 1..27: mol_type = other DNA
cctcagctgcataatgaagctggggtc

SEQ ID NO: 68 - DNA, 25 bp, synthetic construct
  misc_feature 1..25: note = Synthetic Oligonucleotide
  source 1..25: mol_type = other DNA
aacaacaacaacaacaaaaaacaga

SEQ ID NO: 69 - DNA, 26 bp, synthetic construct
  misc_feature 1..26: note = Synthetic Oligonucleotide
  source 1..26: mol_type = other DNA
cctcaaaagcaggaataaaaaaaata

SEQ ID NO: 70 - DNA, 23 bp, synthetic construct
  misc_feature 1..23: note = Synthetic Oligonucleotide
  source 1..23: mol_type = other DNA
gctgtgacacatagcccaactgt

SEQ ID NO: 71 - DNA, 25 bp, synthetic construct
  misc_feature 1..25: note = Synthetic Oligonucleotide
  source 1..25: mol_type = other DNA
ggaggcaacacattctttctacaga

SEQ ID NO: 72 - DNA, 15 bp, synthetic construct
  misc_feature 1..15: note = Synthetic Oligonucleotide
  source 1..15: mol_type = other DNA
ctattaatattactg

SEQ ID NO: 73 - DNA, 16 bp, synthetic construct
  misc_feature 1..16: note = Synthetic Oligonucleotide
  source 1..16: mol_type = other DNA
cattatgtgtatgtat

SEQ ID NO: 74 - DNA, 15 bp, synthetic construct
  misc_feature 1..15: note = Synthetic Oligonucleotide
  source 1..15: mol_type = other DNA
tttatctatgttatt

SEQ ID NO: 75 - DNA, 15 bp, synthetic construct
  misc_feature 1..15: note = Synthetic Oligonucleotide
  source 1..15: mol_type = other DNA
ctaatttgaagttct

SEQ ID NO: 76 - DNA, 15 bp, synthetic construct
  misc_feature 1..15: note = Synthetic Oligonucleotide
  source 1..15: mol_type = other DNA
ttcgaacttgcgcgg

SEQ ID NO: 77 - DNA, 14 bp, synthetic construct
  misc_feature 1..14: note = Synthetic Oligonucleotide
  source 1..14: mol_type = other DNA
tagagagcctgggt

SEQ ID NO: 78 - DNA, 15 bp, synthetic construct
  misc_feature 1..15: note = Synthetic Oligonucleotide
  source 1..15: mol_type = other DNA
acaccactcccaaag

SEQ ID NO: 79 - DNA, 15 bp, synthetic construct
  misc_feature 1..15: note = Synthetic Oligonucleotide
  source 1..15: mol_type = other DNA
aggtccactacatac

SEQ ID NO: 80 - DNA, 15 bp, synthetic construct
  misc_feature 1..15: note = Synthetic Oligonucleotide
  source 1..15: mol_type = other DNA
cgttaacctggatgg

SEQ ID NO: 81 - DNA, 15 bp, synthetic construct
  misc_feature 1..15: note = Synthetic Oligonucleotide
  source 1..15: mol_type = other DNA
aaagccttaaaaacc

SEQ ID NO: 82 - DNA, 15 bp, synthetic construct
  misc_feature 1..15: note = Synthetic Oligonucleotide
  source 1..15: mol_type = other DNA
tcaggccaagacccc

SEQ ID NO: 83 - DNA, 15 bp, synthetic construct
  misc_feature 1..15: note = Synthetic Oligonucleotide
  source 1..15: mol_type = other DNA
cccagcttcattatg

SEQ ID NO: 84 - DNA, 15 bp, synthetic construct
  misc_feature 1..15: note = Synthetic Oligonucleotide
  source 1..15: mol_type = other DNA
aatgtgttgcctcct

SEQ ID NO: 85 - DNA, 15 bp, synthetic construct
  misc_feature 1..15: note = Synthetic Oligonucleotide
  source 1..15: mol_type = other DNA
aaaaagcaaaataat

SEQ ID NO: 86 - DNA, 15 bp, synthetic construct
  misc_feature 1..15: note = Synthetic Oligonucleotide
  source 1..15: mol_type = other DNA
ccaggagggaaaatg

SEQ ID NO: 87 - DNA, 15 bp, synthetic construct
  misc_feature 1..15: note = Synthetic Oligonucleotide
  source 1..15: mol_type = other DNA
taaagggtagcctac

SEQ ID NO: 88 - DNA, 15 bp, synthetic construct
  misc_feature 1..15: note = Synthetic Oligonucleotide
  source 1..15: mol_type = other DNA
aaagcaacaagcctc

SEQ ID NO: 89 - DNA, 15 bp, synthetic construct
  misc_feature 1..15: note = Synthetic Oligonucleotide
  source 1..15: mol_type = other DNA
ataacatcatacatg

SEQ ID NO: 90 - DNA, 15 bp, synthetic construct
  misc_feature 1..15: note = Synthetic Oligonucleotide
  source 1..15: mol_type = other DNA
gatactatcttcctc

SEQ ID NO: 91 - DNA, 15 bp, synthetic construct
  misc_feature 1..15: note = Synthetic Oligonucleotide
  source 1..15: mol_type = other DNA
atgggggacggggca

SEQ ID NO: 92 - DNA, 15 bp, synthetic construct
  misc_feature 1..15: note = Synthetic Oligonucleotide
  source 1..15: mol_type = other DNA
ggttgagactgggtg

SEQ ID NO: 93 - DNA, 15 bp, synthetic construct
  misc_feature 1..15: note = Synthetic Oligonucleotide
  source 1..15: mol_type = other DNA
agactgaagaggtgc

SEQ ID NO: 94 - DNA, 15 bp, synthetic construct
  misc_feature 1..15: note = Synthetic Oligonucleotide
  source 1..15: mol_type = other DNA
cgggacggctgtgtt

SEQ ID NO: 95 - DNA, 15 bp, synthetic construct
  misc_feature 1..15: note = Synthetic Oligonucleotide
  source 1..15: mol_type = other DNA
tctgtgtgggcagca

SEQ ID NO: 96 - DNA, 15 bp, synthetic construct
  misc_feature 1..15: note = Synthetic Oligonucleotide
  source 1..15: mol_type = other DNA
aaagccttaaaaacc

SEQ ID NO: 97 - DNA, 15 bp, synthetic construct
  misc_feature 1..15: note = Synthetic Oligonucleotide
  source 1..15: mol_type = other DNA
tcaggccaagacccc

SEQ ID NO: 98 - DNA, 15 bp, synthetic construct
  misc_feature 1..15: note = Synthetic Oligonucleotide
  source 1..15: mol_type = other DNA
cccagcttcattatg

SEQ ID NO: 99 - DNA, 15 bp, synthetic construct
  misc_feature 1..15: note = Synthetic Oligonucleotide
  source 1..15: mol_type = other DNA
aatgtgttgcctcct

SEQ ID NO: 100 - DNA, 15 bp, synthetic construct
  misc_feature 1..15: note = Synthetic Oligonucleotide
  source 1..15: mol_type = other DNA
aaaaagcaaaataat

SEQ ID NO: 101 - DNA, 15 bp, synthetic construct
  misc_feature 1..15: note = Synthetic Oligonucleotide
  source 1..15: mol_type = other DNA
ccaggagggaaaatg

SEQ ID NO: 102 - DNA, 15 bp, synthetic construct
  misc_feature 1..15: note = Synthetic Oligonucleotide
  source 1..15: mol_type = other DNA
taaagggtagcctac

SEQ ID NO: 103 - DNA, 15 bp, synthetic construct
  misc_feature 1..15: note = Synthetic Oligonucleotide
  source 1..15: mol_type = other DNA
aaagcaacaagcctc

SEQ ID NO: 104 - DNA, 15 bp, synthetic construct
  misc_feature 1..15: note = Synthetic Oligonucleotide
  source 1..15: mol_type = other DNA
ataacatcatacatg

SEQ ID NO: 105 - DNA, 15 bp, synthetic construct
  misc_feature 1..15: note = Synthetic Oligonucleotide
  source 1..15: mol_type = other DNA
gatactatcttcctc

SEQ ID NO: 106 - DNA, 15 bp, synthetic construct
  misc_feature 1..15: note = Synthetic Oligonucleotide
  source 1..15: mol_type = other DNA
atgggggacggggca

SEQ ID NO: 107 - DNA, 15 bp, synthetic construct
  misc_feature 1..15: note = Synthetic Oligonucleotide
  source 1..15: mol_type = other DNA
ggttgagactgggtg

SEQ ID NO: 108 - DNA, 15 bp, synthetic construct
  misc_feature 1..15: note = Synthetic Oligonucleotide
  source 1..15: mol_type = other DNA
agactgaagaggtgc

SEQ ID NO: 109 - DNA, 15 bp, synthetic construct
  misc_feature 1..15: note = Synthetic Oligonucleotide
  source 1..15: mol_type = other DNA
cgggacggctgtgtt

SEQ ID NO: 110 - DNA, 15 bp, synthetic construct
  misc_feature 1..15: note = Synthetic Oligonucleotide
  source 1..15: mol_type = other DNA
tctgtgtgggcagca

SEQ ID NO: 111 - DNA, 15 bp, synthetic construct
  misc_feature 1..15: note = Synthetic Oligonucleotide
  source 1..15: mol_type = other DNA
gaagaagaagaagaa

SEQ ID NO: 112 - DNA, 15 bp, synthetic construct
  misc_feature 1..15: note = Synthetic Oligonucleotide
  source 1..15: mol_type = other DNA
ttcttcttcttcttc

SEQ ID NO: 113 - DNA, 20 bp, synthetic construct
  misc_feature 1..20: note = Synthetic Oligonucleotide
  source 1..20: mol_type = other DNA
cggcgcccgagagtccacat

SEQ ID NO: 114 - DNA, 20 bp, synthetic construct
  misc_feature 1..20: note = Synthetic Oligonucleotide
  source 1..20: mol_type = other DNA
acggcggccgcagagtgggg

SEQ ID NO: 115 - DNA, 26 bp, synthetic construct
  misc_feature 1..26: note = Synthetic Oligonucleotide
  source 1..26: mol_type = other DNA
cctcaaaagcaggaataaaaaaaata

SEQ ID NO: 116 - DNA, 15 bp, synthetic construct
  misc_feature 1..15: note = Synthetic Oligonucleotide
  source 1..15: mol_type = other DNA
atgggggacggggca

SEQ ID NO: 117 - DNA, 15 bp, synthetic construct
  misc_feature 1..15: note = Synthetic Oligonucleotide
  source 1..15: mol_type = other DNA
ggttgagactgggtg

SEQ ID NO: 118 - DNA, 15 bp, synthetic construct
  misc_feature 1..15: note = Synthetic Oligonucleotide
  source 1..15: mol_type = other DNA
atgggggacggggca

SEQ ID NO: 119 - DNA, 35 bp, synthetic construct
  misc_feature 1..35: note = Synthetic Oligonucleotide
  source 1..35: mol_type = other DNA
tgacccaagggagactttttggtttttaaggcttt

SEQ ID NO: 120 - DNA, 35 bp, synthetic construct
  misc_feature 1..35: note = Synthetic Oligonucleotide
  source 1..35: mol_type = other DNA
tggccactggccgcatttttggtttttaaggcttt

SEQ ID NO: 121 - DNA, 35 bp, synthetic construct
  misc_feature 1..35: note = Synthetic Oligonucleotide
  source 1..35: mol_type = other DNA
cggcgacccctggtgtttttggtttttaaggcttt

SEQ ID NO: 122 - DNA, 35 bp, synthetic construct
  misc_feature 1..35: note = Synthetic Oligonucleotide
  source 1..35: mol_type = other DNA
cgccctccagcgctgtttttggtttttaaggcttt

SEQ ID NO: 123 - DNA, 35 bp, synthetic construct
  misc_feature 1..35: note = Synthetic Oligonucleotide
  source 1..35: mol_type = other DNA
cgctccgccctccagtttttggtttttaaggcttt

SEQ ID NO: 124 - DNA, 35 bp, synthetic construct
  misc_feature 1..35: note = Synthetic Oligonucleotide
  source 1..35: mol_type = other DNA
tgacccaagggagactttttggggtcttggcctga

SEQ ID NO: 125 - DNA, 35 bp, synthetic construct
  misc_feature 1..35: note = Synthetic Oligonucleotide
  source 1..35: mol_type = other DNA
tggccactggccgcatttttggggtcttggcctga

SEQ ID NO: 126 - DNA, 35 bp, synthetic construct
  misc_feature 1..35: note = Synthetic Oligonucleotide
  source 1..35: mol_type = other DNA
cggcgacccctggtgtttttggggtcttggcctga

SEQ ID NO: 127 - DNA, 35 bp, synthetic construct
  misc_feature 1..35: note = Synthetic Oligonucleotide
  source 1..35: mol_type = other DNA
cgccctccagcgctgtttttggggtcttggcctga

SEQ ID NO: 128 - DNA, 35 bp, synthetic construct
  misc_feature 1..35: note = Synthetic Oligonucleotide
  source 1..35: mol_type = other DNA
cgctccgccctccagtttttggggtcttggcctga

SEQ ID NO: 129 - DNA, 35 bp, synthetic construct
  misc_feature 1..35: note = Synthetic Oligonucleotide
  source 1..35: mol_type = other DNA
tgacccaagggagactttttcataatgaagctggg

SEQ ID NO: 130 - DNA, 35 bp, synthetic construct
  misc_feature 1..35: note = Synthetic Oligonucleotide
  source 1..35: mol_type = other DNA
tggccactggccgcatttttcataatgaagctggg

SEQ ID NO: 131 - DNA, 35 bp, synthetic construct
  misc_feature 1..35: note = Synthetic Oligonucleotide
  source 1..35: mol_type = other DNA
cggcgacccctggtgtttttcataatgaagctggg

SEQ ID NO: 132 - DNA, 35 bp, synthetic construct
  misc_feature 1..35: note = Synthetic Oligonucleotide
  source 1..35: mol_type = other DNA
cgccctccagcgctgtttttcataatgaagctggg

SEQ ID NO: 133 - DNA, 35 bp, synthetic construct
  misc_feature 1..35: note = Synthetic Oligonucleotide
  source 1..35: mol_type = other DNA
cgctccgccctccagtttttcataatgaagctggg

SEQ ID NO: 134 - DNA, 35 bp, synthetic construct
  misc_feature 1..35: note = Synthetic Oligonucleotide
  source 1..35: mol_type = other DNA
tgacccaagggagactttttaggaggcaacacatt

SEQ ID NO: 135 - DNA, 35 bp, synthetic construct
  misc_feature 1..35: note = Synthetic Oligonucleotide
  source 1..35: mol_type = other DNA
tggccactggccgcatttttaggaggcaacacatt

SEQ ID NO: 136 - DNA, 35 bp, synthetic construct
  misc_feature 1..35: note = Synthetic Oligonucleotide
  source 1..35: mol_type = other DNA
cggcgacccctggtgtttttaggaggcaacacatt

SEQ ID NO: 137 - DNA, 35 bp, synthetic construct
  misc_feature 1..35: note = Synthetic Oligonucleotide
  source 1..35: mol_type = other DNA
cgccctccagcgctgtttttaggaggcaacacatt

SEQ ID NO: 138 - DNA, 35 bp, synthetic construct
  misc_feature 1..35: note = Synthetic Oligonucleotide
  source 1..35: mol_type = other DNA
cgctccgccctccagtttttaggaggcaacacatt

SEQ ID NO: 139 - DNA, 35 bp, synthetic construct
  misc_feature 1..35: note = Synthetic Oligonucleotide
  source 1..35: mol_type = other DNA
tgacccaagggagactttttattattttgcttttt

SEQ ID NO: 140 - DNA, 35 bp, synthetic construct
  misc_feature 1..35: note = Synthetic Oligonucleotide
  source 1..35: mol_type = other DNA
tggccactggccgcatttttattattttgcttttt

SEQ ID NO: 141 - DNA, 35 bp, synthetic construct
  misc_feature 1..35: note = Synthetic Oligonucleotide
  source 1..35: mol_type = other DNA
cggcgacccctggtgtttttattattttgcttttt

SEQ ID NO: 142 - DNA, 35 bp, synthetic construct
  misc_feature 1..35: note = Synthetic Oligonucleotide
  source 1..35: mol_type = other DNA
cgccctccagcgctgtttttattattttgcttttt

SEQ ID NO: 143 - DNA, 35 bp, synthetic construct
  misc_feature 1..35: note = Synthetic Oligonucleotide
  source 1..35: mol_type = other DNA
cgctccgccctccagtttttattattttgcttttt

SEQ ID NO: 144 - DNA, 35 bp, synthetic construct
  misc_feature 1..35: note = Synthetic Oligonucleotide
  source 1..35: mol_type = other DNA
tgacccaagggagactttttcattttccctcctgg

SEQ ID NO: 145 - DNA, 35 bp, synthetic construct
  misc_feature 1..35: note = Synthetic Oligonucleotide
  source 1..35: mol_type = other DNA
tggccactggccgcatttttcattttccctcctgg

SEQ ID NO: 146 - DNA, 35 bp, synthetic construct
  misc_feature 1..35: note = Synthetic Oligonucleotide
  source 1..35: mol_type = other DNA
cggcgacccctggtgtttttcattttccctcctgg

SEQ ID NO: 147 - DNA, 35 bp, synthetic construct
  misc_feature 1..35: note = Synthetic Oligonucleotide
  source 1..35: mol_type = other DNA
cgccctccagcgctgtttttcattttccctcctgg

SEQ ID NO: 148 - DNA, 35 bp, synthetic construct
  misc_feature 1..35: note = Synthetic Oligonucleotide
  source 1..35: mol_type = other DNA
cgctccgccctccagtttttcattttccctcctgg

SEQ ID NO: 149 - DNA, 35 bp, synthetic construct
  misc_feature 1..35: note = Synthetic Oligonucleotide
  source 1..35: mol_type = other DNA
tgacccaagggagactttttgtaggctacccttta

SEQ ID NO: 150 - DNA, 35 bp, synthetic construct
  misc_feature 1..35: note = Synthetic Oligonucleotide
  source 1..35: mol_type = other DNA
tggccactggccgcatttttgtaggctacccttta

SEQ ID NO: 151 - DNA, 35 bp, synthetic construct
  misc_feature 1..35: note = Synthetic Oligonucleotide
  source 1..35: mol_type = other DNA
cggcgacccctggtgtttttgtaggctacccttta

SEQ ID NO: 152 - DNA, 35 bp, synthetic construct
  misc_feature 1..35: note = Synthetic Oligonucleotide
  source 1..35: mol_type = other DNA
cgccctccagcgctgtttttgtaggctacccttta

SEQ ID NO: 153 - DNA, 35 bp, synthetic construct
  misc_feature 1..35: note = Synthetic Oligonucleotide
  source 1..35: mol_type = other DNA
cgctccgccctccagtttttgtaggctacccttta

SEQ ID NO: 154 - DNA, 35 bp, synthetic construct
  misc_feature 1..35: note = Synthetic Oligonucleotide
  source 1..35: mol_type = other DNA
tgacccaagggagactttttgaggcttgttgcttt

SEQ ID NO: 155 - DNA, 35 bp, synthetic construct
  misc_feature 1..35: note = Synthetic Oligonucleotide
  source 1..35: mol_type = other DNA
tggccactggccgcatttttgaggcttgttgcttt

SEQ ID NO: 156 - DNA, 35 bp, synthetic construct
  misc_feature 1..35: note = Synthetic Oligonucleotide
  source 1..35: mol_type = other DNA
cggcgacccctggtgtttttgaggcttgttgcttt

SEQ ID NO: 157 - DNA, 35 bp, synthetic construct
  misc_feature 1..35: note = Synthetic Oligonucleotide
  source 1..35: mol_type = other DNA
cgccctccagcgctgtttttgaggcttgttgcttt

SEQ ID NO: 158 - DNA, 35 bp, synthetic construct
  misc_feature 1..35: note = Synthetic Oligonucleotide
  source 1..35: mol_type = other DNA
cgctccgccctccagtttttgaggcttgttgcttt

SEQ ID NO: 159 - DNA, 35 bp, synthetic construct
  misc_feature 1..35: note = Synthetic Oligonucleotide
  source 1..35: mol_type = other DNA
tgacccaagggagactttttcatgtatgatgttat

SEQ ID NO: 160 - DNA, 35 bp, synthetic construct
  misc_feature 1..35: note = Synthetic Oligonucleotide
  source 1..35: mol_type = other DNA
tggccactggccgcatttttcatgtatgatgttat

SEQ ID NO: 161 - DNA, 35 bp, synthetic construct
  misc_feature 1..35: note = Synthetic Oligonucleotide
  source 1..35: mol_type = other DNA
cggcgacccctggtgtttttcatgtatgatgttat

SEQ ID NO: 162 - DNA, 35 bp, synthetic construct
  misc_feature 1..35: note = Synthetic Oligonucleotide
  source 1..35: mol_type = other DNA
cgccctccagcgctgtttttcatgtatgatgttat

SEQ ID NO: 163 - DNA, 35 bp, synthetic construct
  misc_feature 1..35: note = Synthetic Oligonucleotide
  source 1..35: mol_type = other DNA
cgctccgccctccagtttttcatgtatgatgttat

SEQ ID NO: 164 - DNA, 25 bp, synthetic construct
  misc_feature 1..25: note = Synthetic Oligonucleotide
  source 1..25: mol_type = other DNA
cgccctccagtttttggtttttaag

SEQ ID NO: 165 - DNA, 25 bp, synthetic construct
  misc_feature 1..25: note = Synthetic Oligonucleotide
  source 1..25: mol_type = other DNA
cgccctccagtttttggggtcttgg

SEQ ID NO: 166 - DNA, 25 bp, synthetic construct
  misc_feature 1..25: note = Synthetic Oligonucleotide
  source 1..25: mol_type = other DNA
cgccctccagtttttcataatgaag

SEQ ID NO: 167 - DNA, 25 bp, synthetic construct
  misc_feature 1..25: note = Synthetic Oligonucleotide
  source 1..25: mol_type = other DNA
cgccctccagtttttaggaggcaac

SEQ ID NO: 168 - DNA, 25 bp, synthetic construct
  misc_feature 1..25: note = Synthetic Oligonucleotide
  source 1..25: mol_type = other DNA
cgccctccagtttttattattttgc

SEQ ID NO: 169 - DNA, 25 bp, synthetic construct
  misc_feature 1..25: note = Synthetic Oligonucleotide
  source 1..25: mol_type = other DNA
cgccctccagtttttcattttccct

SEQ ID NO: 170 - DNA, 25 bp, synthetic construct
  misc_feature 1..25: note = Synthetic Oligonucleotide
  source 1..25: mol_type = other DNA
cgccctccagtttttgtaggctacc

SEQ ID NO: 171 - DNA, 25 bp, synthetic construct
  misc_feature 1..25: note = Synthetic Oligonucleotide
  source 1..25: mol_type = other DNA
cgccctccagtttttgaggcttgtt

SEQ ID NO: 172 - DNA, 25 bp, synthetic construct
  misc_feature 1..25: note = Synthetic Oligonucleotide
  source 1..25: mol_type = other DNA
cgccctccagtttttcatgtatgat

SEQ ID NO: 173 - DNA, 27 bp, synthetic construct
  misc_feature 1..27: note = Synthetic Oligonucleotide
  source 1..27: mol_type = other DNA
tgacccaagggagactttttttttttt

SEQ ID NO: 174 - DNA, 27 bp, synthetic construct
  misc_feature 1..27: note = Synthetic Oligonucleotide
  source 1..27: mol_type = other DNA
tggccactggccgcatttttttttttt

SEQ ID NO: 175 - DNA, 27 bp, synthetic construct
  misc_feature 1..27: note = Synthetic Oligonucleotide
  source 1..27: mol_type = other DNA
cggcgacccctggtgtttttttttttt

SEQ ID NO: 176 - DNA, 27 bp, synthetic construct
  misc_feature 1..27: note = Synthetic Oligonucleotide
  source 1..27: mol_type = other DNA
cgccctccagcgctgtttttttttttt

SEQ ID NO: 177 - DNA, 27 bp, synthetic construct
  misc_feature 1..27: note = Synthetic Oligonucleotide
  source 1..27: mol_type = other DNA
cgctccgccctccagtttttttttttt

SEQ ID NO: 178 - DNA, 15 bp, synthetic construct
  misc_feature 1..15: note = Synthetic Oligonucleotide
  source 1..15: mol_type = other DNA
aaaataaacaacaac

SEQ ID NO: 179 - DNA, 16 bp, synthetic construct
  misc_feature 1..16: note = Synthetic Oligonucleotide
  source 1..16: mol_type = other DNA
aggaataaaaaaaata

SEQ ID NO: 180 - DNA, 15 bp, synthetic construct
  misc_feature 1..15: note = Synthetic Oligonucleotide
  source 1..15: mol_type = other DNA
tcaaaagcaggaata

SEQ ID NO: 181 - DNA, 15 bp, synthetic construct
  misc_feature 1..15: note = Synthetic Oligonucleotide
  source 1..15: mol_type = other DNA
actgtcctcaaaagc

SEQ ID NO: 182 - DNA, 15 bp, synthetic construct
  misc_feature 1..15: note = Synthetic Oligonucleotide
  source 1..15: mol_type = other DNA
agcccaactgtcctc

SEQ ID NO: 183 - DNA, 15 bp, synthetic construct
  misc_feature 1..15: note = Synthetic Oligonucleotide
  source 1..15: mol_type = other DNA
tgacacatagcccaa

SEQ ID NO: 184 - DNA, 15 bp, synthetic construct
  misc_feature 1..15: note = Synthetic Oligonucleotide
  source 1..15: mol_type = other DNA
gagctgtgacacata

SEQ ID NO: 185 - DNA, 15 bp, synthetic construct
  misc_feature 1..15: note = Synthetic Oligonucleotide
  source 1..15: mol_type = other DNA
tctgggcctgggctg

SEQ ID NO: 186 - DNA, 15 bp, synthetic construct
  misc_feature 1..15: note = Synthetic Oligonucleotide
  source 1..15: mol_type = other DNA
ggtgagggtctgggc

SEQ ID NO: 187 - DNA, 15 bp, synthetic construct
  misc_feature 1..15: note = Synthetic Oligonucleotide
  source 1..15: mol_type = other DNA
gggacccgggtgagg

SEQ ID NO: 188 - DNA, 15 bp, synthetic construct
  misc_feature 1..15: note = Synthetic Oligonucleotide
  source 1..15: mol_type = other DNA
ccggccgcgggaccc

SEQ ID NO: 189 - DNA, 15 bp, synthetic construct
  misc_feature 1..15: note = Synthetic Oligonucleotide
  source 1..15: mol_type = other DNA
caactctgccggccg

SEQ ID NO: 190 - DNA, 15 bp, synthetic construct
  misc_feature 1..15: note = Synthetic Oligonucleotide
  source 1..15: mol_type = other DNA
agtggggccaactct

SEQ ID NO: 191 - DNA, 15 bp, synthetic construct
  misc_feature 1..15: note = Synthetic Oligonucleotide
  source 1..15: mol_type = other DNA
ggccgcagagtgggg

SEQ ID NO: 192 - DNA, 15 bp, synthetic construct
  misc_feature 1..15: note = Synthetic Oligonucleotide
  source 1..15: mol_type = other DNA
gccacggcggccgca

SEQ ID NO: 193 - DNA, 15 bp, synthetic construct
  misc_feature 1..15: note = Synthetic Oligonucleotide
  source 1..15: mol_type = other DNA
gtgcgcaggccacgg

SEQ ID NO: 194 - DNA, 15 bp, synthetic construct
  misc_feature 1..15: note = Synthetic Oligonucleotide
  source 1..15: mol_type = other DNA
gggggacggggcagg

SEQ ID NO: 195 - DNA, 15 bp, synthetic construct
  misc_feature 1..15: note = Synthetic Oligonucleotide
  source 1..15: mol_type = other DNA
gggacggggcaggtt

SEQ ID NO: 196 - DNA, 15 bp, synthetic construct
  misc_feature 1..15: note = Synthetic Oligonucleotide
  source 1..15: mol_type = other DNA
gacggggcaggttga

SEQ ID NO: 197 - DNA, 15 bp, synthetic construct
  misc_feature 1..15: note = Synthetic Oligonucleotide
  source 1..15: mol_type = other DNA
cggggcaggttgaga

SEQ ID NO: 198 - DNA, 15 bp, synthetic construct
  misc_feature 1..15: note = Synthetic Oligonucleotide
  source 1..15: mol_type = other DNA
gggcaggttgagact

SEQ ID NO: 199 - DNA, 15 bp, synthetic construct
  misc_feature 1..15: note = Synthetic Oligonucleotide
  source 1..15: mol_type = other DNA
gcaggttgagactgg

SEQ ID NO: 200 - DNA, 15 bp, synthetic construct
  misc_feature 1..15: note = Synthetic Oligonucleotide
  source 1..15: mol_type = other DNA
aggttgagactgggt

SEQ ID NO: 201 - DNA, 15 bp, synthetic construct
  misc_feature 1..15: note = Synthetic Oligonucleotide
  source 1..15: mol_type = other DNA
ggaaaaattccagga

SEQ ID NO: 202 - DNA, 15 bp, synthetic construct
  misc_feature 1..15: note = Synthetic Oligonucleotide
  source 1..15: mol_type = other DNA
aattccaggagggaa

SEQ ID NO: 203 - DNA, 15 bp, synthetic construct
  misc_feature 1..15: note = Synthetic Oligonucleotide
  source 1..15: mol_type = other DNA
gagggaaaatgaatt

SEQ ID NO: 204 - DNA, 17 bp, synthetic construct
  misc_feature 1..17: note = Synthetic Oligonucleotide
  source 1..17: mol_type = other DNA
gaaaatgaattgtcttc

SEQ ID NO: 205 - DNA, 15 bp, synthetic construct
  misc_feature 1..15: note = Synthetic Oligonucleotide
  source 1..15: mol_type = other DNA
gggggacggggcagg

SEQ ID NO: 206 - DNA, 15 bp, synthetic construct
  misc_feature 1..15: note = Synthetic Oligonucleotide
  source 1..15: mol_type = other DNA
gggacggggcaggtt

SEQ ID NO: 207 - DNA, 15 bp, synthetic construct
  misc_feature 1..15: note = Synthetic Oligonucleotide
  source 1..15: mol_type = other DNA
gacggggcaggttga

SEQ ID NO: 208 - DNA, 15 bp, synthetic construct
  misc_feature 1..15: note = Synthetic Oligonucleotide
  source 1..15: mol_type = other DNA
cggggcaggttgaga

SEQ ID NO: 209 - DNA, 15 bp, synthetic construct
  misc_feature 1..15: note = Synthetic Oligonucleotide
  source 1..15: mol_type = other DNA
gggcaggttgagact

SEQ ID NO: 210 - DNA, 15 bp, synthetic construct
  misc_feature 1..15: note = Synthetic Oligonucleotide
  source 1..15: mol_type = other DNA
gcaggttgagactgg

SEQ ID NO: 211 - DNA, 15 bp, synthetic construct
  misc_feature 1..15: note = Synthetic Oligonucleotide
  source 1..15: mol_type = other DNA
aggttgagactgggt

SEQ ID NO: 212 - DNA, 15 bp, synthetic construct
  misc_feature 1..15: note = Synthetic Oligonucleotide
  source 1..15: mol_type = other DNA
ggaaaaattccagga

SEQ ID NO: 213 - DNA, 15 bp, synthetic construct
  misc_feature 1..15: note = Synthetic Oligonucleotide
  source 1..15: mol_type = other DNA
aattccaggagggaa

SEQ ID NO: 214 - DNA, 15 bp, synthetic construct
  misc_feature 1..15: note = Synthetic Oligonucleotide
  source 1..15: mol_type = other DNA
gagggaaaatgaatt

SEQ ID NO: 215 - DNA, 17 bp, synthetic construct
  misc_feature 1..17: note = Synthetic Oligonucleotide
  source 1..17: mol_type = other DNA
gaaaatgaattgtcttc

SEQ ID NO: 216 - DNA, 15 bp, synthetic construct
  misc_feature 1..15: note = Synthetic Oligonucleotide
  source 1..15: mol_type = other DNA
ggtggtttcagttct

SEQ ID NO: 217 - DNA, 20 bp, synthetic construct
  misc_feature 1..20: note = Synthetic Oligonucleotide
  source 1..20: mol_type = other DNA
tttttggtggtttcagttct

SEQ ID NO: 218 - DNA, 15 bp, synthetic construct
  misc_feature 1..15: note = Synthetic Oligonucleotide
  source 1..15: mol_type = other DNA
agcgtgctatctggg

SEQ ID NO: 219 - DNA, 15 bp, synthetic construct
  misc_feature 1..15: note = Synthetic Oligonucleotide
  source 1..15: mol_type = other DNA
tggcccagggactct

SEQ ID NO: 220 - DNA, 14 bp, synthetic construct
  misc_feature 1..14: note = Synthetic Oligonucleotide
  source 1..14: mol_type = other DNA
tctgcggctctggc

SEQ ID NO: 221 - DNA, 15 bp, synthetic construct
  misc_feature 1..15: note = Synthetic Oligonucleotide
  source 1..15: mol_type = other DNA
cggtccggctctggg

SEQ ID NO: 222 - DNA, 15 bp, synthetic construct
  misc_feature 1..15: note = Synthetic Oligonucleotide
  source 1..15: mol_type = other DNA
tcatcccgggaagct

SEQ ID NO: 223 - DNA, 15 bp, synthetic construct
  misc_feature 1..15: note = Synthetic Oligonucleotide
  source 1..15: mol_type = other DNA
ccccaagtccccgct

SEQ ID NO: 224 - DNA, 15 bp, synthetic construct
  misc_feature 1..15: note = Synthetic Oligonucleotide
  source 1..15: mol_type = other DNA
ccaaccatgcaagca

SEQ ID NO: 225 - DNA, 17 bp, synthetic construct
  misc_feature 1..17: note = Synthetic Oligonucleotide
  source 1..17: mol_type = other DNA
tggcccagggactcttc

SEQ ID NO: 226 - DNA, 18 bp, synthetic construct
  misc_feature 1..18: note = Synthetic Oligonucleotide
  source 1..18: mol_type = other DNA
cggtccggctctgggttc

SEQ ID NO: 227 - DNA, 17 bp, synthetic construct
  misc_feature 1..17: note = Synthetic Oligonucleotide
  source 1..17: mol_type = other DNA
ccaaccatgcaagcacc

SEQ ID NO: 228 - DNA, 26 bp, synthetic construct
  misc_feature 1..26: note = Synthetic Oligonucleotide
  source 1..26: mol_type = other DNA
tggcccagggactctcacaaagtgac

SEQ ID NO: 229 - DNA, 26 bp, synthetic construct
  misc_feature 1..26: note = Synthetic Oligonucleotide
  source 1..26: mol_type = other DNA
cggtccggctctgggaagaaactttc

SEQ ID NO: 230 - DNA, 26 bp, synthetic construct
  misc_feature 1..26: note = Synthetic Oligonucleotide
  source 1..26: mol_type = other DNA
ccaaccatgcaagcactcaaagagtc

SEQ ID NO: 231 - DNA, 34 bp, synthetic construct
  misc_feature 1..34: note = Synthetic Oligonucleotide
  source 1..34: mol_type = other DNA
tggcccagggactctttttggtggtttcagttct

SEQ ID NO: 232 - DNA, 35 bp, synthetic construct
  misc_feature 1..35: note = Synthetic Oligonucleotide
  source 1..35: mol_type = other DNA
cggtccggctctgggtttttggtggtttcagttct

SEQ ID NO: 233 - DNA, 35 bp, synthetic construct
  misc_feature 1..35: note = Synthetic Oligonucleotide
  source 1..35: mol_type = other DNA
ccaaccatgcaagcatttttggtggtttcagttct

SEQ ID NO: 234 - DNA, 24 bp, synthetic construct
  misc_feature 1..24: note = Synthetic Oligonucleotide
  source 1..24: mol_type = other DNA
cagggactctttttggtggtttca

SEQ ID NO: 235 - DNA, 25 bp, synthetic construct
  misc_feature 1..25: note = Synthetic Oligonucleotide
  source 1..25: mol_type = other DNA
cggctctgggtttttggtggtttca

SEQ ID NO: 236 - DNA, 25 bp, synthetic construct
  misc_feature 1..25: note = Synthetic Oligonucleotide
  source 1..25: mol_type = other DNA
catgcaagcatttttggtggtttca

SEQ ID NO: 237 - DNA, 29 bp, synthetic construct
  misc_feature 1..29: note = Synthetic Oligonucleotide
  source 1..29: mol_type = other DNA
tggcccagggactcggtggtttcagttct

SEQ ID NO: 238 - DNA, 30 bp, synthetic construct
  misc_feature 1..30: note = Synthetic Oligonucleotide
  source 1..30: mol_type = other DNA
cggtccggctctggtggtggtttcagttct

SEQ ID NO: 239 - DNA, 30 bp, synthetic construct
  misc_feature 1..30: note = Synthetic Oligonucleotide
  source 1..30: mol_type = other DNA
ccaaccatgcaagcaggtggtttcagttct

SEQ ID NO: 240 - DNA, 20 bp, synthetic construct
  misc_feature 1..20: note = Synthetic Oligonucleotide
  source 1..20: mol_type = other DNA
tttttagataaaatattata

SEQ ID NO: 241 - DNA, 19 bp, synthetic construct
  misc_feature 1..19: note = Synthetic Oligonucleotide
  source 1..19: mol_type = other DNA
tttttattcagataaaata

SEQ ID NO: 242 - DNA, 20 bp, synthetic construct
  misc_feature 1..20: note = Synthetic Oligonucleotide
  source 1..20: mol_type = other DNA
tttttggtttatttaaaact

SEQ ID NO: 243 - DNA, 20 bp, synthetic construct
  misc_feature 1..20: note = Synthetic Oligonucleotide
  source 1..20: mol_type = other DNA
tttttaaatttatattacat

SEQ ID NO: 244 - DNA, 20 bp, synthetic construct
  misc_feature 1..20: note = Synthetic Oligonucleotide
  source 1..20: mol_type = other DNA
tttttcttaaatttatatta

SEQ ID NO: 245 - DNA, 20 bp, synthetic construct
  misc_feature 1..20: note = Synthetic Oligonucleotide
  source 1..20: mol_type = other DNA
tttttcacaaaatgttcatt

SEQ ID NO: 246 - DNA, 15 bp, synthetic construct
  misc_feature 1..15: note = Synthetic Oligonucleotide
  source 1..15: mol_type = other DNA
cctccgccttctccc

SEQ ID NO: 247 - DNA, 15 bp, synthetic construct
  misc_feature 1..15: note = Synthetic Oligonucleotide
  source 1..15: mol_type = other DNA
tctggtcgggaaact

SEQ ID NO: 248 - DNA, 15 bp, synthetic construct
  misc_feature 1..15: note = Synthetic Oligonucleotide
  source 1..15: mol_type = other DNA
gctacagccttttcc

SEQ ID NO: 249 - DNA, 16 bp, synthetic construct
  misc_feature 1..16: note = Synthetic Oligonucleotide
  source 1..16: mol_type = other DNA
cctccgccttctcccc

SEQ ID NO: 250 - DNA, 17 bp, synthetic construct
  misc_feature 1..17: note = Synthetic Oligonucleotide
  source 1..17: mol_type = other DNA
tctggtcgggaaactcc

SEQ ID NO: 251 - DNA, 16 bp, synthetic construct
  misc_feature 1..16: note = Synthetic Oligonucleotide
  source 1..16: mol_type = other DNA
gctacagccttttccc

SEQ ID NO: 252 - DNA, 24 bp, synthetic construct
  misc_feature 1..24: note = Synthetic Oligonucleotide
  source 1..24: mol_type = other DNA
cctccgccttctccctctttgatc

SEQ ID NO: 253 - DNA, 26 bp, synthetic construct
  misc_feature 1..26: note = Synthetic Oligonucleotide
  source 1..26: mol_type = other DNA
tctggtcgggaaactcaattattgtc

SEQ ID NO: 254 - DNA, 24 bp, synthetic construct
  misc_feature 1..24: note = Synthetic Oligonucleotide
  source 1..24: mol_type = other DNA
gctacagccttttccactttgttc

SEQ ID NO: 255 - DNA, 35 bp, synthetic construct
  misc_feature 1..35: note = Synthetic Oligonucleotide
  source 1..35: mol_type = other DNA
cctccgccttctccctttttagataaaatattata

SEQ ID NO: 256 - DNA, 34 bp, synthetic construct
  misc_feature 1..34: note = Synthetic Oligonucleotide
  source 1..34: mol_type = other DNA
tctggtcgggaaactttttagataaaatattata

SEQ ID NO: 257 - DNA, 35 bp, synthetic construct
  misc_feature 1..35: note = Synthetic Oligonucleotide
  source 1..35: mol_type = other DNA
gctacagccttttcctttttagataaaatattata

SEQ ID NO: 258 - DNA, 35 bp, synthetic construct
  misc_feature 1..35: note = Synthetic Oligonucleotide
  source 1..35: mol_type = other DNA
cctccgccttctccctttttggtttatttaaaact

SEQ ID NO: 259 - DNA, 34 bp, synthetic construct
  misc_feature 1..34: note = Synthetic Oligonucleotide
  source 1..34: mol_type = other DNA
tctggtcgggaaactttttggtttatttaaaact

SEQ ID NO: 260 - DNA, 35 bp, synthetic construct
  misc_feature 1..35: note = Synthetic Oligonucleotide
  source 1..35: mol_type = other DNA
gctacagccttttcctttttggtttatttaaaact

SEQ ID NO: 261 - DNA, 35 bp, synthetic construct
  misc_feature 1..35: note = Synthetic Oligonucleotide
  source 1..35: mol_type = other DNA
cctccgccttctccctttttaaatttatattacat

SEQ ID NO: 262 - DNA, 34 bp, synthetic construct
  misc_feature 1..34: note = Synthetic Oligonucleotide
  source 1..34: mol_type = other DNA
tctggtcgggaaactttttaaatttatattacat

SEQ ID NO: 263 - DNA, 35 bp, synthetic construct
  misc_feature 1..35: note = Synthetic Oligonucleotide
  source 1..35: mol_type = other DNA
gctacagccttttcctttttaaatttatattacat

SEQ ID NO: 264 - DNA, 25 bp, synthetic construct
  misc_feature 1..25: note = Synthetic Oligonucleotide
  source 1..25: mol_type = other DNA
gccttctccctttttagataaaata

SEQ ID NO: 265 - DNA, 24 bp, synthetic construct
  misc_feature 1..24: note = Synthetic Oligonucleotide
  source 1..24: mol_type = other DNA
tcgggaaactttttagataaaata

SEQ ID NO: 266 - DNA, 25 bp, synthetic construct
  misc_feature 1..25: note = Synthetic Oligonucleotide
  source 1..25: mol_type = other DNA
agccttttcctttttagataaaata

SEQ ID NO: 267 - DNA, 25 bp, synthetic construct
  misc_feature 1..25: note = Synthetic Oligonucleotide
  source 1..25: mol_type = other DNA
gccttctccctttttggtttattta

SEQ ID NO: 268 - DNA, 24 bp, synthetic construct
  misc_feature 1..24: note = Synthetic Oligonucleotide
  source 1..24: mol_type = other DNA
tcgggaaactttttggtttattta

SEQ ID NO: 269 - DNA, 25 bp, synthetic construct
  misc_feature 1..25: note = Synthetic Oligonucleotide
  source 1..25: mol_type = other DNA
agccttttcctttttggtttattta

SEQ ID NO: 270 - DNA, 25 bp, synthetic construct
  misc_feature 1..25: note = Synthetic Oligonucleotide
  source 1..25: mol_type = other DNA
gccttctccctttttaaatttatat

SEQ ID NO: 271 - DNA, 24 bp, synthetic construct
  misc_feature 1..24: note = Synthetic Oligonucleotide
  source 1..24: mol_type = other DNA
tcgggaaactttttaaatttatat

SEQ ID NO: 272 - DNA, 25 bp, synthetic construct
  misc_feature 1..25: note = Synthetic Oligonucleotide
  source 1..25: mol_type = other DNA
agccttttcctttttaaatttatat

SEQ ID NO: 273 - DNA, 15 bp, synthetic construct
  misc_feature 1..15: note = Synthetic Oligonucleotide
  source 1..15: mol_type = other DNA
aggtgtgcactttta

SEQ ID NO: 274 - DNA, 15 bp, synthetic construct
  misc_feature 1..15: note = Synthetic Oligonucleotide
  source 1..15: mol_type = other DNA
tcatttttaaggtgt

SEQ ID NO: 275 - DNA, 20 bp, synthetic construct
  misc_feature 1..20: note = Synthetic Oligonucleotide
  source 1..20: mol_type = other DNA
tttttaggtgtgcactttta

SEQ ID NO: 276 - DNA, 19 bp, synthetic construct
  misc_feature 1..19: note = Synthetic Oligonucleotide
  source 1..19: mol_type = other DNA
tttttcatttttaaggtgt

SEQ ID NO: 277 - DNA, 15 bp, synthetic construct
  misc_feature 1..15: note = Synthetic Oligonucleotide
  source 1..15: mol_type = other DNA
cgcggtctcggcggt

SEQ ID NO: 278 - DNA, 15 bp, synthetic construct
  misc_feature 1..15: note = Synthetic Oligonucleotide
  source 1..15: mol_type = other DNA
atcatccatggtgag

SEQ ID NO: 279 - DNA, 34 bp, synthetic construct
  misc_feature 1..34: note = Synthetic Oligonucleotide
  source 1..34: mol_type = other DNA
cgcggtctcggcggtttttaggtgtgcactttta

SEQ ID NO: 280 - DNA, 35 bp, synthetic construct
  misc_feature 1..35: note = Synthetic Oligonucleotide
  source 1..35: mol_type = other DNA
atcatccatggtgagtttttaggtgtgcactttta

SEQ ID NO: 281 - DNA, 33 bp, synthetic construct
  misc_feature 1..33: note = Synthetic Oligonucleotide
  source 1..33: mol_type = other DNA
cgcggtctcggcggtttttcatttttaaggtgt

SEQ ID NO: 282 - DNA, 34 bp, synthetic construct
  misc_feature 1..34: note = Synthetic Oligonucleotide
  source 1..34: mol_type = other DNA
atcatccatggtgagtttttcatttttaaggtgt

SEQ ID NO: 283 - DNA, 24 bp, synthetic construct
  misc_feature 1..24: note = Synthetic Oligonucleotide
  source 1..24: mol_type = other DNA
tctcggcggtttttaggtgtgcac

SEQ ID NO: 284 - DNA, 25 bp, synthetic construct
  misc_feature 1..25: note = Synthetic Oligonucleotide
  source 1..25: mol_type = other DNA
ccatggtgagtttttaggtgtgcac

SEQ ID NO: 285 - DNA, 23 bp, synthetic construct
  misc_feature 1..23: note = Synthetic Oligonucleotide
  source 1..23: mol_type = other DNA
tctcggcggtttttcatttttaa

SEQ ID NO: 286 - DNA, 24 bp, synthetic construct
  misc_feature 1..24: note = Synthetic Oligonucleotide
  source 1..24: mol_type = other DNA
ccatggtgagtttttcatttttaa

SEQ ID NO: 287 - DNA, 30 bp, synthetic construct
  misc_feature 1..30: note = Synthetic Oligonucleotide
  source 1..30: mol_type = other DNA
cgcggtctcggcggtaggtgtgcactttta

SEQ ID NO: 288 - DNA, 30 bp, synthetic construct
  misc_feature 1..30: note = Synthetic Oligonucleotide
  source 1..30: mol_type = other DNA
atcatccatggtgagaggtgtgcactttta

SEQ ID NO: 289 - DNA, 30 bp, synthetic construct
  misc_feature 1..30: note = Synthetic Oligonucleotide
  source 1..30: mol_type = other DNA
cgcggtctcggcggttcatttttaaggtgt

SEQ ID NO: 290 - DNA, 30 bp, synthetic construct
  misc_feature 1..30: note = Synthetic Oligonucleotide
  source 1..30: mol_type = other DNA
atcatccatggtgagtcatttttaaggtgt

SEQ ID NO: 291 - DNA, 15 bp, synthetic construct
  misc_feature 1..15: note = Synthetic Oligonucleotide
  source 1..15: mol_type = other DNA
tggagccgagcgctg

SEQ ID NO: 292 - DNA, 15 bp, synthetic construct
  misc_feature 1..15: note = Synthetic Oligonucleotide
  source 1..15: mol_type = other DNA
gggcctgcccctttg

SEQ ID NO: 293 - DNA, 15 bp, synthetic construct
  misc_feature 1..15: note = Synthetic Oligonucleotide
  source 1..15: mol_type = other DNA
ccccaagtcacctga

SEQ ID NO: 294 - DNA, 15 bp, synthetic construct
  misc_feature 1..15: note = Synthetic Oligonucleotide
  source 1..15: mol_type = other DNA
gacatcaatacctaa

SEQ ID NO: 295 - DNA, 15 bp, synthetic construct
  misc_feature 1..15: note = Synthetic Oligonucleotide
  source 1..15: mol_type = other DNA
aaactttaccaagtc

SEQ ID NO: 296 - DNA, 17 bp, synthetic construct
  misc_feature 1..17: note = Synthetic Oligonucleotide
  source 1..17: mol_type = other DNA
tggagccgagcgctgcc

SEQ ID NO: 297 - DNA, 17 bp, synthetic construct
  misc_feature 1..17: note = Synthetic Oligonucleotide
  source 1..17: mol_type = other DNA
gggcctgcccctttgcc

SEQ ID NO: 298 - DNA, 17 bp, synthetic construct
  misc_feature 1..17: note = Synthetic Oligonucleotide
  source 1..17: mol_type = other DNA
ccccaagtcacctgacc

SEQ ID NO: 299 - DNA, 17 bp, synthetic construct
  misc_feature 1..17: note = Synthetic Oligonucleotide
  source 1..17: mol_type = other DNA
gacatcaatacctaacc

SEQ ID NO: 300 - DNA, 17 bp, synthetic construct
  misc_feature 1..17: note = Synthetic Oligonucleotide
  source 1..17: mol_type = other DNA
aaactttaccaagtccc

SEQ ID NO: 301 - DNA, 24 bp, synthetic construct
  misc_feature 1..24: note = Synthetic Oligonucleotide
  source 1..24: mol_type = other DNA
tggagccgagcgctgggaaaccac

SEQ ID NO: 302 - DNA, 24 bp, synthetic construct
  misc_feature 1..24: note = Synthetic Oligonucleotide
  source 1..24: mol_type = other DNA
gggcctgcccctttgggaaaccac

SEQ ID NO: 303 - DNA, 24 bp, synthetic construct
  misc_feature 1..24: note = Synthetic Oligonucleotide
  source 1..24: mol_type = other DNA
ccccaagtcacctgaggaaaccac

SEQ ID NO: 304 - DNA, 24 bp, synthetic construct
  misc_feature 1..24: note = Synthetic Oligonucleotide
  source 1..24: mol_type = other DNA
gacatcaatacctaaggaaaccac

SEQ ID NO: 305 - DNA, 24 bp, synthetic construct
  misc_feature 1..24: note = Synthetic Oligonucleotide
  source 1..24: mol_type = other DNA
aaactttaccaagtcggaaaccac

SEQ ID NO: 306 - DNA, 15 bp, synthetic construct
  misc_feature 1..15: note = Synthetic Oligonucleotide
  source 1..15: mol_type = other DNA
actgcaatatatttc

SEQ ID NO: 307 - DNA, 15 bp, synthetic construct
  misc_feature 1..15: note = Synthetic Oligonucleotide
  source 1..15: mol_type = other DNA
gtgttaaaattactt

SEQ ID NO: 308 - DNA, 20 bp, synthetic construct
  misc_feature 1..20: note = Synthetic Oligonucleotide
  source 1..20: mol_type = other DNA
tttttactgcaatatatttc

SEQ ID NO: 309 - DNA, 20 bp, synthetic construct
  misc_feature 1..20: note = Synthetic Oligonucleotide
  source 1..20: mol_type = other DNA
tttttgtgttaaaattactt

SEQ ID NO: 310 - DNA, 25 bp, synthetic construct
  misc_feature 1..25: note = Synthetic Oligonucleotide
  source 1..25: mol_type = other DNA
ccgagcgctgtttttactgcaatat

SEQ ID NO: 311 - DNA, 25 bp, synthetic construct
  misc_feature 1..25: note = Synthetic Oligonucleotide
  source 1..25: mol_type = other DNA
tgcccctttgtttttactgcaatat

SEQ ID NO: 312 - DNA, 25 bp, synthetic construct
  misc_feature 1..25: note = Synthetic Oligonucleotide
  source 1..25: mol_type = other DNA
agtcacctgatttttactgcaatat

SEQ ID NO: 313 - DNA, 25 bp, synthetic construct
  misc_feature 1..25: note = Synthetic Oligonucleotide
  source 1..25: mol_type = other DNA
caatacctaatttttactgcaatat

SEQ ID NO: 314 - DNA, 25 bp, synthetic construct
  misc_feature 1..25: note = Synthetic Oligonucleotide
  source 1..25: mol_type = other DNA
ttaccaagtctttttactgcaatat

SEQ ID NO: 315 - DNA, 25 bp, synthetic construct
  misc_feature 1..25: note = Synthetic Oligonucleotide
  source 1..25: mol_type = other DNA
ccgagcgctgtttttgtgttaaaat

SEQ ID NO: 316 - DNA, 25 bp, synthetic construct
  misc_feature 1..25: note = Synthetic Oligonucleotide
  source 1..25: mol_type = other DNA
tgcccctttgtttttgtgttaaaat

SEQ ID NO: 317 - DNA, 25 bp, synthetic construct
  misc_feature 1..25: note = Synthetic Oligonucleotide
  source 1..25: mol_type = other DNA
agtcacctgatttttgtgttaaaat

SEQ ID NO: 318 - DNA, 25 bp, synthetic construct
  misc_feature 1..25: note = Synthetic Oligonucleotide
  source 1..25: mol_type = other DNA
caatacctaatttttgtgttaaaat

SEQ ID NO: 319 - DNA, 25 bp, synthetic construct
  misc_feature 1..25: note = Synthetic Oligonucleotide
  source 1..25: mol_type = other DNA
ttaccaagtctttttgtgttaaaat

SEQ ID NO: 320 - DNA, 15 bp, synthetic construct
  misc_feature 1..15: note = Synthetic Oligonucleotide
  source 1..15: mol_type = other DNA
tgtctgtagctccag

SEQ ID NO: 321 - DNA, 15 bp, synthetic construct
  misc_feature 1..15: note = Synthetic Oligonucleotide
  source 1..15: mol_type = other DNA
tagctccagtgaggc

SEQ ID NO: 322 - DNA, 15 bp, synthetic construct
  misc_feature 1..15: note = Synthetic Oligonucleotide
  source 1..15: mol_type = other DNA
tttcttctcccacca

SEQ ID NO: 323 - DNA, 17 bp, synthetic construct
  misc_feature 1..17: note = Synthetic Oligonucleotide
  source 1..17: mol_type = other DNA
tgtctgtagctccagcc

SEQ ID NO: 324 - DNA, 17 bp, synthetic construct
  misc_feature 1..17: note = Synthetic Oligonucleotide
  source 1..17: mol_type = other DNA
tagctccagtgaggccc

SEQ ID NO: 325 - DNA, 17 bp, synthetic construct
  misc_feature 1..17: note = Synthetic Oligonucleotide
  source 1..17: mol_type = other DNA
tttcttctcccaccacc

SEQ ID NO: 326 - DNA, 24 bp, synthetic construct
  misc_feature 1..24: note = Synthetic Oligonucleotide
  source 1..24: mol_type = other DNA
tgtctgtagctccagggaaaccac

SEQ ID NO: 327 - DNA, 24 bp, synthetic construct
  misc_feature 1..24: note = Synthetic Oligonucleotide
  source 1..24: mol_type = other DNA
tagctccagtgaggcggaaaccac

SEQ ID NO: 328 - DNA, 24 bp, synthetic construct
  misc_feature 1..24: note = Synthetic Oligonucleotide
  source 1..24: mol_type = other DNA
tttcttctcccaccaggaaaccac

SEQ ID NO: 329 - DNA, 20 bp, synthetic construct
  misc_feature 1..20: note = Synthetic Oligonucleotide
  source 1..20: mol_type = other DNA
tttttgtgtgatctcttagc

SEQ ID NO: 330 - DNA, 20 bp, synthetic construct
  misc_feature 1..20: note = Synthetic Oligonucleotide
  source 1..20: mol_type = other DNA
tttttgtgatctcttagcag

SEQ ID NO: 331 - DNA, 20 bp, synthetic construct
  misc_feature 1..20: note = Synthetic Oligonucleotide
  source 1..20: mol_type = other DNA
ttttttgatctcttagcaga

SEQ ID NO: 332 - DNA, 15 bp, synthetic construct
  misc_feature 1..15: note = Synthetic Oligonucleotide
  source 1..15: mol_type = other DNA
atttctctcaatcct

SEQ ID NO: 333 - DNA, 15 bp, synthetic construct
  misc_feature 1..15: note = Synthetic Oligonucleotide
  source 1..15: mol_type = other DNA
ggcgtgtatattttt

SEQ ID NO: 334 - DNA, 15 bp, synthetic construct
  misc_feature 1..15: note = Synthetic Oligonucleotide
  source 1..15: mol_type = other DNA
ggttatcgccctccc

SEQ ID NO: 335 - DNA, 15 bp, synthetic construct
  misc_feature 1..15: note = Synthetic Oligonucleotide
  source 1..15: mol_type = other DNA
acgacttccgccgcc

SEQ ID NO: 336 - DNA, 17 bp, synthetic construct
  misc_feature 1..17: note = Synthetic Oligonucleotide
  source 1..17: mol_type = other DNA
atttctctcaatcctcc

SEQ ID NO: 337 - DNA, 17 bp, synthetic construct
  misc_feature 1..17: note = Synthetic Oligonucleotide
  source 1..17: mol_type = other DNA
ggcgtgtatatttttcc

SEQ ID NO: 338 - DNA, 17 bp, synthetic construct
  misc_feature 1..17: note = Synthetic Oligonucleotide
  source 1..17: mol_type = other DNA
ggttatcgccctccccc

SEQ ID NO: 339 - DNA, 17 bp, synthetic construct
  misc_feature 1..17: note = Synthetic Oligonucleotide
  source 1..17: mol_type = other DNA
acgacttccgccgcccc

SEQ ID NO: 340 - DNA, 24 bp, synthetic construct
  misc_feature 1..24: note = Synthetic Oligonucleotide
  source 1..24: mol_type = other DNA
atttctctcaatcctggaaaccac

SEQ ID NO: 341 - DNA, 24 bp, synthetic construct
  misc_feature 1..24: note = Synthetic Oligonucleotide
  source 1..24: mol_type = other DNA
ggcgtgtatatttttggaaaccac

SEQ ID NO: 342 - DNA, 24 bp, synthetic construct
  misc_feature 1..24: note = Synthetic Oligonucleotide
  source 1..24: mol_type = other DNA
ggttatcgccctcccggaaaccac

SEQ ID NO: 343 - DNA, 24 bp, synthetic construct
  misc_feature 1..24: note = Synthetic Oligonucleotide
  source 1..24: mol_type = other DNA
acgacttccgccgccggaaaccac

SEQ ID NO: 344 - DNA, 20 bp, synthetic construct
  misc_feature 1..20: note = Synthetic Oligonucleotide
  source 1..20: mol_type = other DNA
ttttttaatttttttttaaa

SEQ ID NO: 345 - DNA, 20 bp, synthetic construct
  misc_feature 1..20: note = Synthetic Oligonucleotide
  source 1..20: mol_type = other DNA
tttttatatgcaaaaaagaa

SEQ ID NO: 346 - DNA, 20 bp, synthetic construct
  misc_feature 1..20: note = Synthetic Oligonucleotide
  source 1..20: mol_type = other DNA
tttttcaaaatatgggccaa

SEQ ID NO: 347 - DNA, 15 bp, synthetic construct
  misc_feature 1..15: note = Synthetic Oligonucleotide
  source 1..15: mol_type = other DNA
ttcaccacatgtaaa

SEQ ID NO: 348 - DNA, 19 bp, synthetic construct
  misc_feature 1..19: note = Synthetic Oligonucleotide
  source 1..19: mol_type = other DNA
ttttttcaccacatgtaaa

SEQ ID NO: 349 - DNA, 17 bp, synthetic construct
  misc_feature 1..17: note = Synthetic Oligonucleotide
  source 1..17: mol_type = other DNA
aaatcagggcagaatgt

SEQ ID NO: 350 - DNA, 19 bp, synthetic construct
  misc_feature 1..19: note = Synthetic Oligonucleotide
  source 1..19: mol_type = other DNA
aaatcagggcagaatgtcc

SEQ ID NO: 351 - DNA, 26 bp, synthetic construct
  misc_feature 1..26: note = Synthetic Oligonucleotide
  source 1..26: mol_type = other DNA
aaatcagggcagaatgtccaaaggtc

SEQ ID NO: 352 - DNA, 36 bp, synthetic construct
  misc_feature 1..36: note = Synthetic Oligonucleotide
  source 1..36: mol_type = other DNA
aaatcagggcagaatgtttttttcaccacatgtaaa

SEQ ID NO: 353 - DNA, 15 bp, synthetic construct
  misc_feature 1..15: note = Synthetic Oligonucleotide
  source 1..15: mol_type = other DNA
ttattgtctgagccc

SEQ ID NO: 354 - DNA, 18 bp, synthetic construct
  misc_feature 1..18: note = Synthetic Oligonucleotide
  source 1..18: mol_type = other DNA
tttttattgtctgagccc

SEQ ID NO: 355 - DNA, 15 bp, synthetic construct
  misc_feature 1..15: note = Synthetic Oligonucleotide
  source 1..15: mol_type = other DNA
tcaggtgacggatgt

SEQ ID NO: 356 - DNA, 17 bp, synthetic construct
  misc_feature 1..17: note = Synthetic Oligonucleotide
  source 1..17: mol_type = other DNA
tcaggtgacggatgtcc

SEQ ID NO: 357 - DNA, 24 bp, synthetic construct
  misc_feature 1..24: note = Synthetic Oligonucleotide
  source 1..24: mol_type = other DNA
tcaggtgacggatgtccaaaggtc

SEQ ID NO: 358 - DNA, 33 bp, synthetic construct
  misc_feature 1..33: note = Synthetic Oligonucleotide
  source 1..33: mol_type = other DNA
tcaggtgacggatgttttttattgtctgagccc

SEQ ID NO: 359 - DNA, 15 bp, synthetic construct
  misc_feature 1..15: note = Synthetic Oligonucleotide
  source 1..15: mol_type = other DNA
tgtggggagctcggc

SEQ ID NO: 360 - DNA, 15 bp, synthetic construct
  misc_feature 1..15: note = Synthetic Oligonucleotide
  source 1..15: mol_type = other DNA
ggggagctcggctgc

SEQ ID NO: 361 - DNA, 19 bp, synthetic construct
  misc_feature 1..19: note = Synthetic Oligonucleotide
  source 1..19: mol_type = other DNA
tttttgtggggagctcggc

SEQ ID NO: 362 - DNA, 19 bp, synthetic construct
  misc_feature 1..19: note = Synthetic Oligonucleotide
  source 1..19: mol_type = other DNA
ttttggggagctcggctgc

SEQ ID NO: 363 - DNA, 15 bp, synthetic construct
  misc_feature 1..15: note = Synthetic Oligonucleotide
  source 1..15: mol_type = other DNA
ttgtccaagggcagg

SEQ ID NO: 364 - DNA, 15 bp, synthetic construct
  misc_feature 1..15: note = Synthetic Oligonucleotide
  source 1..15: mol_type = other DNA
tcgatgagtgtgtgc

SEQ ID NO: 365 - DNA, 15 bp, synthetic construct
  misc_feature 1..15: note = Synthetic Oligonucleotide
  source 1..15: mol_type = other DNA
agaagaaaaaccacg

SEQ ID NO: 366 - DNA, 15 bp, synthetic construct
  misc_feature 1..15: note = Synthetic Oligonucleotide
  source 1..15: mol_type = other DNA
aatatgatttcttcc

SEQ ID NO: 367 - DNA, 15 bp, synthetic construct
  misc_feature 1..15: note = Synthetic Oligonucleotide
  source 1..15: mol_type = other DNA
gagatgggggacatg

SEQ ID NO: 368 - DNA, 15 bp, synthetic construct
  misc_feature 1..15: note = Synthetic Oligonucleotide
  source 1..15: mol_type = other DNA
ttcagtttattcaag

SEQ ID NO: 369 - DNA, 15 bp, synthetic construct
  misc_feature 1..15: note = Synthetic Oligonucleotide
  source 1..15: mol_type = other DNA
ctgtctccacttttt

SEQ ID NO: 370 - DNA, 14 bp, synthetic construct
  misc_feature 1..14: note = Synthetic Oligonucleotide
  source 1..14: mol_type = other DNA
tggaataaaacggg

SEQ ID NO: 371 - DNA, 15 bp, synthetic construct
  misc_feature 1..15: note = Synthetic Oligonucleotide
  source 1..15: mol_type = other DNA
acaattgagaaaaca

SEQ ID NO: 372 - DNA, 15 bp, synthetic construct
  misc_feature 1..15: note = Synthetic Oligonucleotide
  source 1..15: mol_type = other DNA
cagttttaagtggag

SEQ ID NO: 373 - DNA, 15 bp, synthetic construct
  misc_feature 1..15: note = Synthetic Oligonucleotide
  source 1..15: mol_type = other DNA
tgacaagaatgagac

SEQ ID NO: 374 - DNA, 15 bp, synthetic construct
  misc_feature 1..15: note = Synthetic Oligonucleotide
  source 1..15: mol_type = other DNA
ccgggcgaggggagg

SEQ ID NO: 375 - DNA, 15 bp, synthetic construct
  misc_feature 1..15: note = Synthetic Oligonucleotide
  source 1..15: mol_type = other DNA
ccgccggcctgcccg

SEQ ID NO: 376 - DNA, 15 bp, synthetic construct
  misc_feature 1..15: note = Synthetic Oligonucleotide
  source 1..15: mol_type = other DNA
cgagcgcgtatcctg

SEQ ID NO: 377 - DNA, 15 bp, synthetic construct
  misc_feature 1..15: note = Synthetic Oligonucleotide
  source 1..15: mol_type = other DNA
ctgcttctcctcagc

SEQ ID NO: 378 - DNA, 17 bp, synthetic construct
  misc_feature 1..17: note = Synthetic Oligonucleotide
  source 1..17: mol_type = other DNA
ttttcagtttattcaag

SEQ ID NO: 379 - DNA, 19 bp, synthetic construct
  misc_feature 1..19: note = Synthetic Oligonucleotide
  source 1..19: mol_type = other DNA
ttttctgtctccacttttt

SEQ ID NO: 380 - DNA, 18 bp, synthetic construct
  misc_feature 1..18: note = Synthetic Oligonucleotide
  source 1..18: mol_type = other DNA
tttttggaataaaacggg

SEQ ID NO: 381 - DNA, 19 bp, synthetic construct
  misc_feature 1..19: note = Synthetic Oligonucleotide
  source 1..19: mol_type = other DNA
ttttacaattgagaaaaca

SEQ ID NO: 382 - DNA, 19 bp, synthetic construct
  misc_feature 1..19: note = Synthetic Oligonucleotide
  source 1..19: mol_type = other DNA
ttttcagttttaagtggag

SEQ ID NO: 383 - DNA, 19 bp, synthetic construct
  misc_feature 1..19: note = Synthetic Oligonucleotide
  source 1..19: mol_type = other DNA
tttttgacaagaatgagac

SEQ ID NO: 384 - DNA, 15 bp, synthetic construct
  misc_feature 1..15: note = Synthetic Oligonucleotide
  source 1..15: mol_type = other DNA
aacagtcataataat

SEQ ID NO: 385 - DNA, 15 bp, synthetic construct
  misc_feature 1..15: note = Synthetic Oligonucleotide
  source 1..15: mol_type = other DNA
taatttaacagtcat

SEQ ID NO: 386 - DNA, 15 bp, synthetic construct
  misc_feature 1..15: note = Synthetic Oligonucleotide
  source 1..15: mol_type = other DNA
gcacgctataaagca

SEQ ID NO: 387 - DNA, 15 bp, synthetic construct
  misc_feature 1..15: note = Synthetic Oligonucleotide
  source 1..15: mol_type = other DNA
cccggggctgggctt

SEQ ID NO: 388 - DNA, 15 bp, synthetic construct
  misc_feature 1..15: note = Synthetic Oligonucleotide
  source 1..15: mol_type = other DNA
ccccgctccgcctcc

SEQ ID NO: 389 - DNA, 15 bp, synthetic construct
  misc_feature 1..15: note = Synthetic Oligonucleotide
  source 1..15: mol_type = other DNA
gcgcctccctgattt

SEQ ID NO: 390 - DNA, 15 bp, synthetic construct
  misc_feature 1..15: note = Synthetic Oligonucleotide
  source 1..15: mol_type = other DNA
tcgccgcggtggctg

SEQ ID NO: 391 - DNA, 15 bp, synthetic construct
  misc_feature 1..15: note = Synthetic Oligonucleotide
  source 1..15: mol_type = other DNA
cagcgaatggtcgcg

SEQ ID NO: 392 - DNA, 20 bp, synthetic construct
  misc_feature 1..20: note = Synthetic Oligonucleotide
  source 1..20: mol_type = other DNA
tttttaacagtcataataat

SEQ ID NO: 393 - DNA, 19 bp, synthetic construct
  misc_feature 1..19: note = Synthetic Oligonucleotide
  source 1..19: mol_type = other DNA
tttttaatttaacagtcat

SEQ ID NO: 394 - DNA, 15 bp, synthetic construct
  misc_feature 1..15: note = Synthetic Oligonucleotide
  source 1..15: mol_type = other DNA
gcggcggctgctcta

SEQ ID NO: 395 - DNA, 15 bp, synthetic construct
  misc_feature 1..15: note = Synthetic Oligonucleotide
  source 1..15: mol_type = other DNA
ttatcggccgctgcc

SEQ ID NO: 396 - DNA, 15 bp, synthetic construct
  misc_feature 1..15: note = Synthetic Oligonucleotide
  source 1..15: mol_type = other DNA
gcgtcggggacggct

SEQ ID NO: 397 - DNA, 15 bp, synthetic construct
  misc_feature 1..15: note = Synthetic Oligonucleotide
  source 1..15: mol_type = other DNA
gcggaggaaactgcg

SEQ ID NO: 398 - DNA, 15 bp, synthetic construct
  misc_feature 1..15: note = Synthetic Oligonucleotide
  source 1..15: mol_type = other DNA
gccgcacgcccgaca

SEQ ID NO: 399 - DNA, 15 bp, synthetic construct
  misc_feature 1..15: note = Synthetic Oligonucleotide
  source 1..15: mol_type = other DNA
cctgacccaccctcc

SEQ ID NO: 400 - DNA, 15 bp, synthetic construct
  misc_feature 1..15: note = Synthetic Oligonucleotide
  source 1..15: mol_type = other DNA
agggcaggccgcggc

SEQ ID NO: 401 - DNA, 15 bp, synthetic construct
  misc_feature 1..15: note = Synthetic Oligonucleotide
  source 1..15: mol_type = other DNA
ctgaatcaccccgcg

SEQ ID NO: 402 - DNA, 15 bp, synthetic construct
  misc_feature 1..15: note = Synthetic Oligonucleotide
  source 1..15: mol_type = other DNA
ggccccgagctccgc

SEQ ID NO: 403 - DNA, 15 bp, synthetic construct
  misc_feature 1..15: note = Synthetic Oligonucleotide
  source 1..15: mol_type = other DNA
gcggctgctctaata

SEQ ID NO: 404 - DNA, 15 bp, synthetic construct
  misc_feature 1..15: note = Synthetic Oligonucleotide
  source 1..15: mol_type = other DNA
cgccgcggcatgtgg

SEQ ID NO: 405 - DNA, 15 bp, synthetic construct
  misc_feature 1..15: note = Synthetic Oligonucleotide
  source 1..15: mol_type = other DNA
ccctcctcctcttgc

SEQ ID NO: 406 - DNA, 15 bp, synthetic construct
  misc_feature 1..15: note = Synthetic Oligonucleotide
  source 1..15: mol_type = other DNA
ggccgcgggctcgtg

SEQ ID NO: 407 - DNA, 15 bp, synthetic construct
  misc_feature 1..15: note = Synthetic Oligonucleotide
  source 1..15: mol_type = other DNA
gttatttttctctgt

SEQ ID NO: 408 - DNA, 15 bp, synthetic construct
  misc_feature 1..15: note = Synthetic Oligonucleotide
  source 1..15: mol_type = other DNA
atttaaaatgtttta

SEQ ID NO: 409 - DNA, 15 bp, synthetic construct
  misc_feature 1..15: note = Synthetic Oligonucleotide
  source 1..15: mol_type = other DNA
tctctgtccatttaa

SEQ ID NO: 410 - DNA, 15 bp, synthetic construct
  misc_feature 1..15: note = Synthetic Oligonucleotide
  source 1..15: mol_type = other DNA
tcatttggtcatgtg

SEQ ID NO: 411 - DNA, 15 bp, synthetic construct
  misc_feature 1..15: note = Synthetic Oligonucleotide
  source 1..15: mol_type = other DNA
tagttctctgtacat

SEQ ID NO: 412 - DNA, 15 bp, synthetic construct
  misc_feature 1..15: note = Synthetic Oligonucleotide
  source 1..15: mol_type = other DNA
tctgctggctcaact

SEQ ID NO: 413 - DNA, 15 bp, synthetic construct
  misc_feature 1..15: note = Synthetic Oligonucleotide
  source 1..15: mol_type = other DNA
atcatagaatagatt

SEQ ID NO: 414 - DNA, 15 bp, synthetic construct
  misc_feature 1..15: note = Synthetic Oligonucleotide
  source 1..15: mol_type = other DNA
ttatcatagaataga

SEQ ID NO: 415 - DNA, 15 bp, synthetic construct
  misc_feature 1..15: note = Synthetic Oligonucleotide
  source 1..15: mol_type = other DNA
aattgacatttagca

SEQ ID NO: 416 - DNA, 15 bp, synthetic construct
  misc_feature 1..15: note = Synthetic Oligonucleotide
  source 1..15: mol_type = other DNA
gacatttagcatttt

SEQ ID NO: 417 - DNA, 15 bp, synthetic construct
  misc_feature 1..15: note = Synthetic Oligonucleotide
  source 1..15: mol_type = other DNA
ttaaccattcaacac

SEQ ID NO: 418 - DNA, 15 bp, synthetic construct
  misc_feature 1..15: note = Synthetic Oligonucleotide
  source 1..15: mol_type = other DNA
cttggccggggaact

SEQ ID NO: 419 - DNA, 15 bp, synthetic construct
  misc_feature 1..15: note = Synthetic Oligonucleotide
  source 1..15: mol_type = other DNA
gccggggaactgccg

SEQ ID NO: 420 - DNA, 15 bp, synthetic construct
  misc_feature 1..15: note = Synthetic Oligonucleotide
  source 1..15: mol_type = other DNA
cgcccggagccgcgc

SEQ ID NO: 421 - DNA, 17 bp, synthetic construct
  misc_feature 1..17: note = Synthetic Oligonucleotide
  source 1..17: mol_type = other DNA
cttggccggggaactcc

SEQ ID NO: 422 - DNA, 16 bp, synthetic construct
  misc_feature 1..16: note = Synthetic Oligonucleotide
  source 1..16: mol_type = other DNA
gccggggaactgccgc

SEQ ID NO: 423 - DNA, 16 bp, synthetic construct
  misc_feature 1..16: note = Synthetic Oligonucleotide
  source 1..16: mol_type = other DNA
cgcccggagccgcgcc

SEQ ID NO: 424 - DNA, 24 bp, synthetic construct
  misc_feature 1..24: note = Synthetic Oligonucleotide
  source 1..24: mol_type = other DNA
cttggccggggaactataaaattc

SEQ ID NO: 425 - DNA, 34 bp, synthetic construct
  misc_feature 1..34: note = Synthetic Oligonucleotide
  source 1..34: mol_type = other DNA
cttggccggggaactttttgtcgttcagataaaa

SEQ ID NO: 426 - DNA, 32 bp, synthetic construct
  misc_feature 1..32: note = Synthetic Oligonucleotide
  source 1..32: mol_type = other DNA
cttggccggggaactttttcagataaaatatt

SEQ ID NO: 427 - DNA, 30 bp, synthetic construct
  misc_feature 1..30: note = Synthetic Oligonucleotide
  source 1..30: mol_type = other DNA
cttggccggggaactgtcgttcagataaaa

SEQ ID NO: 428 - DNA, 30 bp, synthetic construct
  misc_feature 1..30: note = Synthetic Oligonucleotide
  source 1..30: mol_type = other DNA
cttggccggggaactttcagataaaatatt

SEQ ID NO: 429 - DNA, 24 bp, synthetic construct
  misc_feature 1..24: note = Synthetic Oligonucleotide
  source 1..24: mol_type = other DNA
ccggggaactttttgtcgttcaga

SEQ ID NO: 430 - DNA, 21 bp, synthetic construct
  misc_feature 1..21: note = Synthetic Oligonucleotide
  source 1..21: mol_type = other DNA
cggggaactttttcagataaa

SEQ ID NO: 431 - DNA, 19 bp, synthetic construct
  misc_feature 1..19: note = Synthetic Oligonucleotide
  source 1..19: mol_type = other DNA
cggggaactgtcgttcaga

SEQ ID NO: 432 - DNA, 20 bp, synthetic construct
  misc_feature 1..20: note = Synthetic Oligonucleotide
  source 1..20: mol_type = other DNA
ccggggaactttcagataaa

SEQ ID NO: 433 - DNA, 15 bp, synthetic construct
  misc_feature 1..15: note = Synthetic Oligonucleotide
  source 1..15: mol_type = other DNA
gtcgttcagataaaa

SEQ ID NO: 434 - DNA, 15 bp, synthetic construct
  misc_feature 1..15: note = Synthetic Oligonucleotide
  source 1..15: mol_type = other DNA
ttcagataaaatatt

SEQ ID NO: 435 - DNA, 20 bp, synthetic construct
  misc_feature 1..20: note = Synthetic Oligonucleotide
  source 1..20: mol_type = other DNA
tttttgtcgttcagataaaa

SEQ ID NO: 436 - DNA, 18 bp, synthetic construct
  misc_feature 1..18: note = Synthetic Oligonucleotide
  source 1..18: mol_type = other DNA
tttttcagataaaatatt

SEQ ID NO: 437 - DNA, 15 bp, synthetic construct
  misc_feature 1..15: note = Synthetic Oligonucleotide
  source 1..15: mol_type = other DNA
ctccgcggccgctcc

SEQ ID NO: 438 - DNA, 15 bp, synthetic construct
  misc_feature 1..15: note = Synthetic Oligonucleotide
  source 1..15: mol_type = other DNA
gcccacatgctactc

SEQ ID NO: 439 - DNA, 15 bp, synthetic construct
  misc_feature 1..15: note = Synthetic Oligonucleotide
  source 1..15: mol_type = other DNA
tccgaacgcccacat

SEQ ID NO: 440 - DNA, 15 bp, synthetic construct
  misc_feature 1..15: note = Synthetic Oligonucleotide
  source 1..15: mol_type = other DNA
cgaggactcggtggt

SEQ ID NO: 441 - DNA, 15 bp, synthetic construct
  misc_feature 1..15: note = Synthetic Oligonucleotide
  source 1..15: mol_type = other DNA
ccagctccgcggccg

SEQ ID NO: 442 - DNA, 16 bp, synthetic construct
  misc_feature 1..16: note = Synthetic Oligonucleotide
  source 1..16: mol_type = other DNA
ctccgcggccgctccc

SEQ ID NO: 443 - DNA, 16 bp, synthetic construct
  misc_feature 1..16: note = Synthetic Oligonucleotide
  source 1..16: mol_type = other DNA
gcccacatgctactcc

SEQ ID NO: 444 - DNA, 24 bp, synthetic construct
  misc_feature 1..24: note = Synthetic Oligonucleotide
  source 1..24: mol_type = other DNA
ctccgcggccgctcctcaaagatc

SEQ ID NO: 445 - DNA, 24 bp, synthetic construct
  misc_feature 1..24: note = Synthetic Oligonucleotide
  source 1..24: mol_type = other DNA
gcccacatgctactcccaaaggtc

SEQ ID NO: 446 - DNA, 35 bp, synthetic construct
  misc_feature 1..35: note = Synthetic Oligonucleotide
  source 1..35: mol_type = other DNA
ctccgcggccgctcctttttgggagggaacacact

SEQ ID NO: 447 - DNA, 35 bp, synthetic construct
  misc_feature 1..35: note = Synthetic Oligonucleotide
  source 1..35: mol_type = other DNA
gcccacatgctactctttttgggagggaacacact

SEQ ID NO: 448 - DNA, 30 bp, synthetic construct
  misc_feature 1..30: note = Synthetic Oligonucleotide
  source 1..30: mol_type = other DNA
ctccgcggccgctccgggagggaacacact

SEQ ID NO: 449 - DNA, 30 bp, synthetic construct
  misc_feature 1..30: note = Synthetic Oligonucleotide
  source 1..30: mol_type = other DNA
gcccacatgctactcgggagggaacacact

SEQ ID NO: 450 - DNA, 20 bp, synthetic construct
  misc_feature 1..20: note = Synthetic Oligonucleotide
  source 1..20: mol_type = other DNA
cggccgctccgggagggaac

SEQ ID NO: 451 - DNA, 20 bp, synthetic construct
  misc_feature 1..20: note = Synthetic Oligonucleotide
  source 1..20: mol_type = other DNA
catgctactcgggagggaac

SEQ ID NO: 452 - DNA, 15 bp, synthetic construct
  misc_feature 1..15: note = Synthetic Oligonucleotide
  source 1..15: mol_type = other DNA
gggagggaacacact

SEQ ID NO: 453 - DNA, 15 bp, synthetic construct
  misc_feature 1..15: note = Synthetic Oligonucleotide
  source 1..15: mol_type = other DNA
ggggtcttcacctga

SEQ ID NO: 454 - DNA, 15 bp, synthetic construct
  misc_feature 1..15: note = Synthetic Oligonucleotide
  source 1..15: mol_type = other DNA
ggctgttatatcatg

SEQ ID NO: 455 - DNA, 15 bp, synthetic construct
  misc_feature 1..15: note = Synthetic Oligonucleotide
  source 1..15: mol_type = other DNA
ggcattttaagatgg

SEQ ID NO: 456 - DNA, 20 bp, synthetic construct
  misc_feature 1..20: note = Synthetic Oligonucleotide
  source 1..20: mol_type = other DNA
tttttgggagggaacacact

SEQ ID NO: 457 - DNA, 20 bp, synthetic construct
  misc_feature 1..20: note = Synthetic Oligonucleotide
  source 1..20: mol_type = other DNA
tttttggctgttatatcatg

SEQ ID NO: 458 - DNA, 18 bp, synthetic construct
  misc_feature 1..18: note = Synthetic Oligonucleotide
  source 1..18: mol_type = other DNA
ttttttttttggttttcc

SEQ ID NO: 459 - DNA, 15 bp, synthetic construct
  misc_feature 1..15: note = Synthetic Oligonucleotide
  source 1..15: mol_type = other DNA
tgtctcatttggaga

SEQ ID NO: 460 - DNA, 14 bp, synthetic construct
  misc_feature 1..14: note = Synthetic Oligonucleotide
  source 1..14: mol_type = other DNA
ataatgaagctggg

SEQ ID NO: 461 - DNA, 15 bp, synthetic construct
  misc_feature 1..15: note = Synthetic Oligonucleotide
  source 1..15: mol_type = other DNA
ttttccctcctggaa

SEQ ID NO: 462 - DNA, 15 bp, synthetic construct
  misc_feature 1..15: note = Synthetic Oligonucleotide
  source 1..15: mol_type = other DNA
tgcataatgaagctg

SEQ ID NO: 463 - DNA, 15 bp, synthetic construct
  misc_feature 1..15: note = Synthetic Oligonucleotide
  source 1..15: mol_type = other DNA
aaatccttcaaagaa

SEQ ID NO: 464 - DNA, 15 bp, synthetic construct
  misc_feature 1..15: note = Synthetic Oligonucleotide
  source 1..15: mol_type = other DNA
ttggaagattttttg

SEQ ID NO: 465 - DNA, 15 bp, synthetic construct
  misc_feature 1..15: note = Synthetic Oligonucleotide
  source 1..15: mol_type = other DNA
gcattcttgtagcag

SEQ ID NO: 466 - DNA, 15 bp, synthetic construct
  misc_feature 1..15: note = Synthetic Oligonucleotide
  source 1..15: mol_type = other DNA
acaacaaaaaacaga

SEQ ID NO: 467 - DNA, 15 bp, synthetic construct
  misc_feature 1..15: note = Synthetic Oligonucleotide
  source 1..15: mol_type = other DNA
tgaagctggggtctt

SEQ ID NO: 468 - DNA, 15 bp, synthetic construct
  misc_feature 1..15: note = Synthetic Oligonucleotide
  source 1..15: mol_type = other DNA
cctgaaaacatttgt

SEQ ID NO: 469 - DNA, 15 bp, synthetic construct
  misc_feature 1..15: note = Synthetic Oligonucleotide
  source 1..15: mol_type = other DNA
ttcattttccctcct

SEQ ID NO: 470 - DNA, 15 bp, synthetic construct
  misc_feature 1..15: note = Synthetic Oligonucleotide
  source 1..15: mol_type = other DNA
ttattattattatat

SEQ ID NO: 471 - DNA, 15 bp, synthetic construct
  misc_feature 1..15: note = Synthetic Oligonucleotide
  source 1..15: mol_type = other DNA
taactttgcatgaat

SEQ ID NO: 472 - DNA, 15 bp, synthetic construct
  misc_feature 1..15: note = Synthetic Oligonucleotide
  source 1..15: mol_type = other DNA
atacaaacatgtatg

SEQ ID NO: 473 - DNA, 15 bp, synthetic construct
  misc_feature 1..15: note = Synthetic Oligonucleotide
  source 1..15: mol_type = other DNA
attgtaaacctataa

SEQ ID NO: 474 - DNA, 15 bp, synthetic construct
  misc_feature 1..15: note = Synthetic Oligonucleotide
  source 1..15: mol_type = other DNA
tggagttggggttat

SEQ ID NO: 475 - DNA, 15 bp, synthetic construct
  misc_feature 1..15: note = Synthetic Oligonucleotide
  source 1..15: mol_type = other DNA
gttggggttatttag

SEQ ID NO: 476 - DNA, 13 bp, synthetic construct
  misc_feature 1..13: note = Synthetic Oligonucleotide
  source 1..13: mol_type = other DNA
ctccgccctccag

SEQ ID NO: 477 - DNA, 11 bp, synthetic construct
  misc_feature 1..11: note = Synthetic Oligonucleotide
  source 1..11: mol_type = other DNA
ccgccctccag

SEQ ID NO: 478 - skipped

SEQ ID NO: 479 - DNA, 15 bp, synthetic construct
  misc_feature 1..15: note = Synthetic Oligonucleotide
  source 1..15: mol_type = other DNA
cccgctccgccctcc

SEQ ID NO: 480 - DNA, 13 bp, synthetic construct
  misc_feature 1..13: note = Synthetic Oligonucleotide
  source 1..13: mol_type = other DNA
cgctccgccctcc

SEQ ID NO: 481 - DNA, 11 bp, synthetic construct
  misc_feature 1..11: note = Synthetic Oligonucleotide
  source 1..11: mol_type = other DNA
ctccgccctcc

SEQ ID NO: 482 - skipped

SEQ ID NO: 483 - DNA, 13 bp, synthetic construct
  misc_feature 1..13: note = Synthetic Oligonucleotide
  source 1..13: mol_type = other DNA
gccactggccgca

SEQ ID NO: 484 - DNA, 11 bp, synthetic construct
  misc_feature 1..11: note = Synthetic Oligonucleotide
  source 1..11: mol_type = other DNA
cactggccgca

SEQ ID NO: 485 - DNA, 13 bp, synthetic construct
  misc_feature 1..13: note = Synthetic Oligonucleotide
  source 1..13: mol_type = other DNA
gcgacccctggtg

SEQ ID NO: 486 - DNA, 11 bp, synthetic construct
  misc_feature 1..11: note = Synthetic Oligonucleotide
  source 1..11: mol_type = other DNA
gacccctggtg

SEQ ID NO: 487 - DNA, 13 bp, synthetic construct
  misc_feature 1..13: note = Synthetic Oligonucleotide
  source 1..13: mol_type = other DNA
ctggccgcaggca

SEQ ID NO: 488 - DNA, 13 bp, synthetic construct
  misc_feature 1..13: note = Synthetic Oligonucleotide
  source 1..13: mol_type = other DNA
ggccactggccgc

SEQ ID NO: 489 - DNA, 13 bp, synthetic construct
  misc_feature 1..13: note = Synthetic Oligonucleotide
  source 1..13: mol_type = other DNA
ctggtggccactg

SEQ ID NO: 490 - DNA, 13 bp, synthetic construct
  misc_feature 1..13: note = Synthetic Oligonucleotide
  source 1..13: mol_type = other DNA
gacccctggtggc

SEQ ID NO: 491 - DNA, 13 bp, synthetic construct
  misc_feature 1..13: note = Synthetic Oligonucleotide
  source 1..13: mol_type = other DNA
gcggcgacccctg

SEQ ID NO: 492 - DNA, 13 bp, synthetic construct
  misc_feature 1..13: note = Synthetic Oligonucleotide
  source 1..13: mol_type = other DNA
gtgctgcggcgac

SEQ ID NO: 493 - DNA, 13 bp, synthetic construct
  misc_feature 1..13: note = Synthetic Oligonucleotide
  source 1..13: mol_type = other DNA
gctgggtgctgcg

SEQ ID NO: 494 - DNA, 13 bp, synthetic construct
  misc_feature 1..13: note = Synthetic Oligonucleotide
  source 1..13: mol_type = other DNA
ccagcgctgggtg

SEQ ID NO: 495 - DNA, 13 bp, synthetic construct
  misc_feature 1..13: note = Synthetic Oligonucleotide
  source 1..13: mol_type = other DNA
gccctccagcgct

SEQ ID NO: 496 - DNA, 13 bp, synthetic construct
  misc_feature 1..13: note = Synthetic Oligonucleotide
  source 1..13: mol_type = other DNA
cgcccgctccgcc

SEQ ID NO: 497 - DNA, 35 bp, synthetic construct; identical to SEQ ID NO: 142
  misc_feature 1..35: note = Synthetic Oligonucleotide
  source 1..35: mol_type = other DNA

SEQ ID NO: 498 - DNA, 35 bp, synthetic construct; identical to SEQ ID NO: 143
  misc_feature 1..35: note = Synthetic Oligonucleotide
  source 1..35: mol_type = other DNA

SEQ ID NO: 499 - DNA, 17 bp, synthetic construct
  misc_feature 1..17: note = Synthetic Oligonucleotide
  source 1..17: mol_type = other DNA
caagtccagtttggttt

SEQ ID NO: 500 - DNA, 17 bp, synthetic construct
  misc_feature 1..17: note = Synthetic Oligonucleotide
  source 1..17: mol_type = other DNA
gaataggccaaggaaga

SEQ ID NO: 501 - DNA, 17 bp, synthetic construct
  misc_feature 1..17: note = Synthetic Oligonucleotide
  source 1..17: mol_type = other DNA
atcaagcatcttttccg

SEQ ID NO: 502 - DNA, 17 bp, synthetic construct
  misc_feature 1..17: note = Synthetic Oligonucleotide
  source 1..17: mol_type = other DNA
ttaaaacggggctgggc

SEQ ID NO: 503 - DNA, 17 bp, synthetic construct
  misc_feature 1..17: note = Synthetic Oligonucleotide
  source 1..17: mol_type = other DNA
gatagcttttaatgtcc

SEQ ID NO: 504 - DNA, 17 bp, synthetic construct
  misc_feature 1..17: note = Synthetic Oligonucleotide
  source 1..17: mol_type = other DNA
agctggggtcttggcct

SEQ ID NO: 505 - DNA, 17 bp, synthetic construct
  misc_feature 1..17: note = Synthetic Oligonucleotide
  source 1..17: mol_type = other DNA
cctcagctgcataatga

SEQ ID NO: 506 - DNA, 17 bp, synthetic construct
  misc_feature 1..17: note = Synthetic Oligonucleotide
  source 1..17: mol_type = other DNA
caacaacaaaaaacaga

SEQ ID NO: 507 - DNA, 17 bp, synthetic construct
  misc_feature 1..17: note = Synthetic Oligonucleotide
  source 1..17: mol_type = other DNA
aaaaaaataaacaacaa

SEQ ID NO: 508 - DNA, 17 bp, synthetic construct
  misc_feature 1..17: note = Synthetic Oligonucleotide
  source 1..17: mol_type = other DNA
cctcaaaagcaggaata

SEQ ID NO: 509 - DNA, 17 bp, synthetic construct
  misc_feature 1..17: note = Synthetic Oligonucleotide
  source 1..17: mol_type = other DNA
acacatagcccaactgt

SEQ ID NO: 510 - DNA, 17 bp, synthetic construct
  misc_feature 1..17: note = Synthetic Oligonucleotide
  source 1..17: mol_type = other DNA
ctttctacagagctgtg

SEQ ID NO: 511 - DNA, 17 bp, synthetic construct
  misc_feature 1..17: note = Synthetic Oligonucleotide
  source 1..17: mol_type = other DNA
gtaggaggcaacacatt

SEQ ID NO: 512 - DNA, 17 bp, synthetic construct
  misc_feature 1..17: note = Synthetic Oligonucleotide
  source 1..17: mol_type = other DNA
cagaacttgggggcaag

SEQ ID NO: 513 - DNA, 17 bp, synthetic construct
  misc_feature 1..17: note = Synthetic Oligonucleotide
  source 1..17: mol_type = other DNA
ccatagaaattaaaaat

SEQ ID NO: 514 - DNA, 17 bp, synthetic construct
  misc_feature 1..17: note = Synthetic Oligonucleotide
  source 1..17: mol_type = other DNA
acaatccaaaaaatctt

SEQ ID NO: 515 - DNA, 17 bp, synthetic construct
  misc_feature 1..17: note = Synthetic Oligonucleotide
  source 1..17: mol_type = other DNA
gtgagggaggaaatccg

SEQ ID NO: 516 - DNA, 17 bp, synthetic construct
  misc_feature 1..17: note = Synthetic Oligonucleotide
  source 1..17: mol_type = other DNA
aagataaggggtatcat

SEQ ID NO: 517 - DNA, 17 bp, synthetic construct
  misc_feature 1..17: note = Synthetic Oligonucleotide
  source 1..17: mol_type = other DNA
ggcataagacattataa

SEQ ID NO: 518 - DNA, 17 bp, synthetic construct
  misc_feature 1..17: note = Synthetic Oligonucleotide
  source 1..17: mol_type = other DNA
tgttatattcaggtata

SEQ ID NO: 519 - DNA, 17 bp, synthetic construct
  misc_feature 1..17: note = Synthetic Oligonucleotide
  source 1..17: mol_type = other DNA
tttgcttttttaaaggt

SEQ ID NO: 520 - DNA, 17 bp, synthetic construct
  misc_feature 1..17: note = Synthetic Oligonucleotide
  source 1..17: mol_type = other DNA
tttttccttcttattat

SEQ ID NO: 521 - DNA, 17 bp, synthetic construct
  misc_feature 1..17: note = Synthetic Oligonucleotide
  source 1..17: mol_type = other DNA
cattttccctcctggaa

SEQ ID NO: 522 - DNA, 17 bp, synthetic construct
  misc_feature 1..17: note = Synthetic Oligonucleotide
  source 1..17: mol_type = other DNA
gaagagtgaagacaatt

SEQ ID NO: 523 - DNA, 17 bp, synthetic construct
  misc_feature 1..17: note = Synthetic Oligonucleotide
  source 1..17: mol_type = other DNA
taaatccttcaaagaat

SEQ ID NO: 524 - DNA, 17 bp, synthetic construct
  misc_feature 1..17: note = Synthetic Oligonucleotide
  source 1..17: mol_type = other DNA
tcatgtacttcttgcag

SEQ ID NO: 525 - DNA, 17 bp, synthetic construct
  misc_feature 1..17: note = Synthetic Oligonucleotide
  source 1..17: mol_type = other DNA
ggttgaccagctgctct

SEQ ID NO: 526 - DNA, 17 bp, synthetic construct
  misc_feature 1..17: note = Synthetic Oligonucleotide
  source 1..17: mol_type = other DNA
agatagaacagtgagca

SEQ ID NO: 527 - DNA, 17 bp, synthetic construct
  misc_feature 1..17: note = Synthetic Oligonucleotide
  source 1..17: mol_type = other DNA
taatgtgtctcatttgg

SEQ ID NO: 528 - DNA, 17 bp, synthetic construct
  misc_feature 1..17: note = Synthetic Oligonucleotide
  source 1..17: mol_type = other DNA
atttgtaggctaccctt

SEQ ID NO: 529 - DNA, 17 bp, synthetic construct
  misc_feature 1..17: note = Synthetic Oligonucleotide
  source 1..17: mol_type = other DNA
gaaagaagcctgaaaac

SEQ ID NO: 530 - DNA, 17 bp, synthetic construct
  misc_feature 1..17: note = Synthetic Oligonucleotide
  source 1..17: mol_type = other DNA
agaagtgcttacacttt

SEQ ID NO: 531 - DNA, 17 bp, synthetic construct
  misc_feature 1..17: note = Synthetic Oligonucleotide
  source 1..17: mol_type = other DNA
tcaatgctaaagagctc

SEQ ID NO: 532 - DNA, 13 bp, synthetic construct
  misc_feature 1..13: note = Synthetic Oligonucleotide
  source 1..13: mol_type = other DNA
agtctgggtgtcc

SEQ ID NO: 533 - DNA, 13 bp, synthetic construct
  misc_feature 1..13: note = Synthetic Oligonucleotide
  source 1..13: mol_type = other DNA
ccgacagtctggg

SEQ ID NO: 534 - DNA, 13 bp, synthetic construct
  misc_feature 1..13: note = Synthetic Oligonucleotide
  source 1..13: mol_type = other DNA
ctccgacagtctg

SEQ ID NO: 535 - DNA, 13 bp, synthetic construct
  misc_feature 1..13: note = Synthetic Oligonucleotide
  source 1..13: mol_type = other DNA
gacagtctgggtg

SEQ ID NO: 536 - DNA, 11 bp, synthetic construct
  misc_feature 1..11: note = Synthetic Oligonucleotide
  source 1..11: mol_type = other DNA
cagtctgggtg

SEQ ID NO: 537 - DNA, 15 bp, synthetic construct
  misc_feature 1..15: note = Synthetic Oligonucleotide
  source 1..15: mol_type = other DNA
ctcagcctggccctg

SEQ ID NO: 538 - DNA, 15 bp, synthetic construct
  misc_feature 1..15: note = Synthetic Oligonucleotide
  source 1..15: mol_type = other DNA
agttcaaggatcagc

SEQ ID NO: 539 - DNA, 15 bp, synthetic construct
  misc_feature 1..15: note = Synthetic Oligonucleotide
  source 1..15: mol_type = other DNA
gctctccgacagtct

SEQ ID NO: 540 - DNA, 13 bp, synthetic construct
  misc_feature 1..13: note = Synthetic Oligonucleotide
  source 1..13: mol_type = other DNA
tctccgacagtct

SEQ ID NO: 541 - DNA, 11 bp, synthetic construct
  misc_feature 1..11: note = Synthetic Oligonucleotide
  source 1..11: mol_type = other DNA
tccgacagtct

SEQ ID NO: 542 - DNA, 15 bp, synthetic construct
  misc_feature 1..15: note = Synthetic Oligonucleotide
  source 1..15: mol_type = other DNA
cggagctctccgaca

SEQ ID NO: 543 - DNA, 13 bp, synthetic construct
  misc_feature 1..13: note = Synthetic Oligonucleotide
  source 1..13: mol_type = other DNA
gagctctccgaca

SEQ ID NO: 544 - DNA, 11 bp, synthetic construct
  misc_feature 1..11: note = Synthetic Oligonucleotide
  source 1..11: mol_type = other DNA
gctctccgaca

SEQ ID NO: 545 - DNA, 15 bp, synthetic construct
  misc_feature 1..15: note = Synthetic Oligonucleotide
  source 1..15: mol_type = other DNA
ctattccattttgga

SEQ ID NO: 546 - DNA, 13 bp, synthetic construct
  misc_feature 1..13: note = Synthetic Oligonucleotide
  source 1..13: mol_type = other DNA
ctattccattttg

SEQ ID NO: 547 - DNA, 15 bp, synthetic construct
  misc_feature 1..15: note = Synthetic Oligonucleotide
  source 1..15: mol_type = other DNA
attccattttggaaa

SEQ ID NO: 548 - DNA, 15 bp, synthetic construct
  misc_feature 1..15: note = Synthetic Oligonucleotide
  source 1..15: mol_type = other DNA
ccattttggaaaggt

SEQ ID NO: 549 - DNA, 13 bp, synthetic construct
  misc_feature 1..13: note = Synthetic Oligonucleotide
  source 1..13: mol_type = other DNA
ccattttggaaag

SEQ ID NO: 550 - DNA, 15 bp, synthetic construct
  misc_feature 1..15: note = Synthetic Oligonucleotide
  source 1..15: mol_type = other DNA
cattttggaaaggtt

SEQ ID NO: 551 - DNA, 13 bp, synthetic construct
  misc_feature 1..13: note = Synthetic Oligonucleotide
  source 1..13: mol_type = other DNA
cattttggaaagg

SEQ ID NO: 552 - DNA, 15 bp, synthetic construct
  misc_feature 1..15: note = Synthetic Oligonucleotide
  source 1..15: mol_type = other DNA
ggaaaggtttattgt

SEQ ID NO: 553 - DNA, 22 bp, synthetic construct
  misc_feature 1..22: note = Synthetic Oligonucleotide
  source 1..22: mol_type = other DNA
tccgacagtctccattttggaa

SEQ ID NO: 554 - DNA, 22 bp, synthetic construct
  misc_feature 1..22: note = Synthetic Oligonucleotide
  source 1..22: mol_type = other DNA
gctctccgacaccattttggaa

SEQ ID NO: 555 - DNA, 22 bp, synthetic construct
  misc_feature 1..22: note = Synthetic Oligonucleotide
  source 1..22: mol_type = other DNA
tccgacagtctcattttggaaa

SEQ ID NO: 556 - DNA, 22 bp, synthetic construct
  misc_feature 1..22: note = Synthetic Oligonucleotide
  source 1..22: mol_type = other DNA
gctctccgacacattttggaaa

SEQ ID NO: 557 - DNA, 15 bp, synthetic construct
  misc_feature 1..15: note = Synthetic Oligonucleotide
  source 1..15: mol_type = other DNA
cctcaaaagcaggaa

SEQ ID NO: 558 - DNA, 13 bp, synthetic construct
  misc_feature 1..13: note = Synthetic Oligonucleotide
  source 1..13: mol_type = other DNA
cctcaaaagcagg

SEQ ID NO: 559 - DNA, 11 bp, synthetic construct
  misc_feature 1..11: note = Synthetic Oligonucleotide
  source 1..11: mol_type = other DNA
cctcaaaagca

SEQ ID NO: 560 - DNA, 13 bp, synthetic construct
  misc_feature 1..13: note = Synthetic Oligonucleotide
  source 1..13: mol_type = other DNA
tcaaaagcaggaa

SEQ ID NO: 561 - DNA, 11 bp, synthetic construct
  misc_feature 1..11: note = Synthetic Oligonucleotide
  source 1..11: mol_type = other DNA
caaaagcagga

SEQ ID NO: 562 - DNA, 27 bp, synthetic construct
  misc_feature 1..27: note = Synthetic Oligonucleotide
  source 1..27: mol_type = other DNA
ccgccctccagcctcaaaagcaggaat

SEQ ID NO: 563 - DNA, 25 bp, synthetic construct
  misc_feature 1..25: note = Synthetic Oligonucleotide
  source 1..25: mol_type = other DNA
ccgccctccagcctcaaaagcagga

SEQ ID NO: 564 - DNA, 23 bp, synthetic construct
  misc_feature 1..23: note = Synthetic Oligonucleotide
  source 1..23: mol_type = other DNA
ccgccctccagcctcaaaagcag

SEQ ID NO: 565 - DNA, 21 bp, synthetic construct
  misc_feature 1..21: note = Synthetic Oligonucleotide
  source 1..21: mol_type = other DNA
ccgccctccagcctcaaaagc

SEQ ID NO: 566 - DNA, 25 bp, synthetic construct
  misc_feature 1..25: note = Synthetic Oligonucleotide
  source 1..25: mol_type = other DNA
gccctccagcctcaaaagcaggaat

SEQ ID NO: 567 - DNA, 23 bp, synthetic construct
  misc_feature 1..23: note = Synthetic Oligonucleotide
  source 1..23: mol_type = other DNA
gccctccagcctcaaaagcagga

SEQ ID NO: 568 - DNA, 21 bp, synthetic construct
  misc_feature 1..21: note = Synthetic Oligonucleotide
  source 1..21: mol_type = other DNA
gccctccagcctcaaaagcag

SEQ ID NO: 569 - DNA, 19 bp, synthetic construct
  misc_feature 1..19: note = Synthetic Oligonucleotide
  source 1..19: mol_type = other DNA
gccctccagcctcaaaagc

SEQ ID NO: 570 - DNA, 17 bp, synthetic construct
  misc_feature 1..17: note = Synthetic Oligonucleotide
  source 1..17: mol_type = other DNA
ccctccagcctcaaaag

SEQ ID NO: 571 - DNA, 15 bp, synthetic construct
  misc_feature 1..15: note = Synthetic Oligonucleotide
  source 1..15: mol_type = other DNA
cctccagcctcaaaa

SEQ ID NO: 572 - DNA, 21 bp, synthetic construct
  misc_feature 1..21: note = Synthetic Oligonucleotide
  source 1..21: mol_type = other DNA
gccctccagtcaaaagcagga

SEQ ID NO: 573 - DNA, 19 bp, synthetic construct
  misc_feature 1..19: note = Synthetic Oligonucleotide
  source 1..19: mol_type = other DNA
gccctccagcaaaagcagg

SEQ ID NO: 574 - DNA, 23 bp, synthetic construct
  misc_feature 1..23: note = Synthetic Oligonucleotide
  source 1..23: mol_type = other DNA
ccgccctccagtcaaaagcagga

SEQ ID NO: 575 - DNA, 21 bp, synthetic construct
  misc_feature 1..21: note = Synthetic Oligonucleotide
  source 1..21: mol_type = other DNA
ccgccctccagcaaaagcagg

SEQ ID NO: 576 - DNA, 13 bp, synthetic construct
  misc_feature 1..13: note = Synthetic Oligonucleotide
  source 1..13: mol_type = other DNA
ctccgccctccag

SEQ ID NO: 577 - DNA, 11 bp, synthetic construct
  misc_feature 1..11: note = Synthetic Oligonucleotide
  source 1..11: mol_type = other DNA
ccgccctccag

SEQ ID NO: 578 - skipped

SEQ ID NO: 579 - DNA, 15 bp, synthetic construct
  misc_feature 1..15: note = Synthetic Oligonucleotide
  source 1..15: mol_type = other DNA
cccgctccgccctcc

SEQ ID NO: 580 - DNA, 13 bp, synthetic construct
  misc_feature 1..13: note = Synthetic Oligonucleotide
  source 1..13: mol_type = other DNA
cgctccgccctcc

SEQ ID NO: 581 - DNA, 11 bp, synthetic construct
  misc_feature 1..11: note = Synthetic Oligonucleotide
  source 1..11: mol_type = other DNA
ctccgccctcc

SEQ ID NO: 582 - skipped

SEQ ID NO: 583 - DNA, 15 bp, synthetic construct
  misc_feature 1..15: note = Synthetic Oligonucleotide
  source 1..15: mol_type = other DNA
gcctttgagaaagca

SEQ ID NO: 584 - DNA, 15 bp, synthetic construct
  misc_feature 1..15: note = Synthetic Oligonucleotide
  source 1..15: mol_type = other DNA
gactgtggggccttt

SEQ ID NO: 585 - DNA, 15 bp, synthetic construct
  misc_feature 1..15: note = Synthetic Oligonucleotide
  source 1..15: mol_type = other DNA
aggaagtggaggact

SEQ ID NO: 586 - DNA, 15 bp, synthetic construct
  misc_feature 1..15: note = Synthetic Oligonucleotide
  source 1..15: mol_type = other DNA
tgcattttcactgaa

SEQ ID NO: 587 - DNA, 15 bp, synthetic construct
  misc_feature 1..15: note = Synthetic Oligonucleotide
  source 1..15: mol_type = other DNA
cattttcactgaagc

SEQ ID NO: 588 - DNA, 15 bp, synthetic construct
  misc_feature 1..15: note = Synthetic Oligonucleotide
  source 1..15: mol_type = other DNA
actgaagcatttatt

SEQ ID NO: 589 - DNA, 15 bp, synthetic construct
  misc_feature 1..15: note = Synthetic Oligonucleotide
  source 1..15: mol_type = other DNA
cacacaaatgtatgg

SEQ ID NO: 590 - DNA, 15 bp, synthetic construct
  misc_feature 1..15: note = Synthetic Oligonucleotide
  source 1..15: mol_type = other DNA
ggattttattgacaa

SEQ ID NO: 591 - DNA, 15 bp, synthetic construct
  misc_feature 1..15: note = Synthetic Oligonucleotide
  source 1..15: mol_type = other DNA
aaaacaacaaagttt

SEQ ID NO: 592 - DNA, 15 bp, synthetic construct
  misc_feature 1..15: note = Synthetic Oligonucleotide
  source 1..15: mol_type = other DNA
agtgccataaaaagt

SEQ ID NO: 593 - DNA, 15 bp, synthetic construct
  misc_feature 1..15: note = Synthetic Oligonucleotide
  source 1..15: mol_type = other DNA
tcaaatataaaaatt

SEQ ID NO: 594 - DNA, 15 bp, synthetic construct
  misc_feature 1..15: note = Synthetic Oligonucleotide
  source 1..15: mol_type = other DNA
ttccccccacccacc

SEQ ID NO: 595 - DNA, 15 bp, synthetic construct
  misc_feature 1..15: note = Synthetic Oligonucleotide
  source 1..15: mol_type = other DNA
catttgcttccaatt

SEQ ID NO: 596 - DNA, 15 bp, synthetic construct
  misc_feature 1..15: note = Synthetic Oligonucleotide
  source 1..15: mol_type = other DNA
gctcaaccctttttc

SEQ ID NO: 597 - DNA, 15 bp, synthetic construct
  misc_feature 1..15: note = Synthetic Oligonucleotide
  source 1..15: mol_type = other DNA
agacctactactctg

SEQ ID NO: 598 - DNA, 15 bp, synthetic construct
  misc_feature 1..15: note = Synthetic Oligonucleotide
  source 1..15: mol_type = other DNA
ccctccaccggaagt

SEQ ID NO: 599 - DNA, 15 bp, synthetic construct
  misc_feature 1..15: note = Synthetic Oligonucleotide
  source 1..15: mol_type = other DNA
gcccgcgctcgccgt

SEQ ID NO: 600 - DNA, 15 bp, synthetic construct
  misc_feature 1..15: note = Synthetic Oligonucleotide
  source 1..15: mol_type = other DNA
acgccccctggcagc

SEQ ID NO: 601 - DNA, 15 bp, synthetic construct
  misc_feature 1..15: note = Synthetic Oligonucleotide
  source 1..15: mol_type = other DNA
gctcagcccctcggc

SEQ ID NO: 602 - DNA, 15 bp, synthetic construct
  misc_feature 1..15: note = Synthetic Oligonucleotide
  source 1..15: mol_type = other DNA
agcagaggaagatca

SEQ ID NO: 603 - DNA, 15 bp, synthetic construct
  misc_feature 1..15: note = Synthetic Oligonucleotide
  source 1..15: mol_type = other DNA
cagaggaagatcaaa

SEQ ID NO: 604 - DNA, 15 bp, synthetic construct
  misc_feature 1..15: note = Synthetic Oligonucleotide
  source 1..15: mol_type = other DNA
cagatttttgaaact

SEQ ID NO: 605 - DNA, 15 bp, synthetic construct
  misc_feature 1..15: note = Synthetic Oligonucleotide
  source 1..15: mol_type = other DNA
cagactaattttttg

SEQ ID NO: 606 - DNA, 15 bp, synthetic construct
  misc_feature 1..15: note = Synthetic Oligonucleotide
  source 1..15: mol_type = other DNA
tttttgctttttcat

SEQ ID NO: 607 - DNA, 15 bp, synthetic construct
  misc_feature 1..15: note = Synthetic Oligonucleotide
  source 1..15: mol_type = other DNA
aattttttgcttttt

SEQ ID NO: 608 - DNA, 15 bp, synthetic construct
  misc_feature 1..15: note = Synthetic Oligonucleotide
  source 1..15: mol_type = other DNA
atgtttggcaatact

SEQ ID NO: 609 - DNA, 15 bp, synthetic construct
  misc_feature 1..15: note = Synthetic Oligonucleotide
  source 1..15: mol_type = other DNA
ttggcaatacttttt

SEQ ID NO: 610 - DNA, 15 bp, synthetic construct
  misc_feature 1..15: note = Synthetic Oligonucleotide
  source 1..15: mol_type = other DNA
gctgccctggccccg

SEQ ID NO: 611 - DNA, 15 bp, synthetic construct
  misc_feature 1..15: note = Synthetic Oligonucleotide
  source 1..15: mol_type = other DNA
cggacacacccctcg

SEQ ID NO: 612 - DNA, 15 bp, synthetic construct
  misc_feature 1..15: note = Synthetic Oligonucleotide
  source 1..15: mol_type = other DNA
acgggacgcgagtcc

SEQ ID NO: 613 - DNA, 15 bp, synthetic construct
  misc_feature 1..15: note = Synthetic Oligonucleotide
  source 1..15: mol_type = other DNA
gtctggggagaaagc

SEQ ID NO: 614 - DNA, 15 bp, synthetic construct
  misc_feature 1..15: note = Synthetic Oligonucleotide
  source 1..15: mol_type = other DNA
ccactcggtgggtct

SEQ ID NO: 615 - DNA, 15 bp, synthetic construct
  misc_feature 1..15: note = Synthetic Oligonucleotide
  source 1..15: mol_type = other DNA
tgatctgttatcatc

SEQ ID NO: 616 - DNA, 15 bp, synthetic construct
  misc_feature 1..15: note = Synthetic Oligonucleotide
  source 1..15: mol_type = other DNA
ctgttatcatctgta

SEQ ID NO: 617 - DNA, 15 bp, synthetic construct
  misc_feature 1..15: note = Synthetic Oligonucleotide
  source 1..15: mol_type = other DNA
gtgtataaagatttt

SEQ ID NO: 618 - DNA, 15 bp, synthetic construct
  misc_feature 1..15: note = Synthetic Oligonucleotide
  source 1..15: mol_type = other DNA
caatttacattttag

SEQ ID NO: 619 - DNA, 15 bp, synthetic construct
  misc_feature 1..15: note = Synthetic Oligonucleotide
  source 1..15: mol_type = other DNA
tacattttagaccat

SEQ ID NO: 620 - DNA, 15 bp, synthetic construct
  misc_feature 1..15: note = Synthetic Oligonucleotide
  source 1..15: mol_type = other DNA
tgctataagatgtaa

SEQ ID NO: 621 - DNA, 15 bp, synthetic construct
  misc_feature 1..15: note = Synthetic Oligonucleotide
  source 1..15: mol_type = other DNA
aaggaagccggcaag

SEQ ID NO: 622 - DNA, 15 bp, synthetic construct
  misc_feature 1..15: note = Synthetic Oligonucleotide
  source 1..15: mol_type = other DNA
cgccacaactcattc

SEQ ID NO: 623 - DNA, 15 bp, synthetic construct
  misc_feature 1..15: note = Synthetic Oligonucleotide
  source 1..15: mol_type = other DNA
atgggagcattgtgg

SEQ ID NO: 624 - DNA, 15 bp, synthetic construct
  misc_feature 1..15: note = Synthetic Oligonucleotide
  source 1..15: mol_type = other DNA
cgcccgcccagcccc

SEQ ID NO: 625 - DNA, 15 bp, synthetic construct
  misc_feature 1..15: note = Synthetic Oligonucleotide
  source 1..15: mol_type = other DNA
cccctcccccgcccg

SEQ ID NO: 626 - DNA, 15 bp, synthetic construct
  misc_feature 1..15: note = Synthetic Oligonucleotide
  source 1..15: mol_type = other DNA
cttccgctgctgctg

SEQ ID NO: 627 - DNA, 15 bp, synthetic construct
  misc_feature 1..15: note = Synthetic Oligonucleotide
  source 1..15: mol_type = other DNA
cttcttagtaccaac

SEQ ID NO: 628 - DNA, 15 bp, synthetic construct
  misc_feature 1..15: note = Synthetic Oligonucleotide
  source 1..15: mol_type = other DNA
tttagagcaaaatcg

SEQ ID NO: 629 - DNA, 15 bp, synthetic construct
  misc_feature 1..15: note = Synthetic Oligonucleotide
  source 1..15: mol_type = other DNA
ggtagttaaatgttt

SEQ ID NO: 630 - DNA, 15 bp, synthetic construct
  misc_feature 1..15: note = Synthetic Oligonucleotide
  source 1..15: mol_type = other DNA
tacttaagaaagaga

SEQ ID NO: 631 - DNA, 15 bp, synthetic construct
  misc_feature 1..15: note = Synthetic Oligonucleotide
  source 1..15: mol_type = other DNA
tatacttaagaaaga

SEQ ID NO: 632 - DNA, 15 bp, synthetic construct
  misc_feature 1..15: note = Synthetic Oligonucleotide
  source 1..15: mol_type = other DNA
cgccgccgacgccgg

SEQ ID NO: 633 - DNA, 15 bp, synthetic construct
  misc_feature 1..15: note = Synthetic Oligonucleotide
  source 1..15: mol_type = other DNA
ctctctccgagagga

SEQ ID NO: 634 - DNA, 15 bp, synthetic construct
  misc_feature 1..15: note = Synthetic Oligonucleotide
  source 1..15: mol_type = other DNA
cgccccgccctcttg

SEQ ID NO: 635 - DNA, 15 bp, synthetic construct
  misc_feature 1..15: note = Synthetic Oligonucleotide
  source 1..15: mol_type = other DNA
ccgcgcgctgctgca

SEQ ID NO: 636 - DNA, 15 bp, synthetic construct
  misc_feature 1..15: note = Synthetic Oligonucleotide
  source 1..15: mol_type = other DNA
cactttcacagagag

SEQ ID NO: 637 - DNA, 16 bp, synthetic construct
  misc_feature 1..16: note = Synthetic Oligonucleotide
  source 1..16: mol_type = other DNA
ctttcacatgtattaa

SEQ ID NO: 638 - DNA, 15 bp, synthetic construct
  misc_feature 1..15: note = Synthetic Oligonucleotide
  source 1..15: mol_type = other DNA
atgtattaaaaaact

SEQ ID NO: 639 - DNA, 15 bp, synthetic construct
  misc_feature 1..15: note = Synthetic Oligonucleotide
  source 1..15: mol_type = other DNA
gacatttttatgtaa

SEQ ID NO: 640 - DNA, 15 bp, synthetic construct
  misc_feature 1..15: note = Synthetic Oligonucleotide
  source 1..15: mol_type = other DNA
catttttatgtaaat

SEQ ID NO: 641 - DNA, 15 bp, synthetic construct
  misc_feature 1..15: note = Synthetic Oligonucleotide
  source 1..15: mol_type = other DNA
aaatttataaggcaa

SEQ ID NO: 642 - DNA, 15 bp, synthetic construct
  misc_feature 1..15: note = Synthetic Oligonucleotide
  source 1..15: mol_type = other DNA
aggcaaactctttat

SEQ ID NO: 643 - DNA, 15 bp, synthetic construct
  misc_feature 1..15: note = Synthetic Oligonucleotide
  source 1..15: mol_type = other DNA
gtctctggaacaatt

SEQ ID NO: 644 - DNA, 15 bp, synthetic construct
  misc_feature 1..15: note = Synthetic Oligonucleotide
  source 1..15: mol_type = other DNA
cagttcaaacacaga

SEQ ID NO: 645 - DNA, 15 bp, synthetic construct
  misc_feature 1..15: note = Synthetic Oligonucleotide
  source 1..15: mol_type = other DNA
caaacacagaagaga

SEQ ID NO: 646 - DNA, 15 bp, synthetic construct
  misc_feature 1..15: note = Synthetic Oligonucleotide
  source 1..15: mol_type = other DNA
aacacagaagagatt

SEQ ID NO: 647 - DNA, 15 bp, synthetic construct
  misc_feature 1..15: note = Synthetic Oligonucleotide
  source 1..15: mol_type = other DNA
gggggagaagaaagg

SEQ ID NO: 648 - DNA, 15 bp, synthetic construct
  misc_feature 1..15: note = Synthetic Oligonucleotide
  source 1..15: mol_type = other DNA
tcgtttttttttctt

SEQ ID NO: 649 - DNA, 15 bp, synthetic construct
  misc_feature 1..15: note = Synthetic Oligonucleotide
  source 1..15: mol_type = other DNA
cttttttttcttttt

SEQ ID NO: 650 - DNA, 15 bp, synthetic construct
  misc_feature 1..15: note = Synthetic Oligonucleotide
  source 1..15: mol_type = other DNA
cctatgctatggtta

SEQ ID NO: 651 - DNA, 15 bp, synthetic construct
  misc_feature 1..15: note = Synthetic Oligonucleotide
  source 1..15: mol_type = other DNA
agtttactgaaagaa

SEQ ID NO: 652 - DNA, 15 bp, synthetic construct
  misc_feature 1..15: note = Synthetic Oligonucleotide
  source 1..15: mol_type = other DNA
actgaaagaaaaaaa

SEQ ID NO: 653 - DNA, 15 bp, synthetic construct
  misc_feature 1..15: note = Synthetic Oligonucleotide
  source 1..15: mol_type = other DNA
ccttattcatatttt

SEQ ID NO: 654 - DNA, 15 bp, synthetic construct
  misc_feature 1..15: note = Synthetic Oligonucleotide
  source 1..15: mol_type = other DNA
cttccttattcatat

SEQ ID NO: 655 - DNA, 15 bp, synthetic construct
  misc_feature 1..15: note = Synthetic Oligonucleotide
  source 1..15: mol_type = other DNA
caatccttcaatatt

SEQ ID NO: 656 - DNA, 15 bp, synthetic construct
  misc_feature 1..15: note = Synthetic Oligonucleotide
  source 1..15: mol_type = other DNA
ggcatttcattttac

SEQ ID NO: 657 - DNA, 15 bp, synthetic construct
  misc_feature 1..15: note = Synthetic Oligonucleotide
  source 1..15: mol_type = other DNA
cattttacaaatatt

SEQ ID NO: 658 - DNA, 15 bp, synthetic construct
  misc_feature 1..15: note = Synthetic Oligonucleotide
  source 1..15: mol_type = other DNA
gaaatgaaataagta

SEQ ID NO: 659 - DNA, 15 bp, synthetic construct
  misc_feature 1..15: note = Synthetic Oligonucleotide
  source 1..15: mol_type = other DNA
agatatgcaagataa

SEQ ID NO: 660 - DNA, 15 bp, synthetic construct
  misc_feature 1..15: note = Synthetic Oligonucleotide
  source 1..15: mol_type = other DNA
gcgggcccagcaggt

SEQ ID NO: 661 - DNA, 15 bp, synthetic construct
  misc_feature 1..15: note = Synthetic Oligonucleotide
  source 1..15: mol_type = other DNA
cagtgagtgccgagt

SEQ ID NO: 662 - DNA, 15 bp, synthetic construct
  misc_feature 1..15: note = Synthetic Oligonucleotide
  source 1..15: mol_type = other DNA
gcccgggcagtgagt

SEQ ID NO: 663 - DNA, 15 bp, synthetic construct
  misc_feature 1..15: note = Synthetic Oligonucleotide
  source 1..15: mol_type = other DNA
tgtccgggcggcccg

SEQ ID NO: 664 - DNA, 15 bp, synthetic construct
  misc_feature 1..15: note = Synthetic Oligonucleotide
  source 1..15: mol_type = other DNA
cgcgcgtgtgcgagt

SEQ ID NO: 665 - DNA, 15 bp, synthetic construct
  misc_feature 1..15: note = Synthetic Oligonucleotide
  source 1..15: mol_type = other DNA
cttcagacaggctgc

SEQ ID NO: 666 - DNA, 15 bp, synthetic construct
  misc_feature 1..15: note = Synthetic Oligonucleotide
  source 1..15: mol_type = other DNA
acctctgcacttcag

SEQ ID NO: 667 - DNA, 15 bp, synthetic construct
  misc_feature 1..15: note = Synthetic Oligonucleotide
  source 1..15: mol_type = other DNA
cggcgcgggtccctt

SEQ ID NO: 668 - DNA, 15 bp, synthetic construct
  misc_feature 1..15: note = Synthetic Oligonucleotide
  source 1..15: mol_type = other DNA
tggtattcgaattat

SEQ ID NO: 669 - DNA, 15 bp, synthetic construct
  misc_feature 1..15: note = Synthetic Oligonucleotide
  source 1..15: mol_type = other DNA
cggcctgccctggta

SEQ ID NO: 670 - DNA, 15 bp, synthetic construct
  misc_feature 1..15: note = Synthetic Oligonucleotide
  source 1..15: mol_type = other DNA
tcagagattatgaaa

SEQ ID NO: 671 - DNA, 15 bp, synthetic construct
  misc_feature 1..15: note = Synthetic Oligonucleotide
  source 1..15: mol_type = other DNA
tgttttcagagatta

SEQ ID NO: 672 - DNA, 15 bp, synthetic construct
  misc_feature 1..15: note = Synthetic Oligonucleotide
  source 1..15: mol_type = other DNA
catgtagaaatgctt

SEQ ID NO: 673 - DNA, 15 bp, synthetic construct
  misc_feature 1..15: note = Synthetic Oligonucleotide
  source 1..15: mol_type = other DNA
aaacatgtagaaatg

SEQ ID NO: 674 - DNA, 15 bp, synthetic construct
  misc_feature 1..15: note = Synthetic Oligonucleotide
  source 1..15: mol_type = other DNA
ttgataccatttatg

SEQ ID NO: 675 - DNA, 15 bp, synthetic construct
  misc_feature 1..15: note = Synthetic Oligonucleotide
  source 1..15: mol_type = other DNA
gaactcaattattat

SEQ ID NO: 676 - DNA, 15 bp, synthetic construct
  misc_feature 1..15: note = Synthetic Oligonucleotide
  source 1..15: mol_type = other DNA
aaaacgactccacaa

SEQ ID NO: 677 - DNA, 15 bp, synthetic construct
  misc_feature 1..15: note = Synthetic Oligonucleotide
  source 1..15: mol_type = other DNA
ctccgaggaaaaacg

SEQ ID NO: 678 - DNA, 15 bp, synthetic construct
  misc_feature 1..15: note = Synthetic Oligonucleotide
  source 1..15: mol_type = other DNA
gctccgaggaaaaac

SEQ ID NO: 679 - DNA, 15 bp, synthetic construct
  misc_feature 1..15: note = Synthetic Oligonucleotide
  source 1..15: mol_type = other DNA
ctcggcgggagaaag

SEQ ID NO: 680 - DNA, 13 bp, synthetic construct
  misc_feature 1..13: note = Synthetic Oligonucleotide
  source 1..13: mol_type = other DNA
gaaccgaaatttt

SEQ ID NO: 681 - DNA, 15 bp, synthetic construct
  misc_feature 1..15: note = Synthetic Oligonucleotide
  source 1..15: mol_type = other DNA
gagaagggtgcagat

SEQ ID NO: 682 - DNA, 15 bp, synthetic construct
  misc_feature 1..15: note = Synthetic Oligonucleotide
  source 1..15: mol_type = other DNA
ctctccagatgagaa

SEQ ID NO: 683 - DNA, 15 bp, synthetic construct
  misc_feature 1..15: note = Synthetic Oligonucleotide
  source 1..15: mol_type = other DNA
caggggtccgctctc

SEQ ID NO: 684 - DNA, 15 bp, synthetic construct
  misc_feature 1..15: note = Synthetic Oligonucleotide
  source 1..15: mol_type = other DNA
tccgggcagccaggg

SEQ ID NO: 685 - DNA, 15 bp, synthetic construct
  misc_feature 1..15: note = Synthetic Oligonucleotide
  source 1..15: mol_type = other DNA
ggggctcgcctccgg

SEQ ID NO: 686 - DNA, 15 bp, synthetic construct
  misc_feature 1..15: note = Synthetic Oligonucleotide
  source 1..15: mol_type = other DNA
cccccgggaaggggc

SEQ ID NO: 687 - DNA, 15 bp, synthetic construct
  misc_feature 1..15: note = Synthetic Oligonucleotide
  source 1..15: mol_type = other DNA
cccaccccccgggaa

SEQ ID NO: 688 - DNA, 16 bp, synthetic construct
  misc_feature 1..16: note = Synthetic Oligonucleotide
  source 1..16: mol_type = other DNA
gcgttgccgcccccac

SEQ ID NO: 689 - DNA, 15 bp, synthetic construct
  misc_feature 1..15: note = Synthetic Oligonucleotide
  source 1..15: mol_type = other DNA
gctgggtcgcgcgtt

SEQ ID NO: 690 - DNA, 15 bp, synthetic construct
  misc_feature 1..15: note = Synthetic Oligonucleotide
  source 1..15: mol_type = other DNA
gcgcaggaccgctgg

SEQ ID NO: 691 - DNA, 15 bp, synthetic construct
  misc_feature 1..15: note = Synthetic Oligonucleotide
  source 1..15: mol_type = other DNA
aggagggagggtggg

SEQ ID NO: 692 - DNA, 15 bp, synthetic construct
  misc_feature 1..15: note = Synthetic Oligonucleotide
  source 1..15: mol_type = other DNA
cgctggaggcggagg

SEQ ID NO: 693 - DNA, 15 bp, synthetic construct
  misc_feature 1..15: note = Synthetic Oligonucleotide
  source 1..15: mol_type = other DNA
tggagccgagcgctg

SEQ ID NO: 694 - DNA, 15 bp, synthetic construct
  misc_feature 1..15: note = Synthetic Oligonucleotide
  source 1..15: mol_type = other DNA
ctgcccctttgttgg

SEQ ID NO: 695 - DNA, 15 bp, synthetic construct
  misc_feature 1..15: note = Synthetic Oligonucleotide
  source 1..15: mol_type = other DNA
ctccccgctgcgggc

SEQ ID NO: 696 - DNA, 15 bp, synthetic construct
  misc_feature 1..15: note = Synthetic Oligonucleotide
  source 1..15: mol_type = other DNA
cggctcctcctcctc

SEQ ID NO: 697 - DNA, 15 bp, synthetic construct
  misc_feature 1..15: note = Synthetic Oligonucleotide
  source 1..15: mol_type = other DNA
ggctcgctccttcgg

SEQ ID NO: 698 - DNA, 15 bp, synthetic construct
  misc_feature 1..15: note = Synthetic Oligonucleotide
  source 1..15: mol_type = other DNA
tttgtgcgcgagaga

SEQ ID NO: 699 - DNA, 15 bp, synthetic construct
  misc_feature 1..15: note = Synthetic Oligonucleotide
  source 1..15: mol_type = other DNA
acgactccacaactt

SEQ ID NO: 700 - DNA, 15 bp, synthetic construct
  misc_feature 1..15: note = Synthetic Oligonucleotide
  source 1..15: mol_type = other DNA
gcccgcttccctgct

SEQ ID NO: 701 - DNA, 15 bp, synthetic construct
  misc_feature 1..15: note = Synthetic Oligonucleotide
  source 1..15: mol_type = other DNA
cggccggctgctgct

SEQ ID NO: 702 - DNA, 15 bp, synthetic construct
  misc_feature 1..15: note = Synthetic Oligonucleotide
  source 1..15: mol_type = other DNA
gcgggagaaagcccg

SEQ ID NO: 703 - DNA, 15 bp, synthetic construct
  misc_feature 1..15: note = Synthetic Oligonucleotide
  source 1..15: mol_type = other DNA
cctcctcgcccctcg

SEQ ID NO: 704 - DNA, 15 bp, synthetic construct
  misc_feature 1..15: note = Synthetic Oligonucleotide
  source 1..15: mol_type = other DNA
agaggctcctcctcg

SEQ ID NO: 705 - DNA, 15 bp, synthetic construct
  misc_feature 1..15: note = Synthetic Oligonucleotide
  source 1..15: mol_type = other DNA
tcggcttctggagcc

SEQ ID NO: 706 - DNA, 15 bp, synthetic construct
  misc_feature 1..15: note = Synthetic Oligonucleotide
  source 1..15: mol_type = other DNA
ccgtgattccccaat

SEQ ID NO: 707 - DNA, 15 bp, synthetic construct
  misc_feature 1..15: note = Synthetic Oligonucleotide
  source 1..15: mol_type = other DNA
aggggggcgccgctc

SEQ ID NO: 708 - DNA, 15 bp, synthetic construct
  misc_feature 1..15: note = Synthetic Oligonucleotide
  source 1..15: mol_type = other DNA
aaatgacccaaaaga

SEQ ID NO: 709 - DNA, 15 bp, synthetic construct
  misc_feature 1..15: note = Synthetic Oligonucleotide
  source 1..15: mol_type = other DNA
gttttccgtttgcag

SEQ ID NO: 710 - DNA, 15 bp, synthetic construct
  misc_feature 1..15: note = Synthetic Oligonucleotide
  source 1..15: mol_type = other DNA
ccaaacgctacagag

SEQ ID NO: 711 - DNA, 15 bp, synthetic construct
  misc_feature 1..15: note = Synthetic Oligonucleotide
  source 1..15: mol_type = other DNA
caggcaccaactttg

SEQ ID NO: 712 - DNA, 15 bp, synthetic construct
  misc_feature 1..15: note = Synthetic Oligonucleotide
  source 1..15: mol_type = other DNA
cctggaaggggcgcg

SEQ ID NO: 713 - DNA, 15 bp, synthetic construct
  misc_feature 1..15: note = Synthetic Oligonucleotide
  source 1..15: mol_type = other DNA
cagtcaaagcgcaaa

SEQ ID NO: 714 - DNA, 15 bp, synthetic construct
  misc_feature 1..15: note = Synthetic Oligonucleotide
  source 1..15: mol_type = other DNA
ccaaaaacaaaacag

SEQ ID NO: 715 - DNA, 15 bp, synthetic construct
  misc_feature 1..15: note = Synthetic Oligonucleotide
  source 1..15: mol_type = other DNA
ttccgccaaaaacaa

SEQ ID NO: 716 - DNA, 15 bp, synthetic construct
  misc_feature 1..15: note = Synthetic Oligonucleotide
  source 1..15: mol_type = other DNA
ggaggagggagggtg

SEQ ID NO: 717 - DNA, 15 bp, synthetic construct
  misc_feature 1..15: note = Synthetic Oligonucleotide
  source 1..15: mol_type = other DNA
cgagcgctggaggcg

SEQ ID NO: 718 - DNA, 15 bp, synthetic construct
  misc_feature 1..15: note = Synthetic Oligonucleotide
  source 1..15: mol_type = other DNA
cctgcccctttgttg

SEQ ID NO: 719 - DNA, 15 bp, synthetic construct
  misc_feature 1..15: note = Synthetic Oligonucleotide
  source 1..15: mol_type = other DNA
ggcggctcctcctcc

SEQ ID NO: 720 - DNA, 15 bp, synthetic construct
  misc_feature 1..15: note = Synthetic Oligonucleotide
  source 1..15: mol_type = other DNA
tagacacttccagaa

SEQ ID NO: 721 - DNA, 15 bp, synthetic construct
  misc_feature 1..15: note = Synthetic Oligonucleotide
  source 1..15: mol_type = other DNA
ttccagaattgtcct

SEQ ID NO: 722 - DNA, 15 bp, synthetic construct
  misc_feature 1..15: note = Synthetic Oligonucleotide
  source 1..15: mol_type = other DNA
cagaattgtccttta

SEQ ID NO: 723 - DNA, 15 bp, synthetic construct
  misc_feature 1..15: note = Synthetic Oligonucleotide
  source 1..15: mol_type = other DNA
ctgctggaactcggc

SEQ ID NO: 724 - DNA, 15 bp, synthetic construct
  misc_feature 1..15: note = Synthetic Oligonucleotide
  source 1..15: mol_type = other DNA
ggccaggctcagctg

SEQ ID NO: 725 - DNA, 15 bp, synthetic construct
  misc_feature 1..15: note = Synthetic Oligonucleotide
  source 1..15: mol_type = other DNA
gcagccaggagcctg

SEQ ID NO: 726 - DNA, 15 bp, synthetic construct
  misc_feature 1..15: note = Synthetic Oligonucleotide
  source 1..15: mol_type = other DNA
actcggccaggctca

SEQ ID NO: 727 - DNA, 15 bp, synthetic construct
  misc_feature 1..15: note = Synthetic Oligonucleotide
  source 1..15: mol_type = other DNA
gctggcctgctggaa

SEQ ID NO: 728 - DNA, 15 bp, synthetic construct
  misc_feature 1..15: note = Synthetic Oligonucleotide
  source 1..15: mol_type = other DNA
tttaaattgtatcgg

SEQ ID NO: 729 - DNA, 15 bp, synthetic construct
  misc_feature 1..15: note = Synthetic Oligonucleotide
  source 1..15: mol_type = other DNA
attgtatcgggcaaa

SEQ ID NO: 730 - DNA, 15 bp, synthetic construct
  misc_feature 1..15: note = Synthetic Oligonucleotide
  source 1..15: mol_type = other DNA
gattaaaacaaaaga

SEQ ID NO: 731 - DNA, 15 bp, synthetic construct
  misc_feature 1..15: note = Synthetic Oligonucleotide
  source 1..15: mol_type = other DNA
aaaacaaaagaaacc

SEQ ID NO: 732 - DNA, 15 bp, synthetic construct
  misc_feature 1..15: note = Synthetic Oligonucleotide
  source 1..15: mol_type = other DNA
gggataaaggaaggg

SEQ ID NO: 733 - DNA, 15 bp, synthetic construct
  misc_feature 1..15: note = Synthetic Oligonucleotide
  source 1..15: mol_type = other DNA
cactgggataaagga

SEQ ID NO: 734 - DNA, 15 bp, synthetic construct
  misc_feature 1..15: note = Synthetic Oligonucleotide
  source 1..15: mol_type = other DNA
gagccgcccgctttg

SEQ ID NO: 735 - DNA, 14 bp, synthetic construct
  misc_feature 1..14: note = Synthetic Oligonucleotide
  source 1..14: mol_type = other DNA
tctgggccccactg

SEQ ID NO: 736 - DNA, 15 bp, synthetic construct
  misc_feature 1..15: note = Synthetic Oligonucleotide
  source 1..15: mol_type = other DNA
caaaaggtcttagct

SEQ ID NO: 737 - DNA, 15 bp, synthetic construct
  misc_feature 1..15: note = Synthetic Oligonucleotide
  source 1..15: mol_type = other DNA
tagctattattactg

SEQ ID NO: 738 - DNA, 15 bp, synthetic construct
  misc_feature 1..15: note = Synthetic Oligonucleotide
  source 1..15: mol_type = other DNA
actgttgttgtttta

SEQ ID NO: 739 - DNA, 15 bp, synthetic construct
  misc_feature 1..15: note = Synthetic Oligonucleotide
  source 1..15: mol_type = other DNA
accttagaggttgta

SEQ ID NO: 740 - DNA, 15 bp, synthetic construct
  misc_feature 1..15: note = Synthetic Oligonucleotide
  source 1..15: mol_type = other DNA
tacctgaaattgcag

SEQ ID NO: 741 - DNA, 15 bp, synthetic construct
  misc_feature 1..15: note = Synthetic Oligonucleotide
  source 1..15: mol_type = other DNA
gtcagaaaagctacc

SEQ ID NO: 742 - DNA, 15 bp, synthetic construct
  misc_feature 1..15: note = Synthetic Oligonucleotide
  source 1..15: mol_type = other DNA
cacgcttggtgtgca

SEQ ID NO: 743 - DNA, 15 bp, synthetic construct
  misc_feature 1..15: note = Synthetic Oligonucleotide
  source 1..15: mol_type = other DNA
ctgtgaatgtgtgaa

SEQ ID NO: 744 - DNA, 15 bp, synthetic construct
  misc_feature 1..15: note = Synthetic Oligonucleotide
  source 1..15: mol_type = other DNA
aacaggaagcacctg

SEQ ID NO: 745 - DNA, 39 bp, synthetic construct
  misc_feature 1..39: note = Synthetic Oligonucleotide
  source 1..39: mol_type = other DNA
cgctccgccctccagtttttttttaggaggcaacacatt

SEQ ID NO: 746 - DNA, 41 bp, synthetic construct
  misc_feature 1..41: note = Synthetic Oligonucleotide
  source 1..41: mol_type = other DNA
cgctccgccctccagcctttttttttaggaggcaacacatt

SEQ ID NO: 747 - DNA, 15 bp, Homo sapiens
  source 1..15: mol_type = genomic DNA
ctgttataagctttt

SEQ ID NO: 748 - DNA, 15 bp, Homo sapiens
  source 1..15: mol_type = genomic DNA
gttataagctttttc

SEQ ID NO: 749 - DNA, 15 bp, Homo sapiens
  source 1..15: mol_type = genomic DNA
tatctgttataagct

SEQ ID NO: 750 - DNA, 15 bp, Homo sapiens
  source 1..15: mol_type = genomic DNA
agtagatgtttattt

SEQ ID NO: 751 - DNA, 15 bp, Homo sapiens
  source 1..15: mol_type = genomic DNA
taggcaaaggaatag

SEQ ID NO: 752 - DNA, 15 bp, Homo sapiens
  source 1..15: mol_type = genomic DNA
ggtaggcaaaggaat

SEQ ID NO: 753 - DNA, 15 bp, Homo sapiens
  source 1..15: mol_type = genomic DNA
ggcaaaggaatagtt

SEQ ID NO: 754 - DNA, 15 bp, Homo sapiens
  source 1..15: mol_type = genomic DNA
gaaatgacacccagt

SEQ ID NO: 755 - DNA, 15 bp, Homo sapiens
  source 1..15: mol_type = genomic DNA
aaatgacacccagta

SEQ ID NO: 756 - DNA, 15 bp, Homo sapiens
  source 1..15: mol_type = genomic DNA
ggcaatggaatgaaa

SEQ ID NO: 757 - DNA, 15 bp, Homo sapiens
  source 1..15: mol_type = genomic DNA
caatggaatgaaatg

SEQ ID NO: 758 - DNA, 15 bp, Homo sapiens
  source 1..15: mol_type = genomic DNA
atggaatgaaatgac

SEQ ID NO: 759 - DNA, 15 bp, Homo sapiens
  source 1..15: mol_type = genomic DNA
gtttcaagtacccgc

SEQ ID NO: 760 - DNA, 15 bp, Homo sapiens
  source 1..15: mol_type = genomic DNA
gaccggagaacgaaa

SEQ ID NO: 761 - DNA, 15 bp, Homo sapiens
  source 1..15: mol_type = genomic DNA
ctttggaaggtgttt

SEQ ID NO: 762 - DNA, 15 bp, Homo sapiens
  source 1..15: mol_type = genomic DNA
tttctttggaaggtg

SEQ ID NO: 763 - DNA, 15 bp, Homo sapiens
  source 1..15: mol_type = genomic DNA
agttaatccccgccg

SEQ ID NO: 764 - DNA, 15 bp, Homo sapiens
  source 1..15: mol_type = genomic DNA
tcctgcaaaatgtca

SEQ ID NO: 765 - DNA, 15 bp, Homo sapiens
  source 1..15: mol_type = genomic DNA
ccccgcagtctccac

SEQ ID NO: 766 - DNA, 15 bp, Homo sapiens
  source 1..15: mol_type = genomic DNA
tctccaccctcctcc

SEQ ID NO: 767 - DNA, 15 bp, Homo sapiens
  source 1..15: mol_type = genomic DNA
gagcgccggcgactg

SEQ ID NO: 768 - DNA, 15 bp, Homo sapiens
  source 1..15: mol_type = genomic DNA
aggatgtgcgccttc

SEQ ID NO: 769 - DNA, 14 bp, Homo sapiens
  source 1..14: mol_type = genomic DNA
ggcgcagcgaggaa

SEQ ID NO: 770 - DNA, 15 bp, Homo sapiens
  source 1..15: mol_type = genomic DNA
tttcactgacatctc

SEQ ID NO: 771 - DNA, 15 bp, Homo sapiens
  source 1..15: mol_type = genomic DNA
ggtggtctgagcccc

SEQ ID NO: 772 - DNA, 15 bp, Homo sapiens
  source 1..15: mol_type = genomic DNA
gggcctgccagggga

SEQ ID NO: 773 - DNA, 15 bp, Homo sapiens
  source 1..15: mol_type = genomic DNA
accgagtgacagtcg

SEQ ID NO: 774 - DNA, 15 bp, Homo sapiens
  source 1..15: mol_type = genomic DNA
gtctgggaccgagtg

SEQ ID NO: 775 - DNA, 15 bp, Homo sapiens
  source 1..15: mol_type = genomic DNA
tgggaccgagtgaca

SEQ ID NO: 776 - DNA, 15 bp, Homo sapiens
  source 1..15: mol_type = genomic DNA
gcagaggtgtgttca

SEQ ID NO: 777 - DNA, 15 bp, Homo sapiens
  source 1..15: mol_type = genomic DNA
cggcagaggtgtgtt

SEQ ID NO: 778 - DNA, 15 bp, Homo sapiens
  source 1..15: mol_type = genomic DNA
gggcagacggggtca

SEQ ID NO: 779 - DNA, 15 bp, Homo sapiens
  source 1..15: mol_type = genomic DNA
ctctggtttggaaaa

SEQ ID NO: 780 - DNA, 15 bp, Homo sapiens
  source 1..15: mol_type = genomic DNA
ctggtttggaaaaca

SEQ ID NO: 781 - DNA, 15 bp, Homo sapiens
  source 1..15: mol_type = genomic DNA
gtttggaaaacaaaa

SEQ ID NO: 782 - DNA, 15 bp, Homo sapiens
  source 1..15: mol_type = genomic DNA
ggaaaacaaaagaac

SEQ ID NO: 783 - DNA, 15 bp, Homo sapiens
  source 1..15: mol_type = genomic DNA
gaaaacaaaagaacc

SEQ ID NO: 784 - DNA, 15 bp, Homo sapiens
  source 1..15: mol_type = genomic DNA
tttggaaaacaaaag

SEQ ID NO: 785 - DNA, 15 bp, Homo sapiens
  source 1..15: mol_type = genomic DNA
tggaaaacaaaagaa

SEQ ID NO: 786 - DNA, 15 bp, Homo sapiens
  source 1..15: mol_type = genomic DNA
tctggggcagcagga

SEQ ID NO: 787 - DNA, 15 bp, Homo sapiens
  source 1..15: mol_type = genomic DNA
atagaaaggaacctt

SEQ ID NO: 788 - DNA, 15 bp, Homo sapiens
  source 1..15: mol_type = genomic DNA
tagaaaggaaccttg

SEQ ID NO: 789 - DNA, 15 bp, Homo sapiens
  source 1..15: mol_type = genomic DNA
attaacaatccttcc

SEQ ID NO: 790 - DNA, 15 bp, Homo sapiens
  source 1..15: mol_type = genomic DNA
cttccctgctaaatg

SEQ ID NO: 791 - DNA, 15 bp, Homo sapiens
  source 1..15: mol_type = genomic DNA
ccctgctaaatgata

SEQ ID NO: 792 - DNA, 15 bp, Homo sapiens
  source 1..15: mol_type = genomic DNA
tgatataaacataga

SEQ ID NO: 793 - DNA, 15 bp, Homo sapiens
  source 1..15: mol_type = genomic DNA
tccccgggactgaac

SEQ ID NO: 794 - DNA, 16 bp, Homo sapiens
  source 1..16: mol_type = genomic DNA
tacaacttctttgaat

SEQ ID NO: 795 - DNA, 15 bp, Homo sapiens
  source 1..15: mol_type = genomic DNA
tctatcacttccccg

SEQ ID NO: 796 - DNA, 15 bp, Homo sapiens
  source 1..15: mol_type = genomic DNA
tcctgggcacccgta

SEQ ID NO: 797 - DNA, 15 bp, Homo sapiens
  source 1..15: mol_type = genomic DNA
tttctgtacacatca

SEQ ID NO: 798 - DNA, 15 bp, Homo sapiens
  source 1..15: mol_type = genomic DNA
acagccactgaaaat

SEQ ID NO: 799 - DNA, 15 bp, Homo sapiens
  source 1..15: mol_type = genomic DNA
ttcacagccactgaa

SEQ ID NO: 800 - DNA, 15 bp, Homo sapiens
  source 1..15: mol_type = genomic DNA
taaagaaatgagttt

SEQ ID NO: 801 - DNA, 15 bp, Homo sapiens
  source 1..15: mol_type = genomic DNA
acatttaaagaaatg

SEQ ID NO: 802 - DNA, 15 bp, Homo sapiens
  source 1..15: mol_type = genomic DNA
aagaaatgagtttgt

SEQ ID NO: 803 - DNA, 15 bp, Homo sapiens
  source 1..15: mol_type = genomic DNA
tgccattatgtttgc

SEQ ID NO: 804 - DNA, 15 bp, Homo sapiens
  source 1..15: mol_type = genomic DNA
tcctgttgccattat

SEQ ID NO: 805 - DNA, 15 bp, Homo sapiens
  source 1..15: mol_type = genomic DNA
gaaaatcctgttgcc

SEQ ID NO: 806 - DNA, 15 bp, Homo sapiens
  source 1..15: mol_type = genomic DNA
ctgttgccattatgt

SEQ ID NO: 807 - DNA, 15 bp, Homo sapiens
  source 1..15: mol_type = genomic DNA
tagaattgaagtaac

SEQ ID NO: 808 - DNA, 15 bp, Homo sapiens
  source 1..15: mol_type = genomic DNA
tgaagtaacaatcaa

SEQ ID NO: 809 - DNA, 15 bp, Homo sapiens
  source 1..15: mol_type = genomic DNA
aagtaacaatcaatt

SEQ ID NO: 810 - DNA, 15 bp, Homo sapiens
  source 1..15: mol_type = genomic DNA
tcatcaagatttctt

SEQ ID NO: 811 - DNA, 15 bp, Homo sapiens
  source 1..15: mol_type = genomic DNA
tatctagcccaatat

SEQ ID NO: 812 - DNA, 15 bp, Homo sapiens
  source 1..15: mol_type = genomic DNA
ttctatctagcccaa

SEQ ID NO: 813 - DNA, 33 bp, Homo sapiens
  source 1..33: mol_type = genomic DNA
ctccgccctccagtttttattattttgcttttt

SEQ ID NO: 814 - DNA, 31 bp, Homo sapiens
  source 1..31: mol_type = genomic DNA
ccgccctccagtttttattattttgcttttt

SEQ ID NO: 815 - DNA, 29 bp, Homo sapiens
  source 1..29: mol_type = genomic DNA
gccctccagtttttattattttgcttttt

SEQ ID NO: 816 - DNA, 33 bp, Homo sapiens
  source 1..33: mol_type = genomic DNA
cgctccgccctccagtttttattattttgcttt

SEQ ID NO: 817 - DNA, 31 bp, Homo sapiens
  source 1..31: mol_type = genomic DNA
cgctccgccctccagtttttattattttgct

SEQ ID NO: 818 - DNA, 29 bp, Homo sapiens
  source 1..29: mol_type = genomic DNA
cgctccgccctccagtttttattattttg

SEQ ID NO: 819 - DNA, 31 bp, Homo sapiens
  source 1..31: mol_type = genomic DNA
ctccgccctccagtttttattattttgcttt

SEQ ID NO: 820 - DNA, 27 bp, Homo sapiens
  source 1..27: mol_type = genomic DNA
ccgccctccagtttttattattttgct

SEQ ID NO: 821 - DNA, 25 bp, Homo sapiens
  source 1..25: mol_type = genomic DNA
gccctccagtttttattattttgct

SEQ ID NO: 822 - DNA, 23 bp, Homo sapiens
  source 1..23: mol_type = genomic DNA
ccctccagtttttattattttgc

SEQ ID NO: 823 - DNA, 21 bp, Homo sapiens
  source 1..21: mol_type = genomic DNA
cctccagtttttattattttg

SEQ ID NO: 824 - DNA, 29 bp, Homo sapiens
  source 1..29: mol_type = genomic DNA
gctccgccctccagattattttgcttttt

SEQ ID NO: 825 - DNA, 27 bp, Homo sapiens
  source 1..27: mol_type = genomic DNA
tccgccctccagattattttgcttttt

SEQ ID NO: 826 - DNA, 25 bp, Homo sapiens
  source 1..25: mol_type = genomic DNA
cgccctccagattattttgcttttt

SEQ ID NO: 827 - DNA, 23 bp, Homo sapiens
  source 1..23: mol_type = genomic DNA
ccctccagattattttgcttttt

SEQ ID NO: 828 - DNA, 27 bp, Homo sapiens
  source 1..27: mol_type = genomic DNA
gctccgccctccagattattttgcttt

SEQ ID NO: 829 - DNA, 25 bp, Homo sapiens
  source 1..25: mol_type = genomic DNA
gctccgccctccagattattttgct

SEQ ID NO: 830 - DNA, 23 bp, Homo sapiens
  source 1..23: mol_type = genomic DNA
gctccgccctccagattattttg

SEQ ID NO: 831 - DNA, 25 bp, Homo sapiens
  source 1..25: mol_type = genomic DNA
tccgccctccagattattttgcttt

SEQ ID NO: 832 - DNA, 21 bp, Homo sapiens
  source 1..21: mol_type = genomic DNA
cgccctccagattattttgct

SEQ ID NO: 833 - DNA, 19 bp, Homo sapiens
  source 1..19: mol_type = genomic DNA
gccctccagattattttgc

SEQ ID NO: 834 - DNA, 17 bp, Homo sapiens
  source 1..17: mol_type = genomic DNA
ccctccagattattttg

SEQ ID NO: 835 - DNA, 15 bp, Homo sapiens
  source 1..15: mol_type = genomic DNA
ctccagattattttg

SEQ ID NO: 836 - DNA, 35 bp, Homo sapiens; identical to SEQ ID NO: 143
  source 1..35: mol_type = genomic DNA

SEQ ID NO: 837 - DNA, 28 bp, Mus musculus
  source 1..28: mol_type = genomic DNA
agttcaaggatcagccattttggaaagg

SEQ ID NO: 838 - DNA, 25 bp, Mus musculus
  source 1..25: mol_type = genomic DNA
tcaaggatcagccattttggaaagg

SEQ ID NO: 839 - DNA, 23 bp, Mus musculus
  source 1..23: mol_type = genomic DNA
aaggatcagccattttggaaagg

SEQ ID NO: 840 - DNA, 21 bp, Mus musculus
  source 1..21: mol_type = genomic DNA
ggatcagccattttggaaagg

SEQ ID NO: 841 - DNA, 25 bp, Mus musculus
  source 1..25: mol_type = genomic DNA
agttcaaggatcagccattttggaa

SEQ ID NO: 842 - DNA, 23 bp, Mus musculus
  source 1..23: mol_type = genomic DNA
agttcaaggatcagccattttgg

SEQ ID NO: 843 - DNA, 27 bp, Mus musculus
  source 1..27: mol_type = genomic DNA
gttcaaggatcagccattttggaaagg

SEQ ID NO: 844 - DNA, 23 bp, Mus musculus
  source 1..23: mol_type = genomic DNA
tcaaggatcagccattttggaaa

SEQ ID NO: 845 - DNA, 21 bp, Mus musculus
  source 1..21: mol_type = genomic DNA
aaggatcagccattttggaaa

SEQ ID NO: 846 - DNA, 19 bp, Mus musculus
  source 1..19: mol_type = genomic DNA
aggatcagccattttggaa

SEQ ID NO: 847 - DNA, 17 bp, Mus musculus
  source 1..17: mol_type = genomic DNA
ggatcagccattttgga

SEQ ID NO: 848 - DNA, 28 bp, Mus musculus
  source 1..28: mol_type = genomic DNA
ctccgacagtctgccattttggaaaggt

SEQ ID NO: 849 - DNA, 25 bp, Mus musculus
  source 1..25: mol_type = genomic DNA
cgacagtctgccattttggaaaggt

SEQ ID NO: 850 - DNA, 23 bp, Mus musculus
  source 1..23: mol_type = genomic DNA
acagtctgccattttggaaaggt

SEQ ID NO: 851 - DNA, 25 bp, Mus musculus
  source 1..25: mol_type = genomic DNA
ctccgacagtctgccattttggaaa

SEQ ID NO: 852 - DNA, 23 bp, Mus musculus
  source 1..23: mol_type = genomic DNA
ctccgacagtctgccattttgga

SEQ ID NO: 853 - DNA, 21 bp, Mus musculus
  source 1..21: mol_type = genomic DNA
ctccgacagtctgccattttg

SEQ ID NO: 854 - DNA, 27 bp, Mus musculus
  source 1..27: mol_type = genomic DNA
ctccgacagtctgccattttggaaagg

SEQ ID NO: 855 - DNA, 23 bp, Mus musculus
  source 1..23: mol_type = genomic DNA
ccgacagtctgccattttggaaa

SEQ ID NO: 856 - DNA, 19 bp, Mus musculus
  source 1..19: mol_type = genomic DNA
gacagtctgccattttgga

SEQ ID NO: 857 - DNA, 17 bp, Mus musculus
  source 1..17: mol_type = genomic DNA
acagtctgccattttgg

SEQ ID NO: 858 - DNA, 30 bp, Mus musculus
  source 1..30: mol_type = genomic DNA
cggagctctccgacacattttggaaaggtt

SEQ ID NO: 859 - DNA, 29 bp, Mus musculus
  source 1..29: mol_type = genomic DNA
ggagctctccgacacattttggaaaggtt

SEQ ID NO: 860 - DNA, 25 bp, Mus musculus
  source 1..25: mol_type = genomic DNA
agctctccgacacattttggaaagg

SEQ ID NO: 861 - DNA, 21 bp, Mus musculus
  source 1..21: mol_type = genomic DNA
ctctccgacacattttggaaa

SEQ ID NO: 862 - DNA, 19 bp, Mus musculus
  source 1..19: mol_type = genomic DNA
tctccgacacattttggaa

SEQ ID NO: 863 - DNA, 17 bp, Mus musculus
  source 1..17: mol_type = genomic DNA
ctccgacacattttgga

SEQ ID NO: 864 - DNA, 15 bp, Mus musculus
  source 1..15: mol_type = genomic DNA
tccgacacattttgg

SEQ ID NO: 865 - DNA, 15 bp, Homo sapiens
  source 1..15: mol_type = genomic DNA
cgctccgccctccag

SEQ ID NO: 866 - DNA, 15 bp, Homo sapiens
  source 1..15: mol_type = genomic DNA
attattttgcttttt

SEQ ID NO: 867 - RNA, 29 nt, synthetic construct
  misc_feature 1..29: note = Synthetic Oligonucleotide
  source 1..29: mol_type = other RNA
aaaaaaaaaaaaaaaaaaaaaaaaaaaaa

SEQ ID NO: 868 - RNA, 11 nt, synthetic construct
  misc_feature 1..11: note = Synthetic Oligonucleotide
  source 1..11: mol_type = other RNA
aaaaaaaaaaa